(19) United States
(12) Patent Application Publication
(10) Pub. No.: US 2025/0345425 A1
(43) Pub. Date: Nov. 13, 2025

(54) CELLS ENGINEERED WITH AN HLA-E AND HLA-G TRANSGENE

(71) Applicant: Century Therapeutics, Inc., Philadelphia, PA (US)

(72) Inventors: Buddha GURUNG, Chalfont, PA (US); Sumei LU, Ardmore, PA (US); Michael Francis NASO, Swarthmore, PA (US); Luis Ghira BORGES, Philadelphia, PA (US)

(21) Appl. No.: 18/862,373

(22) PCT Filed: May 4, 2023
(86) PCT No.: PCT/US2023/066599

§ 371 Date: Nov. 1, 2024

Related U.S. Application Data
(60) Provisional application No. 63/338,329, filed on May 4, 2022.

(51) Int. Cl. A61K 40/11; A61K 35/15; A61K 35/17; A61K 35/28; A61K 40/15; C07K 14/74; C12N 5/0775; C12N 5/0783; C12N 5/0786; C12N 5/10; C12N 15/113; C12N 15/86

(52) U.S. Cl. CPC A61K 40/11; A61K 35/15; A61K 35/17; A61K 35/28; A61K 40/15; C07K 14/70539; C12N 5/0636; C12N 5/0645; C12N 5/0646; C12N 5/0662; C12N 5/10; C12N 15/113; C12N 15/86; C07K 2319/02; C07K 2319/03; C12N 2510/00; C12N 2800/10

(57) ABSTRACT

The present disclosure provides genetically engineered cells and derivatives thereof, particularly cells and derivatives thereof modified with HLA-E and HLA-G transgenes. Also further provided are related vectors, nuclease complexes, polypeptides, polynucleotides, and pharmaceutical compositions. Methods for treating subjects using the genetically engineered cells and/or pharmaceutical compositions are also provided.

### CROSS-REFERENCE TO RELATED APPLICATION

This application claims the benefit of Provisional U.S. Application No. 63/338,329, filed May 4, 2022, the contents of which is incorporated by reference in its entirety for all purposes.

### SEQUENCE LISTING

The instant application contains a Sequence Listing which has been submitted electronically in XML format and is hereby incorporated by reference in its entirety. Said XML copy, created on May 4, 2023, is named 256805_000030_SL.xml and is 210,228 bytes in size.

### FIELD

The present disclosure provides genetically engineered cells and derivatives thereof, particularly cells and derivatives thereof modified with HLA-E and HLA-G transgenes. Further provided are related vectors, nuclease complexes, polypeptides, polynucleotides, and pharmaceutical compositions. Methods for treating subjects using the genetically engineered cells and/or pharmaceutical compositions are also provided.

### BACKGROUND

Autologous, patient-specific immunological therapy has emerged as a powerful and potentially curative therapy. Autologous immune cells, however, must be generated on a custom-made basis, which remains a significant limiting factor for large-scale clinical application due to the production costs and the risk of production failure. Moreover, in the case of cancer patients, especially those that have gone through multiple rounds of chemotherapy and drug treatments before becoming eligible for immune therapy, the quality and counts of the cells to be engineered might be low. Furthermore, there is a risk of contamination with malignant cells in the final therapeutic composition. Equally, as the heterogeneity is large from composition to composition, critical quality attributes are difficult to maintain, which can reduce the safety and efficacy of the treatment.

Allogeneic immunotherapy (i.e., using cells from a donor who is not the patient) offers advantages as compared with autologous immunotherapy that makes it an appealing option. In many cases, allogeneic immunotherapy relies on an “off-the-shelf” product, which means the patient receives cells that originated from a healthy donor genetically engineered to elicit the therapeutic response required. These allogeneic immunotherapy compositions will comprise consistent batches that can be stored and shipped to patients as needed. As such, the patient receives the immunotherapy on demand, which saves precious time and resources. Although allogeneic immunotherapies have many advantages, there are still major challenges to overcome. Two of the biggest hurdles involve cytokine release syndrome (CRS), wherein the patient's immune cells are activated by the donor cells thereby releasing large amounts of cytokines into the body, and graft-versus-host disease (GvHD), wherein the patient's T cells attack the donor cells and cause a life-threatening reaction.

Therefore, there is an unmet need for therapeutically sufficient and functional allogenic immune cells for effective use in immunotherapy.

Further, in engineering cell therapies, it is desirable to minimize the number of genetic edits that need to be made to the cells. Thus, there is a need for engineered cell therapies with multiple functionalities that can be engineered with a minimum number of edits.

### BRIEF SUMMARY

The present invention describes compositions and methods for use in genome engineering of cells, such as induced pluripotent stem cells (iPSCs). Specifically, the methods and compositions described relate to compositions and methods for introducing HLA-E and HLA-G transgenes into iPSCs such as pluripotent hematopoietic stem cells and/or progenitor cells (HSCs/PCs) and preparing immune-effector cells derived from the iPSCs.

Polymorphisms in the human leukocyte antigen (HLA) class I and class II genes can cause the rejection of iPSC derived products in allogeneic recipients. Disruption of the Beta-2 Microglobulin (B2M) gene eliminates surface expression of all class I molecules and disruption of the CIITA gene can eliminate expression of all class II molecules. However, since HLA class I molecules function as inhibitory ligands for NK cells, cells that do not display HLA class I molecules are attacked and killed by NK cells. Accordingly, disruption of the HLA I gene leaves the cells vulnerable to lysis by natural killer (NK) cells. This ‘missing self’ response can be prevented by forced expression of minimally polymorphic HLA-E and HLA-G molecules. As such, gene editing can be employed to knock in HLA-E and/or HLA-G genes in human iPSCs in a manner that confers inducible, regulated, surface expression of HLA-E and/or HLA-G single-chain dimers. See, e.g. Nat Biotechnol. 2017 August; 35(8): 765-772. By doing this, these HLA-engineered iPSCs and their differentiated derivatives are resistant to NK-mediated lysis. Further, NK-mediated lysis inhibition may be enhanced by combining both HLA-E and HLA-G. By combining HLA-E and HLA-G components in a single transgene in accordance with the present invention, the number of genetic edits that need to be made to the cells is minimized. In addition, combining HLA-E and HLA-G components in one construct might result in lower expression of one or the other coding sequences, which could limit function. Therefore, linkage between the HLA-E and/or HLA-G components needs to be optimized to achieve desirable expression.

In one aspect, the present invention provides a chimeric single-chain HLA-E and HLA-G molecule comprising: (a) a first molecule comprising an HLA-E heavy chain and (b) a second molecule comprising an HLA-G heavy chain, and (c) a linking peptide between (a) and (b).

In some embodiments, the order of the chimeric single-chain HLA-E and HLA-G molecule may be (i) (a)-(c)-(b) or (ii) (b)-(c)-(a).

In some embodiments, the HLA-E heavy chain polypeptide may comprise the amino acid sequence of SEQ ID NO: 15, or an amino acid sequence having at least 80% sequence identity thereof.

In some embodiments, the nucleotide sequence encoding the HLA-E heavy chain polypeptide may comprise the sequence of SEQ ID NO: 16, or a nucleotide sequence having at least 80% sequence identity thereof.

In some embodiments, the HLA-G heavy chain polypeptide may comprise the amino acid sequence of SEQ ID NO: 25, or an amino acid sequence having at least 80% sequence identity thereof.

In some embodiments, the nucleotide sequence encoding the HLA-G heavy chain polypeptide may comprise the sequence of SEQ ID NO: 26, or a nucleotide sequence having at least 80% sequence identity thereof.

In some embodiments, the linking peptide may comprise an autoprotease peptide and optionally one or two autoprotease peptide linkers.

In some embodiments, at least one of the autoprotease peptide linkers may be 5′ to the autoprotease peptide, 3′ to the autoprotease peptide, or both 5′ and 3′ to the autoprotease peptide.

In some embodiments, the autoprotease peptide may comprise an amino acid sequence set forth in Table 3, or an amino acid sequence having at least 80% sequence identity thereof.

In some embodiments, the autoprotease peptide may be a 2A peptide.

In some embodiments of any of the various chimeric single-chain HLA-E and HLA-G molecules disclosed herein, the 2A peptide may be a P2A, F2A, E2A, T2A, GF2A, GP2A, GE2A, GT2A, BmCPV2A, or BmIFV2A peptide.

In some embodiments, the 2A peptide may be a P2A peptide.

In some embodiments, the P2A peptide may comprise the amino acid sequence of SEQ ID NO: 21, or an amino acid sequence having at least 80% sequence identity thereof.

In some embodiments, the nucleotide sequence encoding the P2A peptide may comprise the sequence of SEQ ID NO: 22, or a nucleotide sequence having at least 80% sequence identity thereof.

In some embodiments of any of the various chimeric single-chain HLA-E and HLA-G molecules disclosed herein, (a) the first molecule may comprise a first B2M polypeptide fused to the HLA-E heavy chain via a first linker and/or (b) the second molecule may comprise a second B2M polypeptide fused to the HLA-G heavy chain via a second linker.

In some embodiments, the first B2M polypeptide may be 5′ to the HLA-E heavy chain polypeptide.

In some embodiments, the first B2M polypeptide may be 3′ to the HLA-E heavy chain polypeptide.

In some embodiments, the second B2M polypeptide may be 5′ to the HLA-G heavy chain polypeptide.

In some embodiments, the second B2M polypeptide may be 3′ to the HLA-G heavy chain polypeptide.

In some embodiments, the first B2M polypeptide and/or the second B2M polypeptide may comprise the amino acid sequence of SEQ ID NO: 9, or an amino acid sequence having at least 80% sequence identity thereof.

In some embodiments, the polynucleotide sequence encoding the first B2M polypeptide and/or the second B2M polypeptide may comprise the sequence of SEQ ID NO: 10 or 11, or a nucleotide sequence having at least 80% sequence identity thereof.

In some embodiments of any of the various chimeric single-chain HLA-E and HLA-G molecules disclosed herein, (a) the first molecule further may comprise a first presentation peptide fused to the first B2M polypeptide via a third linker and/or (b) the second molecule further may comprise a second presentation peptide fused to the second B2M polypeptide via a fourth linker.

In some embodiments of the various chimeric single-chain HLA-E and HLA-G molecules disclosed herein, (a) the first presentation peptide may be fused to the first B2M polypeptide and (a) the second presentation peptide may be fused to the second B2M polypeptide.

In some embodiments, the first presentation peptide and/or a second presentation peptide may be the same.

In some embodiments, the first presentation peptide and/or a second presentation peptide may be different.

In some embodiments, the first presentation peptide and/or the second presentation peptide may comprise the amino acid sequence of SEQ ID NOs: 4 or 23, or an amino acid sequence having at least 80% sequence identity thereof.

In some embodiments, the polynucleotide sequence encoding the first presentation peptide and/or the second peptide may comprise the sequence of SEQ ID NOs: 5 or 24, or a nucleotide sequence having at least 80% sequence identity thereof.

In some embodiments, the first, second, third, fourth, and/or autoprotease peptide linker may each separately comprise an amino acid sequence set forth in Table 4, or an amino acid sequence having at least 80% sequence identity thereof.

In some embodiments, the first peptide linker sequence and/or the second peptide linker sequence may comprise the amino acid sequence of SEQ ID NO: 6, 39, or 41, or an amino acid sequence having at least 80% sequence identity to thereof.

In some embodiments, the nucleotide sequence encoding the first peptide linker sequence and/or the second peptide linker sequence may comprise the sequence of SEQ ID NO: 7 or 8, or a nucleotide sequence having at least 80% sequence identity thereof.

In some embodiments, the third peptide linker sequence and/or the fourth peptide linker sequence may comprise the amino acid sequence of SEQ ID NO: 12, or an amino acid sequence having at least 80% sequence identity thereof.

In some embodiments, the nucleotide sequence encoding the third peptide linker sequence and/or the fourth peptide linker sequence may comprise the sequence of SEQ ID NO: 13 or 14, or a nucleotide sequence having at least 80% sequence identity thereof.

In some embodiments of any of the various chimeric single-chain HLA-E and HLA-G molecules disclosed herein, (a) the first molecule further may comprise a first signal peptide operably linked to the HLA-E heavy chain and/or (b) the second molecule further may comprise a second signal peptide operably linked to the HLA-G heavy chain.

In some embodiments, the first signal peptide and the second signal peptides may be the same.

In some embodiments, the first signal peptide and the second signal peptides may be different.

In some embodiments, the first signal peptide and/or the second signal peptide may comprise the amino acid sequence of SEQ ID NO: 1, or an amino acid sequence having at least 80% sequence identity thereof.

In some embodiments, the first signal peptide and the second signal peptide may comprise the amino acid sequence of SEQ ID NO: 1, or an amino acid sequence having at least 80% sequence identity thereof.

In some embodiments, the polynucleotide sequence encoding the first signal peptide and/or the second signal peptide may comprise the sequence of SEQ ID NO: 2 or 3, or a polynucleotide sequence having at least 80% sequence identity thereof.

In some embodiments, the first molecule may comprise the amino acid sequence of SEQ ID NO: 17 or 19, or an amino acid sequence having at least 80% sequence identity thereof.

In some embodiments, the second molecule may comprise the amino acid sequence of SEQ ID NO: 27 or 29, or an amino acid sequence having at least 80% sequence identity thereof.

In some embodiments, the chimeric single-chain HLA-E and HLA-G molecule of the present disclosure may comprise the amino acid sequence of SEQ ID NO: 31, 165, or 168.

In another aspect, the present invention provides a polynucleotide encoding any of the various chimeric single-chain HLA-E and HLA-G molecules disclosed herein.

In some embodiments, the polynucleotide sequence encoding the first molecule may comprise the nucleotide sequence of SEQ ID NO: 18 or 20, or a nucleotide sequence having at least 80% sequence identity thereof.

In some embodiments, the polynucleotide sequence encoding the second molecule may comprise the nucleotide sequence of SEQ ID NO: 28 or 30, or a nucleotide sequence having at least 80% sequence identity thereof.

In some embodiments, the polynucleotide encoding the single-chain HLA-E and HLA-G molecule of the present disclosure may comprise the nucleotide sequence of SEQ ID NO: 32, 120, 166, 167, or 169.

In some embodiments, the polynucleotide sequence encoding the single-chain HLA-E and HLA-G molecule of the present disclosure may be operably linked to a single promoter.

In some embodiments, the promoter may be an inducible promoter.

In some embodiments of the above-described polynucleotide, the polynucleotide may be a DNA molecule.

In some embodiments of the above-described polynucleotide, the polynucleotide may be an RNA molecule.

In another aspect, the present invention provides a recombinant vector comprising any of the various polynucleotides disclosed herein.

In some embodiments, the vector may be a viral vector.

In some embodiments, the viral vector may be a retroviral vector, a lentiviral vector, an adenoviral vector, an adeno-associated virus vector, an alphaviral vector, a herpes virus vector, a baculoviral vector, or a vaccinia virus vector.

In some embodiments, the vector may be a non-viral vector.

In some embodiments, the non-viral vector may be a minicircle plasmid, a Sleeping Beauty transposon, a piggyBac transposon, or a single or double stranded DNA molecule that is used as a template for homology directed repair (HDR) based gene editing.

In another aspect, the present invention provides an isolated host cell comprising any of the various polynucleotides disclosed herein or any of the various the recombinant vectors disclosed herein.

In another aspect, the present invention provides an isolated host cell comprising any of the chimeric single-chain HLA-E and HLA-G molecules disclosed herein encoded by any of the various polynucleotides disclosed herein.

In some embodiments, the host cell may be an iPSC or a population thereof.

In some embodiments, the host cell may be an immune-effector cell.

In another aspect, the present invention provides an immune-effector cell, or a population thereof, derived from any of the various iPSCs disclosed herein.

In some embodiments of the isolated host cell, immune-effector cell, or population disclosed herein, the host cell, immune-effector cell, or population thereof may be a T cell, a natural killer (NK) cell, a natural killer T cell (NKT cell), a mesenchymal stem cell (MSC), or a macrophage.

In some embodiments, the host cell, immune-effector cell, or population thereof may be a T cell.

In some embodiments, the host cell, immune-effector cell, or population thereof may be an αβ T-cell receptor (TCR) T-cell, a γδ T-cell, a CD8+ T-cell, a CD4+ T-cell, a cytotoxic T-cell, an invariant natural killer T (INKT) cell, a memory T-cell, a memory stem T-cell (TSCM), a naïve T-cell, an effector T-cell, a T-helper cell, or a regulatory T-cell (Treg).

In some embodiments, the host cell, immune-effector cell, or population thereof may be an NK cell.

In another aspect, the present invention provides a MAD7/gRNA ribonucleoprotein (RNP) complex composition for insertion of an HLA-E and HLA-G transgene, comprising: (I) a MAD7 nuclease; (II) a guide RNA (gRNA) specific for the MAD7 nuclease, wherein the gRNA may comprise a guide sequence capable of hybridizing to a target sequence of an AAVS1, B2M, CIITA, NKG2A, TRAC, CD70, CD38, CD33, or CLYBL locus in a cell, wherein the guide sequence is selected from SEQ ID NOs: 109-119, wherein when the gRNA is complexed with the MAD7 nuclease, the guide sequence directs sequence-specific binding of the MAD7 nuclease to the target sequence; and (III) a transgene vector comprising: (1) left and right polynucleotide sequences that are homologous to left and right arms of the target sequence of the AAVS1, B2M, CIITA, NKG2A, TRAC, CD70, CD38, CD33, or CLYBL locus, (2) a promoter which is operably linked to (3) a polynucleotide encoding the HLA-E and HLA-G transgene comprising any of various polynucleotides disclosed herein, and (4) a transcription terminator sequence.

In another aspect, the present invention provides a MAD7/gRNA ribonucleoprotein (RNP) complex composition for insertion of an HLA-E and HLA-G transgene, comprising: I) a MAD7 nuclease system, wherein the system is encoded by one or more vectors comprising (a) a sequence encoding a guide RNA (gRNA) operably linked to a first regulatory element, wherein the gRNA may comprise a guide sequence capable of hybridizing to a target sequence of the AAVS1, B2M, CIITA, NKG2A, TRAC, CD70, CD38, or CLYBL locus in a cell, wherein the guide sequence is selected from SEQ ID NOs: 109-119, and wherein when transcribed, the guide sequence directs sequence-specific binding of the MAD7 complex to the target sequence, (b) a sequence encoding a MAD7 nuclease, wherein the sequence is operably linked to a second regulatory element; and (II) a HLA-E and HLA-G transgene vector comprising: (1) left and right polynucleotide sequences that are homologous to the left and right arms of the target sequence of the AAVS1, B2M, CIITA, NKG2A, TRAC, CD70, CD38, CD33 or CLYBL locus, (2) a promoter which is operably linked to (3) a polynucleotide encoding the HLA-E and HLA-G transgene comprising any of the various polynucleotides disclosed herein, and (4) a transcription terminator sequence.

In another aspect, the present invention provides a MAD7/gRNA ribonucleoprotein (RNP)-based vector system, comprising: (I) one or more vectors comprising (a) a sequence encoding a guide RNA (gRNA), wherein the sequence is operably linked to a first regulatory element, wherein the gRNA may comprise a guide sequence capable of hybridizing to a target sequence of the AAVS1, B2M, CIITA, NKG2A, TRAC, CD70, CD38, CD33 or CLYBL locus in a cell, wherein the gRNA guide sequence is selected from SEQ ID NOs: 109-119, wherein when transcribed, the guide sequence directs sequence-specific binding of the MAD7 complex to the target sequence; (b) a sequence encoding a MAD7 nuclease, wherein the sequence is operably linked to a second regulatory element; and (II) a HLA-E and HLA-G transgene vector comprising: (1) left and right polynucleotide sequences that are homologous to the left and right arms of the target sequence of the AAVS1, B2M, CIITA, NKG2A, TRAC, CD70, CD38, CD33, or CLYBL locus, (2) a promoter which is operably linked to (3) a polynucleotide encoding a HLA-E and HLA-G transgene comprising any of the various polynucleotides disclosed herein, and (4) a transcription terminator sequence.

In some embodiments of the above-described composition(s) or the above-described vector system(s), the cell may be an induced pluripotent stem cell (iPSC).

In some embodiments, the first and/or second regulatory element may be a promoter.

In some embodiments, the first and second regulatory element may be the same.

In some embodiments, the first and second regulatory element may be different.

In some embodiments, the gRNA guide sequence may be specific for the AAVS1 locus.

In some embodiments, the gRNA guide sequence may comprise SEQ ID NO: 109.

In some embodiments, the gRNA guide sequence may be specific for the B2M locus.

In some embodiments, the gRNA guide sequence may comprise SEQ ID NO: 110.

In some embodiments, the gRNA guide sequence may be specific for the CIITA locus.

In some embodiments, the gRNA guide sequence may comprise SEQ ID NO: 111 or 112.

In some embodiments, the gRNA guide sequence may be specific for the NKG2A locus.

In some embodiments, the gRNA guide sequence may comprise SEQ ID NO: 114.

In some embodiments, the gRNA guide sequence may be specific for the TRAC locus.

In some embodiments, the gRNA guide sequence may comprise SEQ ID NO: 115.

In some embodiments, the gRNA guide sequence may be specific for the CD70 locus.

In some embodiments, the gRNA guide sequence may comprise SEQ ID NO: 116.

In some embodiments, the gRNA guide sequence may be specific for the CD38 locus.

In some embodiments, the gRNA guide sequence may comprise SEQ ID NO: 117.

In some embodiments, the gRNA guide sequence may be specific for the CD33 locus.

In some embodiments, the gRNA guide sequence may be specific for the CD33 locus and may comprise SEQ ID NO: 118 or 119.

In some embodiments, the gRNA guide sequence may be specific for the CLYBL locus.

In some embodiments, the gRNA guide may comprise SEQ ID NO: 113.

In some embodiments, the left and right polynucleotide sequences that are homologous to the left and right arms of the target sequence of the AAVS1 may comprise the nucleotide sequence of SEQ ID NOs: 73 and 74, respectively, or a fragment thereof.

In some embodiments, the left and right polynucleotide sequences that are homologous to the left and right arms of the target sequence of the B2M may comprise the nucleotide sequence of SEQ ID NOs: 76 and 77, respectively, or a fragment thereof.

In some embodiments, the left and right polynucleotide sequences that are homologous to the left and right arms of the target sequence of the CIITA may comprise the nucleotide sequence of (i) SEQ ID NOs: 79 and 80, respectively, or (ii) SEQ ID NOs: 95 and 96, respectively, or a fragment thereof.

In some embodiments, the left and right polynucleotide sequences that are homologous to the left and right arms of the target sequence of the NKG2A may comprise the nucleotide sequence of SEQ ID NOs: 85 and 86, respectively, or a fragment thereof.

In some embodiments, the left and right polynucleotide sequences that are homologous to the left and right arms of the target sequence of the TRAC may comprise the nucleotide sequence of SEQ ID NOs: 88 and 89, respectively, or a fragment thereof.

In some embodiments, the left and right polynucleotide sequences that are homologous to the left and right arms of the target sequence of the CD70 may comprise the nucleotide sequence of SEQ ID NOs: 98 and 99, respectively, or a fragment thereof.

In some embodiments, the left and right polynucleotide sequences that are homologous to the left and right arms of the target sequence of the CLYBL may comprise the nucleotide sequence of SEQ ID NOs: 82 and 83, respectively, or a fragment thereof.

In some embodiments, when the RNP complex is introduced into the cell, expression of an endogenous gene comprising the target sequence complementary to the guide sequence of the gRNA molecule may be reduced or eliminated in said cell.

In another aspect, the present invention provides one or more retroviruses comprising any of the various vector systems disclosed herein.

In another aspect, the present invention provides an isolated host cell transformed with any of the various vector systems disclosed herein or any of the various retroviruses disclosed herein.

In some embodiments, the host cell may be an iPSC or a population thereof.

In some embodiments, in the host cell may be an immune-effector cell.

In another aspect, the present invention provides an immune-effector cell, or a population thereof, derived from any of the iPSCs disclosed herein.

In some embodiments, the host cell, immune-effector cell, or population thereof may be a T cell, a natural killer (NK) cell, a natural killer T cell (NKT cell), a mesenchymal stem cell (MSC), or a macrophage.

In some embodiments, the host cell, immune-effector cell, or population thereof may be a T cell.

In some embodiments, the host cell, immune-effector cell, or population thereof may be an αβ T-cell receptor (TCR) T-cell, a γδ T-cell, a CD8+ T-cell, a CD4+ T-cell, a cytotoxic T-cell, an invariant natural killer T (INKT) cell, a memory T-cell, a memory stem T-cell (TSCM), a naïve T-cell, an effector T-cell, a T-helper cell, or a regulatory T-cell (Treg).

In some embodiments, the host cell may be an NK cell.

In another aspect, the present invention provides a pharmaceutical composition comprising any of the various isolated host cells or immune-effector cells derived from the iPSCs disclosed herein.

In another aspect, the present invention provides a method for preventing or treating a cancer, the method comprising administering to an individual in need thereof, a therapeutically effective amount of any of the various host cells, immune-effector cells, and/or populations disclosed herein, or any of the various pharmaceutical compositions disclosed herein.

In some embodiments, the cancer may be selected from the group consisting of lung cancer, pancreatic cancer, liver cancer, melanoma, bone cancer, breast cancer, colon cancer, leukemia, uterine cancer, ovarian cancer, lymphoma, and brain cancer.

In some embodiments, the cancer may be selected from the group consisting of leukemias, such as AML, CML, ALL and CLL, lymphomas, such as Hodgkin lymphoma, non-Hodgkin lymphoma and multiple myeloma, and solid cancers such as sarcomas, skin cancer, melanoma, bladder cancer, brain cancer, breast cancer, uterus cancer, ovary cancer, prostate cancer, lung cancer, colorectal cancer, cervical cancer, liver cancer, head and neck cancer, esophageal cancer, pancreatic cancer, renal cancer, adrenal cancer, stomach cancer, testicular cancer, cancer of the gall bladder and biliary tracts, thyroid cancer, thymus cancer, cancer of bone, and cerebral cancer, as well as cancer of unknown primary (CUP).

In some embodiments, the individual may have minimal residual disease (MRD) after an initial cancer treatment.

In some embodiments, the individual may have no minimal residual disease (MRD) after one or more cancer treatments or repeated dosing.

In some embodiments, the isolated host cell, immune-effector cell, or population thereof disclosed herein, has improved protective effect against allogeneic cytolysis compared to cells that does not express a chimeric single-chain HLA-E and HLA-G molecule of the present disclosure.

In some embodiments, the present application provides a method of protecting an immune-effector cell from allogeneic cytolysis, said method comprising introducing into the immune-effector cell a polynucleotide encoding a chimeric single-chain HLA-E and HLA-G molecule described herein, or a recombinant vector thereof, or a MAD7/gRNA ribonucleoprotein (RNP) complex composition described herein, or a MAD7/gRNA ribonucleoprotein (RNP)-based vector system described herein. In some embodiments, the immune-effector cell is a T cell, a natural killer (NK) cell, a natural killer T cell (NKT cell), a mesenchymal stem cell (MSC), or a macrophage. In some embodiments, the immune-effector cell is a T cell, such as an αβ T-cell receptor (TCR) T-cell, a γδ T-cell, a CD8+ T-cell, a CD4+ T-cell, a cytotoxic T-cell, an invariant natural killer T (INKT) cell, a memory T-cell, a memory stem T-cell (T_{SCM}), a naïve T-cell, an effector T-cell, a T-helper cell, or a regulatory T-cell (Treg). In some embodiments, the immune-effector cell is an NK cell. In some embodiments, the immune-effector cell is derived from an iPSC.

### BRIEF DESCRIPTION OF THE DRAWINGS

FIG. 1 depicts an exemplary schematic representation of an HLA-E and HLA-G transgene. Figure discloses “(G4S)4” as SEQ ID NO: 39 and “(G4S)3” as SEQ ID NO: 6.

FIG. 2 depicts an AAVS1 targeting vector map.

FIG. 3 depicts a B2M targeting vector map.

FIG. 4 depicts a CIITA targeting vector map.

FIG. 5 depicts a CLYBL targeting vector map.

FIG. 6 depicts a NKG2A targeting vector map.

FIG. 7 depicts a TRAC targeting vector map.

FIG. 8 depicts an CIITA targeting vector map.

FIG. 9 depicts an CD70 targeting vector map.

FIG. 10 depicts flow cytometry analysis of engineered cells showing expression of both HLA-E (see, e.g., top panel) and HLA-G (see, e.g., bottom panel) in induced pluripotent stem cells (iPSCs) after homology directed repair (HDR) into the iPSCs.

FIG. 11 depicts flow cytometry analysis of engineered cells expression of both HLA-E and HLA-G engineered by HDR into iPSCs and enriched for HLA-E expression only.

FIG. 12A-12B depicts an exemplary HLA-E and HLA-G transgene amino acid sequence (FIG. 12A) and a corresponding nucleic acid sequence (FIG. 12B).

FIGS. 13A-13B show that HLA-E expression on K562 cells offered improved protection from killing than HLA-G, but the combination of both HLA-E and HLA-G confers improved protective effect against peripheral blood mononuclear cell (PBMC) cytolysis. FIG. 13A shows a cytotoxicity assay using engineered K562 cells expressing HLA-G, HLA-E, HLA-E and HLA-G or parental WT control K562 cells as target cells and using healthy donor derived PBMCs as effector cells. Cells were co-cultured at multiple effector to target ratios (E:T) for 72 hrs. Dashed straight line indicates representative of calculated IC₅₀ values (shown on FIG. 13B description). FIG. 13B shows relative cytolysis calculated by dividing IC₅₀ values of parental control by IC₅₀ values of engineered K562 lines. Each data point represents different individual PBMC donor.

FIGS. 14A-14B shows that iNK cells edited with HLA-E and HLA-G were consistently protective compared to iNK cells lacking HLA. Cytotoxicity assay using B2M KO iNK or B2M KO iNK with HLA-E and HLA-G using healthy donor derived PBMC as effector cells. Cells were co-cultured at multiple effector to target ratios (E:T) for 72 hrs. Dashed straight line indicates representative of calculated IC₅₀ values (shown on FIG. 14B description) (FIG. 14A). Relative cytolysis calculated by dividing IC₅₀ values of B2M KO iNK cells by IC₅₀ values of B2M KO iNK cells with HLA-E and HLA-G. Each data point represents different individual PBMC donor (FIG. 14B).

FIGS. 15A-15D show that iPSCs edited with HLA-E and HLA-G joined by different peptide linkers are expressed in iPSCs. FIGS. 15A-15B show HLA-E expression, and FIGS. 15C-15D show HLA-G expression.

FIGS. 16A-16B show comparison of HLA-E (FIG. 16A) and HLA-G (FIG. 16B) expression in iPSCs edited with HLA-E and HLA-G joined by different peptide linkers.

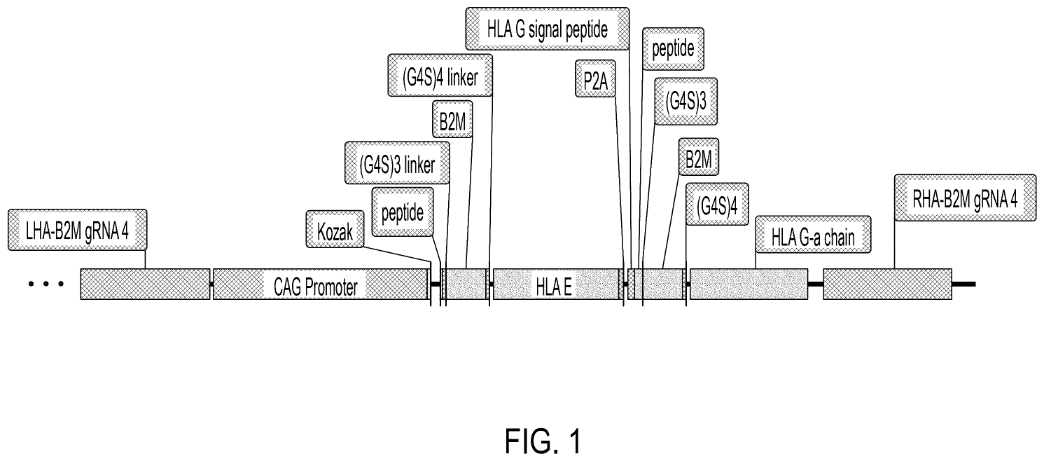

### DETAILED DESCRIPTION

The present application provides, among other things, HLA-E and HLA-G transgenes, compositions, and methods for use in genome engineering of cells, such as iPSCs. Specifically, the methods and compositions described relate to introducing nucleic acids encoding HLA-E and HLA-G transgenes into iPSCs such as pluripotent hematopoietic stem cells and/or progenitor cells (HSC/PC) and preparing immune-effector cells such as T cells, NK cells, macrophages and dendritic cells derived from iPSCs. Specifically, disclosed are DNA sequences encoding chimeric single-chain HLA-E and HLA-G molecules, gene transfer vectors for the genomic engineering of human cell lines, and the methods of use thereof. The gene transfer vectors are designed for inserting transgenes into the AAVS1, B2M, CIITA, NKG2A, TRAC, CD70, CD38, CD33, and/or CLYBL loci of human cells (e.g., iPSC) and include promoter sequences, terminator sequences and homology arms specific for the loci in question. The gene transfer vectors can be used with a CRISPR nuclease-based system, such as the MAD7 nuclease-based system. Also included are guide sequences for use with CRISPR nuclease-based systems for insertion of the transgenes, particularly with the MAD7 nuclease-based system. In some embodiments, MAD7 nuclease-based system includes a non-naturally occurring or engineered MAD7 nuclease.

### I. Definitions

Various publications, articles and patents are cited or described in the background and throughout the specification; each of these references is herein incorporated by reference in its entirety for all intended purposes. Discussion of documents, acts, materials, devices, articles or the like which has been included in the present specification is for the purpose of providing context for embodiments of the present disclosure. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this application pertains. Otherwise, certain terms used herein have the meanings as set forth in the specification.

It must be noted that as used herein and in the appended claims, the singular forms “a,” “an,” and “the” include plural reference unless the context clearly dictates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments of the application described herein. Such equivalents are intended to be encompassed by the application.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “consists of,” or variations such as “consist of” or “consisting of,” as used throughout the specification and claims, indicate the inclusion of any recited integer or group of integers, but that no additional integer or group of integers can be added to the specified method, structure, or composition.

As used herein, the term “consists essentially of,” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure or composition. See M.P.E.P. § 2111.03.

As used herein, “subject” means any animal, preferably a mammal, most preferably a human. The term “mammal” as used herein, encompasses any mammal. Examples of mammals include, but are not limited to, cows, horses, sheep, pigs, cats, dogs, mice, rats, rabbits, guinea pigs, monkeys, humans, etc., more preferably a human. In some embodiments, the subject is a patient. In some embodiments, the subject is an individual.

It should also be understood that the terms “about,” “approximately,” “generally,” “substantially,” and like terms, used herein when referring to a dimension or characteristic (e.g., concentration or concentration range) of a component of the invention, indicate that the described dimension/characteristic is not a strict boundary or parameter and does not exclude minor variations therefrom that are functionally the same or similar, as would be understood by one having ordinary skill in the art. Unless otherwise stated, any numerical values, such as a concentration or a concentration range described herein, are to be understood as being modified in all instances by the term “about.” At a minimum, such references that include a numerical parameter would include variations that, using mathematical and industrial principles accepted in the art (e.g., rounding, measurement or other systematic errors, manufacturing tolerances, etc.), would not vary the least significant digit. In some embodiments, a numerical value typically includes ±10% of the recited value. For example, a concentration of 1 mg/mL includes 0.9 mg/ml to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

The terms “identical” or percent “identity,” in the context of two or more nucleic acids (e.g., guide RNA sequences or homology arm sequences) or polypeptide sequences (e.g., chimeric single-chain HLA-E and HLA-G molecule), refer to two or more sequences or subsequences that are the same or have a specified percentage of amino acid residues or nucleotides that are the same, when compared and aligned for maximum correspondence, as measured using one of the following sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by visual inspection (see generally, Current Protocols in Molecular Biology, F. M. Ausubel et al., eds., Current Protocols, a joint venture between Greene Publishing Associates, Inc. and John Wiley & Sons, Inc. (1995 Supplement) (Ausubel)).

Examples of algorithms that are suitable for determining percent sequence identity and sequence similarity are the BLAST and BLAST 2.0 algorithms, which are described in Altschul et al. (1990) J. Mol. Biol. 215:403-410 and Altschul et al. (1997) Nucleic Acids Res. 25:3389-3402, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased.

Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions.

As used herein, the term “isolated” means a biological component (such as a nucleic acid, peptide, protein, or cell) has been substantially separated, produced apart from, or purified away from other biological components of the organism in which the component naturally occurs, i.e., other chromosomal and extrachromosomal DNA and RNA, proteins, cells, and tissues. Nucleic acids, peptides, proteins, and cells that have been “isolated” thus include nucleic acids, peptides, proteins, and cells purified by standard purification methods and purification methods described herein. “Isolated” nucleic acids, peptides, proteins, and cells can be part of a composition and still be isolated if the composition is not part of the native environment of the nucleic acid, peptide, protein, or cell. The term also embraces nucleic acids, peptides and proteins prepared by recombinant expression in a host cell as well as chemically synthesized nucleic acids.

As used herein, the term “polynucleotide,” synonymously referred to as “nucleic acid molecule,” “nucleotides” or “nucleic acids,” refers to any polyribonucleotide or polydeoxyribonucleotide, which can be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, “polynucleotide” refers to triple-stranded regions comprising RNA or DNA or both RNA and DNA. The term “polynucleotide” also includes DNAs or RNAs containing one or more modified bases and DNAs or RNAs with backbones modified for stability or for other reasons. “Modified” bases include, for example, tritylated bases and unusual bases such as inosine. A variety of modifications can be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as “oligonucleotides”.

A “construct” refers to a macromolecule or complex of molecules comprising a polynucleotide to be delivered to a host cell, either in vitro or in vivo. A “vector,” as used herein refers to any nucleic acid construct capable of directing the delivery or transfer of a foreign genetic material to target cells, where it can be replicated and/or expressed. The term “vector” as used herein comprises the construct to be delivered. A vector can be a linear or a circular molecule. A vector can be integrating or non-integrating. The major types of vectors include, but are not limited to, plasmids, episomal vector, viral vectors, cosmids, and artificial chromosomes. Viral vectors include, but are not limited to, adenovirus vector, adeno-associated virus vector, retrovirus vector, lentivirus vector, Sendai virus vector, and the like.

By “integration” or “insertion” it is meant that one or more sequences or nucleotides of an exogenous construct is stably inserted into the cellular genome, i.e., covalently linked to the nucleic acid sequence within the cell's chromosomal or mitochondrial DNA. By “targeted integration” it is meant that the nucleotide(s) of a construct is inserted into the cell's chromosomal or mitochondrial DNA at a pre-selected site or “integration site”. The term “integration” or “insertion” as used herein further refers to a process involving insertion of one or more sequences or nucleotides of the exogenous construct, with or without deletion of an endogenous sequence or one or more nucleotides at the integration site. In the case, where there is a deletion at the insertion site, “integration” can further comprise replacement of the endogenous sequence or one or more nucleotides that are deleted with the one or more inserted sequences or nucleotides.

As used herein, the term “exogenous” is intended to mean that the referenced molecule or the referenced activity is introduced into, or non-native to, the host cell. The molecule can be introduced, for example, by introduction of an encoding nucleic acid into the host genetic material such as by integration into a host chromosome or as non-chromosomal genetic material such as a plasmid. Therefore, the term as it is used in reference to expression of an encoding nucleic acid refers to introduction of the encoding nucleic acid in an expressible form into the cell. The term “endogenous” refers to a referenced molecule or activity that is present in the host cell in its native form. Similarly, the term “endogenous” when used in reference to expression of an encoding nucleic acid refers to expression of an encoding nucleic acid natively contained within the cell and not exogenously introduced.

As used herein, a “transgene”, “gene of interest” or “a polynucleotide sequence of interest” is a DNA sequence that is transcribed into RNA and in some instances translated into a polypeptide in vivo when placed under the control of appropriate regulatory sequences. A gene or polynucleotide of interest can include, but is not limited to, prokaryotic sequences, cDNA from eukaryotic mRNA, genomic DNA sequences from eukaryotic (e.g., mammalian) DNA, and synthetic DNA sequences. For example, a gene of interest may encode an miRNA, an shRNA, a native polypeptide (i.e. a polypeptide found in nature) or fragment thereof; a variant polypeptide (i.e. a mutant of the native polypeptide having less than 100% sequence identity with the native polypeptide) or fragment thereof; an engineered polypeptide or peptide fragment, a therapeutic peptide or polypeptide, an imaging marker, a selectable marker, and the like.

“Operably linked” refers to the operational linkage of nucleic acid sequences or amino acid sequences so that they are placed in functional relationships with each other. For example, a promoter is operably linked with a coding sequence or functional RNA when it is capable of affecting the expression of that coding sequence or functional RNA (i.e., the coding sequence or functional RNA is under the transcriptional control of the promoter). Coding sequences can be operably linked to regulatory sequences in sense or antisense orientation.

The term “expression” as used herein, refers to the biosynthesis of a gene product. The term encompasses the transcription of a gene into RNA. The term also encompasses translation of RNA into one or more polypeptides, and further encompasses all naturally occurring post-transcriptional and post-translational modifications. The expressed polypeptides (e.g., single-chain HLA-E and HLA-G molecule polypeptides and/or CAR polypeptides) can be within the cytoplasm of a host cell, into the extracellular milieu such as the growth medium of a cell culture or anchored to the cell membrane.

The term “regulatory element” refers to any cis-acting genetic element that controls some aspect of the expression of nucleic acid sequences. In some embodiments, the term “promoter” comprises essentially the minimal sequences required to initiate transcription. In some embodiments, the term “promoter” includes the sequences to start transcription, and in addition, also include sequences that can upregulate or downregulate transcription, commonly termed “enhancer elements” and “repressor elements”, respectively.

In some embodiments, the term “promoter” comprises essentially the minimal sequences required to initiate transcription. In some embodiments, the term “promoter” includes the sequences to start transcription, and in addition, also include sequences that can upregulate or downregulate transcription, commonly termed “enhancer elements” and “repressor elements”, respectively.

As used herein, the terms “peptide,” “polypeptide,” or “protein” can refer to a molecule comprised of amino acids and can be recognized as a protein by those of skill in the art. The conventional one-letter or three-letter code for amino acid residues is used herein. The terms “peptide,” “polypeptide,” and “protein” can be used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

The peptide sequences described herein are written according to the usual convention whereby the N-terminal region of the peptide is on the left and the C-terminal region is on the right. Although isomeric forms of the amino acids are known, it is the L-form of the amino acid that is represented unless otherwise expressly indicated.

### II. Induced Pluripotent Stem Cells (IPSCs) And Immune-Effector Cells

Induced pluripotent stem cells, commonly abbreviated as iPS cells or iPSCs, are a type of pluripotent stem cells artificially derived from non-pluripotent cells, typically adult somatic cells, by inserting certain genes. Induced pluripotent stem cells are believed to be identical to natural pluripotent stem cells, such as embryonic stem cells in many respects, for example, in the expression of certain stem cell genes and proteins, chromatin methylation patterns, doubling time, embryoid body formation, teratoma formation, viable chimera formation, and potency and differentiability, but the full extent of the relation to natural pluripotent stem cells is still being assessed. IPS cells were first produced in 2006 (Takahashi et al., 2006) from mouse cells and in 2007 from human cells (Takahashi et al., 2007; Yu et al, 2007). This has been cited as an important advancement in stem cell research, as it has allowed researchers to obtain pluripotent stem cells, which are important in research and potentially have therapeutic uses, without the controversial use of embryos.

Without wishing to be bound by theory, human iPSC technology represents a promising and potentially unlimited source of therapeutically viable hematopoietic cells for the treatment of numerous hematological and non-hematological malignancies including cancer. To advance the promise of human iPSC and genomically engineered human iPSC technology as an allogeneic source of hematopoietic cellular therapeutics, it is desired to be able to both efficiently and reproducibly generate not only hematopoietic stem and progenitor cells (HSCs), but also immune-effector populations, including, e.g., the diverse subsets of T, B, NKT, and NK lymphoid cells, and progenitor cells thereof having desired genetic modifications. Thus there is a need for methods and complexes for the efficient insertion of genetic elements in human iPSCs for therapeutic use.

IPSCs have unlimited self-renewing capacity. Use of iPSCs enables cellular engineering to produce a controlled cell bank of modified cells that can be expanded and differentiated into desired immune-effector cells, supplying large amounts of homogeneous allogeneic therapeutic products.

Provided herein are genetically engineered iPSCs and derivative cells thereof. The selected genomic modifications provided herein enhance the therapeutic properties of the derivative cells. The derivative cells are functionally improved and suitable for allogenic off-the-shelf cell therapies following a combination of selective modalities being introduced to the cells at the level of iPSC through genomic engineering. This approach can help to reduce the side effects mediated by cytokine release syndrome CRS/graft-versus-host disease (GVHD) and prevent long-term autoimmunity while providing excellent efficacy.

As used herein, the term “differentiation” is the process by which an unspecialized (“uncommitted”) or less specialized cell acquires the features of a specialized cell. Specialized cells include, for example, a blood cell or a muscle cell. A differentiated or differentiation-induced cell is one that has taken on a more specialized (“committed”) position within the lineage of a cell. The term “committed”, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type. As used herein, the term “pluripotent” refers to the ability of a cell to form all lineages of the body or soma or the embryo proper. For example, embryonic stem cells are a type of pluripotent stem cells that are able to form cells from each of the three germs layers, the ectoderm, the mesoderm, and the endoderm. Pluripotency is a continuum of developmental potencies ranging from the incompletely or partially pluripotent cell (e.g., an epiblast stem cell or EpiSC), which is unable to give rise to a complete organism to the more primitive, more pluripotent cell, which is able to give rise to a complete organism (e.g., an embryonic stem cell).

As used herein, the term “induced pluripotent stem cells” or, iPSCs, means that the stem cells are produced from differentiated adult, neonatal or fetal cells that have been induced or changed or reprogrammed into cells capable of differentiating into tissues of all three germ or dermal layers: mesoderm, endoderm, and ectoderm. The iPSCs produced do not refer to cells as they are found in nature.

The term “hematopoietic stem and progenitor cells,” “hematopoietic stem cells,” “hematopoietic progenitor cells,” or “hematopoietic precursor cells” refers to cells which are committed to a hematopoietic lineage but are capable of further hematopoietic differentiation. Hematopoietic stem cells include, for example, multipotent hematopoietic stem cells (hematoblasts), myeloid progenitors, megakaryocyte progenitors, erythrocyte progenitors, and lymphoid progenitors. Hematopoietic stem and progenitor cells (HSCs) are multipotent stem cells that give rise to all the blood cell types including myeloid (monocytes and macrophages, neutrophils, basophils, eosinophils, erythrocytes, megakaryocytes/platelets, dendritic cells), and lymphoid lineages (T cells, B cells, NK cells).

As used herein, the term “immune cell” or “immune-effector cell” refers to a cell that is involved in an immune response. Immune response includes, for example, the promotion of an immune effector response. Examples of immune cells include T cells, B cells, natural killer (NK) cells, mast cells, and myeloid-derived phagocytes. In some embodiments, the immune cell may be, for example, a T cell, a natural killer (NK) cell, a natural killer T cell (NKT cell), a mesenchymal stem cell (MSC), or a macrophage, or a population thereof.

As used herein, the term “engineered immune cell” or “engineered immune-effector cell” refers to an immune cell that has been genetically modified by the addition of exogenous genetic material in the form of DNA or RNA to the total genetic material of the cell. As used herein, the terms “T lymphocyte” and “T cell” are used interchangeably and refer to a type of white blood cell that completes maturation in the thymus and that has various roles in the immune system. A T cell can have the roles including, e.g., the identification of specific foreign antigens in the body and the activation and deactivation of other immune cells. A T cell can be any T cell, such as a cultured T cell, e.g., a primary T cell, or a T cell from a cultured T cell line, e.g., Jurkat, SupT1, etc., or a T cell obtained from a mammal. The T cell can be CD3+ cells. The T cell can be any type of T cell and can be of any developmental stage, including but not limited to, CD4+/CD8+ double positive T cells, CD4+ helper T cells (e.g., Th1 and Th2 cells), CD8+ T cells (e.g., cytotoxic T cells), peripheral blood mononuclear cells (PBMCs), peripheral blood leukocytes (PBLs), tumor infiltrating lymphocytes (TILs), memory T cells, naive T cells, regulator T cells, gamma delta T cells (gd T cells; γδ T cells), and the like. Additional types of helper T cells include cells such as Th3 (Treg), Th17, Th9, or Tfh cells. Additional types of memory T cells include cells such as central memory T cells (Tcm cells), effector memory T cells (Tern cells and TEMRA cells). The T cell can also refer to a genetically engineered T cell, such as a T cell modified to express a T cell receptor (TCR) or a chimeric antigen receptor (CAR). The T cell can also be differentiated from a stem cell or progenitor cell.

“CD4+ T cells” refers to a subset of T cells that express CD4 on their surface and are associated with cell-mediated immune response. They are characterized by the secretion profiles following stimulation, which may include secretion of cytokines such as IFN-gamma, TNF-alpha, IL2, IL4 and IL10. “CD4” are 55-kD glycoproteins originally defined as differentiation antigens on T-lymphocytes, but also found on other cells including monocytes/macrophages. CD4 antigens are members of the immunoglobulin supergene family and are implicated as associative recognition elements in MHC (major histocompatibility complex) class II-restricted immune responses. On T-lymphocytes they define the helper/inducer subset.

“CD8+ T cells” refers to a subset of T cells which express CD8 on their surface, are MHC class I-restricted, and function as cytotoxic T cells. “CD8” molecules are differentiation antigens found on thymocytes and on cytotoxic and suppressor T-lymphocytes. CD8 antigens are members of the immunoglobulin supergene family and are associative recognition elements in major histocompatibility complex class I-restricted interactions.

As used herein, the term “NK cell” or “Natural Killer cell” refers to a subset of peripheral blood lymphocytes defined by the expression of CD56 or CD16 and the absence of the T cell receptor (CD3). The NK cell can also refer to a genetically engineered NK cell, such as a NK cell modified to express a chimeric antigen receptor (CAR). The NK cell can also be differentiated from a stem cell or progenitor cell.

The induced pluripotent stem cell (iPSC) parental cell lines may be generated from peripheral blood mononuclear cells (PBMCs) or T-cells using any known method for introducing re-programming factors into non-pluripotent cells such as the episomal plasmid-based process as previously described in U.S. Pat. Nos. 8,546,140; 9,644,184; 9,328,332; and 8,765,470, the complete disclosures of which are incorporated herein by reference in their entirety for all intended purposes. The reprogramming factors may be in a form of polynucleotides, and thus are introduced to the non-pluripotent cells by vectors such as a retrovirus, a Sendai virus, an adenovirus, an episome, and a mini-circle. In particular embodiments, the one or more polynucleotides encoding at least one reprogramming factor are introduced by a lentiviral vector. In some embodiments, the one or more polynucleotides introduced by an episomal vector. In various other embodiments, the one or more polynucleotides are introduced by a Sendai viral vector. In some embodiments, the iPSCs are clonal iPSCs or are obtained from a pool of iPSCs and the genome edits are introduced by making one or more targeted integration and/or in/del at one or more selected sites. In another embodiment, the iPSCs are obtained from human T cells having antigen specificity and a reconstituted TCR gene (hereinafter, also refer to as “T-iPS” cells) as described in U.S. Pat. Nos. 9,206,394, and 10,787,642 hereby incorporated by reference into the present application in their entirety for all intended purposes.

### III. Derivative Immune-Effector Cells

In another aspect, this disclosure relates to a cell derived from differentiation of an iPSC, a derivative immune-effector cell. As described above, the genomic edits introduced into the iPSC are retained in the derivative immune-effector cell. In certain embodiments of the derivative cell obtained from iPSC differentiation, the derivative cell is a hematopoietic cell, including, but not limited to, HSCs (hematopoietic stem and progenitor cells), hematopoietic multipotent progenitor cells, T cell progenitors, NK cell progenitors, T cells, NKT cells, NK cells, and B cells. In certain embodiments, the derivative cell is an immune-effector cell, such as a NK cell or a T cell.

In certain embodiments, the application provides a natural killer (NK) cell or a T cell derived from an iPSC with one or more transgene inserts prepared in accordance with this disclosure.

Also provided is a method of manufacturing the derivative cell. The method comprises differentiating the iPSC under conditions for cell differentiation to thereby obtain the derivative cell.

An iPSC of the application can be differentiated by any method known in the art. Exemplary methods are described in U.S. Pat. Nos. 8,846,395, 8,945,922, 8,318,491, and Int. Pat. Publ. Nos. WO2010/099539, WO2012/109208, WO2017/070333, WO2017/179720, WO2016/010148, WO2018/048828 and WO2019/157597, each of which are herein incorporated by reference in its entirety for all intended purposes.

### IV. Targeted Genome Editing at Selected Loci in iPSCs

According to embodiments of the application, one or more of the exogenous polynucleotides are inserted at one or more loci on one or more chromosomes of an iPSC.

Genome editing, or genomic editing, or genetic editing, as used interchangeably herein, is a type of genetic engineering in which DNA is inserted, deleted, and/or replaced in the genome of a targeted cell. Targeted genome editing (interchangeable with “targeted genomic editing” or “targeted genetic editing”) enables insertion, deletion, and/or substitution at pre-selected sites in the genome. When an endogenous sequence is deleted or disrupted at the insertion site during targeted editing, an endogenous gene comprising the affected sequence can be knocked-out or knocked-down due to the sequence deletion or disruption. Therefore, targeted editing can also be used to disrupt endogenous gene expression with precision. Similarly used herein are the terms “targeted integration” and “targeted insertion”, referring to a process involving insertion of one or more exogenous sequences at pre-selected sites in the genome, with or without deletion of an endogenous sequence at the insertion site.

Various methods and compositions for targeted cleavage of genomic DNA have been described. Such targeted cleavage events can be used, for example, to induce targeted mutagenesis, induce targeted deletions of cellular DNA sequences, and facilitate targeted recombination at a predetermined chromosomal locus. These methods often involve the use of engineered cleavage systems to induce a double strand break (DSB) or a nick in a target DNA sequence such that repair of the break by an error-prone process such as non-homologous end joining (NHEJ) or repair using a repair template (homology directed repair or HDR) can result in the knock-out of a gene or the insertion of a sequence of interest (targeted integration). Cleavage can occur through the use of specific nucleases such as engineered zinc finger nucleases (ZFN), transcription-activator like effector nucleases (TALENs) or CRISPR/Cas systems with an engineered crRNA/tracr RNA (“single guide RNA”) to guide specific cleavage.

Targeted editing can be achieved either through a nuclease-independent approach, or through a nuclease-dependent approach. In the nuclease-independent targeted editing approach, homologous recombination is guided by homologous sequences flanking an exogenous polynucleotide to be inserted, through the enzymatic machinery of the host cell.

Alternatively, targeted editing could be achieved with higher frequency through specific introduction of double strand breaks (DSBs) by specific rare-cutting endonucleases. Such nuclease-dependent targeted editing utilizes DNA repair mechanisms including non-homologous end joining (NHEJ), which occurs in response to DSBs. Without a donor vector containing exogenous genetic material, the NHEJ often leads to random insertions or deletions (in/dels) of a small number of endogenous nucleotides. In comparison, when a donor vector containing exogenous genetic material flanked by a pair of homology arms is present, the exogenous genetic material can be introduced into the genome during homology directed repair (HDR) by homologous recombination, resulting in a “targeted integration”.

Targeted nucleases include naturally occurring and recombinant nucleases such as CRISPR related nucleases from families including Cas, Cpf, Cse, Csy, Csn, Csd, Cst, Csh, Csa, Csm, and Cmr; restriction endonucleases; meganucleases; homing endonucleases, and the like. As an example, CRISPR/Cpf1 comprises two major components: (1) a Cpf1 endonuclease and (2) a guide nucleic acid, which can be DNA or RNA. When co-expressed, the two components form a ribonucleoprotein (RNP) complex that is recruited to a target DNA sequence comprising PAM and a seeding region near PAM. The guide nucleic acid can be used to guide Cpf1 to target selected sequences. These two components can then be delivered to mammalian cells via transfection or transduction.

One type of alternative CRISPR nuclease family, Cpf1 (also known as Cas12a), has been used for genome editing since the first report in 2015 (Zetsche et al Cell, 163 (3), 759-771). Cpf1 nucleases exhibit different characteristics to Cas9 nucleases, such as a staggered DSB, a T-rich PAM and the native use of only 1 guide RNA molecule to form a complex with Cpf1 and target the DNA. These characteristics enable Cpf1 nucleases to be used in target organisms or regions within an organism's genome where a lower GC content makes the use of Cas9 less feasible.

Recently, an alternative CRISPR nuclease referred to as MAD7 has been disclosed in U.S. Pat. Nos. 9,982,279 and 10,337,028, the contents of which are hereby incorporated in their entirety for all intended purposes. The company Inscripta has made this nuclease free for all commercial or academic research. As such, its use for commercial genome editing is of great interest. Inscripta reports that MAD7 was developed from Eubacterium rectale and has proven its functionality in E. coli, S. cerevisiae and in the human HEK293T cell line. MAD7 has only 31% identity with Acidaminococcus sp. BV3L6 Cpf1 (AsCpf1), to which it also shares a T-rich PAM site (5′-YTTN-3′), and a protospacer (the region of the gRNA which associates the nuclease to the DNA target) length of 21 nucleotides. Certain embodiments of the present disclosure are particularly suitable for use with the endonuclease MAD7. This nuclease only requires a crRNA for gene editing and allows for specific targeting of AT rich regions of the genome. MAD7 cleaves DNA with a staggered cut as compared to S. pyogenes which has blunt cutting.

Exemplary MAD7 sequences and scaffold sequences for guide nucleic acid are provided in Table 1. In general, a “scaffold sequence” includes any sequence that has sufficient sequence to promote formation of a targetable ribonucleoprotein complex. The targetable ribonucleoprotein complex can comprise a nucleic acid-guided nuclease (e.g., MAD7) and a guide nucleic acid comprising a scaffold sequence and a guide sequence. Sufficient sequence within the scaffold sequence to promote formation of a targetable ribonucleoprotein complex may include a degree of complementarity along the length of two sequence regions within the scaffold sequence, such as one or two sequence regions involved in forming a secondary structure (e.g., a pseudoknot region). The one or two sequence regions may be comprised or encoded on the same polynucleotide. Alternatively, the one or two sequence regions may be comprised or encoded on separate polynucleotides. In some embodiments, a scaffold sequence can comprise the sequence of any one of SEQ ID NO: 106-108. In some embodiments, the scaffold sequence comprises the sequence of SEQ ID NO: 106. In some embodiments, the scaffold sequence comprises the sequence of SEQ ID NO: 107. In some embodiments, the scaffold sequence comprises the sequence of SEQ ID NO: 108.

TABLE 1
Exemplary MAD7 Sequences and Scaffold Sequences for Guide Nucleic Acid

| | | | SEQ ID |
|---|---|---|---|
| | Sequence | | NO |
| WT MAD7 | ATGAATAATG GAACAAATAA CTTTCAGAAT TTTATCGGAA TTTCTTCTTT GCAGAAGACT | 60 | 103 |
| nucleic acid | CTTAGGAATG CTCTCATTCC AACCGAAACA ACACAGCAAT TTATTGTTAA AAACGGAATA | 120 | |
| sequence | ATTAAAGAAG ATGAGCTAAG AGGAGAAAAT CGTCAGATAC TTAAAGATAT CATGGATGAT | 180 | |
| | TATTACAGAG GTTTCATTTC AGAAACTTTA TCGTCAATTG ATGATATTGA CTGGACTTCT | 240 | |
| | TTATTTGAGA AAATGGAAAT TCAGTTAAAA AATGGAGATA ACAAAGACAC TCTTATAAAA | 300 | |
| | GAACAGACTG AATACCGTAA GGCAATTCAT AAAAAATTTG CAAATGATGA TAGATTTAAA | 360 | |
| | AATATGTTCA GTGCAAAATT AATCTCAGAT ATTCTTCCTG AATTTGTCAT TCATAACAAT | 420 | |
| | AATTATTCTG CATCAGAAAA GGAAGAAAAA ACACAGGTAA TTAAATTATT TTCCAGATTT | 480 | |
| | GCAACGTCAT TCAAGGACTA TTTTAAAAAC AGGGCTAATT GTTTTTCGGC TGATGATATA | 540 | |
| | TCTTCATCTT CTTGTCATAG AATAGTTAAT GATAATGCAG AGATATTTTT TAGTAATGCA | 600 | |
| | TTGGTGTATA GGAGAATTGT AAAAAGTCTT TCAAATGATG ATATAAATAA AATATCCGGA | 660 | |
| | GATATGAAGG ATTCATTAAA GGAAATGTCT CTGGAAGAAA TTTATTCTTA TGAAAAATAT | 720 | |
| | GGGGAATTTA TTACACAGGA AGGTATATCT TTTTATAATG ATATATGTGG TAAAGTAAAT | 780 | |
| | TCATTTATGA ATTTATATTG CCAGAAAAAT AAAGAAAACA AAAATCTCTA TAAGCTGCAA | 840 | |
| | AAGCTTCATA AACAGATACT GTGCATAGCA GATACTTCTT ATGAGGTGCC GTATAAATTT | 900 | |
| | GAATCAGATG AAGAGGTTTA TCAATCAGTG AATGGATTTT TGGACAATAT TAGTTCGAAA | 960 | |
| | CATATCGTTG AAAGATTGCG TAAGATTGGA GACAACTATA ACGGCTACAA TCTTGATAAG | 1020 | |
| | ATTTATATTG TTAGTAAATT CTATGAATCA GTTTCACAAA AGACATATAG AGATTGGGAA | 1080 | |
| | ACAATAAATA CTGCATTAGA AATTCATTAC AACAATATAT TACCCGGAAA TGGTAAATCT | 1140 | |
| | AAAGCTGACA AGGTAAAAAA AGCGGTAAAG AATGATCTGC AAAAAAGCAT TACTGAAATC | 1200 | |
| | AATGAGCTTG TTAGCAATTA TAAATTATGT TCGGATGATA ATATTAAAGC TGAGACATAT | 1260 | |
| | ATACATGAAA TATCACATAT TTTGAATAAT TTTGAAGCAC AGGAGCTTAA GTATAATCCT | 1320 | |
| | GAAATTCATC TGGTGGAAAG TGAATTGAAA GCATCTGAAT TAAAAAATGT TCTCGATGTA | 1380 | |
| | ATAATGAATG CTTTTCATTG GTGTTCGGTT TTCATGACAG AGGAGCTGGT AGATAAAGAT | 1440 | |
| | AATAATTTTT ATGCCGAGTT AGAAGAGATA TATGACGAAA TATATCCGGT AATTTCATTG | 1500 | |
| | TATAATCTTG TGCGTAATTA TGTAACGCAG AAGCCATATA GTACAAAAAA AATTAAATTG | 1560 | |
| | AATTTTGGTA TTCCTACACT AGCGGATGGA TGGAGTAAAA GTAAAGAATA TAGTAATAAT | 1620 | |
| | GCAATTATTC TCATGCGTGA TAATTTGTAC TATTTAGGAA TATTTAATGC AAAAAATAAG | 1680 | |
| | CCTGACAAAA AGATAATTGA AGGTAATACA TCAGAAAATA AAGGGGATTA TAAGAAGATG | 1740 | |
| | ATTTATAATC TTCTGCCAGG ACCAAATAAA ATGATCCCCA AGGTATTCCT CTCTTCAAAA | 1800 | |
| | ACCGGAGTGG AAACATATAA GCCGTCTGCC TATATATTGG AGGGCTATAA ACAAAACAAG | 1860 | |
| | CATATTAAAT CCTCTAAGGA TTTTGATATA ACATTTTGTC ACGATTTGAT TGATTATTTT | 1920 | |
| | AAGAACTGTA TAGCAATACA TCCTGAATGG AAGAATTTTG GCTTTGATTT TTCTGACACC | 1980 | |
| | TCCACATATG AAGATATCAG CGGATTTTAC AGAGAAGTCG AATTACAAGG TTATAAAATC | 2040 | |
| | GACTGGACAT ATATCAGCGA AAAGGATATT GATTTGTTGC AGGAAAAAGG ACAGTTATAT | 2100 | |
| | TTATTCCAAA TATATAACAA AGATTTTTCC AAGAAAAGTA CCGGAAATGA TAATCTTCAT | 2160 | |
| | ACTATGTATT TGAAGAATTT GTTTAGTGAA GAGAATTTAA AGGATATTGT ACTGAAATTA | 2220 | |
| | AACGGTGAGG CGGAAATCTT CTTTAGAAAA TCAAGCATAA AGAATCCAAT AATTCATAAA | 2280 | |
| | AAAGGCTCTA TTCTTGTTAA TAGAACATAT GAAGCAGAGG AAAAAGATCA ATTTGGAAAT | 2340 | |
| | ATCCAGATAG TCAGAAAAAA CATACCGGAA AATATATATC AGGAGCTTTA TAAATATTTC | 2400 | |
| | AATGATAAAA GTGATAAAGA ACTTTCGGAT GAAGCAGCTA AGCTTAAGAA TGTAGTAGGT | 2460 | |
| | CATCATGAGG CTGCTACAAA CATAGTAAAA GATTATAGAT ATACATATGA TAAATATTTT | 2520 | |
| | CTTCATATGC CTATTACAAT CAATTTTAAA GCCAATAAGA CAGGCTTTAT TAATGACAGA | 2580 | |
| | ATATTACAAT ATATTGCTAA AGAAAAGGAT TTGCATGTAA TAGGCATTGA TCGTGGTGAA | 2640 | |
| | AGAAACCTGA TATATGTTTC AGTAATTGAT ACTTGTGGAA ATATTGTTGA ACAAAAATCG | 2700 | |
| | TTTAACATTG TTAATGGATA TGATTATCAG ATTAAGCTCA AGCAGCAGGA GGGGGCGCGA | 2760 | |
| | CAAATCGCAC GAAAAGAATG GAAAGAAATC GGCAAAATAA AAGAAATTAA AGAAGGCTAT | 2820 | |
| | TTATCTCTTG TAATTCATGA AATTTCAAAG ATGGTTATTA AATATAATGC CATAATTGCA | 2880 | |
| | ATGGAGGATT TAAGCTACGG ATTTAAAAAA GGTCGTTTCA AGGTTGAGCG ACAGGTTTAC | 2940 | |
| | CAGAAGTTTG AGACAATGCT TATCAACAAA CTCAACTATC TGGTATTTAA AGATATATCC | 3000 | |
| | ATAACGGAAA ACGGTGGTCT TCTAAAGGGA TACCAGCTTA CATATATTCC AGATAAACTG | 3060 | |
| | AAAAATGTGG GTCATCAATG TGGCTGTATA TTTTATGTAC CTGCTGCCTA TACATCAAAA | 3120 | |
| | ATAGATCCTA CAACCGGATT TGTAAATATA TTCAAATTTA AAGATTTAAC AGTTGATGCG | 3180 | |
| | AAGAGAGAAT TTATAAAAAA ATTTGACAGT ATCAGATATG ATTCAGAAAA AAATCTGTTT | 3240 | |
| | TGTTTTACAT TCGATTATAA TAACTTTATT ACGCAAAATA CTGTTATGTC AAAGTCAAGC | 3300 | |
| | TGGAGTGTAT ATACGTACGG AGTTAGGATA AAAAGAAGAT TTGTCAATGG CAGGTTCTCA | 3360 | |
| | AATGAATCGG ATACAATTGA TATAACAAAA GATATGGAAA AAACACTCGA AATGACAGAT | 3420 | |
| | ATAAATTGGA GAGATGGTCA TGATCTGAGG CAGGATATTA TTGATTATGA AATCGTACAA | 3480 | |
| | CACATATTTG AGATTTTTAG ATTGACTGTA CAAATGAGAA ACAGTTTAAG TGAATTAGAA | 3540 | |
| | GACAGGGATT ATGACCGTTT GATTTCTCCG GTGCTCAATG AAAATAATAT ATTTTATGAT | 3600 | |
| | TCAGCTAAAG CAGGAGATGC GTTACCTAAA GACGCAGATG CTAATGGTGC ATATTGTATA | 3660 | |
| | GCTCTAAAAG GCTTGTATGA AATCAAACAA ATTACAGAGA ATTGGAAAGA AGACGGTAAG | 3720 | |
| | TTTTCAAGAG ATAAACTTAA AATTTCCAAT AAGGACTGGT TTGACTTTAT TCAAAATAAA | 3780 | |
| | AGGTATTTAT AA | 3792 | |
| Codon | ATGAACAACG GCACAAATAA TTTTCAGAAC TTCATCGGGA TCTCAAGTTT GCAGAAAACG | 60 | 104 |
| optimized | CTGCGCAATG CTCTGATCCC CACGGAAACC ACGCAACAGT TCATCGTCAA GAACGGAATA | 120 | |
| MAD7 nucleic | ATTAAAGAAG ATGAGTTACG TGGCGAGAAC CGCCAGATTC TGAAAGATAT CATGGATGAC | 180 | |
| acid sequence | TACTACCGCG GATTCATCTC TGAGACTCTG AGTTCTATTG ATGACATAGA TTGGACTAGC | 240 | |
| | CTGTTCGAAA AAATGGAAAT TCAGCTGAAA AATGGTGATA ATAAAGATAC CTTAATTAAG | 300 | |
| | GAACAGACAG AGTATCGGAA AGCAATCCAT AAAAAATTTG CGAACGACGA TCGGTTTAAG | 360 | |
| | AACATGTTTA GCGCCAAACT GATTAGTGAC ATATTACCTG AATTTGTCAT CCACAACAAT | 420 | |
| | AATTATTCGG CATCAGAGAA AGAGGAAAAA ACCCAGGTGA TAAAATTGTT TTCGCGCTTT | 480 | |
| | GCGACTAGCT TTAAAGATTA CTTCAAGAAC CGTGCAAATT GCTTTTCAGC GGACGATATT | 540 | |
| | TCATCAAGCA GCTGCCATCG CATCGTCAAC GACAATGCAG AGATATTCTT TTCAAATGCG | 600 | |
| | CTGGTCTACC GCCGGATCGT AAAATCGCTG AGCAATGACG ATATCAACAA AATTTCGGGC | 660 | |
| | GATATGAAAG ATTCATTAAA AGAAATGAGT CTGGAAGAAA TATATTCTTA CGAGAAGTAT | 720 | |
| | GGGGAATTTA TTACCCAGGA AGGCATTAGC TTCTATAATG ATATCTGTGG GAAAGTGAAT | 780 | |
| | TCTTTTATGA ACCTGTATTG TCAGAAAAAT AAAGAAAACA AAAATTTATA CAAACTTCAG | 840 | |
| | AAACTTCACA AACAGATTCT ATGCATTGCG GACACTAGCT ATGAGGTCCC GTATAAATTT | 900 | |
| | GAAAGTGACG AGGAAGTGTA CCAATCAGTT AACGGCTTCC TTGATAACAT TAGCAGCAAA | 960 | |
| | CATATAGTCG AAAGATTACG CAAAATCGGC GATAACTATA ACGGCTACAA CCTGGATAAA | 1020 | |
| | ATTTATATCG TGTCCAAATT TTACGAGAGC GTTAGCCAAA AAACCTACCG CGACTGGGAA | 1080 | |
| | ACAATTAATA CCGCCCTCGA AATTCATTAC AATAATATCT TGCCGGGTAA CGGTAAAAGT | 1140 | |
| | AAAGCCGACA AAGTAAAAAA AGCGGTTAAG AATGATTTAC AGAAATCCAT CACCGAAATA | 1200 | |
| | AATGAACTAG TGTCAAACTA TAAGCTGTGC AGTGACGACA ACATCAAAGC GGAGACTTAT | 1260 | |
| | ATACATGAGA TTAGCCATAT CTTGAATAAC TTTGAAGCAC AGGAATTGAA ATACAATCCG | 1320 | |
| | GAAATTCACC TAGTTGAATC CGAGCTCAAA GCGAGTGAGC TTAAAAACGT GCTGGACGTG | 1380 | |
| | ATCATGAATG CGTTTCATTG GTGTTCGGTT TTTATGACTG AGGAACTTGT TGATAAAGAC | 1440 | |
| | AACAATTTTT ATGCGGAACT GGAGGAGATT TACGATGAAA TTTATCCAGT AATTAGTCTG | 1500 | |
| | TACAACCTGG TTCGTAACTA CGTTACCCAG AAACCGTACA GCACGAAAAA GATTAAATTG | 1560 | |
| | AACTTTGGAA TACCGACGTT AGCAGACGGT TGGTCAAAGT CCAAAGAGTA TTCTAATAAC | 1620 | |
| | GCTATCATAC TGATGCGCGA CAATCTGTAT TATCTGGGCA TCTTTAATGC GAAGAATAAA | 1680 | |
| | CCGGACAAGA AGATTATCGA GGGTAATACG TCAGAAAATA AGGGTGACTA CAAAAAGATG | 1740 | |
| | ATTTATAATT TGCTCCCGGG TCCCAACAAA ATGATCCCGA AAGTTTTCTT GAGCAGCAAG | 1800 | |
| | ACGGGGGTGG AAACGTATAA ACCGAGCGCC TATATCCTAG AGGGGTATAA ACAGAATAAA | 1860 | |
| | CATATCAAGT CTTCAAAAGA CTTTGATATC ACTTTCTGTC ATGATCTGAT CGACTACTTC | 1920 | |
| | AAAAACTGTA TTGCAATTCA TCCCGAGTGG AAAAACTTCG GTTTTGATTT TAGCGACACC | 1980 | |
| | AGTACTTATG AAGACATTTC CGGGTTTTAT CGTGAGGTAG AGTTACAAGG TTACAAGATT | 2040 | |
| | GATTGGACAT ACATTAGCGA AAAAGACATT GATCTGCTGC AGGAAAAAGG TCAACTGTAT | 2100 | |
| | CTGTTCCAGA TATATAACAA AGATTTTTCG AAAAAATCAA CCGGGAATGA CAACCTTCAC | 2160 | |
| | ACCATGTACC TGAAAAATCT TTTCTCAGAA GAAAATCTTA AGGATATCGT CCTGAAACTT | 2220 | |
| | AACGGCGAAG CGGAAATCTT CTTCAGGAAG AGCAGCATAA AGAACCCAAT CATTCATAAA | 2280 | |
| | AAAGGCTCGA TTTTAGTCAA CCGTACCTAC GAAGCAGAAG AAAAAGACCA GTTTGGCAAC | 2340 | |
| | ATTCAAATTG TGCGTAAAAA TATTCCGGAA AACATTTATC AGGAGCTGTA CAAATACTTC | 2400 | |
| | AACGATAAAA GCGACAAAGA GCTGTCTGAT GAAGCAGCCA AACTGAAGAA TGTAGTGGGA | 2460 | |
| | CACCACGAGG CAGCGACGAA TATAGTCAAG GACTATCGCT ACACGTATGA TAAATACTTC | 2520 | |
| | CTTCATATGC CTATTACGAT CAATTTCAAA GCCAATAAAA CGGGTTTTAT TAATGATAGG | 2580 | |
| | ATCTTACAGT ATATCGCTAA AGAAAAAGAC TTACATGTGA TCGGCATTGA TCGGGGCGAG | 2640 | |
| | CGTAACCTGA TCTACGTGTC CGTGATTGAT ACTTGTGGTA ATATAGTTGA ACAGAAAAGC | 2700 | |
| | TTTAACATTG TAAACGGCTA CGACTATCAG ATAAAACTGA AACAACAGGA GGGCGCTAGA | 2760 | |
| | CAGATTGCGC GGAAAGAATG GAAAGAAATT GGTAAAATTA AAGAGATCAA AGAGGGCTAC | 2820 | |
| | CTGAGCTTAG TAATCCACGA GATCTCTAAA ATGGTAATCA AATACAATGC AATTATAGCG | 2880 | |
| | ATGGAGGATT TGTCTTATGG TTTTAAAAAA GGGCGCTTTA AGGTCGAACG GCAAGTTTAC | 2940 | |
| | CAGAAATTTG AAACCATGCT CATCAATAAA CTCAACTATC TGGTATTTAA AGATATTTCG | 3000 | |
| | ATTACCGAGA ATGGCGGTCT CCTGAAAGGT TATCAGCTGA CATACATTCC TGATAAACTT | 3060 | |
| | AAAAACGTGG GTCATCAGTG CGGCTGCATT TTTTATGTGC CTGCTGCATA CACGAGCAAA | 3120 | |
| | ATTGATCCGA CCACCGGCTT TGTGAATATC TTTAAATTTA AAGACCTGAC AGTGGACGCA | 3180 | |
| | AAACGTGAAT TCATTAAAAA ATTTGACTCA ATTCGTTATG ACAGTGAAAA AAATCTGTTC | 3240 | |
| | TGCTTTACAT TTGACTACAA TAACTTTATT ACGCAAAACA CGGTCATGAG CAAATCATCG | 3300 | |
| | TGGAGTGTGT ATACATACGG CGTGCGCATC AAACGTCGCT TTGTGAACGG CCGCTTCTCA | 3360 | |
| | AACGAAAGTG ATACCATTGA CATAACCAAA GATATGGAGA AAACGTTGGA AATGACGGAC | 3420 | |
| | ATTAACTGGC GCGATGGCCA CGATCTTCGT CAAGACATTA TAGATTATGA AATTGTTCAG | 3480 | |
| | CACATATTCG AAATTTTCCG TTTAACAGTG CAAATGCGTA ACTCCTTGTC TGAACTGGAG | 3540 | |
| | GACCGTGATT ACGATCGTCT CATTTCACCT GTACTGAACG AAAATAACAT TTTTTATGAC | 3600 | |
| | AGCGCGAAAG CGGGGGATGC ACTTCCTAAG GATGCCGATG CAAATGGTGC GTATTGTATT | 3660 | |
| | GCATTAAAAG GGTTATATGA AATTAAACAA ATTACCGAAA ATTGGAAAGA AGATGGTAAA | 3720 | |
| | TTTTCGCGCG ATAAACTCAA AATCAGCAAT AAAGATTGGT TCGACTTTAT CCAGAATAAG | 3780 | |
| | CGCTATCTCT AA | 3792 | |
| MAD7 Amino | MNNGTNNFQN FIGISSLQKT LRNALIPTET TQQFIVKNGI IKEDELRGEN RQILKDIMDD | 60 | 105 |
| acid sequence | YYRGFISETL SSIDDIDWTS LFEKMEIQLK NGDNKDTLIK EQTEYRKAIH KKFANDDREK | 120 | |
| | NMFSAKLISD ILPEFVIHNN NYSASEKEEK TQVIKLESRF ATSFKDYFKN RANCESADDI | 180 | |
| | SSSSCHRIVN DNAEIFFSNA LVYRRIVKSL SNDDINKISG DMKDSLKEMS LEEIYSYEKY | 240 | |
| | GEFITQEGIS FYNDICGKVN SEMNLYCQKN KENKNLYKLQ KLHKQILCIA DTSYEVPYKF | 300 | |
| | ESDEEVYQSV NGELDNISSK HIVERLRKIG DNYNGYNLDK IYIVSKFYES VSQKTYRDWE | 360 | |
| | TINTALEIHY NNILPGNGKS KADKVKKAVK NDLQKSITEI NELVSNYKLC SDDNIKAETY | 420 | |
| | IHEISHILNN FEAQELKYNP EIHLVESELK ASELKNVLDV IMNAFHWCSV FMTEELVDKD | 480 | |
| | NNFYAELEEI YDEIYPVISL YNLVRNYVTQ KPYSTKKIKL NFGIPTLADG WSKSKEYSNN | 540 | |
| | AIILMRDNLY YLGIFNAKNK PDKKIIEGNT SENKGDYKKM IYNLLPGPNK MIPKVFLSSK | 600 | |
| | TGVETYKPSA YILEGYKQNK HIKSSKDEDI TFCHDLIDYF KNCIAIHPEW KNFGFDESDT | 660 | |
| | STYEDISGFY REVELQGYKI DWTYISEKDI DLLQEKGQLY LFQIYNKDES KKSTGNDNLH | 720 | |
| | TMYLKNLFSE ENLKDIVLKL NGEAEIFFRK SSIKNPIIHK KGSILVNRTY EAEEKDQFGN | 780 | |
| | IQIVRKNIPE NIYQELYKYF NDKSDKELSD EAAKLKNVVG HHEAATNIVK DYRYTYDKYF | 840 | |
| | LHMPITINFK ANKTGFINDR ILQYIAKEKD LHVIGIDRGE RNLIYVSVID TCGNIVEQKS | 900 | |
| | FNIVNGYDYQ IKLKQQEGAR QIARKEWKEI GKIKEIKEGY LSLVIHEISK MVIKYNAIIA | 960 | |
| | MEDLSYGFKK GRFKVERQVY QKFETMLINK LNYLVFKDIS ITENGGLLKG YQLTYIPDKL | 1020 | |
| | KNVGHQCGCI FYVPAAYTSK IDPTTGFVNI FKFKDLTVDA KREFIKKFDS IRYDSEKNLF | 1080 | |
| | CFTFDYNNFI TQNTVMSKSS WSVYTYGVRI KRRFVNGRFS NESDTIDITK DMEKTLEMTD | 1140 | |
| | INWRDGHDLR QDIIDYEIVQ HIFEIFRLTV QMRNSLSELE DRDYDRLISP VLNENNIFYD | 1200 | |
| | SAKAGDALPK DADANGAYCI ALKGLYEIKQ ITENWKEDGK FSRDKLKISN KDWFDFIQNK | 1260 | |
| | RYL | 1263 | |
| Scaffold | GTTAAGTTAT ATAGAATAAT TTCTACTGTT GTAGA | 35 | 106 |
| sequence for | | | |
| guide nucleic | | | |
| acid | | | |
| Scaffold | CTCTACAACT GATAAAGAAT TTCTACTTTT GTAGAT | 36 | 107 |
| sequence for | | | |
| guide nucleic | | | |
| acid | | | |
| Scaffold | GTCTGGCCCC AAATTTTAAT TTCTACTGTT GTAGAT | 36 | 108 |
| sequence for | | | |
| guide nucleic | | | |
| acid | | | |

Thus, one aspect of the present application provides a construct comprising one or more exogenous polynucleotides for targeted genome insertion utilizing the MAD7 endonuclease. In one embodiment, the construct further comprises a pair of homologous arms specific to a desired insertion site, and the method of targeted insertion comprises introducing the construct to cells to enable site specific homologous recombination by the cell host enzymatic machinery. In another embodiment, the method of targeted insertion in a cell comprises introducing a construct comprising one or more exogenous polynucleotides to the cell, and introducing a CRISPR MAD7 expression cassette comprising a DNA-binding domain specific to a desired insertion site to the cell. Specifically, in accordance with this disclosure, the method of targeted insertion in a cell comprises introducing a construct comprising one or more exogenous polynucleotides to the cell for insertion into a particular locus in an iPSC, by introducing a MAD7 nuclease, and a gRNA comprising a guide sequence specific to a desired insertion site to the cell to enable a MAD7 mediated insertion.

In general, a guide nucleic acid can complex with a compatible nucleic acid-guided nuclease and can hybridize with a target sequence, thereby directing the nuclease to the target sequence. A guide nucleic acid can be DNA. A guide nucleic acid can be RNA. A guide nucleic acid can comprise both DNA and RNA. A guide nucleic acid can comprise modified or non-naturally occurring nucleotides. In cases where the guide nucleic acid comprises RNA, the RNA guide nucleic acid can be encoded by a DNA sequence on a polynucleotide molecule such as a plasmid, linear construct, or editing cassette as disclosed herein. In particular, in certain embodiments of the present disclosure, the guide sequence is for use with a MAD7/gRNA ribonucleoprotein (RNP) complex for insertion of a transgene into the particular loci of an iPSC, comprising: (I) a guide RNA (gRNA) polynucleotide sequence specific for the MAD7 nuclease, wherein the polynucleotide sequence comprises a guide sequence capable of hybridizing to a safe harbor locus (e.g., AAVS1, B2M, CIITA, NKG2A, TRAC, CD70, CD38, CD33, or CLYBL locus) in an iPSC, wherein when associated with MAD7 nuclease, the guide sequence directs sequence-specific binding of the MAD7 complex to the target sequence, (II) a MAD7 enzyme protein, and (III) a transgene vector comprising: (1) left and right polynucleotide sequences that are homologous to the left and right arms of the target sequence of the safe harbor locus (e.g., AAVS1, B2M, CIITA, NKG2A, TRAC, CD70, CD38, CD33, or CLYBL locus), (2) a promoter which is operably linked to (3) a polynucleotide encoding the transgene of interest, and (4) a transcription terminator sequence. In one embodiment, the guide sequence comprises a nucleotide sequence selected from SEQ ID NOs: 109-119.

Sites for targeted insertion include, but are not limited to, genomic safe harbors, which are intragenic or extragenic regions of the human genome that, theoretically, are able to accommodate predictable expression of newly inserted DNA without adverse effects on the host cell or organism. In certain embodiments, the genome safe harbor for the targeted insertion is one or more loci of genes selected from the group consisting of the AAVS1, B2M, CIITA, NKG2A, TRAC, CD70, CD38, CD33 or CLYBL gene loci.

In other embodiments, the site for targeted insertion is selected for deletion or reduced expression of an endogenous gene at the insertion site. As used herein, the term “deletion” with respect to expression of a gene refers to any genetic modification that abolishes the expression of the gene. Examples of “deletion” of expression of a gene include, e.g., a removal or deletion of a DNA sequence of the gene, an insertion of an exogenous polynucleotide sequence at a locus of the gene, and one or more substitutions within the gene, which abolishes the expression of the gene.

Genes for targeted deletion include, but are not limited to, genes of major histocompatibility complex (MHC) class I and MHC class II proteins. Multiple MHC class I and class II proteins must be matched for histocompatibility in allogeneic recipients to avoid allogeneic rejection problems. “MHC deficient”, including MHC-class I deficient, or MHC-class II deficient, or both, refers to cells that either lack, or no longer maintain, or have reduced level of surface expression of a complete MHC complex comprising a MHC class I protein heterodimer and/or a MHC class II heterodimer, such that the diminished or reduced level is less than the level naturally detectable by other cells or by synthetic methods. MHC class I deficiency can be achieved by functional deletion of any region of the MHC class I locus (chromosome 6p21), or deletion or reducing the expression level of one or more MHC class-I associated genes including, not being limited to, beta-2 microglobulin (B2M) gene, TAP 1 gene, TAP 2 gene and Tapasin genes. For example, the B2M gene encodes a common subunit essential for cell surface expression of all MHC class I heterodimers. B2M null cells are MHC-I deficient. MHC class II deficiency can be achieved by functional deletion or reduction of MHC-II associated genes including, not being limited to, RFXANK, CIITA, RFX5 and RFXAP. CIITA is a transcriptional coactivator, functioning through activation of the transcription factor RFX5 required for class II protein expression. CIITA null cells are MHC-II deficient. In certain embodiments, one or more of the exogenous polynucleotides are inserted at one or more loci of genes selected from the group consisting of B2M, TAP 1, TAP 2, Tapasin, RFXANK, CIITA, RFX5 and RFXAP genes to thereby delete or reduce the expression of the gene(s) with the insertion.

In certain embodiments, the exogenous polynucleotides are inserted at one or more loci on the chromosome of the cell. In certain embodiments, the one or more loci are of genes selected from the group consisting of AAVS1, CCR5, ROSA26, collagen, HTRP, H11, GAPDH, RUNX1, B2M, TAPI, TAP2, Tapasin, NLRC5, CIITA, RFXANK, CIITA, RFX5, RFXAP, TCR a or b constant region, NKG2A, NKG2D, CD38, CIS, CBL-B, SOCS2, PD1, CTLA4, LAG3, TIM3, CD70, CD38, CD33, or TIGIT genes, provided at least one of the one or more loci is of a MHC gene, such as a gene selected from the group consisting of B2M, TAP 1, TAP 2, Tapasin, RFXANK, CIITA, RFX5 and RFXAP genes. In certain embodiments, the one or more exogenous polynucleotides are inserted at a locus of an MHC class-I associated gene, such as a beta-2 microglobulin (B2M) gene, TAP 1 gene, TAP 2 gene or Tapasin gene; and at a locus of an MHC-II associated gene, such as a RFXANK, CIITA, RFX5, RFXAP, or CIITA gene; and optionally further at a locus of a safe harbor gene selected from the group consisting of AAVS1, CCR5, ROSA26, collagen, HTRP, H11, GAPDH, TCR and RUNX1 genes. In certain embodiments, the one or more of the exogenous polynucleotides are inserted at the loci of CIITA, AAVS1 and B2M genes.

In certain embodiments, multiple transgenes can be inserted at sites targeted for deletion of complex (MHC) class I and MHC class II proteins. For instance, (a) a first exogenous polynucleotide may be inserted at a locus of AAVS1 gene; (b) a second exogenous polypeptide may be inserted at a locus of CIITA gene; and a third exogenous polypeptide may be inserted at a locus of B2M gene; wherein insertions of the exogenous polynucleotides delete or reduce expression of CIITA and B2M genes.

In certain embodiments, the guide RNA for insertion into the AAVS1 locus comprises a guide sequence of SEQ ID NO: 109 or a variant thereof, the left homology arm comprises the nucleotide sequence of SEQ ID NO: 73 or a fragment thereof, and the right homology arm comprises the nucleotide sequence of SEQ ID NO: 74 or a fragment thereof.

In certain embodiments, the guide RNA for insertion into the B2M locus comprises a guide sequence of SEQ ID NO: 110 or a variant thereof, the left homology arm comprises the nucleotide sequence of SEQ ID NO: 76 or a fragment thereof, and the right homology arm comprises the nucleotide sequence of SEQ ID NO: 77 or a fragment thereof.

In certain embodiments, the guide RNA for insertion into the CIITA locus comprises a guide sequence of SEQ ID NO: 111 or a variant thereof, the left homology arm comprises the nucleotide sequence of SEQ ID NO: 79 or a fragment thereof and the right homology arm comprises the nucleotide sequence of SEQ ID NO: 80 or a fragment thereof. In certain embodiments, the guide RNA for insertion into the CIITA locus comprises a guide sequence of SEQ ID NO: 112 or a variant thereof, the left homology arm comprises the nucleotide sequence of SEQ ID NO: 95 or a fragment thereof and the right homology arm comprises the nucleotide sequence of SEQ ID NO: 96 or a fragment thereof.

In certain embodiments, the guide RNA for insertion into the NKG2A locus comprises a guide sequence of SEQ ID NO: 114 or a variant thereof, the left homology arm comprises the nucleotide sequence of SEQ ID NO: 85 or a fragment thereof and the right homology arm comprises the nucleotide sequence of SEQ ID NO: 86 or a fragment thereof.

In certain embodiments, the guide RNA for insertion into the TRAC locus comprises a guide sequence of SEQ ID NO: 115 or a variant thereof, the left homology arm comprises the nucleotide sequence of SEQ ID NO: 88 or a fragment thereof and the right homology sequence arm comprises the nucleotide sequence of SEQ ID NO: 89 or a fragment thereof.

In certain embodiments, the guide RNA for insertion into the CLYBL locus comprises a guide sequence of SEQ ID NO: 113 or a variant thereof, the left homology arm comprises the nucleotide sequence of SEQ ID NO: 82 or a fragment thereof and the right homology sequence is selected from SEQ ID NO: 83 or a fragment thereof.

In certain embodiments, the guide RNA for insertion into the CD70 locus comprises a guide sequence of SEQ ID NO: 116 or a variant thereof, the left homology arm comprises the nucleotide sequence of SEQ ID NO: 98 or a fragment thereof and the right homology sequence is selected from SEQ ID NO: 99 or a fragment thereof.

In certain embodiments, the guide RNA for insertion into the CD38 locus comprises a guide sequence of SEQ ID NO: 117 or a variant thereof.

In certain embodiments, the guide RNA for insertion into the CD33 locus comprises a guide sequence of SEQ ID NO: 118 or 119 or a variant thereof.

Provided in Table 2 are targeting domain sequences for gRNA molecules (both RNA and DNA sequences are provided) and the corresponding homology arm sequences for use in the compositions and methods of the present disclosure, for example, in altering expression of or altering an iPSC target gene.

TABLE 2
Exemplary Targeting Domain Sequences for gRNAs and Corresponding Homology Arm Sequences

| | | | | Left Homology | Right Homology |
|---|---|---|---|---|---|
| | Genomic | Guide RNA | SEQ ID | Arm | Arm |
| Target | Location | Targeting Domain Sequence | NO: | SEQ ID NO: | SEQ ID NO: |
| AAVSI | Chr19: | UUUAUCUGUCCCCUCCACCCCACA | 109 | 73 | 74 |
| | 55115778 | TTTATCTGTCCCCTCCACCCCACA | 72 | | |
| B2M | Chr15: | UUUACUCACGUCAUCCAGCAGAGA | 110 | 76 | 77 |
| | 44715462 | TTTACTCACGTCATCCAGCAGAGA | 75 | | |
| CIITA | Chr: 16 | UUUACCUUGGGGCUCUGACAGGUA | 111 | 79 | 80 |
| | 10877367 | TTTACCTTGGGGCTCTGACAGGTA | 78 | | |
| CLYBL | Chr: 13 | AGAGUGAUCACAGCUCUGACUAAA | 113 | 82 | 83 |
| | 99822675 | AGAGTGATCACAGCTCTGACTAAA | 81 | | |
| NKG2A | Chr: 12 | CUCAGACCUGAAUCUGCCCCCAAA | 114 | 85 | 86 |
| | 10451131 | CTCAGACCTGAATCTGCCCCCAAA | 84 | | |
| TRAC | Chr: 14 | GUGUACCAGCUGAGAGACUCUAAA | 115 | 88 | 89 |
| | 22547532 | GTGTACCAGCTGAGAGACTCTAAA | 87 | | |
| CIITA | — | UUUCUGCCCAACUUCUGCUGGCAU | 112 | 95 | 96 |
| Exon 5 | | TTTCTGCCCAACTTCTGCTGGCAT | 94 | | |
| CD70 | — | UUUGGUCCCAUUGGUCGCGGGCUU | 116 | 98 | 99 |
| Exon 1 | | TTTGGTCCCATTGGTCGCGGGCTT | 97 | | |
| CD38 | — | UUUCCCGAGACCGUCCUGGCGCG | 117 | — | — |
| Exon 1 | | TTTCCCGAGACCGTCCTGGCGCG | 100 | | |
| CD33 | Chr: 19 | UUUGUCUGCAGGGAAACAAGAGACC | 118 | — | — |
| Exon 5 | 51235170 | TTTGTCTGCAGGGAAACAAGAGACC | 101 | | |
| CD33 | Chr: 19 | UUUGGAGUGGCCGGGUUCUAGAGUG | 119 | — | — |
| Exon 3 | 51225838 | TTTGGAGTGGCCGGGTTCTAGAGTG | 102 | | |

### V. Homology Arms

Whether single-stranded or double-stranded, donor templates generally include one or more regions that are homologous to regions of DNA, e.g., a target nucleic acid, within or near (e.g., flanking or adjoining) a target sequence to be cleaved, e.g., the cleavage site. These homologous regions are referred to here as “homology arms,” and are illustrated schematically below:
[5′ homology arm]-[replacement sequence]-[3′ homology arm].

The homology arms of the donor templates described herein may be of any suitable length, provided such length is sufficient to allow efficient resolution of a cleavage site on a targeted nucleic acid by a DNA repair process requiring a donor template. In certain embodiments, where amplification by, e.g., PCR, of the homology arm is desired, the homology arm is of a length such that the amplification may be performed. In certain embodiments, where sequencing of the homology arm is desired, the homology arm is of a length such that the sequencing may be performed. In certain embodiments, where quantitative assessment of amplicons is desired, the homology arms are of such a length such that a similar number of amplifications of each amplicon is achieved, e.g., by having similar G/C content, amplification temperatures, etc. In certain embodiments, the homology arm is double-stranded. In certain embodiments, the double stranded homology arm is single stranded.

In certain embodiments, the 5′ homology arm is between 50 to 250 nucleotides in length. In certain embodiments, the 5′ homology arm is about 50 nucleotides, about 75 nucleotides, about 100 nucleotides, about 125 nucleotides, about 150 nucleotides, about 175 nucleotides, about 200 nucleotides, about 225 nucleotides, or about 250 nucleotides in length.

In certain embodiments, the 3′ homology arm is between 50 to 250 nucleotides in length. In certain embodiments, the 3′ homology arm is about 50 nucleotides, about 75 nucleotides, about 100 nucleotides, about 125 nucleotides, about 150 nucleotides, about 175 nucleotides, about 200 nucleotides, about 225 nucleotides, or about 250 nucleotides in length.

The 5′ and 3′ homology arms can be of the same length or can differ in length. In certain embodiments, the 5′ and 3′ homology arms are amplified to allow for the quantitative assessment of gene editing events, such as targeted insertion, at a target nucleic acid. In certain embodiments, the quantitative assessment of the gene editing events may rely on the amplification of both the 5′ junction and 3′ junction at the site of targeted insertion by amplifying the whole or a part of the homology arm using a single pair of PCR primers in a single amplification reaction. Accordingly, although the length of the 5′ and 3′ homology arms may differ, the length of each homology arm should be capable of amplification (e.g., using PCR), as desired. Moreover, when amplification of both the 5′ and the difference in lengths of the 5′ and 3′ homology arms in a single PCR reaction is desired, the length difference between the 5′ and 3′ homology arms should allow for PCR amplification using a single pair of PCR primers.

### VI. HLA-E and HLA-G Molecules

The present application provides, among other things, HLA-E and HLA-G transgenes, compositions, and methods for use in genome engineering of cells, such as iPSCs.

HLA-G (HGNC: 4964; NCBI Entrez Gene: 3135; Ensembl: ENSG00000204632; OMIM®: 142871; UniProtKB/Swiss-Prot: P17693) and HLA-E (HGNC: 4962; NCBI Entrez Gene: 3133; Ensembl: ENSG00000204592; OMIM®: 143010; UniProtKB/Swiss-Prot: P13747) are members of the nonclassical HLA class Ib family known, in particular, for their involvement in immune regulatory processes at the maternal-fetal interface, and in immune self-nonself discrimination, respectively. Under allogeneic conditions, e.g., allograft transplantation or pregnancy, the expression of HLA-G has been associated with enhanced acceptance of the allograft or the fetus. Hence, HLA-G is critically involved in immune tolerance. As an example, from a mechanistic standpoint, short-term tolerance may be achieved by HLA-G via interaction of allograft derived β2-associated HLA-G1 and/or HLA-G5 molecules with the Ig-Like Transcript 2 (ILT2) receptor on T, NK, and B cells, thereby resulting in the inhibition of proliferation, cytotoxicity or antibody production. Short-term tolerance may be indirectly elicited by HLA-G via the presentation of, for example, an HLA-G specific leader peptide by HLA-E and its interaction with the inhibitory receptor CD94/NKG2A on T and NK cells, thus inhibiting NK cell lysis against cells that express normal levels of HLA-class I molecules. The interaction of HLA-G5 with CD8 coreceptor on certain T and NK cell population may lead to the deletion of these cells. Long-term tolerance may be achieved by the induction of different types of regulatory T (Treg) cells. Together, HLA-G and HLA-E may contribute to enhanced survival of donor cells, e.g., MHC mismatched donor cells, as universal donor cells capable of inducing “stealth” immune tolerance (i.e, evasion of the immune response), associated with decreased susceptibility to immune rejection.

In certain aspects, the present disclosure provides chimeric single-chain HLA-E and HLA-G molecules. In some embodiments, the chimeric single-chain HLA-E and HLA-G molecule of the present disclosure may comprise (a) a first molecule comprising an HLA-E heavy chain and (b) a second molecule comprising an HLA-G heavy chain, and (c) a linking peptide between (a) and (b). In some embodiments, the order of the chimeric single-chain HLA-E and HLA-G molecule is (i) (a)-(c)-(b) or (ii) (b)-(c)-(a).

In some embodiments, the HLA-E heavy chain polypeptide comprises an amino acid sequence at least 80%, such as at least 80%, 81%, 82%, 83%, 84%, 85%, 86%, 87%, 89%, 90%, 91%, 92%, 93%, 94%, 95%, 96%, 97%, 98%, 99% or 100%, identical to SEQ ID NO: 15, or in certain embodiments the amino acid sequence of SEQ ID NO: 15. In some embodiments, the HLA-E heavy chain polypeptide comprises the amino acid sequence of SEQ ID NO: 15, or an amino acid sequence having at least 80% sequence identity thereof. In some embodiments, the nucleotide sequence encoding the HLA-E heavy chain polypeptide comprises a nucleotide sequence at least 80%, such as at least 80%, 81%, 82%, 83%, 84%, 85%, 86%, 87%, 89%, 90%, 91%, 92%, 93%, 94%, 95%, 96%, 97%, 98%, 99% or 100%, identical to SEQ ID NO: 16, or in certain embodiments the nucleotide sequence of SEQ ID NO: 16. In some embodiments, the nucleotide sequence encoding the HLA-E heavy chain polypeptide comprises the sequence of SEQ ID NO: 16, or a nucleotide sequence having at least 80% sequence identity thereof.

In some embodiments, the HLA-G heavy chain polypeptide comprises an amino acid sequence at least 80%, such as at least 80%, 81%, 82%, 83%, 84%, 85%, 86%, 87%, 89%, 90%, 91%, 92%, 93%, 94%, 95%, 96%, 97%, 98%, 99% or 100%, identical to SEQ ID NO: 25, or in certain embodiments the amino acid sequence of SEQ ID NO: 25. In some embodiments, the HLA-G heavy chain polypeptide comprises the amino acid sequence of SEQ ID NO: 25, or an amino acid sequence having at least 80% sequence identity thereof. In some embodiments, the nucleotide sequence encoding the HLA-G heavy chain polypeptide comprises a nucleotide sequence at least 80%, such as at least 80%, 81%, 82%, 83%, 84%, 85%, 86%, 87%, 89%, 90%, 91%, 92%, 93%, 94%, 95%, 96%, 97%, 98%, 99% or 100%, identical to SEQ ID NO: 26, or in certain embodiments the nucleotide sequence of SEQ ID NO: 26. In some embodiments, the nucleotide sequence encoding the HLA-G heavy chain polypeptide comprises the sequence of SEQ ID NO: 26, or a nucleotide sequence having at least 80% sequence identity thereof.

In certain embodiments, the HLA-E and/or HLA-G heavy chain comprises a mutated transmembrane domain. A transmembrane domain is the portion of a transmembrane protein (e.g., HLA-E and/or HLA-G molecule) that extends across the cell membrane and anchors the molecule to cell membrane. In some instances, the transmembrane domain can be modified by amino acid substitution, deletions, or insertions to minimize interactions with other members of the HLA-E and HLA-G molecules. In some instances, the transmembrane domain can be selected or modified by amino acid substitution, deletions, or insertions to avoid-binding of proteins naturally associated with the transmembrane domain. In certain embodiments, the transmembrane domain includes additional amino acids to allow for flexibility and/or optimal distance between the domains connected to the transmembrane domain.

In some embodiments, the HLA-E heavy chain polypeptide and/or the HLA-G heavy chain polypeptides disclosed herein comprises a fragment of the HLA-E heavy chain and/or HLA-G heavy chain.

In certain embodiments, the HLA-E heavy chain fragment comprises the α₁, α₂, and/or α₃ domains. In certain embodiments, the HLA-E heavy chain fragment comprises at least the α₁ domain. In certain embodiments, the HLA-E heavy chain fragment comprises at least the α₂ domain. In certain embodiments, the HLA-E heavy chain fragment comprises at least the α₃ domain. In certain embodiments, the HLA-E heavy chain fragment comprises the ai and α₂ domains. In certain embodiments, the HLA-E heavy chain fragment comprises the α₁, α₂, and α₃ domains. In certain embodiments, the HLA-G heavy chain fragment comprises the α₁, α₂, and/or α₃ domains. In certain embodiments, the HLA-G heavy chain fragment comprises at least the 1 domain. In certain embodiments, the HLA-G heavy chain fragment comprises at least the α₂ domain. In certain embodiments, the HLA-G heavy chain fragment comprises at least the α₃ domain. In certain embodiments, the HLA-G heavy chain fragment comprises the α₁ and α₂ domains. In certain embodiments, the HLA-G heavy chain fragment comprises the α₁, α₂, and α₃ domains.

In certain embodiments, the HLA-E heavy chain fragment and/or HLA-G heavy chain fragment comprises a heterologous transmembrane domain. The transmembrane domain may be derived either from a natural or from a synthetic source. Where the source is natural, the domain may be derived from any membrane-bound or transmembrane protein.

In some embodiments, the transmembrane domain may be derived from an HLA-E heavy chain polypeptide disclosed herein. In some embodiments, the transmembrane domain may be derived from an HLA-G heavy chain polypeptide disclosed herein. One of skill in the art will appreciate that the transmembrane domains derived from the HLA-E and/or HLA-G heavy chain polypeptides disclosed herein, are interchangeable with other transmembrane domains that are not derived from HLA-E or HLA-G heavy chain polypeptides. In some embodiments, the transmembrane domain derived from the HLA-E heavy chain polypeptide and/or the HLA-G heavy chain polypeptide disclosed herein may be substituted with any number of various transmembrane domains known in the art.

Non-limiting examples of transmembrane domains which may be of particular use in this disclosure may be derived from (i.e. comprise at least the transmembrane region(s) of) the α, β or ζ chain of the T-cell receptor, CD28, CD3 epsilon, CD45, CD4, CD5, CD8, CD8α, CD9, CD16, CD22, CD33, CD37, CD40, CD64, CD80, CD86, CD134, CD137, or CD154. Alternatively, the transmembrane domain may be synthetic, in which case it will comprise predominantly hydrophobic residues such as leucine and valine. For example, a triplet of phenylalanine, tryptophan and/or valine can be found at each end of a synthetic transmembrane domain.

In some embodiments, the transmembrane domain may be derived from CD8α, CD28, CD8, CD4, CD3ζ, CD40, CD134 (OX-40), NKG2A/C/D/E, or CD7. In some embodiments, the transmembrane domain may be derived from CD28.

In certain embodiments, it will be desirable to utilize the transmembrane domain of the ζ, η or FcεR1γ chains which contain a cysteine residue capable of disulfide bonding, so that the resulting chimeric protein will be able to form disulfide linked dimers with itself, or with unmodified versions of the ζ, η or FcεR1γ chains or related proteins. In some instances, the transmembrane domain will be selected or modified by amino acid substitution to avoid-binding of such domains to the transmembrane domains of the same or different surface membrane proteins to minimize interactions with other members of the receptor complex. In other cases, it will be desirable to employ the transmembrane domain of ζ, η or FcεR1γ and -β, MB1 (Igα.), B29 or CD3-γ, ζ, or η, in order to retain physical association with other members of the receptor complex.

In some embodiments, the transmembrane domain is derived from an HLA-E heavy chain. In one embodiment, the HLA-E transmembrane domain comprises the amino acid sequence set forth in SEQ ID NO: 161, or a variant thereof having at least 50%, at least 55%, at least 60%, at least 65%, at least 70%, at least 75%, at least 80%, at least 85%, at least 90%, at least 95%, at least 96%, at least 97%, at least 98% or at least 99%, sequence identity with SEQ ID NO: 161.

In some embodiments, the transmembrane domain is derived from an HLA-G heavy chain. In one embodiment, the HLA-G transmembrane domain comprises the amino acid sequence set forth in SEQ ID NO: 162, or a variant thereof having at least 50%, at least 55%, at least 60%, at least 65%, at least 70%, at least 75%, at least 80%, at least 85%, at least 90%, at least 95%, at least 96%, at least 97%, at least 98% or at least 99%, sequence identity with SEQ ID NO: 162.

In some embodiments, the transmembrane domain is derived from CD8. In one embodiment, the CD8 transmembrane domain comprises the amino acid sequence set forth in SEQ ID NO: 154, or a variant thereof having at least 50%, at least 55%, at least 60%, at least 65%, at least 70%, at least 75%, at least 80%, at least 85%, at least 90%, at least 95%, at least 96%, at least 97%, at least 98% or at least 99%, sequence identity with SEQ ID NO: 154.

In some embodiments, the transmembrane domain is derived from CD8α. In one embodiment, the CD8α transmembrane domain comprises the amino acid sequence set forth in SEQ ID NO: 159, or a variant thereof having at least 50%, at least 55%, at least 60%, at least 65%, at least 70%, at least 75%, at least 80%, at least 85%, at least 90%, at least 95%, at least 96%, at least 97%, at least 98% or at least 99%, sequence identity with SEQ ID NO: 159.

In some embodiments, the transmembrane domain is derived from CD28. In one embodiment, the CD28 transmembrane domain comprises the amino acid sequence set forth in SEQ ID NO: 155, or a variant thereof having at least 50%, at least 55%, at least 60%, at least 65%, at least 70%, at least 75%, at least 80%, at least 85%, at least 90%, at least 95%, at least 96%, at least 97%, at least 98% or at least 99%, sequence identity with SEQ ID NO: 155. In one embodiment, the CD28 transmembrane domain comprises the amino acid sequence set forth in SEQ ID NO: 158, or a variant thereof having at least 50%, at least 55%, at least 60%, at least 65%, at least 70%, at least 75%, at least 80%, at least 85%, at least 90%, at least 95%, at least 96%, at least 97%, at least 98% or at least 99%, sequence identity with SEQ ID NO: 158.

In some embodiments, the transmembrane domain is derived from CD3ζ. In one embodiment, the CD3ζ transmembrane domain comprises the amino acid sequence set forth in SEQ ID NO: 160, or a variant thereof having at least 50%, at least 55%, at least 60%, at least 65%, at least 70%, at least 75%, at least 80%, at least 85%, at least 90%, at least 95%, at least 96%, at least 97%, at least 98% or at least 99%, sequence identity with SEQ ID NO: 160.

In some embodiments, the transmembrane domain which may be of particular use in this disclosure may be an epidermal growth factor receptor (EGFR) transmembrane domain, or fragment or derivative thereof. In some embodiments, the transmembrane domain is derived from EGFR. In one embodiment, the EGFR transmembrane domain comprises the amino acid sequence set forth in SEQ ID NO: 163, or a variant thereof having at least 50%, at least 55%, at least 60%, at least 65%, at least 70%, at least 75%, at least 80%, at least 85%, at least 90%, at least 95%, at least 96%, at least 97%, at least 98% or at least 99%, sequence identity with SEQ ID NO: 163.

In some embodiments of any of the chimeric single-chain HLA-E and HLA-G molecules disclosed herein, the linking peptide comprises an autoprotease peptide. Examples of autoprotease peptides include, but are not limited to, any of the peptide sequences set forth in Table 3, or a combination thereof. Non-limiting examples of autoprotease peptides which may be used in accordance with present disclosure are described in Table 3.

TABLE 3
Autoprotease Peptide Sequences

| | | SEQ ID |
|---|---|---|
| Description | Amino Acid Sequence | NO: |
| Thosea asigna virus 2A | EGRGSLLTCGDVEENPGP | 121 |
| (T2A) | | |
| Thosea asigna virus 2A | AEGRGSLLTCGDVEENPGP | 122 |
| Thosea asigna virus 2A | GSGEGRGSLLTCGDVEENPGP | 123 |
| Foot-and-mouth disease | GSGSRVTELL YRMKRAETYCPRPLLAIHPTEA | 124 |
| virus (FMDV) 2A (F2A) | RHKQKIVAPVKQLLNFDLLKLAGDVESNPGP | |
| Sponge 2A | LLCFLLLLLSGDVELNPGP | 125 |
| Sponge 2A | HHFMFLLLLLAGDIELNPGP | 126 |
| Acorn Worm 2A | WFLVLLSFILSGDIEVNPGP | 127 |
| Amphioxus 2A | KNCAMYMLLLSGDVETNPGP | 128 |
| Amphioxus 2A | MVISQLMLKLAGDVEENPGP | 129 |
| Porcine tesehovirus-1 2A | ATNFSLLKQAGDVEENPGP | 21 |
| (P2A) | | |
| Porcine Teschovirus-1 2A | GSGATNFSLLKQAGDVEENPGP | 130 |
| (P2A) | | |
| Equine Rhinitis A Virus | GSGQCTNYALLKLAGDVESNPGP | 131 |
| (ERAV) 2A (E2A) | | |
| 2A consensus sequence | D-X-E-X-NPGP | 132 |
| Cytoplasmic polyhedrosis | DVFRSNYDLLKLCGDIESNPGP | 156 |
| virus 2A (BmCPV2A) | | |
| Flacherie Virus 2A | TLTRAKIEDELIRAGIESNPGP | 157 |
| (BmIFV2A) | | |

In some embodiments, the autoprotease peptide comprises an amino acid sequence at least 80%, such as at least 80%, 81%, 82%, 83%, 84%, 85%, 86%, 87%, 89%, 90%, 91%, 92%, 93%, 94%, 95%, 96%, 97%, 98%, 99% or 100%, identical to an amino acid set forth in Table 3. In some embodiments, the autoprotease peptide comprises an amino acid sequence set forth in Table 3, or an amino acid sequence having at least 80% sequence identity thereof.

In some embodiments, the autoprotease peptide is a 2A peptide. Non-limiting examples of 2A peptides include P2A, F2A, E2A, T2A, GF2A, GP2A, GE2A, GT2A, BmCPV2A, or BmIFV2A peptide.

In some embodiments, the 2A peptide is a P2A peptide.

In some embodiments, the P2A peptide comprises an amino acid sequence at least 80%, such as at least 80%, 81%, 82%, 83%, 84%, 85%, 86%, 87%, 89%, 90%, 91%, 92%, 93%, 94%, 95%, 96%, 97%, 98%, 99% or 100%, identical to SEQ ID NO: 21, or in certain embodiments the amino acid sequence of SEQ ID NO: 21. In some embodiments, the P2A peptide comprises the amino acid sequence of SEQ ID NO: 21, or an amino acid sequence having at least 80% sequence identity thereof. In some embodiments, the nucleotide sequence encoding the P2A peptide comprises a nucleotide sequence at least 80%, such as at least 80%, 81%, 82%, 83%, 84%, 85%, 86%, 87%, 89%, 90%, 91%, 92%, 93%, 94%, 95%, 96%, 97%, 98%, 99% or 100%, identical to SEQ ID NO: 22, or in certain embodiments the nucleotide sequence of SEQ ID NO: 22. In some embodiments, the nucleotide sequence encoding the P2A peptide comprises the sequence of SEQ ID NO: 22, or a nucleotide sequence having at least 80% sequence identity thereof.

In some embodiments, the linking peptide comprises an autoprotease peptide and optionally one autoprotease peptide linker. In some embodiments, the linking peptide comprises an autoprotease peptide and optionally two autoprotease peptide linkers.

In certain embodiments of any of the chimeric single-chain HLA-E and HLA-G molecules disclosed herein (a) the first molecule may comprise a first B2M polypeptide fused to the HLA-E heavy chain via a first linker and/or (b) the second molecule may comprise a second B2M polypeptide fused to the HLA-G heavy chain via a second linker. In certain embodiments of any of the chimeric single-chain HLA-E and HLA-G molecules disclosed herein (a) the first molecule may comprise a first B2M polypeptide fused to the HLA-E heavy chain via a first linker and (b) the second molecule may comprise a second B2M polypeptide fused to the HLA-G heavy chain via a second linker.

In some embodiments, the first B2M polypeptide may be 5′ to the HLA-E heavy chain polypeptide. In some embodiments, the first B2M polypeptide may be 3′ to the HLA-E heavy chain polypeptide. In some embodiments, the second B2M polypeptide may be 5′ to the HLA-G heavy chain polypeptide. In some embodiments, the second B2M polypeptide may be 3′ to the HLA-G heavy chain polypeptide. In some embodiments, the first B2M polypeptide can be 5′ to the HLA-E heavy chain polypeptide and the second B2M polypeptide can be 5′ to the HLA-G heavy chain polypeptide.

In some embodiments, the first B2M polypeptide and/or the second B2M polypeptide comprises an amino acid sequence at least 80%, such as at least 80%, 81%, 82%, 83%, 84%, 85%, 86%, 87%, 89%, 90%, 91%, 92%, 93%, 94%, 95%, 96%, 97%, 98%, 99% or 100%, identical to SEQ ID NO: 9, or in certain embodiments the amino acid sequence of SEQ ID NO: 9. In some embodiments, the first B2M polypeptide and/or the second B2M polypeptide comprises the amino acid sequence of SEQ ID NO: 9, or an amino acid sequence having at least 80% sequence identity thereof. In some embodiments, the nucleotide sequence encoding the first B2M polypeptide and/or the second B2M polypeptide comprises a nucleotide sequence at least 80%, such as at least 80%, 81%, 82%, 83%, 84%, 85%, 86%, 87%, 89%, 90%, 91%, 92%, 93%, 94%, 95%, 96%, 97%, 98%, 99% or 100%, identical to SEQ ID NO: 10 or 11, or in certain embodiments the nucleotide sequence of SEQ ID NO: 10 or 11. In some embodiments, the nucleotide sequence encoding the first B2M polypeptide and/or the second B2M polypeptide comprises the sequence of SEQ ID NO: 10 or 11, or a nucleotide sequence having at least 80% sequence identity thereof.

In certain embodiments of any of the chimeric single-chain HLA-E and HLA-G molecules disclosed herein (a) the first molecule may further comprise a first presentation peptide fused to the first B2M polypeptide via a third linker and/or (b) the second molecule may further comprise a second presentation peptide fused to the second B2M polypeptide via a fourth linker. As used herein, the term “presentation peptide” may refer to a short, e.g., 8-10 amino acid, polypeptide which can non-covalently associate in a protein groove such as that formed by a class I HLA molecule associated with a B2M.

In some embodiments, the first presentation peptide may be fused to the first B2M polypeptide and (a) the second presentation peptide may be fused to the second B2M polypeptide.

In some embodiments, the first presentation peptide and/or a second presentation peptide are the same. In some embodiments, the first presentation peptide and/or a second presentation peptide are different.

In some embodiments, the first presentation peptide and/or the second presentation peptide comprises an amino acid sequence at least 80%, such as at least 80%, 81%, 82%, 83%, 84%, 85%, 86%, 87%, 89%, 90%, 91%, 92%, 93%, 94%, 95%, 96%, 97%, 98%, 99% or 100%, identical to SEQ ID NO: 4 or 23, or in certain embodiments the amino acid sequence of SEQ ID NO: 4 or 23. In some embodiments, the first presentation peptide and/or the second presentation peptide comprises the amino acid sequence of SEQ ID NO: 4 or 23, or an amino acid sequence having at least 80% sequence identity thereof. In some embodiments, the nucleotide sequence encoding the first presentation peptide and/or the second presentation peptide comprises a nucleotide sequence at least 80%, such as at least 80%, 81%, 82%, 83%, 84%, 85%, 86%, 87%, 89%, 90%, 91%, 92%, 93%, 94%, 95%, 96%, 97%, 98%, 99% or 100%, identical to SEQ ID NO: 5 or 24, or in certain embodiments the nucleotide sequence of SEQ ID NO: 5 or 24. In some embodiments, the nucleotide sequence encoding the first presentation peptide and/or the second presentation peptide comprises the sequence of SEQ ID NO: 5 or 24, or a nucleotide sequence having at least 80% sequence identity thereof.

In various embodiments, chimeric single-chain HLA-E and HLA-G molecule of the present disclosure may comprise, for example a first, second, third, fourth, and/or at least one autoprotease peptide linker. In certain embodiments, the linker can be a peptide linker and may include any naturally occurring amino acid. Exemplary amino acids that may be included into the linker are Gly, Ser Pro, Thr, Glu, Lys, Arg, Ile, Leu, His and Thr. The linker may have a length that is adequate to link any of various domains of the chimeric single-chain HLA-E and HLA-G molecule in such a way that they form the correct conformation relative to one another so that they retain the desired activity, such as insertion into the membrane. The linker may be about 5-50 amino acids long. In some embodiments, the linker is about 10-40 amino acids long. In some embodiments, the linker is about 10-35 amino acids long. In some embodiments, the linker is about 10-30 amino acids long. In some embodiments, the linker is about 10-25 amino acids long. In some embodiments, the linker is about 10-20 amino acids long. In some embodiments, the linker is about 15-20 amino acids long. Exemplary linkers that may be used are Gly rich linkers, Gly and Ser containing linkers, Gly and Ala containing linkers, Ala and Ser containing linkers, and other flexible linkers. Non-limiting examples of peptide linkers which may be used in accordance with present disclosure are described in Table 4 and Table 6. Additional linkers are described for example in Int. Pat. Publ. No. WO2019/060695, incorporated by reference herein in its entirety for all intended purposes.

TABLE 4
Peptide Linker Sequences

| | | SEQ ID |
|---|---|---|
| Description | Amino Acid Sequence | NO: |
| Whitlow Linker | GSTSGSGKPGSGEGSTKG | 33 |
| (G₄S)₃ | GGGGGGGGSGGGGS | 6 |
| Linker | GGGGSGGGGSGGGGSGGGGSGS | 12 |
| Linker 3 | GGSEGKSSGSGSESKSTGGS | 34 |
| Linker 4 | GGGSGGGS | 35 |
| Linker 5 | GGGSGGGSGGGS | 36 |
| Linker 6 | GGGSGGGSGGGSGGGS | 37 |
| Linker 7 | GGGSGGGSGGGSGGGSGGGS | 38 |
| Linker 8 (G₄S)₄ | GGGGSGGGGSGGGGSGGGGS | 39 |
| Linker 9 | GGGGSGGGGSGGGGSGGGGSGGGGS | 40 |
| (G4S)₂ Linker | GGGGSGGGGS | 41 |
| (G4S)₆ Linker | GGGGSGGGGSGGGGSGGGGSGGGGSGGGGS | 42 |
| (G4S)₇ Linker | GGGGSGGGGSGGGGSGGGGSGGGGSGGGGS | 43 |
| | GGGGS | |
| (G4S)₈ Linker | GGGGSGGGGSGGGGSGGGGSGGGGSGGGGS | 44 |
| | GGGGGGGGS | |
| Linker 10 | IRPRAIGGSKPRVA | 45 |
| Linker 11 | GKGGSGKGGSGKGGS | 46 |
| Linker 12 | GGKGSGGKGSGGKGS | 47 |
| Linker 13 | GGGKSGGGKSGGGKS | 48 |
| Linker 14 | GKGKSGKGKSGKGKS | 49 |
| Linker 15 | GGGKSGGKGSGKGGS | 50 |
| Linker 16 | GKPGSGKPGSGKPGS | 51 |
| Linker 17 | GKPGSGKPGSGKPGSGKPGS | 52 |
| Linker 18 | GKGKSGKGKSGKGKSGKGKS | 53 |
| Linker 19 | STAGDTHLGGEDFD | 54 |
| Linker 20 | GEGGSGEGGSGEGGS | 55 |
| Linker 21 | GGEGSGGEGSGGEGS | 56 |
| Linker 22 | GEGESGEGESGEGES | 57 |
| Linker 23 | GGGESGGEGSGEGGS | 58 |
| Linker 24 | GEGESGEGESGEGESGEGES | 59 |
| Linker 25 | PRGASKSGSASQTGSAPGS | 60 |
| Linker 26 | GTAAAGAGAAGGAAAGAAG | 61 |
| Linker 27 | GTSGSSGSGSGGSGSGGGG | 62 |
| Linker 28 | GSGS | 63 |
| Linker 29 | APAPAPAPAP | 64 |
| Linker 30 | APAPAPAPAPAPAPAPAPAP | 65 |
| Linker 31 | AEAAAKEAAAKEAAAAKEAAAAKEAAAAK | 66 |
| | AAA | |
| Linker (consensus) | (GGGGS)n, wherein n is an integer of 1-8 | 67 |
| GSG (Gly-Ser-Gly) Linker | GSG | — |

In some embodiments, the first, second, third, fourth, and/or at least one autoprotease peptide linker each separately comprise an amino acid sequence at least 80%, such as at least 80%, 81%, 82%, 83%, 84%, 85%, 86%, 87%, 89%, 90%, 91%, 92%, 93%, 94%, 95%, 96%, 97%, 98%, 99% or 100%, identical to an amino acid set forth in Table 4. In some embodiments, the first, second, third, fourth, and/or at least one autoprotease peptide linker each separately comprise an amino acid sequence set forth in Table 4, or an amino acid sequence having at least 80% sequence identity thereof.

In some embodiments, the first peptide linker and/or the second peptide linker comprises an amino acid sequence at least 80%, such as at least 80%, 81%, 82%, 83%, 84%, 85%, 86%, 87%, 89%, 90%, 91%, 92%, 93%, 94%, 95%, 96%, 97%, 98%, 99% or 100%, identical to SEQ ID NO: 6, 39, or 41, or in certain embodiments the amino acid sequence of SEQ ID NO: 6, 39, or 41. In some embodiments, the first peptide linker and/or the second peptide linker comprises the amino acid sequence of SEQ ID NO: 6, 39, or 41, or an amino acid sequence having at least 80% sequence identity thereof. In some embodiments, the first peptide linker and/or the second peptide linker comprises an amino acid sequence at least 80%, such as at least 80%, 81%, 82%, 83%, 84%, 85%, 86%, 87%, 89%, 90%, 91%, 92%, 93%, 94%, 95%, 96%, 97%, 98%, 99% or 100%, identical to SEQ ID NO: 6, or in certain embodiments the amino acid sequence of SEQ ID NO: 6. In some embodiments, the first peptide linker and/or the second peptide linker comprises the amino acid sequence of SEQ ID NO: 6, or an amino acid sequence having at least 80% sequence identity thereof. In some embodiments, the nucleotide sequence encoding the first peptide linker and/or the second peptide linker comprises a nucleotide sequence at least 80%, such as at least 80%, 81%, 82%, 83%, 84%, 85%, 86%, 87%, 89%, 90%, 91%, 92%, 93%, 94%, 95%, 96%, 97%, 98%, 99% or 100%, identical to SEQ ID NO: 7 or 8, or in certain embodiments the nucleotide sequence of SEQ ID NO: 7 or 8. In some embodiments, the nucleotide sequence encoding the first peptide linker and/or the second peptide linker comprises the sequence of SEQ ID NO: 7 or 8, or a nucleotide sequence having at least 80% sequence identity thereof.

In some embodiments, the third peptide linker and/or the fourth peptide linker comprises an amino acid sequence at least 80%, such as at least 80%, 81%, 82%, 83%, 84%, 85%, 86%, 87%, 89%, 90%, 91%, 92%, 93%, 94%, 95%, 96%, 97%, 98%, 99% or 100%, identical to SEQ ID NO: 12, or in certain embodiments the amino acid sequence of SEQ ID NO: 12. In some embodiments, the third peptide linker and/or the fourth peptide linker comprises the amino acid sequence of SEQ ID NO: 12, or an amino acid sequence having at least 80% sequence identity thereof. In some embodiments, the nucleotide sequence encoding the third peptide linker and/or the fourth peptide linker comprises a nucleotide sequence at least 80%, such as at least 80%, 81%, 82%, 83%, 84%, 85%, 86%, 87%, 89%, 90%, 91%, 92%, 93%, 94%, 95%, 96%, 97%, 98%, 99% or 100%, identical to SEQ ID NO: 13 or 14, or in certain embodiments the nucleotide sequence of SEQ ID NO: 13 or 14. In some embodiments, the nucleotide sequence encoding the third peptide linker and/or the fourth peptide linker comprises the sequence of SEQ ID NO: 13 or 14, or a nucleotide sequence having at least 80% sequence identity thereof.

In some embodiments, at least one of the autoprotease peptide linkers is 5′ to the autoprotease peptide, 3′ to the autoprotease peptide, or both 5′ and 3′ to the autoprotease peptide.

In some embodiments, the autoprotease peptide linker comprises the amino acid sequence of GSG (Gly-Ser-Gly). In some embodiments, the autoprotease peptide linker comprises a GSG linker. In some embodiments, the autoprotease peptide linker comprises any of the various peptide linker sequences disclosed herein, or combination thereof.

In certain embodiments of any of the chimeric single-chain HLA-E and HLA-G molecules disclosed herein (a) the first molecule further comprises a first signal peptide operably linked to the HLA-E heavy chain and/or (b) the second molecule further comprises a second signal peptide operably linked to the HLA-G heavy chain.

Any of various signal peptides known in the art, or functional fragments or combinations thereof, may be used in the practice of the present disclosure, for example, such as any of those described in signalpeptide.com/index.php?m=listspdb_mammalia, incorporated by reference for all intended purposes.

In some embodiments, the first signal peptide and the second signal peptides are the same. In some embodiments, the first signal peptide and the second signal peptides are different.

In some embodiments, the first signal peptide and/or the second signal peptide comprises an amino acid sequence at least 80%, such as at least 80%, 81%, 82%, 83%, 84%, 85%, 86%, 87%, 89%, 90%, 91%, 92%, 93%, 94%, 95%, 96%, 97%, 98%, 99% or 100%, identical to SEQ ID NO: 1, or in certain embodiments the amino acid sequence of SEQ ID NO: 1. In some embodiments, the first signal peptide and/or the second signal peptide comprises the amino acid sequence of SEQ ID NO: 1, or an amino acid sequence having at least 80% sequence identity thereof. In some embodiments, the nucleotide sequence encoding the first signal peptide and/or the second signal peptide comprises a nucleotide sequence at least 80%, such as at least 80%, 81%, 82%, 83%, 84%, 85%, 86%, 87%, 89%, 90%, 91%, 92%, 93%, 94%, 95%, 96%, 97%, 98%, 99% or 100%, identical to SEQ ID NO: 2 or 3, or in certain embodiments the nucleotide sequence of SEQ ID NO: 2 or 3. In some embodiments, the nucleotide sequence encoding the first signal peptide and/or the second signal peptide comprises the sequence of SEQ ID NO: 2 or 3, or a nucleotide sequence having at least 80% sequence identity thereof.

In some embodiments, the first signal peptide and the second signal peptide comprises an amino acid sequence at least 80%, such as at least 80%, 81%, 82%, 83%, 84%, 85%, 86%, 87%, 89%, 90%, 91%, 92%, 93%, 94%, 95%, 96%, 97%, 98%, 99% or 100%, identical to SEQ ID NO: 1, or in certain embodiments the amino acid sequence of SEQ ID NO: 1. In some embodiments, the first signal peptide and the second signal peptide comprises the amino acid sequence of SEQ ID NO: 1, or an amino acid sequence having at least 80% sequence identity thereof. In some embodiments, the nucleotide sequence encoding the first signal peptide and the second signal peptide comprises a nucleotide sequence at least 80%, such as at least 80%, 81%, 82%, 83%, 84%, 85%, 86%, 87%, 89%, 90%, 91%, 92%, 93%, 94%, 95%, 96%, 97%, 98%, 99% or 100%, identical to SEQ ID NO: 2 or 3, or in certain embodiments the nucleotide sequence of SEQ ID NO: 2 or 3. In some embodiments, the nucleotide sequence encoding the first signal peptide and the second signal peptide comprises the sequence of SEQ ID NO: 2 or 3, or a nucleotide sequence having at least 80% sequence identity thereof.

In some embodiments, the presentation peptide and/or signal peptide disclosed herein may be derived from any of signal peptides of classical HLA I alpha chains (e.g., HLA-A, HLA-B, HLA-C, and HLA-G) known in the art. In some embodiments, the presentation peptide and/or signal peptide disclosed herein may be derived from histone H2A.

In certain embodiments of any of the chimeric single-chain HLA-E and HLA-G molecules disclosed herein, the first molecule comprises an amino acid sequence at least 80%, such as at least 80%, 81%, 82%, 83%, 84%, 85%, 86%, 87%, 89%, 90%, 91%, 92%, 93%, 94%, 95%, 96%, 97%, 98%, 99% or 100%, identical to SEQ ID NO: 17 or 19, or in certain embodiments the amino acid sequence of SEQ ID NO: 17 or 19. In some embodiments, the first molecule comprises the amino acid sequence of SEQ ID NO: 17 or 19, or an amino acid sequence having at least 80% sequence identity thereof. In some embodiments, the nucleotide sequence encoding the first molecule comprises a nucleotide sequence at least 80%, such as at least 80%, 81%, 82%, 83%, 84%, 85%, 86%, 87%, 89%, 90%, 91%, 92%, 93%, 94%, 95%, 96%, 97%, 98%, 99% or 100%, identical to SEQ ID NO: 18 or 20, or in certain embodiments the nucleotide sequence of SEQ ID NO: 18 or 20. In some embodiments, the nucleotide sequence encoding the first molecule comprises the sequence of SEQ ID NO: 18 or 20, or a nucleotide sequence having at least 80% sequence identity thereof.

In some embodiments, the second molecule comprises an amino acid sequence at least 80%, such as at least 80%, 81%, 82%, 83%, 84%, 85%, 86%, 87%, 89%, 90%, 91%, 92%, 93%, 94%, 95%, 96%, 97%, 98%, 99% or 100%, identical to SEQ ID NO: 27 or 29, or in certain embodiments the amino acid sequence of SEQ ID NO: 27 or 29. In some embodiments, the second molecule comprises the amino acid sequence of SEQ ID NO: 27 or 29, or an amino acid sequence having at least 80% sequence identity thereof. In some embodiments, the nucleotide sequence encoding the second molecule comprises a nucleotide sequence at least 80%, such as at least 80%, 81%, 82%, 83%, 84%, 85%, 86%, 87%, 89%, 90%, 91%, 92%, 93%, 94%, 95%, 96%, 97%, 98%, 99% or 100%, identical to SEQ ID NO: 28 or 30, or in certain embodiments the nucleotide sequence of SEQ ID NO: 28 or 30. In some embodiments, the nucleotide sequence encoding the second molecule comprises the sequence of SEQ ID NO: 28 or 30, or a nucleotide sequence having at least 80% sequence identity thereof.

In some embodiments, the chimeric single-chain HLA-E and HLA-G molecule comprises an amino acid sequence at least 80%, such as at least 80%, 81%, 82%, 83%, 84%, 85%, 86%, 87%, 89%, 90%, 91%, 92%, 93%, 94%, 95%, 96%, 97%, 98%, 99% or 100%, identical to SEQ ID NO: 31, 165, or 168, or in certain embodiments the amino acid sequence of SEQ ID NO: 31, 165, or 168. In some embodiments, the chimeric single-chain HLA-E and HLA-G molecule comprises the amino acid sequence of SEQ ID NO: 31, 165, or 168, or an amino acid sequence having at least 80% sequence identity thereof. In some embodiments, the nucleotide sequence encoding the chimeric single-chain HLA-E and HLA-G molecule comprises a nucleotide sequence at least 80%, such as at least 80%, 81%, 82%, 83%, 84%, 85%, 86%, 87%, 89%, 90%, 91%, 92%, 93%, 94%, 95%, 96%, 97%, 98%, 99% or 100%, identical to SEQ ID NO: 32, 120, 166, 167, or 169, or in certain embodiments the nucleotide sequence of SEQ ID NO: 32, 120, 166, 167, or 169. In some embodiments, the nucleotide sequence encoding the chimeric single-chain HLA-E and HLA-G molecule comprises the sequence of SEQ ID NO: 32, 120, 166, 167, or 169, or a nucleotide sequence having at least 80% sequence identity thereof.

In some embodiments, the chimeric single-chain HLA-E and HLA-G molecule comprises an amino acid sequence at least 80%, such as at least 80%, 81%, 82%, 83%, 84%, 85%, 86%, 87%, 89%, 90%, 91%, 92%, 93%, 94%, 95%, 96%, 97%, 98%, 99% or 100%, identical to SEQ ID NO: 31, or in certain embodiments the amino acid sequence of SEQ ID NO: 31. In some embodiments, the chimeric single-chain HLA-E and HLA-G molecule comprises the amino acid sequence of SEQ ID NO: 31, or an amino acid sequence having at least 80% sequence identity thereof. In some embodiments, the nucleotide sequence encoding the chimeric single-chain HLA-E and HLA-G molecule comprises a nucleotide sequence at least 80%, such as at least 80%, 81%, 82%, 83%, 84%, 85%, 86%, 87%, 89%, 90%, 91%, 92%, 93%, 94%, 95%, 96%, 97%, 98%, 99% or 100%, identical to SEQ ID NO: 32, 120, or 167, or in certain embodiments the nucleotide sequence of SEQ ID NO: 32, 120, or 167. In some embodiments, the nucleotide sequence encoding the chimeric single-chain HLA-E and HLA-G molecule comprises the sequence of SEQ ID NO: 32, 120, or 167, or a nucleotide sequence having at least 80% sequence identity thereof.

### Non-Limiting Examples of Polynucleotides Encoding Chimeric Single-Chain HLA-E and HLA-G Molecules

In certain aspects, the present disclosure provides polynucleotide sequences encoding any of the chimeric single-chain HLA-E and HLA-G molecules disclosed herein. The single-chain HLA-E and HLA-G molecule may comprise, for example, (a) a first molecule comprising an HLA-E heavy chain and (b) a second molecule comprising an HLA-G heavy chain, and (c) a linking peptide between (a) and (b). In some embodiments, the polynucleotides may comprise a nucleotide sequence encoding the first molecule. In some embodiments, the polynucleotides may comprise a nucleotide sequence encoding the second molecule. In some embodiments, the polynucleotides may comprise a nucleotide sequence encoding any of the chimeric single-chain HLA-E and HLA-G molecules disclosed herein.

In some embodiments, the polynucleotide sequence encoding the first molecule of the chimeric single-chain HLA-E and HLA-G molecules disclosed herein comprises a nucleotide sequence at least 80%, such as at least 80%, 81%, 82%, 83%, 84%, 85%, 86%, 87%, 89%, 90%, 91%, 92%, 93%, 94%, 95%, 96%, 97%, 98%, 99% or 100%, identical to SEQ ID NO: 18 or 20, or in certain embodiments the nucleotide sequence of SEQ ID NO: 18 or 20. In some embodiments, the polynucleotide sequence encoding the first molecule comprises the sequence of SEQ ID NO: 18 or 20, or a nucleotide sequence having at least 80% sequence identity thereof.

In some embodiments, the polynucleotide sequence encoding the second molecule of the chimeric single-chain HLA-E and HLA-G molecules disclosed herein comprises a nucleotide sequence at least 80%, such as at least 80%, 81%, 82%, 83%, 84%, 85%, 86%, 87%, 89%, 90%, 91%, 92%, 93%, 94%, 95%, 96%, 97%, 98%, 99% or 100%, identical to SEQ ID NO: 28 or 30, or in certain embodiments the nucleotide sequence of SEQ ID NO: 28 or 30. In some embodiments, the polynucleotide sequence encoding the second molecule comprises the sequence of SEQ ID NO: 28 or 30, or a nucleotide sequence having at least 80% sequence identity thereof.

In some embodiments, the polynucleotide sequence encoding the chimeric single-chain HLA-E and HLA-G molecule disclosed herein comprises a nucleotide sequence at least 80%, such as at least 80%, 81%, 82%, 83%, 84%, 85%, 86%, 87%, 89%, 90%, 91%, 92%, 93%, 94%, 95%, 96%, 97%, 98%, 99% or 100%, identical to SEQ ID NO: 32, 120, 166, 167, or 169, or in certain embodiments the nucleotide sequence of SEQ ID NO: 32, 120, 166, 167, or 169. In some embodiments, the polynucleotide sequence encoding the chimeric single-chain HLA-E and HLA-G molecule disclosed herein comprises the sequence of SEQ ID NO: 32, 120, 166, 167, or 169, or a nucleotide sequence having at least 80% sequence identity thereof. In some embodiments, the polynucleotide sequence encoding the chimeric single-chain HLA-E and HLA-G molecule disclosed herein comprises a nucleotide sequence at least 80%, such as at least 80%, 81%, 82%, 83%, 84%, 85%, 86%, 87%, 89%, 90%, 91%, 92%, 93%, 94%, 95%, 96%, 97%, 98%, 99% or 100%, identical to SEQ ID NO: 32, 120, or 167, or in certain embodiments the nucleotide sequence of SEQ ID NO: 32, 120, or 167. In some embodiments, the polynucleotide sequence encoding the chimeric single-chain HLA-E and HLA-G molecule disclosed herein comprises the sequence of SEQ ID NO: 32, 120, or 167, or a nucleotide sequence having at least 80% sequence identity thereof.

In some embodiments, the polynucleotide of the present disclosure may be a DNA molecule.

In some embodiments, the polynucleotide of the present disclosure may be an RNA molecule.

### Vectors

The present disclosure further provides recombinant vectors comprising a polynucleotide encoding a chimeric single-chain HLA-E and HLA-G molecule comprising polynucleotides encoding the proteins disclosed above. In certain embodiments, the polynucleotide is operatively linked to at least one regulatory element disclosed herein.

In some embodiments, recombinant vectors of the disclosure comprise a polynucleotide sequence encoding a chimeric single-chain HLA-E and HLA-G molecule disclosed herein comprising a nucleotide sequence at least 80%, such as at least 80%, 81%, 82%, 83%, 84%, 85%, 86%, 87%, 89%, 90%, 91%, 92%, 93%, 94%, 95%, 96%, 97%, 98%, 99% or 100%, identical to SEQ ID NO: 32, 120, 166, 167, or 169, or in certain embodiments the nucleotide sequence of SEQ ID NO: 32, 120, 166, 167, or 169. In some embodiments, the polynucleotide sequence encoding the chimeric single-chain HLA-E and HLA-G molecule comprises a nucleotide sequence comprising the sequence of SEQ ID NO: 32, 120, 166, 167, or 169, or a nucleotide sequence having at least 80% sequence identity thereof. In some embodiments, the polynucleotide sequence encoding the chimeric single-chain HLA-E and HLA-G molecule disclosed herein comprises a nucleotide sequence at least 80%, such as at least 80%, 81%, 82%, 83%, 84%, 85%, 86%, 87%, 89%, 90%, 91%, 92%, 93%, 94%, 95%, 96%, 97%, 98%, 99% or 100%, identical to SEQ ID NO: 32, 120, or 167, or in certain embodiments the nucleotide sequence of SEQ ID NO: 32, 120, or 167. In some embodiments, the polynucleotide sequence encoding the chimeric single-chain HLA-E and HLA-G molecule disclosed herein comprises the sequence of SEQ ID NO: 32, 120, or 167, or a nucleotide sequence having at least 80% sequence identity thereof.

In some embodiments, the polynucleotide sequence encoding the single-chain HLA-E and HLA-G molecule may be operably linked to one or more promoter(s). In some embodiments, the polynucleotide sequence encoding the encoding the single-chain HLA-E and HLA-G molecule may be operably linked to a one, two, three, four, five or more promoter(s). In some embodiments, the polynucleotide sequence encoding the encoding the single-chain HLA-E and HLA-G molecule may be operably linked to a single promoter.

In some embodiments, the one or more promoters is an exogenous promoter. In some embodiments, the one or more exogenous promoters may comprise, for example, CMV, EF1a, PGK, CAG, UBC, SV40, human beta actin, or other constitutive, inducible, temporal-, tissue-, or cell type-specific promoters. In some embodiments, the one or more promoters is an exogenous promoter. In some embodiments, the endogenous promoter may be comprised in selected sites such as AAVS1, B2M, CIITA, NKG2A, TRAC, CD70, CD38, CD33, or CLYBL, or other locus meeting the criteria of a genome safe harbor.

In some embodiments, the promoter is an inducible promoter.

In some embodiments, the promoter is a CAG promoter. In some embodiments, the CAG promoter comprises the polynucleotide sequence at least 80%, such as at least 80%, 81%, 82%, 83%, 84%, 85%, 86%, 87%, 89%, 90%, 91%, 92%, 93%, 94%, 95%, 96%, 97%, 98%, 99% or 100%, identical to SEQ ID NO: 92.

In some embodiments, the recombinant vector comprising the polynucleotides disclosed herein is a viral vector. As a non-limiting example, the viral vector may be a retroviral vector, a lentiviral vector, an adenoviral vector, an adeno-associated virus vector, an alphaviral vector, a herpes virus vector, a baculoviral vector, or a vaccinia virus vector.

In some embodiments, the recombinant vector comprising the polynucleotides disclosed herein is a non-viral vector. As a non-limiting example, the non-viral vector may be a minicircle plasmid, a Sleeping Beauty transposon, a piggyBac transposon, or a single or double stranded DNA molecule that can be used as a template for homology directed repair (HDR) based gene editing.

### Host Cells

In one aspect, the present disclosure provides an isolated host cell comprising any of the various polynucleotides disclosed herein. In another aspect, the present disclosure provides an isolated host cell comprising any of the various recombinant vectors disclosed herein. In yet another aspect, the present disclosure provides an isolated host cell comprising any of the various HLA-E and HLA-G molecules encoded by the polynucleotide disclosed herein.

In some embodiments, the isolated host cell disclosed herein may comprise two or more polynucleotides or recombinant vectors described herein.

In some embodiments, the host cell may be an iPSC or a population thereof. In some embodiments, the host cell may be an immune cell.

In certain aspects, the present disclosure provides an immune-effector cell, or a population thereof, derived from an induced pluripotent stem cell (iPSC) disclosed herein.

In some embodiments, the isolated host cell, immune-effector cell, or population thereof may be a T cell, a natural killer (NK) cell, a natural killer T cell (NKT cell), a mesenchymal stem cell (MSC), or a macrophage.

In some embodiments, the isolated host cell, immune-effector cell, or population thereof may be a T cell. T-cells may include, for example, without limitation, thymocytes, naïve T lymphocytes, immature T lymphocytes, mature T lymphocytes, resting T lymphocytes, or activated T lymphocytes. A T-cell can be a T helper (Th) cell, for example a T helper 1 (Th1) or a T helper 2 (Th2) cell. The T-cell can be a helper T-cell (HTL; CD4+ T-cell) CD4+ T-cell, a cytotoxic T-cell (CTL; CD8+ T-cell), a tumor infiltrating cytotoxic T-cell (TIL; CD8+ T-cell), CD4+ CD8+ T-cell, or any other subset of T-cells. Other illustrative populations of T-cells suitable for use in particular embodiments include naïve T-cells memory T-cells, and NKT cells.

In some embodiments, the T-cell may be a CD8+ T-cell, a CD4+ T-cell, a cytotoxic T-cell, an αβ T-cell receptor (TCR) T-cell, an invariant natural killer T (INKT) cell, a γδ T-cell, a memory T-cell, a memory stem T-cell (TSCM), a naïve T-cell, an effector T-cell, a T-helper cell, or a regulatory T-cell (Treg).

In some embodiments, the isolated host cell, immune-effector cell, or population thereof of a αβ T-cell receptor (TCR) T-cell, a γδ T-cell, a CD8+ T-cell, a CD4+ T-cell, a cytotoxic T-cell, an invariant natural killer T (INKT) cell, a memory T-cell, a memory stem T-cell (TSCM), a naïve T-cell, an effector T-cell, a T-helper cell, or a regulatory T-cell (Treg).

In some embodiments, the host cell, immune-effector cell, or population thereof may be an NK cell.

### VII. HLA-E and HLA-G Transgenes

According to certain embodiments, an immune cell or iPSC of the application can be modified by introducing an exogenous polynucleotide encoding one or more proteins related to immune evasion, such as non-classical HLA class I proteins (e.g., HLA-E and HLA-G).

In certain embodiments, an iPSC is engineered by the insertion of an HLA-E and HLA-G transgene using the described MAD7/gRNA ribonucleoprotein (RNP) complex disclosed herein. In some embodiments, the HLA-E and HLA-G transgene disclosed herein may be inserted utilizing the RNP complex, guide sequences and homology arms in accordance with this disclosure.

### MAD7/gRNA Ribonucleoprotein (RNP) Complex Compositions and Vector Systems

In certain aspects, the present disclosure provides a MAD7/gRNA ribonucleoprotein (RNP) complex composition for insertion of an HLA-E and HLA-G transgene, comprising: (I) a MAD7 nuclease; (II) a guide RNA (gRNA) specific for the MAD7 nuclease, wherein the gRNA comprises a guide sequence capable of hybridizing to a target sequence of an AAVS1, B2M, CIITA, NKG2A, TRAC, CD70, CD38, CD33, or CLYBL locus in a cell, wherein the guide sequence is selected from SEQ ID NOs: 109-119, wherein when the gRNA is complexed with the MAD7 nuclease, the guide sequence directs sequence-specific binding of the MAD7 nuclease to the target sequence; and (III) a transgene vector comprising: (1) left and right polynucleotide sequences that are homologous to left and right arms of the target sequence of the AAVS1, B2M, CIITA, NKG2A, TRAC, CD70, CD38, CD33, or CLYBL locus, (2) a promoter which is operably linked to (3) a polynucleotide encoding the HLA-E and HLA-G transgene comprising any of the polynucleotides disclosed herein, e.g., polynucleotides encoding chimeric single-chain HLA-E and HLA-G molecules disclosed herein, and (4) a transcription terminator sequence.

In certain aspects, the present disclosure provides a MAD7/gRNA ribonucleoprotein (RNP) complex composition for insertion of an HLA-E and HLA-G transgene, comprising: I) a MAD7 nuclease system, wherein the system is encoded by one or more vectors comprising (a) a sequence encoding a guide RNA (gRNA) operably linked to a first regulatory element, wherein the gRNA comprises a guide sequence capable of hybridizing to a target sequence of the AAVS1, B2M, CIITA, NKG2A, TRAC, CD70, CD38, or CLYBL locus in a cell, wherein the guide sequence is selected from SEQ ID NOs: 109-119, and wherein when transcribed, the guide sequence directs sequence-specific binding of the MAD7 complex to the target sequence, (b) a sequence encoding a MAD7 nuclease, wherein the sequence is operably linked to a second regulatory element; and (II) a HLA-E and HLA-G transgene vector comprising: (1) left and right polynucleotide sequences that are homologous to the left and right arms of the target sequence of the AAVS1, B2M, CIITA, NKG2A, TRAC, CD70, CD38, CD33 or CLYBL locus, (2) a promoter which is operably linked to (3) a polynucleotide encoding the HLA-E and HLA-G transgene comprising any of the polynucleotides disclosed herein, e.g., polynucleotides encoding chimeric single-chain HLA-E and HLA-G molecules disclosed herein, and (4) a transcription terminator sequence.

In certain aspects, the present disclosure provides a MAD7/gRNA ribonucleoprotein (RNP)-based vector system, comprising: (I) one or more vectors comprising (a) a sequence encoding a guide RNA (gRNA), wherein the sequence is operably linked to a first regulatory element, wherein the gRNA comprises a guide sequence capable of hybridizing to a target sequence of the AAVS1, B2M, CIITA, NKG2A, TRAC, CD70, CD38, CD33 or CLYBL locus in a cell, wherein the gRNA guide sequence is selected from SEQ ID NOs: 109-119, wherein when transcribed, the guide sequence directs sequence-specific binding of the MAD7 complex to the target sequence; (b) a sequence encoding a MAD7 nuclease, wherein the sequence is operably linked to a second regulatory element; and (II) a HLA-E and HLA-G transgene vector comprising: (1) left and right polynucleotide sequences that are homologous to the left and right arms of the target sequence of the AAVS1, B2M, CIITA, NKG2A, TRAC, CD70, CD38, CD33, or CLYBL locus, (2) a promoter which is operably linked to (3) a polynucleotide encoding any of the polynucleotides disclosed herein, e.g., polynucleotides encoding chimeric single-chain HLA-E and HLA-G molecules disclosed herein, and (4) a transcription terminator sequence.

In some embodiments of the MAD7/gRNA ribonucleoprotein (RNP) complex compositions and/or vector systems disclosed herein, the cell may be any of the various cells disclosed herein. In some embodiments, the cell may be an induced pluripotent stem cell (iPSC).

In some embodiments, the first and/or second regulatory element is any of the various promoters disclosed herein.

In some embodiments, the first and second regulatory element are the same. In some embodiments, the first and second regulatory element are different.

In some embodiments of the MAD7/gRNA ribonucleoprotein (RNP) complex compositions and/or vector systems disclosed herein, the gRNA guide sequence is specific for the AAVS1 locus. In some embodiments, the gRNA guide sequence comprises SEQ ID NO: 109.

In some embodiments, the gRNA guide sequence is specific for the B2M. In some embodiments, the gRNA guide sequence comprises SEQ ID NO: 110.

In some embodiments, the gRNA guide sequence is specific for the CIITA locus. In some embodiments, the gRNA guide sequence comprises SEQ ID NO: 111 or 112.

In some embodiments, the gRNA guide sequence is specific for the NKG2A locus. In some embodiments, the gRNA guide sequence comprises SEQ ID NO: 114.

In some embodiments, the gRNA guide sequence is specific for the TRAC locus. In some embodiments, the gRNA guide sequence comprises SEQ ID NO: 115

In some embodiments, the gRNA guide sequence is specific for the CD70 locus. In some embodiments, the gRNA guide sequence comprises SEQ ID NO: 116.

In some embodiments, the gRNA guide sequence is specific for the CD38 locus. In some embodiments, the gRNA guide sequence comprises SEQ ID NO: 117.

In some embodiments, the gRNA guide sequence is specific for the CD33 locus. In some embodiments, the gRNA guide sequence is specific for the CD33 locus and comprises SEQ ID NO: 118 or 119.

In some embodiments, the gRNA guide sequence is specific for the CLYBL locus. In some embodiments, wherein the gRNA guide comprises SEQ ID NO: 113.

In some embodiments of the MAD7/gRNA ribonucleoprotein (RNP) complex compositions and/or vector systems disclosed herein, the left and right polynucleotide sequences that are homologous to the left and right arms of the target sequence of the AAVS1 comprise the nucleotide sequence of SEQ ID NOs: 73 and 74, respectively, or a fragment thereof.

In some embodiments of the MAD7/gRNA ribonucleoprotein (RNP) complex compositions and/or vector systems disclosed herein, the left and right polynucleotide sequences that are homologous to the left and right arms of the target sequence of the B2M comprise the nucleotide sequence of SEQ ID NOs: 76 and 77, respectively, or a fragment thereof.

In some embodiments of the MAD7/gRNA ribonucleoprotein (RNP) complex compositions and/or vector systems disclosed herein, the left and right polynucleotide sequences that are homologous to the left and right arms of the target sequence of the CIITA comprise the nucleotide sequence of (i) SEQ ID NOs: 79 and 80, respectively, or (ii) SEQ ID NOs: 95 and 96, respectively, or a fragment thereof.

In some embodiments of the MAD7/gRNA ribonucleoprotein (RNP) complex compositions and/or vector systems disclosed herein, the left and right polynucleotide sequences that are homologous to the left and right arms of the target sequence of the NKG2A comprise the nucleotide sequence of SEQ ID NOs: 85 and 86, respectively, or a fragment thereof.

In some embodiments of the MAD7/gRNA ribonucleoprotein (RNP) complex compositions and/or vector systems disclosed herein, the left and right polynucleotide sequences that are homologous to the left and right arms of the target sequence of the TRAC comprise the nucleotide sequence of SEQ ID NOs: 88 and 89, respectively, or a fragment thereof.

In some embodiments of the MAD7/gRNA ribonucleoprotein (RNP) complex compositions and/or vector systems disclosed herein, the left and right polynucleotide sequences that are homologous to the left and right arms of the target sequence of the CD70 comprise the nucleotide sequence of SEQ ID NOs: 98 and 99, respectively, or a fragment thereof

In some embodiments of the MAD7/gRNA ribonucleoprotein (RNP) complex compositions and/or vector systems disclosed herein, the left and right polynucleotide sequences that are homologous to the left and right arms of the target sequence of the CLYBL comprise the nucleotide sequence of SEQ ID NOs: 82 and 83, respectively, or a fragment thereof.

In some embodiments of the MAD7/gRNA ribonucleoprotein (RNP) complex compositions and/or vector systems disclosed herein, when the RNP complex is introduced into the cell, expression of an endogenous gene comprising the target sequence complementary to the guide sequence of the gRNA molecule is reduced or eliminated in the cell.

In some embodiments, the expression of the endogenous gene comprising the target sequence complementary to the guide sequence of the gRNA molecule is reduced by at least about 5%, 10%, 20%, 30%, 40%, 50%, 60%, 70%, 80%, 90%, or more. In some embodiments, the activity of epiregulin is reduced by about 5%-20%, 10%-30%, 20%-40%, 30%-50%, 40%-60%, 50%-70%, 60%-80%, 70%-90%, 80%-95%, or more. In some embodiments, expression of an endogenous gene comprising the target sequence complementary to the guide sequence of the gRNA molecule is reduced by about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more.

In some embodiments, when the RNP complex is introduced into the cell, expression of an endogenous gene comprising the target sequence complementary to the guide sequence of the gRNA molecule is eliminated in the cell.

In certain aspects, the present disclosure provides one or more retroviruses comprising the vector system.

In certain aspects, the present disclosure provides an isolated host cell disclosed herein may be transformed with the vector system disclosed herein. In certain aspects, the present disclosure provides an isolated host cell disclosed herein transformed with the one or more retroviruses disclosed herein. In some embodiments, the host cell may be any of various host cells disclosed herein.

In some embodiments, the host cell may be an iPSC or a population thereof. In some embodiments, the host cell may be an immune cell.

In certain aspects, the present disclosure provides an immune-effector cell, or a population thereof, derived from an induced pluripotent stem cell (iPSC) disclosed herein.

In some embodiments, the isolated host cell, immune-effector cell, or population thereof may be a T cell, a natural killer (NK) cell, a natural killer T cell (NKT cell), a mesenchymal stem cell (MSC), or a macrophage.

In some embodiments, the isolated host cell, immune-effector cell, or population thereof may be a T cell. T-cells may include, for example, without limitation, thymocytes, naïve T lymphocytes, immature T lymphocytes, mature T lymphocytes, resting T lymphocytes, or activated T lymphocytes. A T-cell can be a T helper (Th) cell, for example a T helper 1 (Th1) or a T helper 2 (Th2) cell. The T-cell can be a helper T-cell (HTL; CD4+ T-cell) CD4+ T-cell, a cytotoxic T-cell (CTL; CD8+ T-cell), a tumor infiltrating cytotoxic T-cell (TIL; CD8+ T-cell), CD4+ CD8+ T-cell, or any other subset of T-cells. Other illustrative populations of T-cells suitable for use in particular embodiments include naive T-cells memory T-cells, and NKT cells.

In some embodiments, the T-cell may be a CD8+ T-cell, a CD4+ T-cell, a cytotoxic T-cell, an αβ T-cell receptor (TCR) T-cell, an invariant natural killer T (iNKT) cell, a γδ T-cell, a memory T-cell, a memory stem T-cell (TSCM), a naïve T-cell, an effector T-cell, a T-helper cell, or a regulatory T-cell (Treg).

In some embodiments, the isolated host cell, immune-effector cell, or population thereof of a αβ T-cell receptor (TCR) T-cell, a γδ T-cell, a CD8+ T-cell, a CD4+ T-cell, a cytotoxic T-cell, an invariant natural killer T (INKT) cell, a memory T-cell, a memory stem T-cell (TSCM), a naïve T-cell, an effector T-cell, a T-helper cell, or a regulatory T-cell (Treg).

In some embodiments, the host cell, immune-effector cell, or population thereof may be an NK cell.

In some embodiments, the isolated host cell, immune-effector cell, or population thereof disclosed herein, has improved protective effect against allogeneic cytolysis compared to cells lacking the chimeric single-chain HLA-E and HLA-G molecule of the present disclosure or the polynucleotide of the present disclosure. The cytolysis may be mediated by allogeneic effector cells present in peripheral mononuclear cells (PBMCs). The effect is primarily generated by NK cells but other immune cells may have additional contributions as well.

### VIII. Chimeric Antigen Receptors (“CARs”) Transgenes

In some embodiments, at least one of the transgenes that may be inserted into a particular locus of an immune cell or iPSC of the application, is one encoding an exogenous chimeric antigen receptor (CAR), such as a CAR targeting a tumor antigen.

As used herein, the term “chimeric antigen receptor” (CAR) refers to a recombinant polypeptide comprising at least an extracellular domain that binds specifically to a target (e.g., an antigen), a transmembrane domain and an intracellular signaling domain. Engagement of the extracellular domain of the CAR with the target antigen on the surface of a target cell results in clustering of the CAR and delivers an activation stimulus to the CAR-containing cell. CARs redirect the specificity of immune-effector cells and trigger proliferation, cytokine production, phagocytosis and/or production of molecules that can mediate cell death of the target antigen-expressing cell in a major histocompatibility (MHC)-independent manner.

As used herein, the term “signal peptide” refers to a leader sequence at the amino-terminus (N-terminus) of a nascent polypeptide, e.g., an HLA-E and HLA-G polypeptide or a chimeric antigen receptor (CAR) polypeptide disclosed herein, which may direct the nascent protein to the endoplasmic reticulum and subsequent surface expression or secretion.

As used herein, the term “extracellular domain,” “extracellular antigen-binding domain,” or “extracellular ligand-binding domain” refers to the part of a CAR that is located outside of the cell membrane and is capable of binding to a target such as an antigen or a ligand.

As used herein, the term “hinge region” or “hinge domain” refers to the part of a CAR that connects two adjacent domains of the CAR, i.e., the extracellular domain and the transmembrane domain of the CAR.

As used herein, the term “transmembrane domain” refers to the portion of a transmembrane molecule (e.g., CAR or HLA-E and HLA-G molecule of the application) that extends across the cell membrane and anchors the molecule to the cell membrane.

As used herein, the term “intracellular signaling domain,” “cytoplasmic signaling domain,” or “intracellular signaling domain” refers to the part of a CAR that is located inside of the cell membrane and is capable of transducing an effector signal.

As used herein, the term “stimulatory molecule” refers to a molecule expressed by an immune cell (e.g., T cell) that provides the primary cytoplasmic signaling sequence(s) that regulate primary activation of receptors in a stimulatory way for at least some aspect of the immune cell signaling pathway. Stimulatory molecules comprise two distinct classes of cytoplasmic signaling sequence, those that initiate antigen-dependent primary activation (referred to as “primary signaling domains”), and those that act in an antigen-independent manner to provide a secondary of co-stimulatory signal (referred to as “co-stimulatory signaling domains”).

In certain embodiments, the extracellular domain comprises an antigen-binding domain. The antigen-binding domain can, for example, be an antibody or antigen-binding fragment thereof that specifically binds a tumor antigen. The antigen-binding domains of the application possess one or more desirable functional properties, including but not limited to high-affinity binding to a tumor antigen, high specificity to a tumor antigen, the ability to stimulate complement-dependent cytotoxicity (CDC), antibody-dependent phagocytosis (ADPC), and/or antibody-dependent cellular-mediated cytotoxicity (ADCC) against cells expressing a tumor antigen, and the ability to inhibit tumor growth in subjects in need thereof and in animal models when administered alone or in combination with other anti-cancer therapies.

As used herein, the term “antibody” is used in a broad sense and includes immunoglobulin or antibody molecules including human, humanized, composite and chimeric antibodies and antibody fragments that are monoclonal or polyclonal. In general, antibodies are proteins or peptide chains that exhibit binding specificity to a specific antigen. Antibody structures are well known. Immunoglobulins can be assigned to five major classes (i.e., IgA, IgD, IgE, IgG and IgM), depending on the heavy chain constant domain amino acid sequence. IgA and IgG are further sub-classified as the isotypes IgA1, IgA2, IgG1, IgG2, IgG3 and IgG4. Accordingly, the antibodies of the application can be of any of the five major classes or corresponding sub-classes. Preferably, the antibodies of the application are IgG1, IgG2, IgG3 or IgG4. Antibody light chains of vertebrate species can be assigned to one of two clearly distinct types, namely kappa and lambda, based on the amino acid sequences of their constant domains. Accordingly, the antibodies of the application can contain a kappa or lambda light chain constant domain. According to particular embodiments, the antibodies of the application include heavy and/or light chain constant regions from rat or human antibodies. In addition to the heavy and light constant domains, antibodies contain an antigen-binding region that is made up of a light chain variable region and a heavy chain variable region, each of which contains three domains (i.e., complementarity determining regions 1-3; CDR1, CDR2, and CDR3). The light chain variable region domains are alternatively referred to as LCDR1, LCDR2, and LCDR3, and the heavy chain variable region domains are alternatively referred to as HCDR1, HCDR2, and HCDR3.

As used herein, the term an “isolated antibody” refers to an antibody which is substantially free of other antibodies having different antigenic specificities (e.g., an isolated antibody that specifically binds to the specific tumor antigen is substantially free of antibodies that do not bind to the tumor antigen). In addition, an isolated antibody is substantially free of other cellular material and/or chemicals.

As used herein, the term “monoclonal antibody” refers to an antibody obtained from a population of substantially homogeneous antibodies, i.e., the individual antibodies comprising the population are identical except for possible naturally occurring mutations that can be present in minor amounts. The monoclonal antibodies of the application can be made by the hybridoma method, phage display technology, single lymphocyte gene cloning technology, or by recombinant DNA methods. For example, the monoclonal antibodies can be produced by a hybridoma which includes a B cell obtained from a transgenic nonhuman animal, such as a transgenic mouse or rat, having a genome comprising a human heavy chain transgene and a light chain transgene.

As used herein, the term “antigen binding fragment” refers to an antibody fragment such as, for example, a diabody, a Fab, a Fab′, a F(ab′)2, an Fv fragment, a disulfide stabilized Fv fragment (dsFv), a (dsFv)2, a bispecific dsFv (dsFv-dsFv′), a disulfide stabilized diabody (ds diabody), a single-chain antibody molecule (scFv), a single domain antibody (sdAb), a scFv dimer (bivalent diabody), a multispecific antibody formed from a portion of an antibody comprising one or more CDRs, a camelized single domain antibody, a minibody, a nanobody, a domain antibody, a bivalent domain antibody, a light chain variable domain (VL), a variable domain (VHH) of a camelid antibody, or any other antibody fragment that binds to an antigen but does not comprise a complete antibody structure. An antigen binding fragment is capable of binding to the same antigen to which the parent antibody or a parent antibody fragment binds.

As used herein, the term “single-chain antibody” refers to a conventional single-chain antibody in the field, which comprises a heavy chain variable region and a light chain variable region connected by a short peptide of about 15 to about 20 amino acids (e.g., a linker peptide).

As used herein, the term “single domain antibody” refers to a conventional single domain antibody in the field, which comprises a heavy chain variable region and a heavy chain constant region or which comprises only a heavy chain variable region.

As used herein, the term “human antibody” refers to an antibody produced by a human or an antibody having an amino acid sequence corresponding to an antibody produced by a human made using any technique known in the art. This definition of a human antibody includes intact or full-length antibodies, fragments thereof, and/or antibodies comprising at least one human heavy and/or light chain polypeptide.

As used herein, the term “humanized antibody” refers to a non-human antibody that is modified to increase the sequence homology to that of a human antibody, such that the antigen-binding properties of the antibody are retained, but its antigenicity in the human body is reduced.

As used herein, the term “chimeric antibody” refers to an antibody wherein the amino acid sequence of the immunoglobulin molecule is derived from two or more species. The variable region of both the light and heavy chains often corresponds to the variable region of an antibody derived from one species of mammal (e.g., mouse, rat, rabbit, etc.) having the desired specificity, affinity, and capability, while the constant regions correspond to the sequences of an antibody derived from another species of mammal (e.g., human) to avoid eliciting an immune response in that species.

As used herein, the term “multispecific antibody” refers to an antibody that comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In an embodiment, the first and second epitopes are on the same antigen, e.g., the same protein (or subunit of a multimeric protein). In an embodiment, the first and second epitopes overlap or substantially overlap. In an embodiment, the first and second epitopes do not overlap or do not substantially overlap. In an embodiment, the first and second epitopes are on different antigens, e.g., the different proteins (or different subunits of a multimeric protein). In an embodiment, a multispecific antibody comprises a third, fourth, or fifth immunoglobulin variable domain. In an embodiment, a multispecific antibody is a bispecific antibody molecule, a trispecific antibody molecule, or a tetraspecific antibody molecule.

As used herein, the term “bispecific antibody” refers to a multispecific antibody that binds no more than two epitopes or two antigens. A bispecific antibody is characterized by a first immunoglobulin variable domain sequence which has binding specificity for a first epitope and a second immunoglobulin variable domain sequence that has binding specificity for a second epitope. In an embodiment, the first and second epitopes are on the same antigen, e.g., the same protein (or subunit of a multimeric protein). In an embodiment, the first and second epitopes overlap or substantially overlap. In an embodiment, the first and second epitopes are on different antigens, e.g., the different proteins (or different subunits of a multimeric protein). In an embodiment, a bispecific antibody comprises a heavy chain variable domain sequence and a light chain variable domain sequence which have binding specificity for a first epitope and a heavy chain variable domain sequence and a light chain variable domain sequence which have binding specificity for a second epitope. In an embodiment, a bispecific antibody comprises a half antibody, or fragment thereof, having binding specificity for a first epitope and a half antibody, or fragment thereof, having binding specificity for a second epitope. In an embodiment, a bispecific antibody comprises a scFv, or fragment thereof, having binding specificity for a first epitope, and a scFv, or fragment thereof, having binding specificity for a second epitope. In an embodiment, a bispecific antibody comprises a V_{H}H having binding specificity for a first epitope, and a V_{H}H having binding specificity for a second epitope.

As used herein, an antigen-binding domain that “specifically binds to a tumor antigen” refers to an antigen-binding domain that binds a tumor antigen, with a KD of 1×10⁻⁷ M or less, preferably 1×10⁻⁸ M or less, more preferably 5×10⁻⁹ M or less, 1×10⁻⁹ M or less, 5×10⁻¹⁰ M or less, or 1×10⁻¹⁰ M or less. The term “KD” refers to the dissociation constant, which is obtained from the ratio of Kd to Ka (i.e., Kd/Ka) and is expressed as a molar concentration (M). KD values for antibodies can be determined using methods in the art in view of the present disclosure. For example, the KD of an antigen-binding domain can be determined by using surface plasmon resonance, such as by using a biosensor system, e.g., a Biacore® system, or by using bio-layer interferometry technology, such as an Octet RED96 system.

The smaller the value of the KD of an antigen-binding domain, the higher affinity that the antigen-binding domain binds to a target antigen.

In various embodiments, antibodies or antibody fragments suitable for use in the CAR of the present disclosure include, but are not limited to, monoclonal antibodies, bispecific antibodies, multispecific antibodies, chimeric antibodies, polypeptide-Fc fusions, single-chain Fvs (scFv), single chain antibodies, Fab fragments, F(ab′) fragments, disulfide-linked Fvs (sdFv), masked antibodies (e.g., Probodies®), Small Modular ImmunoPharmaceuticals (“SMIPs™”), intrabodies, minibodies, single domain antibody variable domains, nanobodies, VHHs, diabodies, tandem diabodies (TandAb®), anti-idiotypic (anti-Id) antibodies (including, e.g., anti-Id antibodies to antigen-specific TCR), and epitope-binding fragments of any of the above. Antibodies and/or antibody fragments may be derived from murine antibodies, rabbit antibodies, human antibodies, fully humanized antibodies, camelid antibody variable domains and humanized versions, shark antibody variable domains and humanized versions, and camelized antibody variable domains.

In some embodiments, the antigen binding fragment is a Fab fragment, a Fab′ fragment, a F(ab′)2 fragment, a scFv fragment, an Fv fragment, a dsFv diabody, a VHH, a VNAR, a single-domain antibody (sdAb) or nanobody, a dAb fragment, a Fd′ fragment, a Fd fragment, a heavy chain variable region, an isolated complementarity determining region (CDR), a diabody, a triabody, or a decabody. In some embodiments, the antigen binding fragment is an scFv fragment. In some embodiments, the antigen binding fragment is a VHH.

In some embodiments, the CAR is a part of a universal chimeric antigen receptor system having an adaptable receptor specificity component (arCAR). The arCAR system may comprise (i) an immune effector cell having a chimeric antigen receptor comprising a first polypeptide comprising: (a) an extracellular tag-binding domain, (b) a transmembrane domain, and (c) at least one intracellular signaling domain; and (ii) a second polypeptide comprising: (a) an antigen-binding domain that binds to at least one antigen on a target cell, and (b) a tag that is recognized by the extracellular tag-binding domain; wherein: (i) the tag comprises an antibody, an antigen-binding fragment thereof, or an alternative scaffold, and the extracellular tag-binding domain comprises an anti-idiotype molecule that binds to the tag; or (ii) the tag comprises an anti-idiotype molecule that binds to the extracellular tag-binding domain, and the extracellular tag-binding domain comprises an antibody, or antigen-binding fragment thereof, or an alternative scaffold.

In some embodiments, at least one of the extracellular tag-binding domain, the antigen-binding domain, or the tag comprises a single-domain antibody or nanobody.

In some embodiments, at least one of the extracellular tag-binding domain, the antigen-binding domain, or the tag comprises a VHH.

In some embodiments, the extracellular tag-binding domain and the tag each comprise a VHH.

In some embodiments, the extracellular tag-binding domain, the tag, and the antigen-binding domain each comprise a VHH.

In some embodiments, at least one of the extracellular tag-binding domain, the antigen-binding domain, or the tag comprises an scFv.

In some embodiments, the extracellular tag-binding domain and the tag each comprise an scFv.

In some embodiments, the extracellular tag-binding domain, the tag, and the antigen-binding domain each comprise a scFv.

Alternative scaffolds to immunoglobulin domains that exhibit similar functional characteristics, such as high-affinity and specific binding of target biomolecules, may also be used in the CARs of the present disclosure. Such scaffolds have been shown to yield molecules with improved characteristics, such as greater stability or reduced immunogenicity. Non-limiting examples of alternative scaffolds that may be used in the CAR of the present disclosure include engineered, tenascin-derived, tenascin type III domain (e.g., Centyrin™); engineered, gamma-B crystallin-derived scaffold or engineered, ubiquitin-derived scaffold (e.g., Affilins); engineered, fibronectin-derived, 10th fibronectin type III (10Fn3) domain (e.g., monobodies, AdNectins™, or AdNexins™); engineered, ankyrin repeat motif containing polypeptide (e.g., DARPins™); engineered, low-density-lipoprotein-receptor-derived, A domain (LDLR-A) (e.g., Avimers™); lipocalin (e.g., anticalins); engineered, protease inhibitor-derived, Kunitz domain (e.g., EETI-II/AGRP, BPTI/LACI-D1/ITI-D2); engineered, Protein-A-derived, Z domain (Affibodies™); Sac7d-derived polypeptides (e.g., Nanoffitins® or affitins); engineered, Fyn-derived, SH2 domain (e.g., Fynomers®); CTLD₃ (e.g., Tetranectin); thioredoxin (e.g., peptide aptamer); KALBITOR®; the β-sandwich (e.g., iMab); miniproteins; C-type lectin-like domain scaffolds; engineered antibody mimics; and any genetically manipulated counterparts of the foregoing that retains its binding functionality (Worn A, Pluckthun A, J Mol Biol 305: 989-1010 (2001); Xu L et al., Chem Biol 9: 933-42 (2002); Wikman M et al., Protein Eng Des Sel 17: 455-62 (2004); Binz H et al., Nat Biotechnol 23: 1257-68 (2005); Hey T et al., Trends Biotechnol 23: 514-522 (2005); Holliger P, Hudson P, Nat Biotechnol 23: 1126-36 (2005); Gill D, Damle N, Curr Opin Biotech 17: 653-8 (2006); Koide A, Koide S, Methods Mol Biol 352: 95-109 (2007); Skerra, Current Opin. in Biotech., 2007 18: 295-304; Byla P et al., J Biol Chem 285: 12096 (2010); Zoller F et al., Molecules 16: 2467-85 (2011), each of which is incorporated by reference in its entirety for all intended purposes).

In some embodiments, the alternative scaffold is Affilin or Centyrin.

In some embodiments, a CAR of the present disclosure comprises a leader sequence. The leader sequence may be positioned at the N-terminus the extracellular antigen-binding domain. The leader sequence may be optionally cleaved from the extracellular antigen-binding domain during cellular processing and localization of the CAR to the cellular membrane. Any of various leader sequences known to one of skill in the art may be used as the leader sequence. Non-limiting examples of peptides from which the leader sequence may be derived include granulocyte-macrophage colony-stimulating factor receptor (GMCSFR), FcεR, human immunoglobulin (IgG) heavy chain (HC) variable region, CD8α, or any of various other proteins secreted by T cells. In various embodiments, the leader sequence is compatible with the secretory pathway of a T cell. In certain embodiments, the leader sequence is derived from human immunoglobulin heavy chain (HC).

In some embodiments, the leader sequence is derived from GMCSFR. In one embodiment, the GMCSFR leader sequence comprises the amino acid sequence set forth in SEQ ID NO: 133, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 133.

In some embodiments, a CAR of the present disclosure comprises a transmembrane domain, fused in frame between the extracellular domain and the cytoplasmic domain.

The transmembrane domain may be derived from the protein contributing to the extracellular domain, the protein contributing the signaling or co-signaling domain, or by a totally different protein. In some instances, the transmembrane domain can be selected or modified by amino acid substitution, deletions, or insertions to minimize interactions with other members of the CAR complex. In some instances, the transmembrane domain can be selected or modified by amino acid substitution, deletions, or insertions to avoid binding of proteins naturally associated with the transmembrane domain. In certain embodiments, the transmembrane domain includes additional amino acids to allow for flexibility and/or optimal distance between the domains connected to the transmembrane domain.

The transmembrane domain may be derived either from a natural or from a synthetic source. Where the source is natural, the domain may be derived from any membrane-bound or transmembrane protein. Non-limiting examples of transmembrane domains of particular use in this disclosure may be derived from (i.e. comprise at least the transmembrane domain(s) of) the α, β or ζ chain of the T cell receptor (TCR), CD28, CD3 epsilon, CD45, CD4, CD5, CD8, CD8α, CD9, CD16, CD22, CD33, CD37, CD40, CD64, CD80, CD86, CD134, CD137, or CD154. Alternatively, the transmembrane domain may be synthetic, in which case it will comprise predominantly hydrophobic residues such as leucine and valine. For example, a triplet of phenylalanine, tryptophan and/or valine can be found at each end of a synthetic transmembrane domain.

In some embodiments, it will be desirable to utilize the transmembrane domain of the ζ, η or FcεR1γ chains which contain a cysteine residue capable of disulfide bonding, so that the resulting chimeric protein will be able to form disulfide linked dimers with itself, or with unmodified versions of the ζ, η or FcεR1γ chains or related proteins. In some instances, the transmembrane domain will be selected or modified by amino acid substitution to avoid binding of such domains to the transmembrane domains of the same or different surface membrane proteins to minimize interactions with other members of the receptor complex. In other cases, it will be desirable to employ the transmembrane domain of ζ, η or FcεR1γ and -β, MB1 (Igα.), B29 or CD3-γ, ζ, or η, in order to retain physical association with other members of the receptor complex.

In some embodiments, the transmembrane domain is derived from CD8 or CD28. In one embodiment, the CD8 transmembrane domain comprises the amino acid sequence set forth in SEQ ID NO: 154, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 154. In one embodiment, the CD28 transmembrane domain comprises the amino acid sequence set forth in SEQ ID NO: 155, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 155.

In some embodiments, a CAR of the present disclosure comprises a hinge region between the extracellular antigen binding domain and the transmembrane domain, wherein the antigen binding domain, hinge region, and the transmembrane domain are in frame with each other.

The hinge region can comprise any oligo- or polypeptide that functions to link the extracellular antigen binding domain to the transmembrane domain. A hinge region can be used to provide more flexibility and accessibility for the antigen binding domain. A hinge region may comprise up to 300 amino acids, preferably 10 to 100 amino acids and most preferably 25 to 50 amino acids. A hinge region may be derived from all or part of naturally occurring molecules, such as from all or part of the extracellular region of CD8, CD4 or CD28, or from all or part of an antibody constant region. Alternatively, the hinge region may be a synthetic sequence that corresponds to a naturally occurring hinge region sequence, or may be an entirely synthetic hinge region sequence. Non-limiting examples of hinge regions which may be used in accordance to the disclosure include a part of human CD8α chain, partial extracellular domain of CD28, FcγRIIIa receptor, IgG, IgM, IgA, IgD, IgE, an Ig hinge, or functional fragment thereof. In some embodiments, additional linking amino acids are added to the hinge region to ensure that the antigen-binding domain is an optimal distance from the transmembrane domain. In some embodiments, when the spacer is derived from an Ig, the spacer may be mutated to prevent Fc receptor binding.

The hinge domain may be derived from CD8α, CD28, or an immunoglobulin (IgG). For example, the IgG hinge may be from IgG1, IgG2, IgG3, IgG4, IgM1, IgM2, IgA1, IgA2, IgD, IgE, or a chimera thereof.

In certain embodiments, the hinge domain comprises an immunoglobulin IgG hinge or functional fragment thereof. In certain embodiments, the IgG hinge is from IgG1, IgG2, IgG3, IgG4, IgM1, IgM2, IgA1, IgA2, IgD, IgE, or a chimera thereof. In certain embodiments, the hinge domain comprises the CH1, CH2, CH3 and/or hinge region of the immunoglobulin. In certain embodiments, the hinge domain comprises the core hinge region of the immunoglobulin. The term “core hinge” can be used interchangeably with the term “short hinge” (a.k.a “SH”). Non-limiting examples of suitable hinge domains are the core immunoglobulin hinge regions include EPKSCDKTHTCPPCP (SEQ ID NO: 68) from IgG1, ERKCCVECPPCP (SEQ ID NO: 69) from IgG2, ELKTPLGDTTHTCPRCP(EPKSCDTPPPCPRCP)₃ (SEQ ID NO: 70) from IgG3, and ESKYGPPCPSCP (SEQ ID NO: 71) from IgG4 (see also Wypych et al., JBC 2008 283(23): 16194-16205, which is incorporated herein by reference in its entirety for all purposes). In certain embodiments, the hinge domain is a fragment of the immunoglobulin hinge.

In some embodiments, the hinge domain is derived from CD8 or CD28. In one embodiment, the CD8 hinge domain comprises the amino acid sequence set forth in SEQ ID NO: 152, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 152. In one embodiment, the CD28 hinge domain comprises the amino acid sequence set forth in SEQ ID NO: 153, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 153.

In some embodiments, the transmembrane domain and/or hinge domain is derived from CD8 or CD28. In some embodiments, both the transmembrane domain and hinge domain are derived from CD8. In some embodiments, both the transmembrane domain and hinge domain are derived from CD28. Non-limiting exemplary hinge sequences are provided in Table 5.

TABLE 5
Hinge Sequences

| | SEQ ID |
|---|---|
| Sequence | NO |
| EPKSCDKTHTCPPCP | 68 |
| ERKCCVECPPCP | 69 |
| ELKTPLGDTTHTCPRCPEPKSCDTPPPCPRCPEPKSCDTPPPCP | 70 |
| RCPEPKSCDTPPPCPRCP | |
| ESKYGPPCPSCP | 71 |

In certain aspects, a CAR of the present disclosure comprise a cytoplasmic domain, which comprises at least one intracellular signaling domain. In some embodiments, cytoplasmic domain also comprises one or more co-stimulatory signaling domains.

The cytoplasmic domain is responsible for activation of at least one of the normal effector functions of the host cell (e.g., T cell) in which the CAR has been placed in. The term “effector function” refers to a specialized function of a cell. Effector function of a T cell, for example, may be cytolytic activity or helper activity including the secretion of cytokines. Thus, the term “signaling domain” refers to the portion of a protein which transduces the effector function signal and directs the cell to perform a specialized function. While usually the entire signaling domain is present, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the signaling domain sufficient to transduce the effector function signal.

Non-limiting examples of signaling domains which can be used in the CARs of the present disclosure include, e.g., signaling domains derived from DAP10, DAP12, Fc epsilon receptor I γ chain (FCER1G), FcR β, CD3δ, CD3ε, CD3γ, CD3ζ, CD5, CD22, CD226, CD66d, CD79A, and CD79B.

In some embodiments, the cytoplasmic domain comprises a CD3ζ signaling domain. In one embodiment, the CD3ζ signaling domain comprises the amino acid sequence set forth in SEQ ID NO: 137, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 137.

In some embodiments, the cytoplasmic domain further comprises one or more co-stimulatory signaling domains. In some embodiments, the one or more co-stimulatory signaling domains are derived from CD28, 41BB, IL2Rb, CD40, OX40 (CD134), CD80, CD86, CD27, ICOS, NKG2D, DAP10, DAP12, 2B4 (CD244), BTLA, CD30, GITR, CD226, CD79A, and HVEM.

In one embodiment, the co-stimulatory signaling domain is derived from 41BB. In one embodiment, the 41BB co-stimulatory signaling domain comprises the amino acid sequence set forth in SEQ ID NO: 139, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 139.

In one embodiment, the co-stimulatory signaling domain is derived from IL2Rb. In one embodiment, the IL2Rb co-stimulatory signaling domain comprises the amino acid sequence set forth in SEQ ID NO: 140, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 140.

In one embodiment, the co-stimulatory signaling domain is derived from CD40. In one embodiment, the CD40 co-stimulatory signaling domain comprises the amino acid sequence set forth in SEQ ID NO: 141, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 141.

In one embodiment, the co-stimulatory signaling domain is derived from OX40. In one embodiment, the OX40 co-stimulatory signaling domain comprises the amino acid sequence set forth in SEQ ID NO: 142, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 142.

In one embodiment, the co-stimulatory signaling domain is derived from CD80. In one embodiment, the CD80 co-stimulatory signaling domain comprises the amino acid sequence set forth in SEQ ID NO: 143, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 143.

In one embodiment, the co-stimulatory signaling domain is derived from CD86. In one embodiment, the CD86 co-stimulatory signaling domain comprises the amino acid sequence set forth in SEQ ID NO: 144, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 144.

In one embodiment, the co-stimulatory signaling domain is derived from CD27. In one embodiment, the CD27 co-stimulatory signaling domain comprises the amino acid sequence set forth in SEQ ID NO: 145, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 145.

In one embodiment, the co-stimulatory signaling domain is derived from ICOS. In one embodiment, the ICOS co-stimulatory signaling domain comprises the amino acid sequence set forth in SEQ ID NO: 146, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 146.

In one embodiment, the co-stimulatory signaling domain is derived from NKG2D. In one embodiment, the NKG2D co-stimulatory signaling domain comprises the amino acid sequence set forth in SEQ ID NO: 147, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 147.

In one embodiment, the co-stimulatory signaling domain is derived from DAP10. In one embodiment, the DAP10 co-stimulatory signaling domain comprises the amino acid sequence set forth in SEQ ID NO: 148, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 148.

In one embodiment, the co-stimulatory signaling domain is derived from DAP12. In one embodiment, the DAP12 co-stimulatory signaling domain comprises the amino acid sequence set forth in SEQ ID NO: 149, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 149.

In one embodiment, the co-stimulatory signaling domain is derived from 2B4 (CD244). In one embodiment, the 2B4 (CD244) co-stimulatory signaling domain comprises the amino acid sequence set forth in SEQ ID NO: 150, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 150.

In one embodiment, the co-stimulatory signaling domain is derived from CD28. In one embodiment, the CD28 co-stimulatory signaling domain comprises the amino acid sequence set forth in SEQ ID NO: 151, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 151.

In one embodiment, the CAR of the present disclosure comprises a hinge region, a transmembrane domain and a co-stimulatory signaling domain all derived from CD28. In one embodiment, the hinge region, transmembrane domain and co-stimulatory signaling domain derived from CD28 comprises the amino acid sequence set forth in SEQ ID NO: 136, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 136.

In some embodiments, the CAR of the present disclosure comprises one costimulatory signaling domains. In some embodiments, the CAR of the present disclosure comprises two or more costimulatory signaling domains. In certain embodiments, the CAR of the present disclosure comprises two, three, four, five, six or more costimulatory signaling domains.

In some embodiments, the signaling domain(s) and costimulatory signaling domain(s) can be placed in any order. In some embodiments, the signaling domain is upstream of the costimulatory signaling domains. In some embodiments, the signaling domain is downstream from the costimulatory signaling domains. In the cases where two or more costimulatory domains are included, the order of the costimulatory signaling domains could be switched.

Non-limiting exemplary CAR regions and sequences are provided in Table 6.

TABLE 6
Non-limiting Exemplary CAR regions and sequences

| | | | |
|---|---|---|---|
| | | | SEQ ID |
| CAR regions | Sequence | UniProt Id | NO |

CD19 CAR:

| | | | |
|---|---|---|---|
| GMCSFR | MLLLVTSLLLCELPHPAFLLIP | | 133 |
| Signal Peptide | | | |
| FMC63 VH | EVKLQESGPGLVAPSQSLSVTCTVSGVSLPDYGVSW | | 134 |
| | IRQPPRKGLEWLGVIWGSETTYYNSALKSRLTIIKDN | | |
| | SKSQVFLKMNSLQTDDTAIYYCAKHYYYGGSYAM | | |
| | DYWGQGTSVTVSS | | |
| Whitlow | GSTSGSGKPGSGEGSTKG | | 33 |
| Linker | | | |
| FMC63 VL | DIQMTQTTSSLSASLGDRVTISCRASQDISKYLNWY | | 135 |
| | QQKPDGTVKLLIYHTSRLHSGVPSRFSGSGSGTDYS | | |
| | LTISNLEQEDIATYFCQQGNTLPYTFGGGTKLEIT | | |
| CD28 | IEVMYPPPYLDNEKSNGTIIHVKGKHLCPSPLFPGPS | P10747-1 | 136 |
| (AA 114-220) | KPFWVLVVVGGVLACYSLLVTVAFIIFWVRSKRSR | | |
| | LLHSDYMNMTPRRPGPTRKHYQPYAPPRDFAAYRS | | |
| CD3-zeta | RVKFSRSADAPAYQQGQNQLYNELNLGRREEYDV | P20963-3 | 137 |
| isoform 3 | LDKRRGRDPEMGGKPRRKNPQEGLYNELQKDKMA | | |
| (AA 52-163) | EAYSEIGMKGERRRGKGHDGLYQGLSTATKDTYD | | |
| | ALHMQALPPR | | |
| FMC63 scFV | EVKLQESGPGLVAPSQSLSVTCTVSGVSLPDYGVS | | 138 |
| | WIRQPPRKGLEWLGVIWGSETTYYNSALKSRLTIIK | | |
| | DNSKSQVFLKMNSLQTDDTAIYYCAKHYYYGGSY | | |
| | AMDYWGQGTSVTVSSGSTSGSGKPGSGEGSTKGDI | | |
| | QMTQTTSSLSASLGDRVTISCRASQDISKYLNWYQQ | | |
| | KPDGTVKLLIYHTSRLHSGVPSRFSGSGSGTDYSLTI | | |
| | SNLEQEDIATYFCQQGNTLPYTFGGGTKLEIT | | |

Signaling Domains:

| | | | |
|---|---|---|---|
| 41BB | KRGRKKLLYIFKQPFMRPVQTTQEEDGCSCRFPEEE | Q07011 | 139 |
| (AA 214-255) | EGGCEL | | |
| IL2Rb | NCRNTGPWLKKVLKCNTPDPSKFFSQLSSEHGGDV | P14784 | 140 |
| (AA 266-551) | QKWLSSPFPSSSFSPGGLAPEISPLEVLERDKVTQLL | | |
| | PLNTDAYLSLQELQGQDPTHLV | | |
| CD40 | KKVAKKPTNKAPHPKQEPQEINFPDDLPGSNTAAPV | P25942 | 141 |
| (AA 216-277) | QETLHGCQPVTQEDGKESRISVQERQ | | |
| OX40 | ALYLLRRDQRLPPDAHKPPGGGSFRTPIQEEQADAH | P43489 | 142 |
| (AA 236-277) | STLAKI | | |
| CD80 | TYCFAPRCRERRRNERLRRESVRPV | P33681 | 143 |
| (AA 264-288) | | | |
| CD86 | KWKKKKRPRNSYKCGTNTMEREESEQTKKREKIHI | P42081 | 144 |
| (AA269-329) | PERSDEAQRVFKSSKTSSCDKSDTCF | | |
| CD27 | QRRKYRSNKGESPVEPAEPCHYSCPREEEGSTIPIQE | P26842 | 145 |
| (AA 213-260) | DYRKPEPACSP | | |
| ICOS | CWLTKKKYSSSVHDPNGEYMFMRAVNTAKKSRLT | Q9Y6W8 | 146 |
| (AA 162-199) | DVTL | | |
| NKG2D | MGWIRGRRSRHSWEMSEFHNYNLDLKKSDF | P26718 | 147 |
| (AA 1-51) | STRWQKQRCPVVKSKCRENAS | | |
| DAP10 | LCARPRRSPAQEDGKVYINMPGRG | Q9UBK5 | 148 |
| (AA 70-93) | | | |
| DAP12 | YFLGRLVPRGRGAAEAATRKQRITETESPYQELQGQ | O54885 | 149 |
| (AA 62-113) | RSDVYSDLNTQRPYYK | | |
| 2B4/CD244 | WRRKRKEKQSETSPKEFLTIYEDVKDLKTRRNHEQ | Q9BZW8 | 150 |
| (AA 251-370) | EQTFPGGGSTIYSMIQSQSSAPTSQEPAYTLYSLIQPS | | |
| | RKSGSRKRNHSPSFNSTIYEVIGKSQPKAQNPARLSR | | |
| | KELENFDVYS | | |
| CD3-zeta | RVKFSRSADAPAYQQGQNQLYNELNLGRREEYDV | P20963-3 | 137 |
| isoform 3 | LDKRRGRDPEMGGKPRRKNPQEGLYNELQKDKMA | | |
| (AA 52-163) | EAYSEIGMKGERRRGKGHDGLYQGLSTATKDTYD | | |
| | ALHMQALPPR | | |
| CD28 | RSKRSRLLHSDYMNMTPRRPGPTRKHYQPYAPPRD | P10747-1 | 151 |
| (AA 180-220) | FAAYRS | | |

Spacer/Hinge:

| | | | |
|---|---|---|---|
| CD8 | TTTPAPRPPTPAPTIASQPLSLRPEACRPAAGGAVHT | P01732 | 152 |
| (AA 136-182) | RGLDFACDIY | | |
| CD28 | IEVMYPPPYLDNEKSNGTIIHVKGKHLCPSPLFPGPS | P10747-1 | 153 |
| (AA 114-151) | KP | | |

Transmembrane:

| | | | |
|---|---|---|---|
| CD8 | IYIWAPLAGTCGVLLLSLVIT | P01732 | 154 |
| (AA 183-203) | | | |
| CD28 | FWVLVVVGGVLACYSLLVTVAFIIFWV | P10747-1 | 155 |
| (AA 153-179) | | | |

Linkers:

| | | |
|---|---|---|
| Whitlow | GSTSGSGKPGSGEGSTKG | 33 |
| Linker | | |
| (G₄S)₃ | GGGGSGGGGSGGGGS | 6 |
| Linker 3 | GGSEGKSSGSGSESKSTGGS | 34 |
| Linker 4 | GGGSGGGS | 35 |
| Linker 5 | GGGSGGGSGGGS | 36 |
| Linker 6 | GGGSGGGSGGGSGGGS | 37 |
| Linker 7 | GGGSGGGSGGGSGGGSGGGS | 38 |
| Linker 8 | GGGGSGGGGSGGGGSGGGGS | 39 |
| Linker 9 | GGGGSGGGGSGGGGSGGGGSGGGGS | 40 |
| Linker 10 | IRPRAIGGSKPRVA | 45 |
| Linker 11 | GKGGSGKGGSGKGGS | 46 |
| Linker 12 | GGKGSGGKGSGGKGS | 47 |
| Linker 13 | GGGKSGGGKSGGGKS | 48 |
| Linker 14 | GKGKSGKGKSGKGKS | 49 |
| Linker 15 | GGGKSGGKGSGKGGS | 50 |
| Linker 16 | GKPGSGKPGSGKPGS | 51 |
| Linker 17 | GKPGSGKPGSGKPGSGKPGS | 52 |
| Linker 18 | GKGKSGKGKSGKGKSGKGKS | 53 |
| Linker 19 | STAGDTHLGGEDFD | 54 |
| Linker 20 | GEGGSGEGGSGEGGS | 55 |
| Linker 21 | GGEGSGGEGSGGEGS | 56 |
| Linker 22 | GEGESGEGESGEGES | 57 |
| Linker 23 | GGGESGGEGSGEGGS | 58 |
| Linker 24 | GEGESGEGESGEGESGEGES | 59 |
| Linker 25 | PRGASKSGSASQTGSAPGS | 60 |
| Linker 26 | GTAAAGAGAAGGAAAGAAG | 61 |
| Linker 27 | GTSGSSGSGSGGSGSGGGG | 62 |
| Linker 28 | GSGS | 63 |
| Linker 29 | APAPAPAPAP | 64 |
| Linker 30 | APAPAPAPAPAPAPAPAPAP | 65 |
| Linker 31 | AEAAAKEAAAKEAAAAKEAAAAKEAAAAKAAA | 66 |

In some embodiments, the antigen-binding domain of a CAR described herein binds to an antigen. The antigen-binding domain of a CAR described herein may bind to more than one antigen or more than one epitope in an antigen. For example, the antigen-binding domain of a CAR described herein may bind to two, three, four, five, six, seven, eight or more antigens. As another example, the antigen-binding domain of a CAR described herein may bind to two, three, four, five, six, seven, eight or more epitopes in the same antigen.

The choice of antigen-binding domain may depend upon the type and number of antigens that define the surface of a target cell. For example, the antigen-binding domain may be chosen to recognize an antigen that acts as a cell surface marker on target cells associated with a particular disease state. In certain embodiments, the CARs of the present disclosure can be genetically modified to target a tumor antigen of interest by way of engineering a desired antigen-binding domain that specifically binds to an antigen (e.g., on a tumor cell). Non-limiting examples of cell surface markers that may act as targets for the antigen-binding domain in the CAR of the disclosure include those associated with tumor cells or autoimmune diseases.

In some embodiments, the antigen-binding domain binds to at least one tumor antigen or autoimmune antigen.

In some embodiments, the antigen-binding domain binds to at least one tumor antigen. In some embodiments, the antigen-binding domain binds to two or more tumor antigens. In some embodiments, the two or more tumor antigens are associated with the same tumor. In some embodiments, the two or more tumor antigens are associated with different tumors.

In some embodiments, the antigen-binding domain binds to at least one autoimmune antigen. In some embodiments, the antigen-binding domain binds to two or more autoimmune antigens. In some embodiments, the two or more autoimmune antigens are associated with the same autoimmune disease. In some embodiments, the two or more autoimmune antigens are associated with different autoimmune diseases.

In some embodiments, the tumor antigen is associated with glioblastoma, ovarian cancer, cervical cancer, head and neck cancer, liver cancer, prostate cancer, pancreatic cancer, renal cell carcinoma, bladder cancer, or hematologic malignancy. Non-limiting examples of tumor antigen associated with glioblastoma include HER2, EGFRVIII, EGFR, CD133, PDGFRA, FGFR1, FGFR3, MET, CD70, ROBO1 and IL13Rα2. Non-limiting examples of tumor antigens associated with ovarian cancer include FOLR1, FSHR, MUC16, MUC1, Mesothelin, CA125, EpCAM, EGFR, PDGFRα, Nectin-4, and B7H4. Non-limiting examples of the tumor antigens associated with cervical cancer or head and neck cancer include GD2, MUC1, Mesothelin, HER2, and EGFR. Non-limiting examples of tumor antigen associated with liver cancer include Claudin 18.2, GPC-3, EpCAM, cMET, and AFP. Non-limiting examples of tumor antigens associated with hematological malignancies include CD22, CD79, BCMA, GPRC5D, SLAM F7, CD33, CLL1, CD123, and CD70. Non-limiting examples of tumor antigens associated with bladder cancer include Nectin-4 and SLITRK6.

Additional examples of antigens that may be targeted by the antigen-binding domain include, but are not limited to, alpha-fetoprotein, A3, antigen specific for A33 antibody, Ba 733, BrE3-antigen, carbonic anhydrase EX, CD1, CD1a, CD3, CD5, CD15, CD16, CD19, CD20, CD21, CD22, CD23, CD25, CD30, CD33, CD38, CD45, CD74, CD79a, CD80, CD123, CD138, colon-specific antigen-p (CSAp), CEA (CEACAM5), CEACAM6, CSAp, EGFR, EGP-I, EGP-2, Ep-CAM, EphA1, EphA2, EphA3, EphA4, EphA5, EphA6, EphA7, EphA8, EphA10, EphB1, EphB2, EphB3, EphB4, EphB6, FIt-I, Flt-3, folate receptor, HLA-DR, human chorionic gonadotropin (HCG) and its subunits, hypoxia inducible factor (HIF-I), Ia, IL-2, IL-6, IL-8, insulin growth factor-1 (IGF-I), KC4-antigen, KS-1-antigen, KS1-4, Le-Y, macrophage inhibition factor (MIF), MAGE, MUC2, MUC3, MUC4, NCA66, NCA95, NCA90, antigen specific for PAM-4 antibody, placental growth factor, p53, prostatic acid phosphatase, PSA, PSMA, RS5, S100, TAC, TAG-72, tenascin, TRAIL receptors, Tn antigen, Thomson-Friedenreich antigens, tumor necrosis antigens, VEGF, ED-B fibronectin, 17-1A-antigen, an angiogenesis marker, an oncogene marker or an oncogene product.

In one embodiment, the antigen targeted by the antigen-binding domain is CD19. In one embodiment, the antigen-binding domain comprises an anti-CD19 scFv. In one embodiment, the anti-CD19 scFv comprises a heavy chain variable region (VH) comprising the amino acid sequence set forth in SEQ ID NO: 134, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 134. In one embodiment, the anti-CD19 scFv comprises a light chain variable region (VL) comprising the amino acid sequence set forth in SEQ ID NO: 135, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 135. In one embodiment, the anti-CD19 scFv comprises the amino acid sequence set forth in SEQ ID NO: 138, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 138.

In some embodiments, the antigen is associated with an autoimmune disease or disorder. Such antigens may be derived from cell receptors and cells which produce “self”-directed antibodies. In some embodiments, the antigen is associated with an autoimmune disease or disorder such as Rheumatoid arthritis (RA), multiple sclerosis (MS), Sjögren's syndrome, Systemic lupus erythematosus, sarcoidosis, Type 1 diabetes mellitus, insulin dependent diabetes mellitus (IDDM), autoimmune thyroiditis, reactive arthritis, ankylosing spondylitis, scleroderma, polymyositis, dermatomyositis, psoriasis, vasculitis, Wegener's granulomatosis, Myasthenia gravis, Hashimoto's thyroiditis, Graves' disease, chronic inflammatory demyelinating polyneuropathy, Guillain-Barre syndrome, Crohn's disease or ulcerative colitis.

In some embodiments, autoimmune antigens that may be targeted by the CAR disclosed herein include but are not limited to platelet antigens, myelin protein antigen, Sm antigens in snRNPs, islet cell antigen, Rheumatoid factor, and anticitrullinated protein. citrullinated proteins and peptides such as CCP-1, CCP-2 (cyclical citrullinated peptides), fibrinogen, fibrin, vimentin, filaggrin, collagen I and II peptides, alpha-enolase, translation initiation factor 4G1, perinuclear factor, keratin, Sa (cytoskeletal protein vimentin), components of articular cartilage such as collagen II, IX, and XI, circulating serum proteins such as RFs (IgG, IgM), fibrinogen, plasminogen, ferritin, nuclear components such as RA33/hnRNP A2, Sm, eukaryotic translation elongation factor 1 alpha 1, stress proteins such as HSP-65, -70, -90, BiP, inflammatory/immune factors such as B7-H1, IL-1 alpha, and IL-8, enzymes such as calpastatin, alpha-enolase, aldolase-A, dipeptidyl peptidase, osteopontin, glucose-6-phosphate isomerase, receptors such as lipocortin 1, neutrophil nuclear proteins such as lactoferrin and 25-35 kD nuclear protein, granular proteins such as bactericidal permeability increasing protein (BPI), elastase, cathepsin G, myeloperoxidase, proteinase 3, platelet antigens, myelin protein antigen, islet cell antigen, rheumatoid factor, histones, ribosomal P proteins, cardiolipin, vimentin, nucleic acids such as dsDNA, ssDNA, and RNA, ribonuclear particles and proteins such as Sm antigens (including but not limited to SmD's and SmB′/B), UIRNP, A2/B1 hnRNP, Ro (SSA), and La (SSB) antigens.

In various embodiments, the scFv fragment used in the CAR of the present disclosure may include a linker between the VH and VL domains. The linker can be a peptide linker and may include any naturally occurring amino acid. Exemplary amino acids that may be included into the linker are Gly, Ser Pro, Thr, Glu, Lys, Arg, Ile, Leu, His and The. The linker should have a length that is adequate to link the VH and the VL in such a way that they form the correct conformation relative to one another so that they retain the desired activity, such as binding to an antigen. The linker may be about 5-50 amino acids long. In some embodiments, the linker is about 10-40 amino acids long. In some embodiments, the linker is about 10-35 amino acids long. In some embodiments, the linker is about 10-30 amino acids long. In some embodiments, the linker is about 10-25 amino acids long. In some embodiments, the linker is about 10-20 amino acids long. In some embodiments, the linker is about 15-20 amino acids long. Exemplary linkers that may be used are Gly rich linkers, Gly and Ser containing linkers, Gly and Ala containing linkers, Ala and Ser containing linkers, and other flexible linkers.

In one embodiment, the linker is a Whitlow linker. In one embodiment, the Whitlow linker comprises the amino acid sequence set forth in SEQ ID NO: 33, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 33. In another embodiment, the linker is a (G₄S)₃ linker (SEQ ID NO: 6). In one embodiment, the (G₄S)₃ linker (SEQ ID NO: 6) comprises the amino acid sequence set forth in SEQ ID NO: 6, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 6. In another embodiment, the linker is a (G₄S)₂ linker (SEQ ID NO: 41). In one embodiment, the (G₄S)₂ linker (SEQ ID NO: 41) comprises the amino acid sequence set forth in SEQ ID NO: 41, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 41. In another embodiment, the linker is a (G₄S)₄ linker (SEQ ID NO: 39). In one embodiment, the (G₄S)₄ linker (SEQ ID NO: 39) comprises the amino acid sequence set forth in SEQ ID NO: 39, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 39.

Other linker sequences may include portions of immunoglobulin hinge area, CL or CH1 derived from any immunoglobulin heavy or light chain isotype. Exemplary linkers that may be used include any of in the sequences set forth in Table 4 or Table 6 of the present disclosure. Additional linkers are described for example in Int. Pat. Publ. No. WO2019/060695, incorporated by reference herein in its entirety for all intended purposes.

### Regulatory Elements

In certain embodiments, the polynucleotide encoding the MAD7 nuclease, the gRNA, or the exogenous polynucleotide, e.g., HLA-E and HLA-G transgene, for insertion is operably linked to at least a regulatory element. The regulatory element can be capable of mediating expression of the MAD7, gRNA, and/or the transgene in the host cell. Regulatory elements include, but are not limited to, promoters, enhancers, initiation sites, polyadenylation (polyA) tails, IRES elements, response elements, and termination signals.

In some embodiments, the exogenous polynucleotides for insertion are operatively linked to (1) one or more exogenous promoters comprising CMV, EF1a, PGK, CAG, UBC, SV40, human beta actin, or other constitutive, inducible, temporal-, tissue-, or cell type-specific promoters; or (2) one or more endogenous promoters comprised in the selected sites such as AAVS1, B2M, CIITA, NKG2A, TRAC, CD70, CD38, CD33, or CLYBL, or other loci meeting the criteria of a genome safe harbor.

In some embodiments, the promoter is a CAG promoter. In some embodiments, the CAG promoter comprises the polynucleotide sequence at least 80%, such as at least 80%, 81%, 82%, 83%, 84%, 85%, 86%, 87%, 89%, 90%, 91%, 92%, 93%, 94%, 95%, 96%, 97%, 98%, 99% or 100%, identical to SEQ ID NO: 92.

In some embodiment, the exogenous polynucleotides for insertion are placed operably under the control of a Kozak consensus sequence. In some embodiments, the Kozak sequence comprises the polynucleotide sequence of GCCACC, or a variant thereof.

In certain embodiments, the exogenous polynucleotides for insertion are operatively linked to a terminator/polyadenylation signal. In some embodiments, the terminator/polyadenylation signal is a SV40 signal. In certain embodiments, the SV40 signal comprises the polynucleotide sequence at least 80%, such as at least 80%, 81%, 82%, 83%, 84%, 85%, 86%, 87%, 89%, 90%, 91%, 92%, 93%, 94%, 95%, 96%, 97%, 98%, 99% or 100%, identical to SEQ ID NO: 93. Other terminator sequences can also be used, examples of which include, but are not limited to BGH, hGH, and PGK.

### Other Optional Genome Edits

In other embodiments of the above described cell, the genomic editing employing the RNP complex of this disclosure may comprise insertions of one or more exogenous polynucleotides encoding other additional artificial cell death polypeptides proteins, targeting modalities, receptors, signaling molecules, transcription factors, pharmaceutically active proteins and peptides, drug target candidates, or proteins promoting engraftment, trafficking, homing, viability, self-renewal, persistence, and/or survival of the genome-engineered iPSCs or derivative cells thereof. Other transgene inserts may include those encoding PET reporters, homeostatic cytokines, and inhibitory checkpoint inhibitory proteins such as PD1, PD-L1, and CTLA4 as well as proteins that target the CD47/signal regulatory protein alpha (SIRPα) axis.

### VIII. Compositions

In another general aspect, the application provides a composition comprising an isolated polynucleotide of the application, a host cell and/or an iPSC or derivative cell thereof of the application.

In certain embodiments, the composition further comprises one or more therapeutic agents selected from the group consisting of a peptide, a cytokine, a checkpoint inhibitor, a mitogen, a growth factor, a small RNA, a dsRNA (double stranded RNA), mononuclear blood cells, feeder cells, feeder cell components or replacement factors thereof, a vector comprising one or more polynucleic acids of interest, an antibody, a chemotherapeutic agent or a radioactive moiety, or an immunomodulatory drug (ImiD).

In certain embodiments, the composition is a pharmaceutical composition comprising an isolated polynucleotide of the application, a host cell and/or an iPSC or derivative cell thereof of the application and a pharmaceutically acceptable carrier. The term “pharmaceutical composition” as used herein means a product comprising an isolated polynucleotide of the application, an isolated polypeptide of the application, a host cell of the application, and/or an iPSC or derivative cell thereof of the application together with a pharmaceutically acceptable carrier. Polynucleotides, polypeptides, host cells, and/or iPSCs or derivative cells thereof of the application and compositions comprising them are also useful in the manufacture of a medicament for therapeutic applications mentioned herein.

In certain aspects, the present invention provides a pharmaceutical composition comprising an isolated host cell disclosed herein. In certain aspects, the present invention provides a pharmaceutical composition comprising an immune-effector cell derived from an iPSC disclosed herein.

As used herein, the term “carrier” refers to any excipient, diluent, filler, salt, buffer, stabilizer, solubilizer, oil, lipid, lipid containing vesicle, microsphere, liposomal encapsulation, or other material well known in the art for use in pharmaceutical formulations. It will be understood that the characteristics of the carrier, excipient or diluent will depend on the route of administration for a particular application. As used herein, the term “pharmaceutically acceptable carrier” refers to a non-toxic material that does not interfere with the effectiveness of a composition described herein or the biological activity of a composition described herein. According to particular embodiments, in view of the present disclosure, any pharmaceutically acceptable carrier suitable for use in a polynucleotide, polypeptide, host cell, and/or iPSC or derivative cell thereof can be used.

The formulation of pharmaceutically active ingredients with pharmaceutically acceptable carriers is known in the art, e.g., Remington: The Science and Practice of Pharmacy (e.g. 21^{st} edition (2005), and any later editions). Non-limiting examples of additional ingredients include: buffers, diluents, solvents, tonicity regulating agents, preservatives, stabilizers, and chelating agents. One or more pharmaceutically acceptable carrier may be used in formulating the pharmaceutical compositions of the application.

### IX. Methods of Use

In another general aspect, the application provides a method of preventing or treating a disease or a condition in a subject in need thereof. The methods comprise administering to the subject in need thereof a therapeutically effective amount of any of the cells, e.g., host cells and/or immune-effector cells or population thereof, of the application, and/or a composition, e.g., a pharmaceutical composition, of the application. In some embodiments, the composition may be, for example, a pharmaceutical composition comprising an isolated host cell or immuno-effector cell derived from an iPSC disclosed herein.

In certain embodiments, the disease or condition is cancer.

In certain aspects, the present disclosure provides a method for preventing or treating a cancer, the method comprising administering to an individual in need thereof, a pharmaceutically effective amount of the host cell, immune-effector cell, or the population thereof disclosed herein, and/or a pharmaceutical composition disclosed herein.

The cancer can, for example, be a solid or a liquid cancer. The cancer, can, for example, be selected from the group consisting of a lung cancer, a gastric cancer, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, an endometrial cancer, a prostate cancer, a thyroid cancer, a glioma, a glioblastoma, and other solid tumors, and a non-Hodgkin's lymphoma (NHL), Hodgkin's lymphoma/disease (HD), an acute lymphocytic leukemia (ALL), a chronic lymphocytic leukemia (CLL), a chronic myelogenous leukemia (CML), a multiple myeloma (MM), an acute myeloid leukemia (AML), and other liquid tumors.

In some embodiments, the cancer may be, for example, without limitation, a lung cancer, pancreatic cancer, liver cancer, melanoma, bone cancer, breast cancer, colon cancer, leukemia, uterine cancer, ovarian cancer, lymphoma, and brain cancer.

In some embodiments, the cancer is selected from the group consisting of leukemias, such as AML, CML, ALL and CLL, lymphomas, such as Hodgkin lymphoma, non-Hodgkin lymphoma and multiple myeloma, and solid cancers such as sarcomas, skin cancer, melanoma, bladder cancer, brain cancer, breast cancer, uterus cancer, ovary cancer, prostate cancer, lung cancer, colorectal cancer, cervical cancer, liver cancer, head and neck cancer, esophageal cancer, pancreatic cancer, renal cancer, adrenal cancer, stomach cancer, testicular cancer, cancer of the gall bladder and biliary tracts, thyroid cancer, thymus cancer, cancer of bone, and cerebral cancer, as well as cancer of unknown primary (CUP).

Primary cancer cells can be readily distinguished from non-cancerous cells by well-established techniques, particularly histological examination. The definition of a “cancer cell”, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g., by procedures such as computed tomography (CT) scan, magnetic resonance imaging (MRI), X-ray, ultrasound or palpation on physical examination, and/or which is detectable because of the expression of one or more cancer-specific antigens in a sample obtainable from a patient.

Cancer conditions may be characterized by the abnormal proliferation of malignant cancer cells and may include leukemias, such as AML, CML, ALL and CLL, lymphomas, such as Hodgkin lymphoma, non-Hodgkin lymphoma and multiple myeloma, and solid cancers such as sarcomas, skin cancer, melanoma, bladder cancer, brain cancer, breast cancer, uterus cancer, ovary cancer, prostate cancer, lung cancer, colorectal cancer, cervical cancer, liver cancer, head and neck cancer, esophageal cancer, pancreatic cancer, renal cancer, adrenal cancer, stomach cancer, testicular cancer, cancer of the gall bladder and biliary tracts, thyroid cancer, thymus cancer, cancer of bone, and cerebral cancer, as well as cancer of unknown primary (CUP).

Cancer cells within an individual may be immunologically distinct from normal somatic cells in the individual (i.e. the cancerous tumor may be immunogenic). For example, the cancer cells may be capable of eliciting a systemic immune response in the individual against one or more antigens expressed by the cancer cells. The tumor antigens that elicit the immune response may be specific to cancer cells or may be shared by one or more normal cells in the individual.

The cancer cells of an individual suitable for treatment as described herein may express the antigen and/or may be of correct HLA type to bind the antigen receptor expressed by the T cells.

An individual suitable for treatment as described above may be a mammal. In preferred embodiments, the individual is a human. In other preferred embodiments, non-human mammals, especially mammals that are conventionally used as models for demonstrating therapeutic efficacy in humans (e.g. murine, primate, porcine, canine, or rabbit animals) may be employed.

In some embodiments, the individual may have minimal residual disease (MRD) after an initial cancer treatment. In some embodiments, the individual may have no minimal residual disease after one or more cancer treatments or repeated dosing.

An individual with cancer may display at least one identifiable sign, symptom, or laboratory finding that is sufficient to make a diagnosis of cancer in accordance with clinical standards known in the art. Examples of such clinical standards can be found in textbooks of medicine such as Harrison's Principles of Internal Medicine, 15^{th} Ed., Fauci A S et al., eds., McGraw-Hill, New York, 2001. In some instances, a diagnosis of a cancer in an individual may include identification of a particular cell type (e.g. a cancer cell) in a sample of a body fluid or tissue obtained from the individual.

An anti-tumor effect is a biological effect which can be manifested by a reduction in the rate of tumor growth, decrease in tumor volume, a decrease in the number of tumor cells, a decrease in the number of metastases, an increase in life expectancy, or amelioration of various physiological symptoms associated with the cancerous condition. An “anti-tumor effect” can also be manifested by the ability of the peptides, polynucleotides, cells and antibodies, also T cells which may be obtained according to the methods of the present invention, as described herein in prevention of the occurrence of tumors in the first place.

Treatment may be any treatment and/or therapy, whether of a human or an animal (e.g. in veterinary applications), in which some desired therapeutic effect is achieved, for example, the inhibition or delay of the progress of the condition, and includes a reduction in the rate of progress, a halt in the rate of progress, amelioration of the condition, cure or remission (whether partial or total) of the condition, preventing, delaying, abating or arresting one or more symptoms and/or signs of the condition or prolonging survival of a subject or patient beyond that expected in the absence of treatment.

Treatment may also be prophylactic (i.e. prophylaxis). For example, an individual susceptible to or at risk of the occurrence or re-occurrence of cancer may be treated as described herein. Such treatment may prevent or delay the occurrence or re-occurrence of cancer in the individual.

In particular, treatment may include inhibiting cancer growth, including complete cancer remission, and/or inhibiting cancer metastasis. Cancer growth generally refers to any one of a number of indices that indicate change within the cancer to a more developed form. Thus, indices for measuring an inhibition of cancer growth include a decrease in cancer cell survival, a decrease in tumor volume or morphology (for example, as determined using computed tomographic (CT), sonography, or other imaging method), a delayed tumor growth, a destruction of tumor vasculature, improved performance in delayed hypersensitivity skin test, an increase in the activity of T cells, and a decrease in levels of tumor-specific antigens. Administration of T cells modified as described herein may improve the capacity of the individual to resist cancer growth, in particular growth of a cancer already present the subject and/or decrease the propensity for cancer growth in the individual.

According to embodiments of the application, the composition comprises a therapeutically effective amount of an isolated polynucleotide, an isolated polypeptide, a host cell, and/or an iPSC or derivative cell thereof. As used herein, the term “therapeutically effective amount” refers to an amount of an active ingredient or component that elicits the desired biological or medicinal response in a subject. A therapeutically effective amount can be determined empirically and in a routine manner, in relation to the stated purpose.

As used herein with reference to a cell of the application and/or a pharmaceutical composition of the application a therapeutically effective amount means an amount of the cells and/or the pharmaceutical composition that modulates an immune response in a subject in need thereof.

According to particular embodiments, a therapeutically effective amount refers to the amount of therapy which is sufficient to achieve one, two, three, four, or more of the following effects: (i) reduce or ameliorate the severity of the disease, disorder or condition to be treated or a symptom associated therewith; (ii) reduce the duration of the disease, disorder or condition to be treated, or a symptom associated therewith; (iii) prevent the progression of the disease, disorder or condition to be treated, or a symptom associated therewith; (iv) cause regression of the disease, disorder or condition to be treated, or a symptom associated therewith; (v) prevent the development or onset of the disease, disorder or condition to be treated, or a symptom associated therewith; (vi) prevent the recurrence of the disease, disorder or condition to be treated, or a symptom associated therewith; (vii) reduce hospitalization of a subject having the disease, disorder or condition to be treated, or a symptom associated therewith; (viii) reduce hospitalization length of a subject having the disease, disorder or condition to be treated, or a symptom associated therewith; (ix) increase the survival of a subject with the disease, disorder or condition to be treated, or a symptom associated therewith; (xi) inhibit or reduce the disease, disorder or condition to be treated, or a symptom associated therewith in a subject; and/or (xii) enhance or improve the prophylactic or therapeutic effect(s) of another therapy.

The therapeutically effective amount or dosage can vary according to various factors, such as the disease, disorder or condition to be treated, the means of administration, the target site, the physiological state of the subject (including, e.g., age, body weight, health), whether the subject is a human or an animal, other medications administered, and whether the treatment is prophylactic or therapeutic. Treatment dosages are optimally titrated to optimize safety and efficacy.

According to particular embodiments, the compositions described herein are formulated to be suitable for the intended route of administration to a subject. For example, the compositions described herein can be formulated to be suitable for intravenous, subcutaneous, or intramuscular administration.

The cells of the application and/or the pharmaceutical compositions of the application can be administered in any convenient manner known to those skilled in the art. For example, the cells of the application can be administered to the subject by aerosol inhalation, injection, ingestion, transfusion, implantation, and/or transplantation. The compositions comprising the cells of the application can be administered transarterially, subcutaneously, intradermally, intratumorally, intranodally, intramedullary, intramuscularly, intrapleurally, by intravenous (i.v.) injection, or intraperitoneally. In certain embodiments, the cells of the application can be administered with or without lymphodepletion of the subject.

The pharmaceutical compositions comprising cells of the application can be provided in sterile liquid preparations, typically isotonic aqueous solutions with cell suspensions, or optionally as emulsions, dispersions, or the like, which are typically buffered to a selected pH. The compositions can comprise carriers, for example, water, saline, phosphate buffered saline, and the like, suitable for the integrity and viability of the cells, and for administration of a cell composition.

Sterile injectable solutions can be prepared by incorporating cells of the application in a suitable amount of the appropriate solvent with various other ingredients, as desired. Such compositions can include a pharmaceutically acceptable carrier, diluent, or excipient such as sterile water, physiological saline, glucose, dextrose, or the like, that are suitable for use with a cell composition and for administration to a subject, such as a human. Suitable buffers for providing a cell composition are well known in the art. Any vehicle, diluent, or additive used is compatible with preserving the integrity and viability of the cells of the application.

The cells of the application and/or the pharmaceutical compositions of the application can be administered in any physiologically acceptable vehicle. A cell population comprising cells of the application can comprise a purified population of cells. Those skilled in the art can readily determine the cells in a cell population using various well-known methods. The ranges in purity in cell populations comprising genetically modified cells of the application can be from about 50% to about 55%, from about 55% to about 60%, from about 60% to about 65%, from about 65% to about 70%, from about 70% to about 75%, from about 75% to about 80%, from about 80% to about 85%, from about 85% to about 90%, from about 90% to about 95%, or from about 95% to about 100%. Dosages can be readily adjusted by those skilled in the art, for example, a decrease in purity could require an increase in dosage.

The cells of the application are generally administered as a dose based on cells per kilogram (cells/kg) of body weight of the subject to which the cells and/or pharmaceutical compositions comprising the cells are administered. Generally, the cell doses are in the range of about 10⁴ to about 10¹⁰ cells/kg of body weight, for example, about 10⁵ to about 10⁹, about 10⁵ to about 10⁸, about 10⁵ to about 10⁷, or about 10⁵ to about 10⁶, depending on the mode and location of administration. In general, in the case of systemic administration, a higher dose is used than in regional administration, where the immune cells of the application are administered in the region of a tumor and/or cancer. Exemplary dose ranges include, but are not limited to, 1×10⁴ to 1×10⁸, 2×10⁴ to 1×10⁸, 3×10⁴ to 1×10⁸, 4×10⁴ to 1×10⁸, 5×10⁴ to 6×10⁸, 7×10⁴ to 1×10⁸, 8×10⁴ to 1×10⁸, 9×10⁴ to 1×10⁸, 1×10⁵ to 1×10⁸, 1×10⁵ to 9×10⁷, 1×10⁵ to 8×10⁷, 1×10⁵ to 7×10⁷, 1×10⁵ to 6×10⁷, 1×10⁵ to 5×10⁷, 1×10⁵ to 4×10⁷, 1×10⁵ to 4×10⁷, 1×10⁵ to 3×10⁷, 1×10⁵ to 2×10⁷, 1×10⁵ to 1×10⁷, 1×10⁵ to 9×10⁶, 1×10⁵ to 8×10⁶, 1×10⁵ to 7×10⁶, 1×10⁵ to 6×10⁶, 1×10⁵ to 5×10⁶, 1×10⁵ to 4×10⁶, 1×10⁵ to 4×10⁶, 1×10⁵ to 3×10⁶, 1×10⁵ to 2×10⁶, 1×10⁵ to 1×10⁶, 2×10⁵ to 9×10⁷, 2×10⁵ to 8×10⁷, 2×10⁵ to 7×10⁷, 2×10⁵ to 6×10⁷, 2×10⁵ to 5×10⁷, 2×10⁵ to 4×10⁷, 2×10⁵ to 4×10⁷, 2×10⁵ to 3×10⁷, 2×10⁵ to 2×10⁷, 2×10⁵ to 1×10⁷, 2×10⁵ to 9×10⁶, 2×10⁵ to 8×10⁶, 2×10⁵ to 7×10⁶, 2×10⁵ to 6×10⁶, 2×10⁵ to 5×10⁶, 2×10⁵ to 4×10⁶, 2×10⁵ to 4×10⁶, 2×10⁵ to 3×10⁶, 2×10⁵ to 2×10⁶, 2×10⁵ to 1×10⁶, 3×10⁵ to 3×10⁶ cells/kg, and the like. Additionally, the dose can be adjusted to account for whether a single dose is being administered or whether multiple doses are being administered. The precise determination of what would be considered an effective dose can be based on factors particular to each subject.

As used herein, the terms “treat,” “treating,” and “treatment” are all intended to refer to an amelioration or reversal of at least one measurable physical parameter related to a cancer, which is not necessarily discernible in the subject, but can be discernible in the subject. The terms “treat,” “treating,” and “treatment,” can also refer to causing regression, preventing the progression, or at least slowing down the progression of the disease, disorder, or condition. In a particular embodiment, “treat,” “treating,” and “treatment” refer to an alleviation, prevention of the development or onset, or reduction in the duration of one or more symptoms associated with the disease, disorder, or condition, such as a tumor or a cancer. In a particular embodiment, “treat,” “treating,” and “treatment” refer to prevention of the recurrence of the disease, disorder, or condition. In a particular embodiment, “treat,” “treating,” and “treatment” refer to an increase in the survival of a subject having the disease, disorder, or condition. In a particular embodiment, “treat,” “treating,” and “treatment” refer to elimination of the disease, disorder, or condition in the subject.

The cells of the application and/or the pharmaceutical compositions of the application can be administered in combination with one or more additional therapeutic agents. In certain embodiments the one or more therapeutic agents are selected from the group consisting of a peptide, a cytokine, a checkpoint inhibitor, a mitogen, a growth factor, a small RNA, a dsRNA (double stranded RNA), mononuclear blood cells, feeder cells, feeder cell components or replacement factors thereof, a vector comprising one or more polynucleic acids of interest, an antibody, a chemotherapeutic agent or a radioactive moiety, or an immunomodulatory drug (ImiD).

### EXAMPLES

The following examples are provided to further describe some of the embodiments disclosed herein. The examples are intended to illustrate, not to limit, the disclosed embodiments.

### Example 1. Site-Specific Engineering of iPSCs

Before starting, ensure that cells are healthy and approximately 70% confluent prior to nucleofection. Add H1152 Rock inhibitor to a final concentration of 1 μM (1:100) and place in incubator for one hour. Add 3 ml of media+1 μM Rock inhibitor to desired number of wells on a 6-well plate and place in incubator.

Prepare primary P3 electroporation buffer:
82 μl Nucleofector solution
18 μl Supplement

Mix together.

In a sterile tube, mix the following RNP components (per sample+1):
1.4 μl PBS
1.6 μl 100 μM guide RNA
2 μl MAD7 (60 μM).

Mix components well, spin down and let incubate at ambient temperature for 15 mins. Place the RNP at 4° C. until cells are ready for electroporation.

Aspirate spent media from flask and add 7 ml of DPBS, swirl gently and aspirate. Add 2 ml of TrypLE and incubate at 37° C. for 3-4 mins. Add 7 ml of E8 media, pipette up and down gently 2-3 times to get cells in single cell suspension. Centrifuge at 300×g for 3 mins and then aspirate and resuspend cells in 10 ml of cold Opti-MEM (4-6 ml if original flask low confluence). Replate some cells before proceeding. Count cells and pipette 1.5×10⁶ cells into a new sterile tube (per electroporation).

Centrifuge at 300×g for 3 min and then resuspend Cells in Primary P3 solution (100 μl per electroporation).

For each electroporation, add the components as described in Table 7 into a sterile Eppendorf tube.

TABLE 7
Components for Electroporation

| Component | Amount | |
|---|---|---|
| Cells (1 × 10⁶) | 100 | μl |
| pDNA | 3 | μg |
| RNP | 5 | μl |
| 100 μM EP enhancer | 0.8 | μl |

Mix well but gently and transfer 100 μl of the above mix into a EP cuvette without introducing any bubbles. Tap gently and place the cuvette into the nucleofector unit and use program CA-137. After electroporation, remove the cuvette. Using the provided sterile pipette transfer the electroporated cells into one well of a 6-well plate containing 3 ml media+1 μM H11252. Place the plate into the incubator and re-feed every day and check engineering status 7-10 days from electroporation.

### Example 2. Editing of B2M Locus

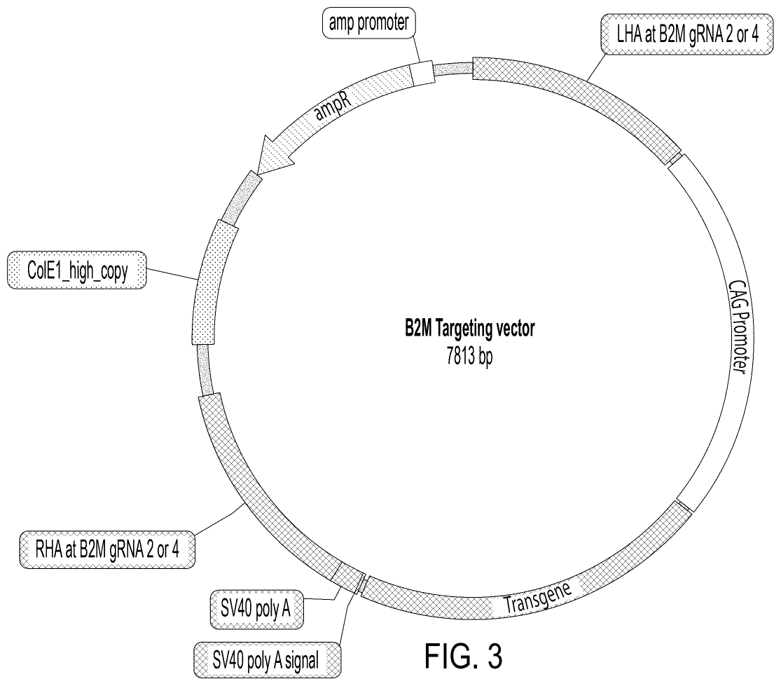

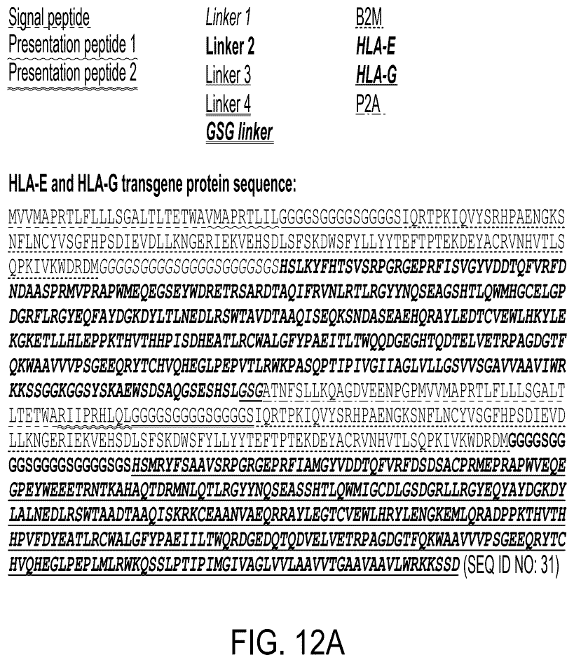

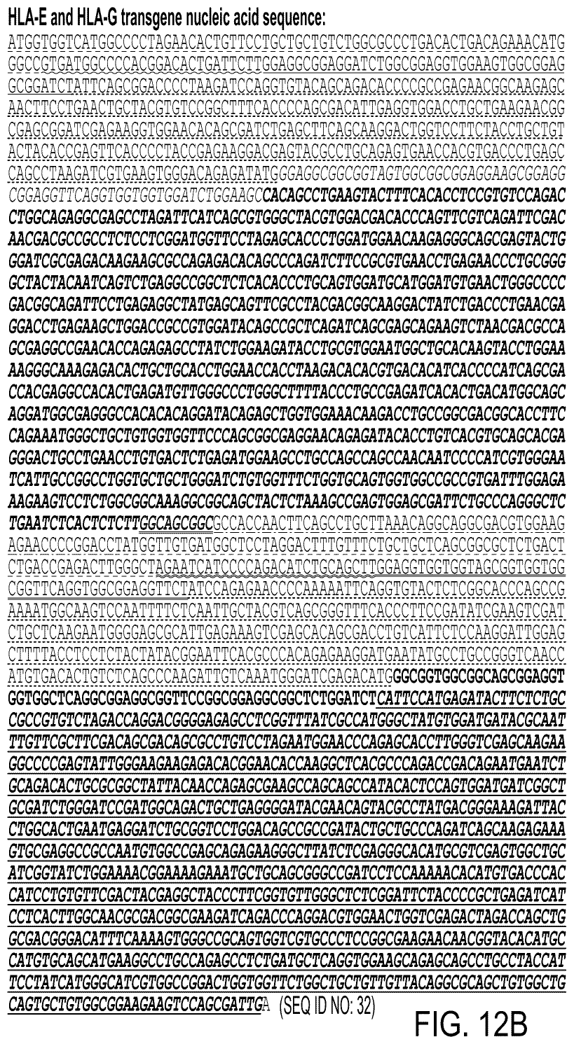

An HLA-E-2A-HLA-G transgene donor plasmid (pDNA referenced above) was specifically engineered to insert the HLA-E-2A-HLA-G transgene (FIG. 12B) into the B2M site (FIG. 3).

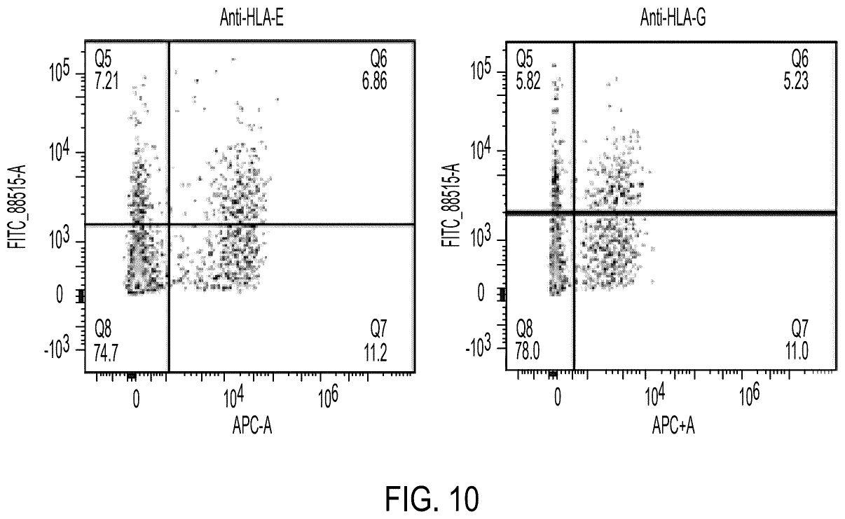

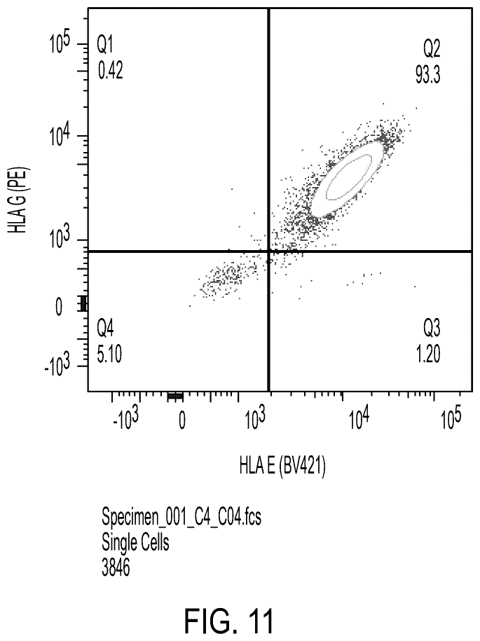

FIG. 10 depicts flow cytometry analysis of iPSCs post-engineering. Flow cytometry analyses of cells comprising post-sorting for HLA-E positive cells and HLA-G positive cells were performed (FIG. 10, top and bottom panels, respectively). The results show expression of both HLA-E and HLA-G in iPSCs after HDR into the B2M locus of iPSCs using the bi-cistronic HDR vector. Additional flow cytometry analysis demonstrated expression of both HLA-E and HLA-G engineered by homology directed repair (HDR) into iPSCs and enriched for HLA-E expression only (FIG. 11), confirming that the bi-cistronic HDR vector encoding both HLA-E and HLA-G can be used to express two transgenes simultaneously.

### Example 3. Editing of AA VS1 Locus

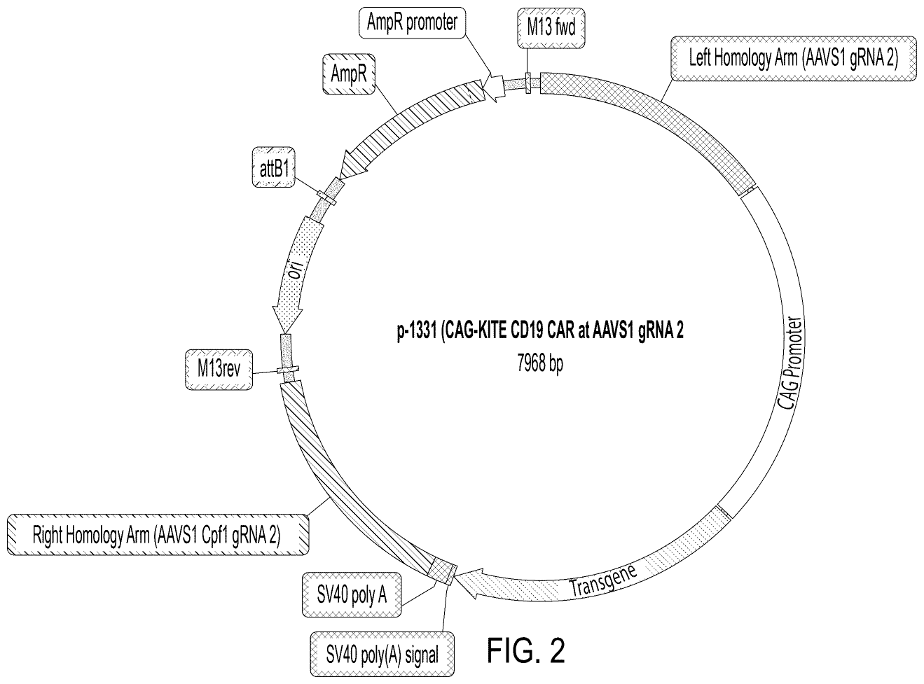

An HLA-E-2A-HLA-G transgene donor plasmid is specifically engineered to insert the HLA-E-2A-HLA-G into the AAVS1 site (e.g., FIG. 2). Various flow cytometry analyses of cells post-engineering are performed. As an example, expression of HLA-E and HLA-G is determined in iPSCs after HDR into the iPSCs.

### Example 4. Editing of CIITA Locus

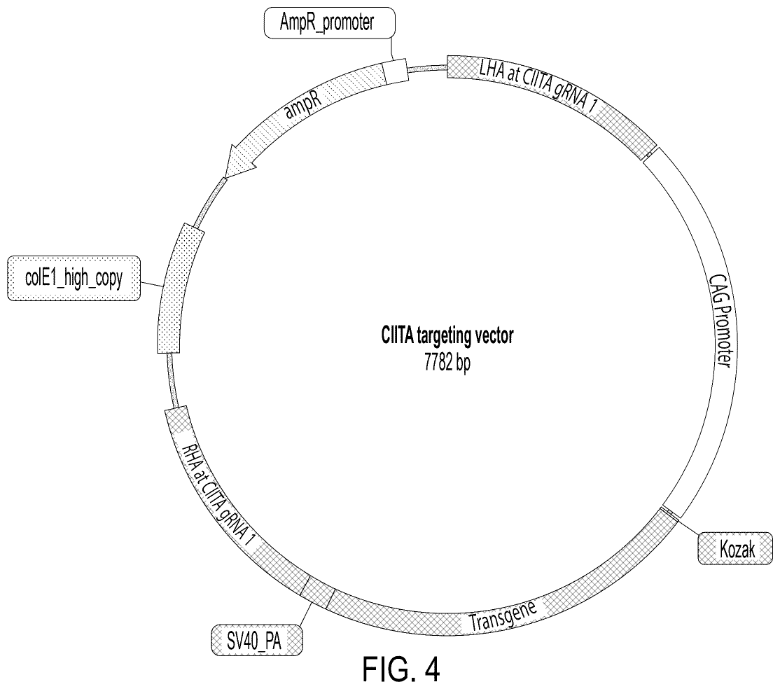

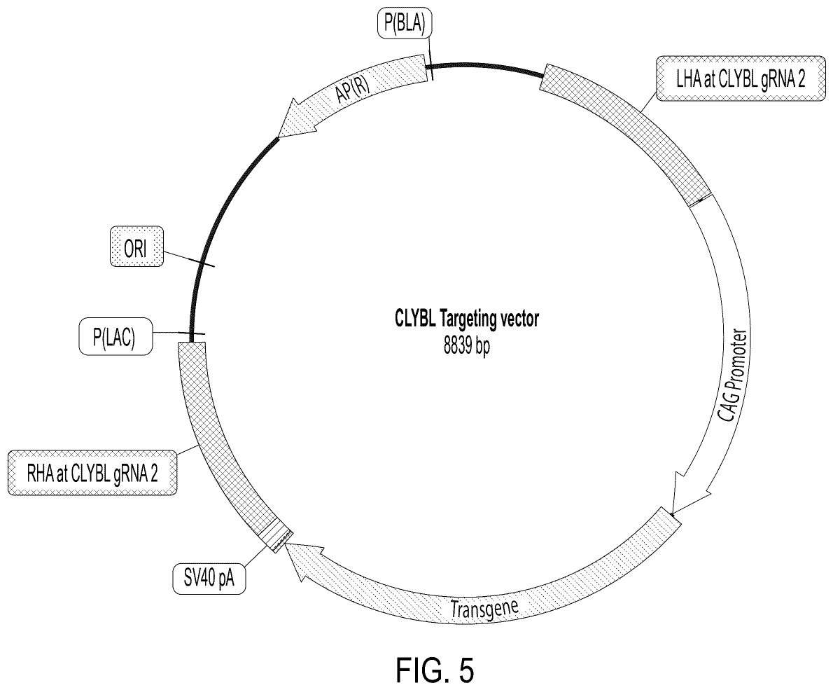

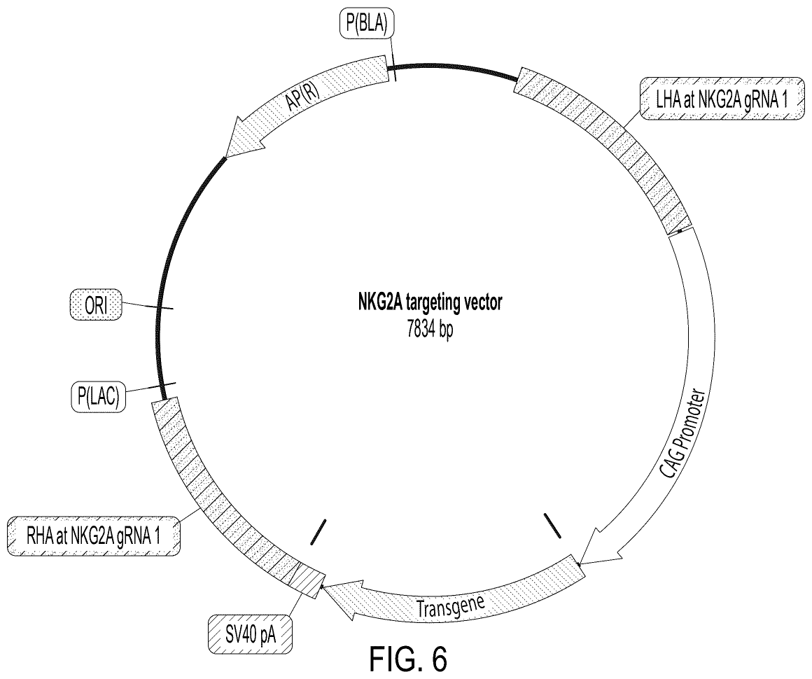

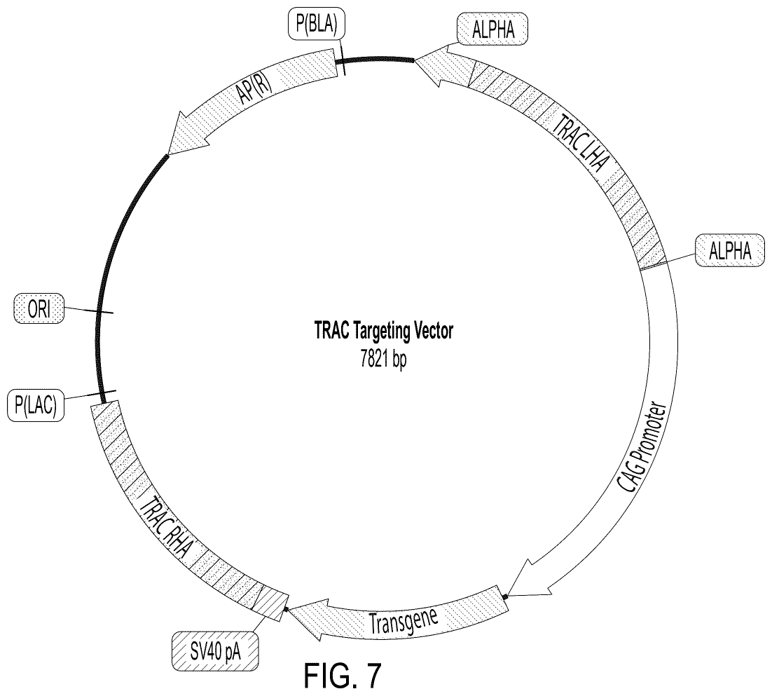

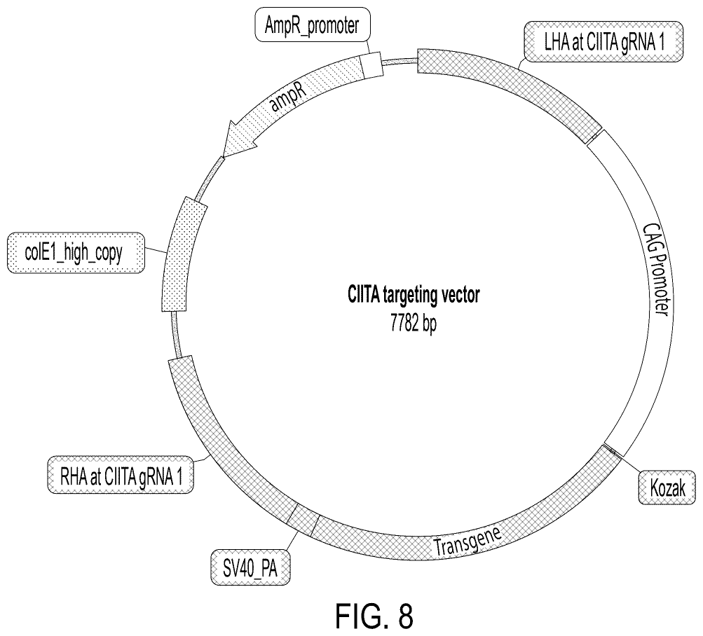

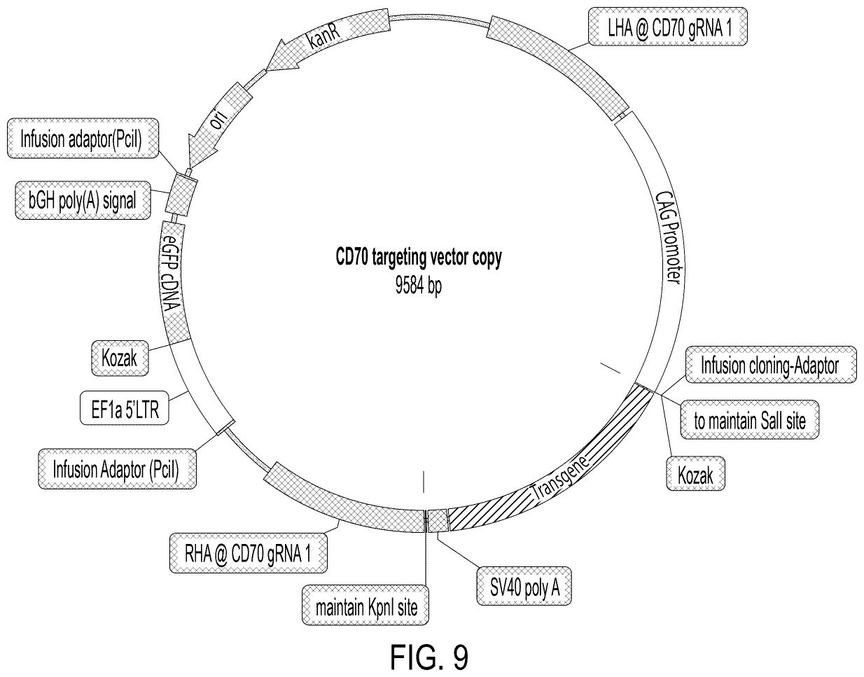

An HLA-E-2A-HLA-G transgene donor plasmid is specifically engineered to insert the HLA-E-2A-HLA-G into the CIITA site (e.g., FIG. 4). Various flow cytometry analyses of cells post-engineering are performed. As an example, expression of HLA-E and HLA-G is determined in iPSCs after HDR into the iPSCs.

### Example 5. Editing of CLYBL Locus

An HLA-E-2A-HLA-G transgene donor plasmid was specifically engineered to insert the HLA-E-2A-HLA-G transgene into the CLYBL site. Various flow cytometry analyses of cells post-engineering are performed. As an example, expression of HLA-E and HLA-G is determined in iPSCs after HDR into the iPSCs.

### Example 6. Editing of NKG2A Locus

An HLA-E-2A-HLA-G transgene donor plasmid was specifically engineered to insert the HLA-E-2A-HLA-G transgene into the NKG2A site. Various flow cytometry analyses of cells post-engineering are performed. As an example, expression of HLA-E and HLA-G is determined in iPSCs after HDR into the iPSCs.

### Example 7. HLA-E and HLA-G Engineered Cells

NK cells receive a balance of activating and inhibitory signals. They can recognize the absence of HLA Class I as a sign of DNA damage or viral infection and kill target cells. Due to the genetic diversity of HLA Class I proteins among the population, these molecules must be removed from iNK cells to prevent recognition and elimination by the recipient. This assay shows the potent killing of iNK cells by primary NK cells (e.g., isolated vs PBMC) when they lack HLA I proteins. To abrogate this phenomenon, iNK cells were engineered to express HLA-E and HLA-G (SEQ ID NO: 31) (which are more conserved in the human population).

### Materials

Donor blood samples (25-50×10⁶ cells/vial; frozen aliquots)
iNK cells
Cell Trace Yellow (CTY), ThermoFisher Scientific Cat #C34567
Cell Trace Violet, ThermoFisher Scientific Cat #C34557
CD56 APC, Biolegend Cat #981204
Dead Cell Removal Kit, Miltenyi #130-090-101.

PBMCs were recovered from donor blood samples and counted. PBMCs were resuspended, passed through 40 μM cell strainers into 50 mL conical tubes. 5 mL of PBS was used to wash the well and the cell strainers. This resulted in 10 mL total volume of cell suspension.

PBMC donor cells were stained with CTY according to the following protocol:
Cells were washed with 1×PBS.
Cells were resuspended in 1×PBS at a concentration of 2×10⁶ cells/mL.
Equivalent volumes of CTY Stain were added (1:2000 concentration in 1×PBS).
Cells were incubated at room temperature (RT) for 15 minutes away from light exposure.
   Stained cells were agitated every 3-5 minutes to ensure even distribution of stain (e.g., by vortexing or agitating the tubes).
After 15 minutes, media was added to each tube to double the initial volume.
Cell suspension of centrifuged, the supernatant was removed from cell pellet.
Cell pellet was resuspended in 10 mL of media to further wash any residual CTY.
Viability of cells was determined.
Cells were resuspended in assay media with necessary supplementation to a final concentration of 2.5×10⁶ cells/mL.

iNK cells were recovered after being rested overnight in 5 mL NK cell complete medium (NKCM)+10 ng/ml IL15. Wells were rinsed with additional 5.2 mL of PBS, and viability was determined.

100 μl of cell suspension was used to check for expression of HLA-E and HLA-G. One well of each sample was stained with only LIVE/DEAD™ Fixable Near-IR Dead Cell Stain (ThermoFisher #L10119) and one with the antibodies described below.

Staining panel:
LD—near-IR—1:1000 for 15 minutes in 100 μl PBS (wash with 150 μl PBS)·
CD56—BV421—1:400
HLA-E—APC—1:200
HLA-G—PE—1:200.

The samples were stained for 15 minutes in 100 μl BSA staining buffer; wash with 150 μl PBS, and then 200 μl PBS before fixing with 200 μl of BD™ stabilizing fixative.

iNK cells were stained with CTV (1:4000 of stock diluted with 50 μL DMSO for 15 minutes at RT in PBS at 1×10⁶ cells/mL). Cells were resuspended to 2×10⁶ cells/mL in PBS, and then 2× of staining solution was added. Staining was stopped by doubling the staining volume with 5% FBS RPMI.

Cell counts after CTV staining:
iNK (Sample 1)—0.60×10⁶ cells/mL*10 mL=6×10⁶ cells total; 58.3% Live
iNK (Sample 2)—0.70×10⁶ cells/mL*10 mL=7×10⁶ cells total; 59.7% Live.

Dead cell removal was performed according to manufacturer's instructions. LS columns (Miltenyi #130-042-401) were washed with 10 mL PBS containing FBS. Cell yields after the dead cell removal:
Sample 1-0.47×10⁶/mL*10 mL; 68.8% live; 2nd count: 0.46×10⁶/mL*9.9 mL; 63.9% live=>4.6×10⁶ total live cells
Sample 2-0.51×10⁶/mL*10.4 mL; 73.3% live=>5.3×10⁶ total live cells.

iNK cells were resuspended to 105/mL in NKCM with 10 ng/mL IL15 for plating in the assay (100 μl or 104/well).

Dead cell removal for the 2nd run (RC01-10) was performed using Miltenyi-provided binding buffer.

1. iNK cells were resuspended to 10 μl per 10⁶ cells with microbeads and incubated for 15 minutes at RT.

2. 500 μl 1× Binding Buffer was added. Cells were passed through LS column and washed with 3 ml 1× Binding Buffer.

3. The column was kept in the magnet and washed to 10 mL total volume in ˜3 mL increments.

Cell counts before dead cell removal:
Sample 1—3.86×10⁶/mL; 64.1% live; 7.72×10⁶ cells total
Sample 2—2.98×10⁶/mL; 63.4% live; 5.96×10⁶ cells total.

Cell counts after dead cell removal:
Sample 1—5.46×10⁶ cells total; 97.8% live
Sample 2—4.11×10⁶ cells total; 96.7% live.

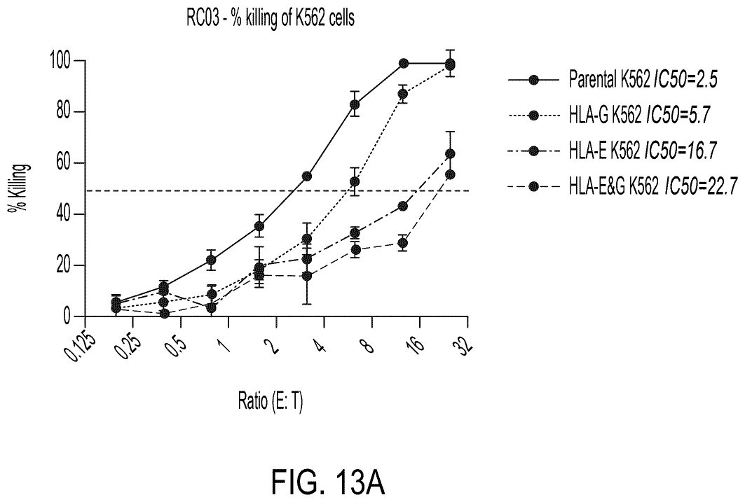

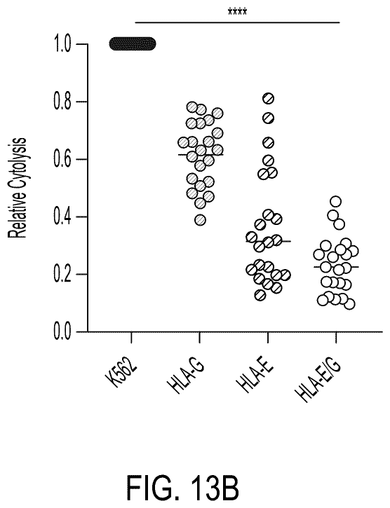

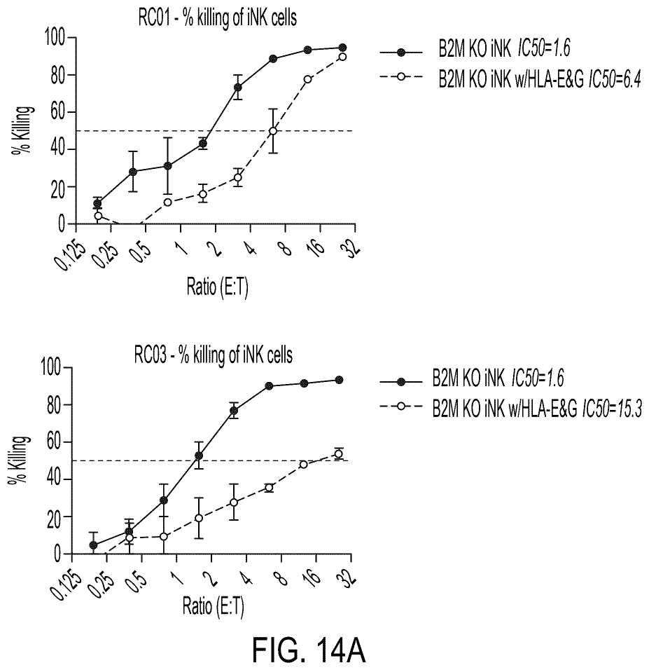

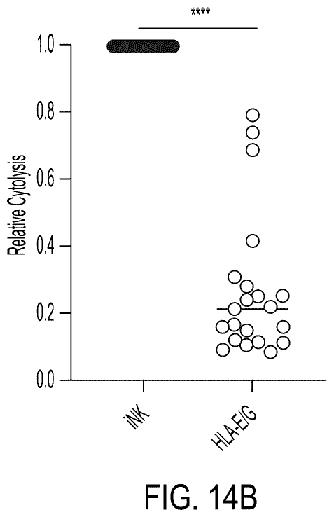

HLA-E expression on K562 cells offered improved protection from killing than HLA-G, but the combination of both HLA-E and HLA-G confers improved protective effect against PBMC cytolysis (FIGS. 13A-13B). iNK cells edited with HLA-E and HLA-G were consistently protective compared to iNK cells lacking HLA (FIGS. 14A-14B).

### Example 8. Expression of HLA-E and HLA-G Constructs with Different Linkers

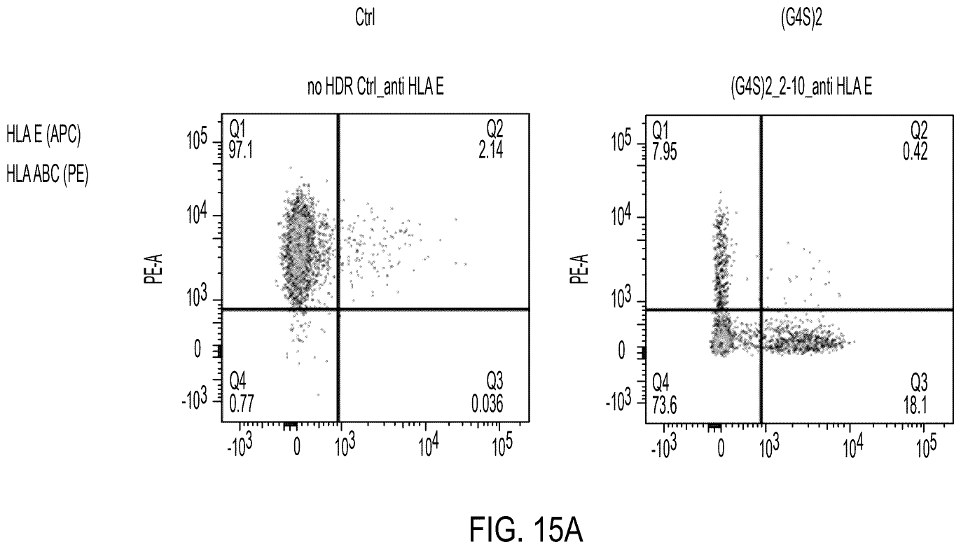

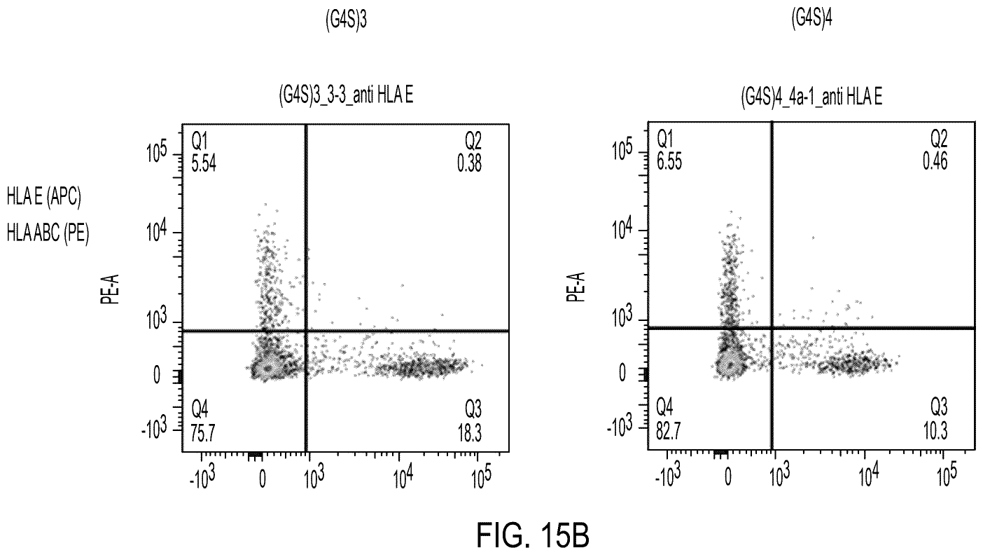

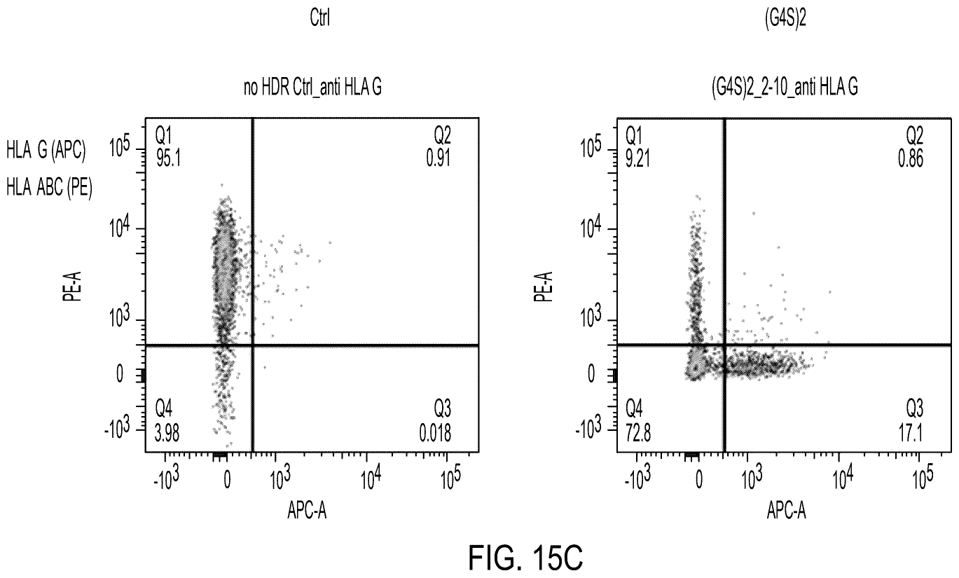

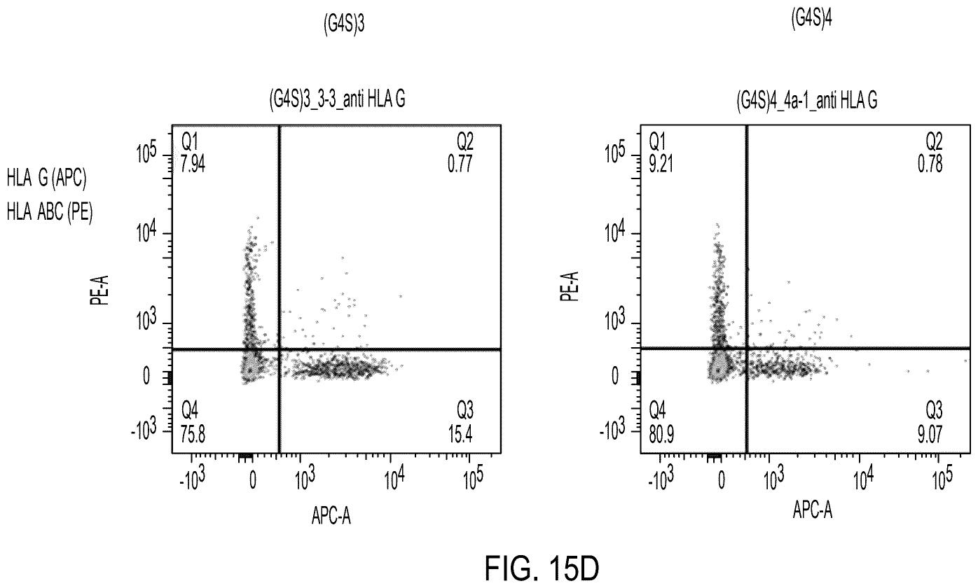

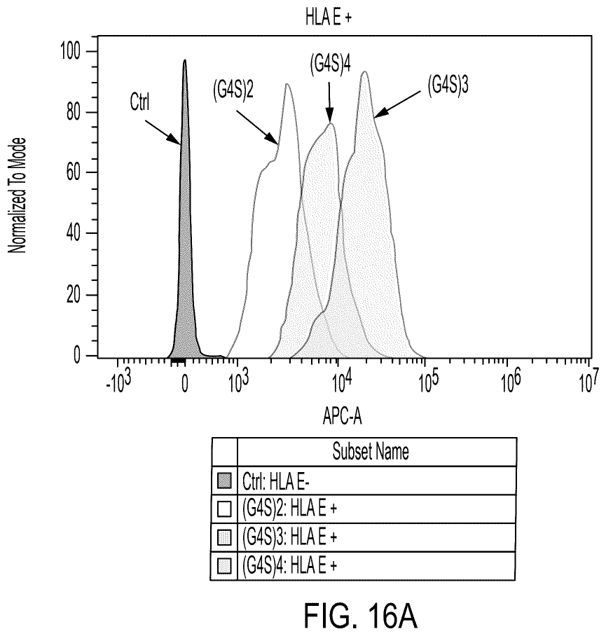

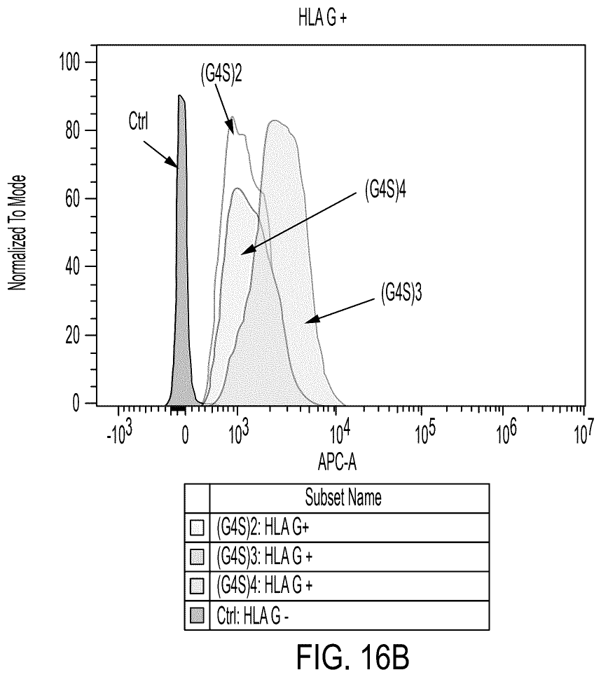

iPSCs were engineered to express HLA-E and HLA-G joined by linkers (G4S)₂ (SEQ ID NO: 165), (G4S)₃ (SEQ ID NO: 31), and (G4S)₄ (SEQ ID NO: 168). Expression of HLA-E and HLA-G joined by (G4S)₂, (G4S)₃, and (G4S)₄ from the iPSCs was analyzed using flow cytometry. Cells were stained with the following antibodies: HLA-E—APC—1:200 and HLA-G—PE—1:200. The results are shown in FIGS. 15A-15D and 16A-16B. The construct with (G4S)₃ linker (SEQ ID NO: 31) showed the highest expression.

The present disclosure is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

### Listing of Sequences

SEQ ID NO: 1 Signal peptide HLA-G amino acid sequence
MVVMAPRTLFLLLSGALTLTETWA
SEQ ID NO: 2 Signal peptide HLA-G nucleotide sequence
ATGGTGGTCATGGCCCCTAGAACACTGTTCCTGCTGCTGTCTGGCGCCCTGACACTG ACAGAAACATGGGCC
SEQ ID NO: 3 Signal peptide HLA-G nucleotide sequence
ATGGTTGTGATGGCTCCTAGGACTTTGTTTCTGCTGCTCAGCGGCGCTCTGACTCTGA CCGAGACTTGGGCT
SEQ ID NO: 4 Presentation peptide 1 amino acid sequence
VMAPRTLIL
SEQ ID NO: 5 Presentation peptide 1 nucleotide sequence
GTGATGGCCCCACGGACACTGATTCTT
SEQ ID NO: 6 Linker (G4S)3 amino acid sequence
GGGGSGGGGSGGGGS
SEQ ID NO: 7 Linker (G4S)3 (SEQ ID NO: 6) nucleotide sequence
GGAGGCGGAGGATCTGGCGGAGGTGGAAGTGGCGGAGGCGGATCT
SEQ ID NO: 8 Linker (G4S)3 (SEQ ID NO: 6) nucleotide sequence
GGAGGTGGTGGTAGCGGTGGTGGCGGTTCAGGTGGCGGAGGTTCT
SEQ ID NO: 9 B2M amino acid sequence
IQRTPKIQVYSRHPAENGKSNFLNCYVSGFHPSDIEVDLLKNGERIEKVEHSDLSFSKDW SFYLLYYTEFTPTEKDEYACRVNHVTLSQPKIVKWDRDM
SEQ ID NO: 10 B2M nucleotide sequence
ATTCAGCGGACCCCTAAGATCCAGGTGTACAGCAGACACCCCGCCGAGAACGGCAA GAGCAACTTCCTGAACTGCTACGTGTCCGGCTTTCACCCCAGCGACATTGAGGTGGA CCTGCTGAAGAACGGCGAGCGGATCGAGAAGGTGGAACACAGCGATCTGAGCTTCA GCAAGGACTGGTCCTTCTACCTGCTGTACTACACCGAGTTCACCCCTACCGAGAAGG ACGAGTACGCCTGCAGAGTGAACCACGTGACCCTGAGCCAGCCTAAGATCGTGAAG TGGGACAGAGATATG
SEQ ID NO: 11 B2M nucleotide sequence
ATCCAGAGAACCCCAAAAATTCAGGTGTACTCTCGGCACCCAGCCGAAAATGGCAA GTCCAATTTTCTCAATTGCTACGTCAGCGGGTTTCACCCTTCCGATATCGAAGTCGAT CTGCTCAAGAATGGGGAGCGCATTGAGAAAGTCGAGCACAGCGACCTGTCATTCTC CAAGGATTGGAGCTTTTACCTCCTCTACTATACGGAATTCACGCCCACAGAGAAGGA TGAATATGCCTGCCGGGTCAACCATGTGACACTGTCTCAGCCCAAGATTGTCAAATG GGATCGAGACATG
SEQ ID NO: 12 Linker (G4S)4GS amino acid sequence
GGGGSGGGGSGGGGSGGGGSGS
SEQ ID NO: 13 Linker (G4S)4GS (SEQ ID NO: 12) nucleotide sequence
GGAGGCGGCGGTAGTGGCGGCGGAGGAAGCGGAGGCGGAGGTTCAGGTGGTGGTG GATCTGGAAGC
SEQ ID NO: 14 Linker (G4S)4GS (SEQ ID NO: 12) nucleotide sequence
GGCGGTGGCGGCAGCGGAGGTGGTGGCTCAGGCGGAGGCGGTTCCGGCGGAGGCG GCTCTGGATCT
SEQ ID NO: 15 HLA-E amino acid sequence
HSLKYFHTSVSRPGRGEPRFISVGYVDDTQFVRFDNDAASPRMVPRAPWMEQEGSEYW DRETRSARDTAQIFRVNLRTLRGYYNQSEAGSHTLQWMHGCELGPDGRFLRGYEQFAY DGKDYLTLNEDLRSWTAVDTAAQISEQKSNDASEAEHQRAYLEDTCVEWLHKYLEKG KETLLHLEPPKTHVTHHPISDHEATLRCWALGFYPAEITLTWQQDGEGHTQDTEL VETR PAGDGTFQKWAAVVVPSGEEQRYTCHVQHEGLPEPVTLRWKPASQPTIPIVGIIAGLVLL GSVVSGAVVAAVIWRKKSSGGKGGSYSKAEWSDSAQGSESHSL
SEQ ID NO: 16 HLA-E nucleotide sequence
CACAGCCTGAAGTACTTTCACACCTCCGTGTCCAGACCTGGCAGAGGCGAGCCTAG ATTCATCAGCGTGGGCTACGTGGACGACACCCAGTTCGTCAGATTCGACAACGACG CCGCCTCTCCTCGGATGGTTCCTAGAGCACCCTGGATGGAACAAGAGGGCAGCGAG TACTGGGATCGCGAGACAAGAAGCGCCAGAGACACAGCCCAGATCTTCCGCGTGAA CCTGAGAACCCTGCGGGGCTACTACAATCAGTCTGAGGCCGGCTCTCACACCCTGCA GTGGATGCATGGATGTGAACTGGGCCCCGACGGCAGATTCCTGAGAGGCTATGAGC AGTTCGCCTACGACGGCAAGGACTATCTGACCCTGAACGAGGACCTGAGAAGCTGG ACCGCCGTGGATACAGCCGCTCAGATCAGCGAGCAGAAGTCTAACGACGCCAGCGA GGCCGAACACCAGAGAGCCTATCTGGAAGATACCTGCGTGGAATGGCTGCACAAGT ACCTGGAAAAGGGCAAAGAGACACTGCTGCACCTGGAACCACCTAAGACACACGTG ACACATCACCCCATCAGCGACCACGAGGCCACACTGAGATGTTGGGCCCTGGGCTTT TACCCTGCCGAGATCACACTGACATGGCAGCAGGATGGCGAGGGCCACACACAGGA TACAGAGCTGGTGGAAACAAGACCTGCCGGCGACGGCACCTTCCAGAAATGGGCTG CTGTGGTGGTTCCCAGCGGCGAGGAACAGAGATACACCTGTCACGTGCAGCACGAG GGACTGCCTGAACCTGTGACTCTGAGATGGAAGCCTGCCAGCCAGCCAACAATCCC CATCGTGGGAATCATTGCCGGCCTGGTGCTGCTGGGATCTGTGGTTTCTGGTGCAGT GGTGGCCGCCGTGATTTGGAGAAAGAAGTCCTCTGGCGGCAAAGGCGGCAGCTACT CTAAAGCCGAGTGGAGCGATTCTGCCCAGGGCTCTGAATCTCACTCTCTT
SEQ ID NO: 17 B2M-HLA-E amino acid sequence
IQRTPKIQVYSRHPAENGKSNFLNCYVSGFHPSDIEVDLLKNGERIEKVEHSDLSFSKDW SFYLLYYTEFTPTEKDEYACRVNHVTLSQPKIVKWDRDMGGGGSGGGGSGGGGSGGG GSGS HSLKYFHTSVSRPGRGEPRFISVGYVDDTQFVRFDNDAASPRMVPRAPWMEQEG SEYWDRETRSARDTAQIFRVNLRTLRGYYNQSEAGSHTLQWMHGCELGPDGRFLRGYE QFAYDGKDYLTLNEDLRSWTAVDTAAQISEQKSNDASEAEHQRAYLEDTCVEWLHKY LEKGKETLLHLEPPKTHVTHHPISDHEATLRCWALGFYPAEITLTWQQDGEGHTQDTEL VETRPAGDGTFQKWAAVVVPSGEEQRYTCHVQHEGLPEPVTLRWKPASQPTIPIVGIIAG LVLLGSVVSGAVVAAVIWRKKSSGGKGGSYSKAEWSDSAQGSESHSL
SEQ ID NO: 18 B2M-HLA-E nucleotide sequence
ATTCAGCGGACCCCTAAGATCCAGGTGTACAGCAGACACCCCGCCGAGAACGGCAA GAGCAACTTCCTGAACTGCTACGTGTCCGGCTTTCACCCCAGCGACATTGAGGTGGA CCTGCTGAAGAACGGCGAGCGGATCGAGAAGGTGGAACACAGCGATCTGAGCTTCA GCAAGGACTGGTCCTTCTACCTGCTGTACTACACCGAGTTCACCCCTACCGAGAAGG ACGAGTACGCCTGCAGAGTGAACCACGTGACCCTGAGCCAGCCTAAGATCGTGAAG TGGGACAGAGATATGGGAGGCGGCGGTAGTGGCGGCGGAGGAAGCGGAGGCGGAG GTTCAGGTGGTGGTGGATCTGGAAGC CACAGCCTGAAGTACTTTCACACCTCCGTGT CCAGACCTGGCAGAGGCGAGCCTAGATTCATCAGCGTGGGCTACGTGGACGACACC CAGTTCGTCAGATTCGACAACGACGCCGCCTCTCCTCGGATGGTTCCTAGAGCACCC TGGATGGAACAAGAGGGCAGCGAGTACTGGGATCGCGAGACAAGAAGCGCCAGAG ACACAGCCCAGATCTTCCGCGTGAACCTGAGAACCCTGCGGGGCTACTACAATCAG TCTGAGGCCGGCTCTCACACCCTGCAGTGGATGCATGGATGTGAACTGGGCCCCGAC GGCAGATTCCTGAGAGGCTATGAGCAGTTCGCCTACGACGGCAAGGACTATCTGAC CCTGAACGAGGACCTGAGAAGCTGGACCGCCGTGGATACAGCCGCTCAGATCAGCG AGCAGAAGTCTAACGACGCCAGCGAGGCCGAACACCAGAGAGCCTATCTGGAAGAT ACCTGCGTGGAATGGCTGCACAAGTACCTGGAAAAGGGCAAAGAGACACTGCTGCA CCTGGAACCACCTAAGACACACGTGACACATCACCCCATCAGCGACCACGAGGCCA CACTGAGATGTTGGGCCCTGGGCTTTTACCCTGCCGAGATCACACTGACATGGCAGC AGGATGGCGAGGGCCACACACAGGATACAGAGCTGGTGGAAACAAGACCTGCCGG CGACGGCACCTTCCAGAAATGGGCTGCTGTGGTGGTTCCCAGCGGCGAGGAACAGA GATACACCTGTCACGTGCAGCACGAGGGACTGCCTGAACCTGTGACTCTGAGATGG AAGCCTGCCAGCCAGCCAACAATCCCCATCGTGGGAATCATTGCCGGCCTGGTGCTG CTGGGATCTGTGGTTTCTGGTGCAGTGGTGGCCGCCGTGATTTGGAGAAAGAAGTCC TCTGGCGGCAAAGGCGGCAGCTACTCTAAAGCCGAGTGGAGCGATTCTGCCCAGGG CTCTGAATCTCACTCTCTT
SEQ ID NO: 19 Signal-presentation-B2M-HLA-E amino acid sequence
MVVMAPRTLFLLLSGALTLTETWAVMAPRTLILGGGGSGGGGSGGGGSIQRTPKIQVYS RHPAENGKSNFLNCYVSGFHPSDIEVDLLKNGERIEKVEHSDLSFSKDWSFYLLYYTEFT PTEKDEYACRVNHVTLSQPKIVKWDRDMGGGGSGGGGSGGGGSGGGGSGSHSLKYFH TSVSRPGRGEPRFISVGYVDDTQFVRFDNDAASPRMVPRAPWMEQEGSEYWDRETRSA RDTAQIFRVNLRTLRGYYNQSEAGSHTLQWMHGCELGPDGRFLRGYEQFAYDGKDYL TLNEDLRSWTAVDTAAQISEQKSNDASEAEHQRAYLEDTCVEWLHKYLEKGKETLLHL EPPKTHVTHHPISDHEATLRCWALGFYPAEITLTWQQDGEGHTQDTEL VETRPAGDGTF QKWAAVVVPSGEEQRYTCHVQHEGLPEPVTLRWKPASQPTIPIVGIIAGLVLLGSVVSG AVVAAVIWRKKSSGGKGGSYSKAEWSDSAQGSESHSL
SEQ ID NO: 20 Signal-presentation-B2M-HLA-E nucleotide sequence
ATGGTGGTCATGGCCCCTAGAACACTGTTCCTGCTGCTGTCTGGCGCCCTGACACTG ACAGAAACATGGGCCGTGATGGCCCCACGGACACTGATTCTTGGAGGCGGAGGATC TGGCGGAGGTGGAAGTGGCGGAGGCGGATCTATTCAGCGGACCCCTAAGATCCAGG TGTACAGCAGACACCCCGCCGAGAACGGCAAGAGCAACTTCCTGAACTGCTACGTG TCCGGCTTTCACCCCAGCGACATTGAGGTGGACCTGCTGAAGAACGGCGAGCGGAT CGAGAAGGTGGAACACAGCGATCTGAGCTTCAGCAAGGACTGGTCCTTCTACCTGCT GTACTACACCGAGTTCACCCCTACCGAGAAGGACGAGTACGCCTGCAGAGTGAACC ACGTGACCCTGAGCCAGCCTAAGATCGTGAAGTGGGACAGAGATATGGGAGGCGGC GGTAGTGGCGGCGGAGGAAGCGGAGGCGGAGGTTCAGGTGGTGGTGGATCTGGAA GCCACAGCCTGAAGTACTTTCACACCTCCGTGTCCAGACCTGGCAGAGGCGAGCCTA GATTCATCAGCGTGGGCTACGTGGACGACACCCAGTTCGTCAGATTCGACAACGAC GCCGCCTCTCCTCGGATGGTTCCTAGAGCACCCTGGATGGAACAAGAGGGCAGCGA GTACTGGGATCGCGAGACAAGAAGCGCCAGAGACACAGCCCAGATCTTCCGCGTGA ACCTGAGAACCCTGCGGGGCTACTACAATCAGTCTGAGGCCGGCTCTCACACCCTGC AGTGGATGCATGGATGTGAACTGGGCCCCGACGGCAGATTCCTGAGAGGCTATGAG CAGTTCGCCTACGACGGCAAGGACTATCTGACCCTGAACGAGGACCTGAGAAGCTG GACCGCCGTGGATACAGCCGCTCAGATCAGCGAGCAGAAGTCTAACGACGCCAGCG AGGCCGAACACCAGAGAGCCTATCTGGAAGATACCTGCGTGGAATGGCTGCACAAG TACCTGGAAAAGGGCAAAGAGACACTGCTGCACCTGGAACCACCTAAGACACACGT GACACATCACCCCATCAGCGACCACGAGGCCACACTGAGATGTTGGGCCCTGGGCT TTTACCCTGCCGAGATCACACTGACATGGCAGCAGGATGGCGAGGGCCACACACAG GATACAGAGCTGGTGGAAACAAGACCTGCCGGCGACGGCACCTTCCAGAAATGGGC TGCTGTGGTGGTTCCCAGCGGCGAGGAACAGAGATACACCTGTCACGTGCAGCACG AGGGACTGCCTGAACCTGTGACTCTGAGATGGAAGCCTGCCAGCCAGCCAACAATC CCCATCGTGGGAATCATTGCCGGCCTGGTGCTGCTGGGATCTGTGGTTTCTGGTGCA GTGGTGGCCGCCGTGATTTGGAGAAAGAAGTCCTCTGGCGGCAAAGGCGGCAGCTA CTCTAAAGCCGAGTGGAGCGATTCTGCCCAGGGCTCTGAATCTCACTCTCTT
SEQ ID NO: 21 P2A amino acid sequence
ATNFSLLKQAGDVEENPGP
SEQ ID NO: 22 P2A nucleotide sequence
GCCACCAACTTCAGCCTGCTTAAACAGGCAGGCGACGTGGAAGAGAACCCCGGACC T
SEQ ID NO: 23 Presentation peptide 2 amino acid sequence
RIIPRHLQL
SEQ ID NO: 24 Presentation peptide 2 nucleotide sequence
AGAATCATCCCCAGACATCTGCAGCTT
SEQ ID NO: 25 HLA-G amino acid sequence
HSMRYFSAAVSRPGRGEPRFIAMGYVDDTQFVRFDSDSACPRMEPRAPWVEQEGPEYW EEETRNTKAHAQTDRMNLQTLRGYYNQSEASSHTLQWMIGCDLGSDGRLLRGYEQYA YDGKDYLALNEDLRSWTAADTAAQISKRKCEAANVAEQRRAYLEGTCVEWLHRYLEN GKEMLQRADPPKTHVTHHPVFDYEATLRCWALGFYPAEIILTWQRDGEDQTQDVELVE TRPAGDGTFQKWAAVVVPSGEEQRYTCHVQHEGLPEPLMLRWKQSSLPTIPIMGIVAGL VVLAAVVTGAAVAAVLWRKKSSD
SEQ ID NO: 26 HLA-G nucleotide sequence
ATGAGATACTTCTCTGCCGCCGTGTCTAGACCAGGACGGGGAGAGCCTCGGTTTATC GCCATGGGCTATGTGGATGATACGCAATTTGTTCGCTTCGACAGCGACAGCGCCTGT CCTAGAATGGAACCCAGAGCACCTTGGGTCGAGCAAGAAGGCCCCGAGTATTGGGA AGAAGAGACACGGAACACCAAGGCTCACGCCCAGACCGACAGAATGAATCTGCAG ACACTGCGCGGCTATTACAACCAGAGCGAAGCCAGCAGCCATACACTCCAGTGGAT GATCGGCTGCGATCTGGGATCCGATGGCAGACTGCTGAGGGGATACGAACAGTACG CCTATGACGGGAAAGATTACCTGGCACTGAATGAGGATCTGCGGTCCTGGACAGCC GCCGATACTGCTGCCCAGATCAGCAAGAGAAAGTGCGAGGCCGCCAATGTGGCCGA GCAGAGAAGGGCTTATCTCGAGGGCACATGCGTCGAGTGGCTGCATCGGTATCTGG AAAACGGAAAAGAAATGCTGCAGCGGGCCGATCCTCCAAAAACACATGTGACCCAC CATCCTGTGTTCGACTACGAGGCTACCCTTCGGTGTTGGGCTCTCGGATTCTACCCCG CTGAGATCATCCTCACTTGGCAACGCGACGGCGAAGATCAGACCCAGGACGTGGAA CTGGTCGAGACTAGACCAGCTGGCGACGGGACATTTCAAAAGTGGGCCGCAGTGGT CGTGCCCTCCGGCGAAGAACAACGGTACACATGCCATGTGCAGCATGAAGGCCTGC CAGAGCCTCTGATGCTCAGGTGGAAGCAGAGCAGCCTGCCTACCATTCCTATCATGG GCATCGTGGCCGGACTGGTGGTTCTGGCTGCTGTTGTTACAGGCGCAGCTGTGGCTG CAGTGCTGTGGCGGAAGAAGTCCAGCGATTG
SEQ ID NO: 27 B2M-HLA-G amino acid sequence
IQRTPKIQVYSRHPAENGKSNFLNCYVSGFHPSDIEVDLLKNGERIEKVEHSDLSFSKDW SFYLLYYTEFTPTEKDEYACRVNHVTLSQPKIVKWDRDMGGGGSGGGGSGGGGSGGG GSGS HSMRYFSAAVSRPGRGEPRFIAMGYVDDTQFVRFDSDSACPRMEPRAPWVEQEG PEYWEEETRNTKAHAQTDRMNLQTLRGYYNQSEASSHTLQWMIGCDLGSDGRLLRGY EQYAYDGKDYLALNEDLRSWTAADTAAQISKRKCEAANVAEQRRAYLEGTCVEWLHR YLENGKEMLQRADPPKTHVTHHPVFDYEATLRCWALGFYPAEIILTWQRDGEDQTQDV ELVETRPAGDGTFQKWAAVVVPSGEEQRYTCHVQHEGLPEPLMLRWKQSSLPTIPIMGI VAGLVVLAAVVTGAAVAAVLWRKKSSD
SEQ ID NO: 28 B2M-HLA-G nucleotide sequence
ATCCAGAGAACCCCAAAAATTCAGGTGTACTCTCGGCACCCAGCCGAAAATGGCAA GTCCAATTTTCTCAATTGCTACGTCAGCGGGTTTCACCCTTCCGATATCGAAGTCGAT CTGCTCAAGAATGGGGAGCGCATTGAGAAAGTCGAGCACAGCGACCTGTCATTCTC CAAGGATTGGAGCTTTTACCTCCTCTACTATACGGAATTCACGCCCACAGAGAAGGA TGAATATGCCTGCCGGGTCAACCATGTGACACTGTCTCAGCCCAAGATTGTCAAATG GGATCGAGACATGGGCGGTGGCGGCAGCGGAGGTGGTGGCTCAGGCGGAGGCGGTT CCGGCGGAGGCGGCTCTGGATCT CATTCCATGAGATACTTCTCTGCCGCCGTGTCTA GACCAGGACGGGGAGAGCCTCGGTTTATCGCCATGGGCTATGTGGATGATACGCAA TTTGTTCGCTTCGACAGCGACAGCGCCTGTCCTAGAATGGAACCCAGAGCACCTTGG GTCGAGCAAGAAGGCCCCGAGTATTGGGAAGAAGAGACACGGAACACCAAGGCTC ACGCCCAGACCGACAGAATGAATCTGCAGACACTGCGCGGCTATTACAACCAGAGC GAAGCCAGCAGCCATACACTCCAGTGGATGATCGGCTGCGATCTGGGATCCGATGG CAGACTGCTGAGGGGATACGAACAGTACGCCTATGACGGGAAAGATTACCTGGCAC TGAATGAGGATCTGCGGTCCTGGACAGCCGCCGATACTGCTGCCCAGATCAGCAAG AGAAAGTGCGAGGCCGCCAATGTGGCCGAGCAGAGAAGGGCTTATCTCGAGGGCAC ATGCGTCGAGTGGCTGCATCGGTATCTGGAAAACGGAAAAGAAATGCTGCAGCGGG CCGATCCTCCAAAAACACATGTGACCCACCATCCTGTGTTCGACTACGAGGCTACCC TTCGGTGTTGGGCTCTCGGATTCTACCCCGCTGAGATCATCCTCACTTGGCAACGCG ACGGCGAAGATCAGACCCAGGACGTGGAACTGGTCGAGACTAGACCAGCTGGCGAC GGGACATTTCAAAAGTGGGCCGCAGTGGTCGTGCCCTCCGGCGAAGAACAACGGTA CACATGCCATGTGCAGCATGAAGGCCTGCCAGAGCCTCTGATGCTCAGGTGGAAGC AGAGCAGCCTGCCTACCATTCCTATCATGGGCATCGTGGCCGGACTGGTGGTTCTGG CTGCTGTTGTTACAGGCGCAGCTGTGGCTGCAGTGCTGTGGCGGAAGAAGTCCAGCG ATTG
SEQ ID NO: 29 Signal-presentation-B2M-HLA-G amino acid sequence
MVVMAPRTLFLLLSGALTLTETWARIIPRHLQLGGGGSGGGGSGGGGSIQRTPKIQVYS RHPAENGKSNFLNCYVSGFHPSDIEVDLLKNGERIEKVEHSDLSFSKDWSFYLLYYTEFT PTEKDEYACRVNHVTLSQPKIVKWDRDMGGGGSGGGGSGGGGSGGGGSGSHSMRYFS AAVSRPGRGEPRFIAMGYVDDTQFVRFDSDSACPRMEPRAPWVEQEGPEYWEEETRNT KAHAQTDRMNLQTLRGYYNQSEASSHTLQWMIGCDLGSDGRLLRGYEQYAYDGKDY LALNEDLRSWTAADTAAQISKRKCEAANVAEQRRAYLEGTCVEWLHRYLENGKEMLQ RADPPKTHVTHHPVFDYEATLRCWALGFYPAEIILTWQRDGEDQTQDVELVETRPAGD GTFQKWAAVVVPSGEEQRYTCHVQHEGLPEPLMLRWKQSSLPTIPIMGIVAGLVVLAA VVTGAAVAAVLWRKKSSD
SEQ ID NO: 30 Signal-presentation-B2M-HLA-G nucleotide sequence
ATGGTTGTGATGGCTCCTAGGACTTTGTTTCTGCTGCTCAGCGGCGCTCTGACTCTGA CCGAGACTTGGGCTAGAATCATCCCCAGACATCTGCAGCTTGGAGGTGGTGGTAGC GGTGGTGGCGGTTCAGGTGGCGGAGGTTCTATCCAGAGAACCCCAAAAATTCAGGT GTACTCTCGGCACCCAGCCGAAAATGGCAAGTCCAATTTTCTCAATTGCTACGTCAG CGGGTTTCACCCTTCCGATATCGAAGTCGATCTGCTCAAGAATGGGGAGCGCATTGA GAAAGTCGAGCACAGCGACCTGTCATTCTCCAAGGATTGGAGCTTTTACCTCCTCTA CTATACGGAATTCACGCCCACAGAGAAGGATGAATATGCCTGCCGGGTCAACCATG TGACACTGTCTCAGCCCAAGATTGTCAAATGGGATCGAGACATGGGCGGTGGCGGC AGCGGAGGTGGTGGCTCAGGCGGAGGCGGTTCCGGCGGAGGCGGCTCTGGATCTCA TTCCATGAGATACTTCTCTGCCGCCGTGTCTAGACCAGGACGGGGAGAGCCTCGGTT TATCGCCATGGGCTATGTGGATGATACGCAATTTGTTCGCTTCGACAGCGACAGCGC CTGTCCTAGAATGGAACCCAGAGCACCTTGGGTCGAGCAAGAAGGCCCCGAGTATT GGGAAGAAGAGACACGGAACACCAAGGCTCACGCCCAGACCGACAGAATGAATCT GCAGACACTGCGCGGCTATTACAACCAGAGCGAAGCCAGCAGCCATACACTCCAGT GGATGATCGGCTGCGATCTGGGATCCGATGGCAGACTGCTGAGGGGATACGAACAG TACGCCTATGACGGGAAAGATTACCTGGCACTGAATGAGGATCTGCGGTCCTGGAC AGCCGCCGATACTGCTGCCCAGATCAGCAAGAGAAAGTGCGAGGCCGCCAATGTGG CCGAGCAGAGAAGGGCTTATCTCGAGGGCACATGCGTCGAGTGGCTGCATCGGTAT CTGGAAAACGGAAAAGAAATGCTGCAGCGGGCCGATCCTCCAAAAACACATGTGAC CCACCATCCTGTGTTCGACTACGAGGCTACCCTTCGGTGTTGGGCTCTCGGATTCTAC CCCGCTGAGATCATCCTCACTTGGCAACGCGACGGCGAAGATCAGACCCAGGACGT GGAACTGGTCGAGACTAGACCAGCTGGCGACGGGACATTTCAAAAGTGGGCCGCAG TGGTCGTGCCCTCCGGCGAAGAACAACGGTACACATGCCATGTGCAGCATGAAGGC CTGCCAGAGCCTCTGATGCTCAGGTGGAAGCAGAGCAGCCTGCCTACCATTCCTATC ATGGGCATCGTGGCCGGACTGGTGGTTCTGGCTGCTGTTGTTACAGGCGCAGCTGTG GCTGCAGTGCTGTGGCGGAAGAAGTCCAGCGATTG
SEQ ID NO: 31 HLA-E-2A-HLA-G transgene amino acid sequence with signal and presentation peptides
MVVMAPRTLFLLLSGALTLTETWAVMAPRTLILGGGGSGGGGSGGGGSIQRTPKIQVYS RHPAENGKSNFLNCYVSGFHPSDIEVDLLKNGERIEKVEHSDLSFSKDWSFYLLYYTEFT PTEKDEYACRVNHVTLSQPKIVKWDRDMGGGGSGGGGSGGGGSGGGGSGSHSLKYFH TSVSRPGRGEPRFISVGYVDDTQFVRFDNDAASPRMVPRAPWMEQEGSEYWDRETRSA RDTAQIFRVNLRTLRGYYNQSEAGSHTLQWMHGCELGPDGRFLRGYEQFAYDGKDYL TLNEDLRSWTAVDTAAQISEQKSNDASEAEHQRAYLEDTCVEWLHKYLEKGKETLLHL EPPKTHVTHHPISDHEATLRCWALGFYPAEITLTWQQDGEGHTQDTEL VETRPAGDGTF QKWAAVVVPSGEEQRYTCHVQHEGLPEPVTLRWKPASQPTIPIVGIIAGLVLLGSVVSG AVVAAVIWRKKSSGGKGGSYSKAEWSDSAQGSESHSLGSGATNFSLLKQAGDVEENPG PMVVMAPRTLFLLLSGALTLTETWARIIPRHLQLGGGGSGGGGSGGGGSIQRTPKIQVYS RHPAENGKSNFLNCYVSGFHPSDIEVDLLKNGERIEKVEHSDLSFSKDWSFYLLYYTEFT PTEKDEYACRVNHVTLSQPKIVKWDRDMGGGGSGGGGSGGGGSGGGGSGSHSMRYFS AAVSRPGRGEPRFIAMGYVDDTQFVRFDSDSACPRMEPRAPWVEQEGPEYWEEETRNT KAHAQTDRMNLQTLRGYYNQSEASSHTLQWMIGCDLGSDGRLLRGYEQYAYDGKDY LALNEDLRSWTAADTAAQISKRKCEAANVAEQRRAYLEGTCVEWLHRYLENGKEMLQ RADPPKTHVTHHPVFDYEATLRCWALGFYPAEIILTWQRDGEDQTQDVELVETRPAGD GTFQKWAAVVVPSGEEQRYTCHVQHEGLPEPLMLRWKQSSLPTIPIMGIVAGLVVLAA VVTGAAVAAVLWRKKSSD
SEQ ID NO: 32 HLA-E-2A-HLA-G transgene nucleotide sequence with signal and presentation peptides
ATGGTGGTCATGGCCCCTAGAACACTGTTCCTGCTGCTGTCTGGCGCCCTGACACTG ACAGAAACATGGGCCGTGATGGCCCCACGGACACTGATTCTTGGAGGCGGAGGATC TGGCGGAGGTGGAAGTGGCGGAGGCGGATCTATTCAGCGGACCCCTAAGATCCAGG TGTACAGCAGACACCCCGCCGAGAACGGCAAGAGCAACTTCCTGAACTGCTACGTG TCCGGCTTTCACCCCAGCGACATTGAGGTGGACCTGCTGAAGAACGGCGAGCGGAT CGAGAAGGTGGAACACAGCGATCTGAGCTTCAGCAAGGACTGGTCCTTCTACCTGCT GTACTACACCGAGTTCACCCCTACCGAGAAGGACGAGTACGCCTGCAGAGTGAACC ACGTGACCCTGAGCCAGCCTAAGATCGTGAAGTGGGACAGAGATATGGGAGGCGGC GGTAGTGGCGGCGGAGGAAGCGGAGGCGGAGGTTCAGGTGGTGGTGGATCTGGAA GCCACAGCCTGAAGTACTTTCACACCTCCGTGTCCAGACCTGGCAGAGGCGAGCCTA GATTCATCAGCGTGGGCTACGTGGACGACACCCAGTTCGTCAGATTCGACAACGAC GCCGCCTCTCCTCGGATGGTTCCTAGAGCACCCTGGATGGAACAAGAGGGCAGCGA GTACTGGGATCGCGAGACAAGAAGCGCCAGAGACACAGCCCAGATCTTCCGCGTGA ACCTGAGAACCCTGCGGGGCTACTACAATCAGTCTGAGGCCGGCTCTCACACCCTGC AGTGGATGCATGGATGTGAACTGGGCCCCGACGGCAGATTCCTGAGAGGCTATGAG CAGTTCGCCTACGACGGCAAGGACTATCTGACCCTGAACGAGGACCTGAGAAGCTG GACCGCCGTGGATACAGCCGCTCAGATCAGCGAGCAGAAGTCTAACGACGCCAGCG AGGCCGAACACCAGAGAGCCTATCTGGAAGATACCTGCGTGGAATGGCTGCACAAG TACCTGGAAAAGGGCAAAGAGACACTGCTGCACCTGGAACCACCTAAGACACACGT GACACATCACCCCATCAGCGACCACGAGGCCACACTGAGATGTTGGGCCCTGGGCT TTTACCCTGCCGAGATCACACTGACATGGCAGCAGGATGGCGAGGGCCACACACAG GATACAGAGCTGGTGGAAACAAGACCTGCCGGCGACGGCACCTTCCAGAAATGGGC TGCTGTGGTGGTTCCCAGCGGCGAGGAACAGAGATACACCTGTCACGTGCAGCACG AGGGACTGCCTGAACCTGTGACTCTGAGATGGAAGCCTGCCAGCCAGCCAACAATC CCCATCGTGGGAATCATTGCCGGCCTGGTGCTGCTGGGATCTGTGGTTTCTGGTGCA GTGGTGGCCGCCGTGATTTGGAGAAAGAAGTCCTCTGGCGGCAAAGGCGGCAGCTA CTCTAAAGCCGAGTGGAGCGATTCTGCCCAGGGCTCTGAATCTCACTCTCTTGGCAG CGGCGCCACCAACTTCAGCCTGCTTAAACAGGCAGGCGACGTGGAAGAGAACCCCG GACCTATGGTTGTGATGGCTCCTAGGACTTTGTTTCTGCTGCTCAGCGGCGCTCTGAC TCTGACCGAGACTTGGGCTAGAATCATCCCCAGACATCTGCAGCTTGGAGGTGGTGG TAGCGGTGGTGGCGGTTCAGGTGGCGGAGGTTCTATCCAGAGAACCCCAAAAATTC AGGTGTACTCTCGGCACCCAGCCGAAAATGGCAAGTCCAATTTTCTCAATTGCTACG TCAGCGGGTTTCACCCTTCCGATATCGAAGTCGATCTGCTCAAGAATGGGGAGCGCA TTGAGAAAGTCGAGCACAGCGACCTGTCATTCTCCAAGGATTGGAGCTTTTACCTCC TCTACTATACGGAATTCACGCCCACAGAGAAGGATGAATATGCCTGCCGGGTCAAC CATGTGACACTGTCTCAGCCCAAGATTGTCAAATGGGATCGAGACATGGGCGGTGG CGGCAGCGGAGGTGGTGGCTCAGGCGGAGGCGGTTCCGGCGGAGGCGGCTCTGGAT CTCATTCCATGAGATACTTCTCTGCCGCCGTGTCTAGACCAGGACGGGGAGAGCCTC GGTTTATCGCCATGGGCTATGTGGATGATACGCAATTTGTTCGCTTCGACAGCGACA GCGCCTGTCCTAGAATGGAACCCAGAGCACCTTGGGTCGAGCAAGAAGGCCCCGAG TATTGGGAAGAAGAGACACGGAACACCAAGGCTCACGCCCAGACCGACAGAATGA ATCTGCAGACACTGCGCGGCTATTACAACCAGAGCGAAGCCAGCAGCCATACACTC CAGTGGATGATCGGCTGCGATCTGGGATCCGATGGCAGACTGCTGAGGGGATACGA ACAGTACGCCTATGACGGGAAAGATTACCTGGCACTGAATGAGGATCTGCGGTCCT GGACAGCCGCCGATACTGCTGCCCAGATCAGCAAGAGAAAGTGCGAGGCCGCCAAT GTGGCCGAGCAGAGAAGGGCTTATCTCGAGGGCACATGCGTCGAGTGGCTGCATCG GTATCTGGAAAACGGAAAAGAAATGCTGCAGCGGGCCGATCCTCCAAAAACACATG TGACCCACCATCCTGTGTTCGACTACGAGGCTACCCTTCGGTGTTGGGCTCTCGGATT CTACCCCGCTGAGATCATCCTCACTTGGCAACGCGACGGCGAAGATCAGACCCAGG ACGTGGAACTGGTCGAGACTAGACCAGCTGGCGACGGGACATTTCAAAAGTGGGCC GCAGTGGTCGTGCCCTCCGGCGAAGAACAACGGTACACATGCCATGTGCAGCATGA AGGCCTGCCAGAGCCTCTGATGCTCAGGTGGAAGCAGAGCAGCCTGCCTACCATTC CTATCATGGGCATCGTGGCCGGACTGGTGGTTCTGGCTGCTGTTGTTACAGGCGCAG CTGTGGCTGCAGTGCTGTGGCGGAAGAAGTCCAGCGATTGA
SEQ ID NO: 33 Whitlow Linker
GSTSGSGKPGSGEGSTKG
SEQ ID NO: 34 Linker 3
GGSEGKSSGSGSESKSTGGS
SEQ ID NO: 35 Linker 4
GGGSGGGS
SEQ ID NO: 36 Linker 5
GGGSGGGSGGGS
SEQ ID NO: 37 Linker 6
GGGSGGGSGGGSGGGS
SEQ ID NO: 38 Linker 7
GGGSGGGSGGGSGGGSGGGS
SEQ ID NO: 39 Linker 8 (G₄S)₄
GGGGSGGGGSGGGGSGGGGS
SEQ ID NO: 40 Linker 9
GGGGSGGGGSGGGGSGGGGSGGGGS
SEQ ID NO: 41 (G4S)₂ Linker
GGGGSGGGGS
SEQ ID NO: 42 (G4S)₆
GGGGSGGGGSGGGGSGGGGSGGGGSGGGGS
SEQ ID NO: 43 (G4S)₇ Linker
GGGGSGGGGSGGGGSGGGGSGGGGSGGGGSGGGGS
SEQ ID NO: 44 (G4S)₈ Linker
GGGGSGGGGSGGGGSGGGGSGGGGSGGGGSGGGGGGGGS
SEQ ID NO: 45 Linker 10
IRPRAIGGSKPRVA
SEQ ID NO: 46 Linker 11
GKGGSGKGGSGKGGS
SEQ ID NO: 47 Linker 12
GGKGSGGKGSGGKGS
SEQ ID NO: 48 Linker 13
GGGKSGGGKSGGGKS
SEQ ID NO: 49 Linker 14
GKGKSGKGKSGKGKS
SEQ ID NO: 50 Linker 15
GGGKSGGKGSGKGGS
SEQ ID NO: 51 Linker 16
GKPGSGKPGSGKPGS
SEQ ID NO: 52 Linker 17
GKPGSGKPGSGKPGSGKPGS
SEQ ID NO: 53 Linker 18
GKGKSGKGKSGKGKSGKGKS
SEQ ID NO: 54 Linker 19
STAGDTHLGGEDFD
SEQ ID NO: 55 Linker 20
GEGGSGEGGSGEGGS
SEQ ID NO: 56 Linker 21
GGEGSGGEGSGGEGS
SEQ ID NO: 57 Linker 22
GEGESGEGESGEGES
SEQ ID NO: 58 Linker 23
GGGESGGEGSGEGGS
SEQ ID NO: 59 Linker 24
GEGESGEGESGEGESGEGES
SEQ ID NO: 60 Linker 25
PRGASKSGSASQTGSAPGS
SEQ ID NO: 61 Linker 26
GTAAAGAGAAGGAAAGAAG
SEQ ID NO: 62 Linker 27
GTSGSSGSGSGGSGSGGGG
SEQ ID NO: 63 Linker 28
GSGS
SEQ ID NO: 64 Linker 29
APAPAPAPAP
SEQ ID NO: 65 Linker 30
APAPAPAPAPAPAPAPAPAP
SEQ ID NO: 66 Linker 31
AEAAAKEAAAKEAAAAKEAAAAKEAAAAKAAA
SEQ ID NO: 67 Linker
(GGGGS)ₙ, wherein n is an integer of 1-8
SEQ ID NO: 68 Hinge
EPKSCDKTHTCPPCP
SEQ ID NO: 69 Hinge
ERKCCVECPPCP
SEQ ID NO: 70 Hinge
ELKTPLGDTTHTCPRCP (EPKSCDTPPPCPRCP) 3
SEQ ID NO: 71 Hinge
ESKYGPPCPSCP
SEQ ID NO: 72 AAVS1 gRNA
TTTATCTGTCCCCTCCACCCCACA
SEQ ID NO: 73 AAVS1 Left Homology Arm
ACTCTGCCCCAGGCCTCCTTACCATTCCCCTTCGACCTACTCTCTTCCGCATTGGAGT CGCTTTAACTGGCCCTGGCTTTGGCAGCCTGTGCTGACCCATGCAGTCCTCCTTACCA TCCCTCCCTCGACTTCCCCTCTTCCGATGTTGAGCCCCTCCAGCCGGTCCTGGACTTT GTCTCCTTCCCTGCCCTGCCCTCTCCTGAACCTGAGCCAGCTCCCATAGCTCAGTCTG GTCTATCTGCCTGGCCCTGGCCATTGTCACTTTGCGCTGCCCTCCTCTCGCCCCCGAG TGCCCTTGCTGTGCCGCCGGAACTCTGCCCTCTAACGCTGCCGTCTCTCTCCTGAGTC CGGACCACTTTGAGCTCTACTGGCTTCTGCGCCGCCTCTGGCCCACTGTTTCCCCTTC CCAGGCAGGTCCTGCTTTCTCTGACCTGCATTCTCTCCCCTGGGCCTGTGCCGCTTTC TGTCTGCAGCTTGTGGCCTGGGTCACCTCTACGGCTGGCCCAGATCCTTCCCTGCCG CCTCCTTCAGGTTCCGTCTTCCTCCACTCCCTCTTCCCCTTGCTCTCTGCTGTGTTGCT GCCCAAGGATGCTCTTTCCGGAGCACTTCCTTCTCGGCGCTGCACCACGTGATGTCC TCTGAGCGGATCCTCCCCGTGTCTGGGTCCTCTCCGGGCATCTCTCCTCCCTCACCCA ACCCCATGCCGTCTTCACTCGCTGGGTTCCCTTTTCCTTCTCCTTCTGGGGCCTGTGC CATCTCTCGTTTCTTAGGATGGCCTTCTCCGACGGATGTCTCCCTTGCGTCCCGCCTC CCCTTCTTGTAGGCCTGCATCATCACCGTTTTTCTGGACAACCCCAAAGTACCCCGTC TCCCTGGCTTTAGCCACCTCTCCATCCTCTTGCTTTCTTTGCCTGGACACCCCGTTCTC CTGTGGATTCGGGTCACCTCTCACTCCTTTCATTTGGGCAGCTCCCCTACCCCCCTTA CCTCTCTAGTCTGTGCTAGCTCTTCCAGCCCCCTGTCATGGCATCTTCCAGGGGTCCG AGAGCTCAGCTAGTCTTCTTCCTCCAACCCGGGCCCCTATGTCCACTTCAGGACAGC ATGTTTGCTGCCTCCAGGGATCCTGTGTCCCCGAGCTGGGACCACCTTATATTCCCA GGGCCGGTTAATGTGGCTCTGGTTCTGGGTACTTTTATCTGTCCCCT
SEQ ID NO: 74 AAVS1 Right Homology Arm
CCACCCCACAGTGGGGCCACTAGGGACAGGATTGGTGACAGAAAAGCCCCATCCTT AGGCCTCCTCCTTCCTAGTCTCCTGATATTGGGTCTAACCCCCACCTCCTGTTAGGCA GATTCCTTATCTGGTGACACACCCCCATTTCCTGGAGCCATCTCTCTCCTTGCCAGAA CCTCTAAGGTTTGCTTACGATGGAGCCAGAGAGGATCCTGGGAGGGAGAGCTTGGC AGGGGGTGGGAGGGAAGGGGGGGATGCGTGACCTGCCCGGTTCTCAGTGGCCACCC TGCGCTACCCTCTCCCAGAACCTGAGCTGCTCTGACGCGGCCGTCTGGTGCGTTTCA CTGATCCTGGTGCTGCAGCTTCCTTACACTTCCCAAGAGGAGAAGCAGTTTGGAAAA ACAAAATCAGAATAAGTTGGTCCTGAGTTCTAACTTTGGCTCTTCACCTTTCTAGTCC CCAATTTATATTGTTCCTCCGTGCGTCAGTTTTACCTGTGAGATAAGGCCAGTAGCCA GCCCCGTCCTGGCAGGGCTGTGGTGAGGAGGGGGGTGTCCGTGTGGAAAACTCCCT TTGTGAGAATGGTGCGTCCTAGGTGTTCACCAGGTCGTGGCCGCCTCTACTCCCTTTC TCTTTCTCCATCCTTCTTTCCTTAAAGAGTCCCCAGTGCTATCTGGGACATATTCCTC CGCCCAGAGCAGGGTCCCGCTTCCCTAAGGCCCTGCTCTGGGCTTCTGGGTTTGAGT CCTTGGCAAGCCCAGGAGAGGCGCTCAGGCTTCCCTGTCCCCCTTCCTCGTCCACCA TCTCATGCCCCTGGCTCTCCTGCCCCTTCCCTACAGGGGTTCCTGGCTCTGCTCTTCA GACTGAGCCCCGTTCCCCTGCATCCCCGTACCCCTGCATCCCCCTTCCCCTGCATCCC CCAGAGGCCCCAGGCCACCTACTTGGCCTGGACCCCACGAGAGGCCACCCCAGCCC TGTCTACCAGGCTGCCTTTTGGGTGGATTCTCCTCCAACTGTGGGGTGACTGCTTGGC AAACTCACTCTTCGGGGTATCCCAGGAGGCCTGGAGCATTGGGGTGGGCTGGGGTTC AGAGAGGAGGGATTCCCTTCTCAGGTTACGTGGCCAAGAAGCAGGGGAGCTGGGTT TGGGTCAGGTCTGGGTGTGGGGTGACCAGCTTATGCTGTTTGCCCAGGACAGCCTAG
SEQ ID NO: 75 B2M gRNA
TTTACTCACGTCATCCAGCAGAGA
SEQ ID NO: 76 B2M Left Homology Arm
GCATATAAAACCTCAGCAGAAATAAAGAGGTTTTGTTGTTTGGTAAGAACATACCTT GGGTTGGTTGGGCACGGTGGCTCGTGCCTGTAATCCCAACACTTTGGGAGGCCAAGG CAGGCTGATCACTTGAAGTTGGGAGTTCAAGACCAGCCTGGCCAACATGGTGAAAT CCCGTCTCTACTGAAAATACAAAAATTAACCAGGCATGGTGGTGTGTGCCTGTAGTC CCAGGAATCACTTGAACCCAGGAGGCGGAGGTTGCAGTGAGCTGAGATCTCACCAC TGCACACTGCACTCCAGCCTGGGCAATGGAATGAGATTCCATCCCAAAAAATAAAA AAATAAAAAAATAAAGAACATACCTTGGGTTGATCCACTTAGGAACCTCAGATAAT AACATCTGCCACGTATAGAGCAATTGCTATGTCCCAGGCACTCTACTAGACACTTCA TACAGTTTAGAAAATCAGATGGGTGTAGATCAAGGCAGGAGCAGGAACCAAAAAG AAAGGCATAAACATAAGAAAAAAAATGGAAGGGGTGGAAACAGAGTACAATAACA TGAGTAATTTGATGGGGGCTATTATGAACTGAGAAATGAACTTTGAAAAGTATCTTG GGGCCAAATCATGTAGACTCTTGAGTGATGTGTTAAGGAATGCTATGAGTGCTGAGA GGGCATCAGAAGTCCTTGAGAGCCTCCAGAGAAAGGCTCTTAAAAATGCAGCGCAA TCTCCAGTGACAGAAGATACTGCTAGAAATCTGCTAGAAAAAAAACAAAAAAGGCA TGTATAGAGGAATTATGAGGGAAAGATACCAAGTCACGGTTTATTCTTCAAAATGG AGGTGGCTTGTTGGGAAGGTGGAAGCTCATTTGGCCAGAGTGGAAATGGAATTGGG AGAAATCGATGACCAAATGTAAACACTTGGTGCCTGATATAGCTTGACACCAAGTTA GCCCCAAGTGAAATACCCTGGCAATATTAATGTGTCTTTTCCCGATATTCCTCAGGT ACTCCAAAGATTCAGGTTTACTCACGTC
SEQ ID NO: 77 B2M Right Homology Arm
ATCCAGCAGAGAATGGAAAGTCAAATTTCCTGAATTGCTATGTGTCTGGGTTTCATC CATCCGACATTGAAGTTGACTTACTGAAGAATGGAGAGAGAATTGAAAAAGTGGAG CATTCAGACTTGTCTTTCAGCAAGGACTGGTCTTTCTATCTCTTGTACTACACTGAAT TCACCCCCACTGAAAAAGATGAGTATGCCTGCCGTGTGAACCATGTGACTTTGTCAC AGCCCAAGATAGTTAAGTGGGGTAAGTCTTACATTCTTTTGTAAGCTGCTGAAAGTT GTGTATGAGTAGTCATATCATAAAGCTGCTTTGATATAAAAAAGGTCTATGGCCATA CTACCCTGAATGAGTCCCATCCCATCTGATATAAACAATCTGCATATTGGGATTGTC AGGGAATGTTCTTAAAGATCAGATTAGTGGCACCTGCTGAGATACTGATGCACAGC ATGGTTTCTGAACCAGTAGTTTCCCTGCAGTTGAGCAGGGAGCAGCAGCAGCACTTG CACAAATACATATACACTCTTAACACTTCTTACCTACTGGCTTCCTCTAGCTTTTGTG GCAGCTTCAGGTATATTTAGCACTGAACGAACATCTCAAGAAGGTATAGGCCTTTGT TTGTAAGTCCTGCTGTCCTAGCATCCTATAATCCTGGACTTCTCCAGTACTTTCTGGC TGGATTGGTATCTGAGGCTAGTAGGAAGGGCTTGTTCCTGCTGGGTAGCTCTAAACA ATGTATTCATGGGTAGGAACAGCAGCCTATTCTGCCAGCCTTATTTCTAACCATTTTA GACATTTGTTAGTACATGGTATTTTAAAAGTAAAACTTAATGTCTTCCTTTTTTTTCT CCACTGTCTTTTTCATAGATCGAGACATGTAAGCAGCATCATGGAGGTAAGTTTTTG ACCTTGAGAAAATGTTTTTGTTTCACTGTCCTGAGGACTATTTATAGACAGCTCTAAC ATGATAACCCTCACTATGTGGAGAACATTGACAGAGTAACATTTTAGCAG
SEQ ID NO: 78 CIITA gRNA
TTTACCTTGGGGCTCTGACAGGTA
SEQ ID NO: 79 CIITA Left Homology Arm
ACAGGAGGTAACCATTTAACAAGAAAGCAGAGTGATGTTAGATTATAGCAAGATAC TGTTGACTGTAGAAGGCTCTGAGGCTAGAGAGCTGCTTTCTATAAAACAGAGTGATC ATATATTAGAAGAGGTGTTAAAGACATGTTCACACCAAGCTGAGACTTCCTCCTTGA TACCACCAGGAGGATGGGCAGAGACTGGAAAAGACACTAACTTTCTCCCTATGGGA GTCAGTATTATTTAGCATCACTTTGGCGGGTCACCCCAAACCATCTGACTACAAGGG TACCATATTTGGGTTAACACTCTTTTGGTATAATTTATGTTTTAGTCCAATGTCTTGG GATGAAAATGACAGGTGGGCCACTTATGATCTCCAGAGAAATTCAGGGCAATTTGG TGTGGGAGTAGGCATGGTAGAGGAGAGCAGCATCTAAGAAGTCCCCAGCAGAGGCT CTCAGCTTGTCTTGAGGCATCTGGGCGGAGGGCTATGATACTGGCCCCATCCTGCAG AAGGTGGCAGATATTGGCAGCTGGCACCAGTGCGGTTCCATTGTGATCATCATTTCT GAACGTCAGACTGTTGAAGGTTCCCCCAACAGACTTTCTGTGCAACTTTCTGTCTTCA CCAAATTCAGTCCACAGTAAGGAAGTGAAATTAATTTCAGAGGTGTGGGGAGGGCT TAAGGGAGTGTGGTAAAATTAGAGGGTGTTCAGAAACAGAAATCTGACCGCTTGGG GCCACCTTGCAGGGAGAGTTTTTTTGATGATCCCTCACTTGTTTCTTTGCATGTTGGC TTAGCTTGGCGGGCTCCCAACTGGTGACTGGTTAGTGATGAGGCTAGTGATGAGGCT GTGTGCTTCTGAGCTGGGCATCCGAAGGCATCCTTGGGGAAGCTGAGGGCACGAGG AGGGGCTGCCAGACTCCGGGAGCTGCTGCCTGGCTGGGATTCCTACACAATGCGTTG CCTGGCTCCACGCCCTGCTGGGTCCTACCTGTCAG
SEQ ID NO: 80 CIITA Right Homology Arm
AGCCCCAAGGTAAAAAGGCCGGGAAAGCATCTTAATTTAGCGTGCAGTCTCAGCTG GTCCTGCCATTCCAGATAAACAGAGAAACCATTCTGAATTGGGGATGGGGGTGAGG ATGGGAACAGGAGTCTGTGTCCTGCTGGGGCAGGCCATTGGAAGATGTGAAAGAGT TGTCTATTTCCTTCCACCGGAGGGAGACTTCAGGTCAGCCAGGTGTCTGGAGTATGA ACCATGTATCAGCACCGAAAGGTTCTAGAAGTCAGACTTTCGGGCAGTGTGTCACTA ACTCTCAGCATGCTGGCCTGGCTCGGCCCACAGCAAGGTCTTCTCGCCTCCCTTTGG GTAAATACTGAGGGGTGCCTCTGCAGGACGGGACCTCTGCCAGACTCCACTCCATAC CCAGAGAAGCAGGGAAACCAAAATTGGAGTCAGCCTTGAGGTGTAGCTGTTGAGCC CTCAGCAGCTGGGGAGAGCTGGCGGATGCTGCCCTCCCCCCAGTTTCCTAATGGTGT TGTTTAAAAAGGGTCAGGGGACGGGGGAACAGATGGTGGGAAGAGCACAGTGCAG ACACCTGGCACCGGCTCTGAAGGCAGCATGGCAGCTACACCGTTGGCTGGGAAGGG TGTGCCCCTGAAGAAGTCGTTTACATTCTCGAGTCAATTTTCCTGGAGTGTACAATG GACCTGTGGGAAAGCCTGTATGAAAGGGTAATGATGAGGGACCTAGCACAGTGTCC AATATTTTATAGGAACTGGAATTGAGCTCATAGGAGCTCAATTTTATTGGCATTGCT GTTGTTGGATGGTTAAAGGGGTGGTATCCCTTTTCTCAGACTCCCCTGAAATGTATG GTTTGCTTTGAACCCAGAGACTGATGACAGGTCTGCCGGTGTGGTTGGGTGCAGCCT TAAGTTGCTACGGGAAAGTGTTGGAGGGGGAGAAGTCAGAGGTAACCTTGCCCCCT CCCTCAATTCCAGATGAGGAAATTCAGGCCTGAAAAGGGA
SEQ ID NO: 81 CLYBL gRNA
AGAGTGATCACAGCTCTGACTAAA
SEQ ID NO: 82 CLYBL Left Homology Arm
GTCCAGACTCAGGAGGACTTAGTTCTCTTCATAAAAAGTTGATCACCTTTGCTTTCTC CTTTGCATGGGCAATAAAGAAGTGAAAAATAAATTCCCAGTGAGCTTGCATTCTGCT TGGGAACAACATAGCACATCCATTTTCTAGAGAGCTGTCCAGTCCCCATTTGAGGGC TGCTAGCCACATGTCAAGCAGGAACCCAGAGTATAACCAGGAAAATGTCTGTGGTA GACCCCAGTGATTCATGCCTCCCATCTCCACACCCTCCTGTAGTCCCCTCCCACGCTG ATTCTGGACTAGGCCACATGGCTTGGCCAGTGGAACACATGCAGGCTTGCACGTCTG GAACTCTGCCACCTAATTTAGGATCCCAGACTCTCCTACTGGAGACACAGGTCCTTA GTGACAGTCTGCACCACCATTCAGACAAGTCAGTAGGGCCATCTTAGATCATCCAGC CCTAGTCAAGCCACCAGATAACTGTACCCACATAAGTGACCCCTGGCGAGACCAGC AGGAGAATCATGCCAATGGGCCAATATACATTCTGACCCACAGTTTCATAATAAAAT AAAATGGTTGTGGTTGTAAGCCACTATGTTTCAGAGTGGTTTGTTACACAGCAATAA ATAACTAATATAGTAGGCATACCATCAAGTCCAAAGTAGGTAGAGAAGAATGTAAA TAGCAGAGCAAAACAGCATGACTGGTGGCTGGGAGGCTTAAAACTGGGACAGGATC AGAGTCATGAAAGAAGTCAAAGAAATGGTTCAGAAGTAAGGCTGAGACTGACTTAC AAAAGCTGAAAGTCCCTTTAAGTTGGTGTTTGGTGCATTGGCAGGGGCAGGTATGGT GACTTAAAAGAGCCATGCTCAACAAGATCAAGCACAACACAATCACGGGTCACCCC AGCAGACCTTAGCGAGTCTAGCCATTTCTTTGGTGGTGGTCACAGTCATGCTTCAGC CCAGTTTCCACTTGGACAAATGGTACATATTTTCAATGAGATGAAAATTAAGATACA ATCCATGTGCTCAGAGAGTGATCA
SEQ ID NO: 83 CLYBL Right Homology Arm
ATCATGTCTGCTCGACAGCTCTGACTAAACACTGTGCCCCAAAGTGTTGAGGAATTG GGAAAACCTAGCTGAGTTAGTGGTCTCTTTTCTGTTACAATAAAGCTCATAATGAAA ATTAGCCTTCTTTGTTCTTCCCCAAGTCTTTCTTTCTAGACGAAACTACTTTCAACTGT TTTAACTTCCTTACTGTTAACTTCCATATTTCTAGATAGTATGGTCAGACTGTTATTTC TTGACTTTTAAATGTAAGATATTATCTACTGACTTCCTTCTATGTAAGATGAGGATTG AGCTCTCTTACCCTTCTCCCATTTCCTCATCCTTCCAACATAAATATATTTTGGGATT ATATCAACATTCAATGTTACTTAAAGTGACCTTGTAAATATTTTCACAACTGAGCCA TGTTTGATTTGTATACTTATGTTTACTTTACTGTTTTTCCTGAAGTTAATAATTGCCTT GAATTTATTTATTTCTTTAAAAATGTTTCATTACTCAGGACTGTAGTTTACATTACGA TTCTTTGTGTTATACAGTTGATGGGTTTCTTTTCTTTCTTAATTTCTTTAAAAAATAGA GATGGGGTCTTACTATATTACCCAGGCTGGTCTTGAAGTCCTGGGCTCAAGTGATCT TCCTGTCTCAGCCTACCAAGTAGCTGAGACTATAGGTGCAAAAAAGCCACTATACCT GGCTAGTTTACAGGTTTTAACAAATGCATTATGCCACGTATCCATTATTACAGGATC ACACAAGATATTTTCATTACCCTGAAAGCATCCCTGTGTTCCACCAATTCATCCTGCC TCCATGAGCCGCTGGCAACCACTGATCTCTATAGTTTTGCCTTTTCTAAAATGTCATA TAATTGGAATCATACAGTCTGTAGCATTTTCAGACTAGCTTTTAAAATTTGGCAATAT GCATTTAAGGTTCCTCCTTAAATGTGAAGGGCATAGCCAATGTGGCTTGATAGCTCA TTTCTTTTTATTGGTGAATATTTCATTGTCTGGATGTTCCACAGTTTGTTTATCCTCGA GAGCTTGGCGT
SEQ ID NO: 84 NKG2A gRNA
CTCAGACCTGAATCTGCCCCCAAA
SEQ ID NO: 85 NKG2A Left Homology Arm
GACCTGGCCTCCGTTTTATGTAGTGTTCCTAATACTATTTAGGTTATGCATAAGTTAA CTGTGTCACATAGCATTTTCTGTCATATACAAATATTTAATTTTTTATAATTTTTAATA AATAATTTTTAATAATTTCTTAGAACATTTCAGCCATTAAGAAAAGACCATTTAATT ACCTCTATTGATTTTCATTAATGTGTATAAATTACTAATTAAAAGTAATGTAGTGCAA GAAATTTTTATATATATATATGTCAACTTTACTATTACTCATGTGCAGACCACATAGT CTTAACCATTACTGGATTTCAAAATTAACATAAAAATGAGTGCAGAAGTTTTTCTAA TACAACATTTTAATTTTTAACACAGGTAAAACATCAGACTTTAAGAAATATATTTTT ATTCCTGAACTTCTTTATTCCTTAGTAATTTATTGCTTCTCATTGCCCCAGCAATAAT ATTTTGTCAAATGCAGAAAATTTATCTTTTTTTTTTGAGTCGGAGTCTCACTCTGTCG CCCAGGCTGGAGTGCAGTGGCACAATCTCCGCTCACTGCAACCTCTGCCGCCCATGT TCAAGAGATTCTCCTGCCTCAGCCTCCCGAGTAGCTAGGACTACAGGCGCCTGACAT CATGCCCGACTACTTTTTGTATTTTTAGTAGAGACGGAGTTTCACCGTGTTAGCCAGG ATGGTCTCGATCTCCTGACCTCGTGATCGGCATGCCTCGGCCTCCCAAAGTGCTGGG ATTACAGGCGTGAGCCACCGCGCCCGGCCTAAAAATCTTTTTTTAAAACAAATATTC ATAAGAAACGTGTTTAGGCTTGAAGAAAATCAGAGAAAGAACTTTAGATTATTTAA TGCAAAATGAGCTCCAATACTCGTTCTCCACCTCACCCTTTTAATTGCACTAGGGAA TCCTGTATATAAACCATTTATTAACTTCTTAACTACTGTTATTATAGAGTACAGTCCC TGACATCACACACTGCAGAGATGGATAACCAAGGAGTAATCTACTCAGACCTGAAC GGTACCAACGCG
SEQ ID NO: 86 NKG2A Right Homology Arm
AATCTGCCCCCAAACCCAAAGAGGCAGCAACGAAAACCTAAAGGCAATAAAAACTC CATTTTAGCAACTGAACAGGAAATAACCTATGCGGAATTAAACCTTCAAAAAGCTTC TCAGGATTTTCAAGGGAATGACAAAACCTATCACTGCAAAGGTAAAGCATTTAAAA GATCCTCAATATAACAGTCTAGGATGTGCAGCTTGGGGTACAGGAATGTGGGGAAA GAGAAGGGAGTGCTCATATATCTTCTATTTGCAAAGATCAGAATTCCAAGTTGAGAT ATGCTATTTCAATGTAAAGTATGAAGACTGATTGAACTCATTGTTGAAGTTTGTAGT CTTTGTCAAATAATTCATGGAGCATTATTTTTCCTGAAAATTCAATGGTATATTATTC TGAGAAAAAGATTACAATGGGAGATGAGGGTTTGGGGTCCAAGTTTCTCTGTATGAT TCCTGTGCATTCAGGTTCTCTTGTCTGTGAATCTTCTAAACGACTGTATCCACCTCTC CTTTCGCACTGTTCCCATTTCTCTCCCTGCAGATTTACCATCAGCTCCAGAGAAGCTC ATTGTTGGGATCCTGGGAATTATCTGTCTTATCTTAATGGCCTCTGTGGTAACGATAG TTGTTATTCCCTGTAAGTCTATTTTCGAAGATTACAAGGGGAATTTTCACGTTAATGA TTGAATGTGCCTCTAAACATTTCATATTTTCAGGGAATAGAGTTCTCATTGTAATGTA TATATTTGGACTAAATGTGGAATGATTATTCTGAATTTGTCAAAGAATAAATGAAAG AATAATTGTTGAAAGTATTCGCTTCTGATGCAATCGTATGTATATATTTGGATTTCAT AACTCAAAAATATGTTCTAGGAGTCTGAAAAACCTTACTGAGAAATAGAAATTAATT TTTGAAAGTAGTTAAATCAAGAATTATAAGAACTATATGAGATGGTGAAATTTGGTT CTTTAGATCTATGAAATACTTTTCCAAAAAACCACCATTACTTTATC
SEQ ID NO: 87 TRAC gRNA
GTGTACCAGCTGAGAGACTCTAAA
SEQ ID NO: 88 TRAC Left Homology Arm
CGACTCTAGACTCGAAGCTGGATGTCTGCATTGCCGAGGCCACCAGGGCTGGCTCA GCAACTGTCGGGGAATCACCAGGGTCTGAGAAATCTTGTGCGCATGTGAGGGGCTG TGGGAGCAGAGAACCACTGGGTGGGAAATTCTAATCCCCACCCTGCTGGAAACTCT CTGGGTGGCCCCAACATGCTAATCCTCCGGCAAACCTCTGTTTCCTCCTCAAAAGGC AGGAGGTCGGAAAGAATAAACAATGAGAGTCACATTAAAAACACAAAATCCTACG GAAATACTGAAGAATGAGTCTCAGCACTAAGGAAAAGCCTCCAGCAGCTCCTGCTT TCTGAGGGTGAAGGATAGACGCTGTGGCTCTGCATGACTCACTAGCACTCTATCACG GCCATATTCTGGCAGGGTCAGTGGCTCCAACTAACATTTGTTTGGTACTTTACAGTTT ATTAAATAGATGTTTATATGGAGAAGCTCTCATTTCTTTCTCAGAAGAGCCTGGCTA GGAAGGTGGATGAGGCACCATATTCATTTTGCAGGTGAAATTCCTGAGATGTAAGG AGCTGCTGTGACTTGCTCAAGGCCTTATATCGAGTAAACGGTAGTGCTGGGGCTTAG ACGCAGGTGTTCTGATTTATAGTTCAAAACCTCTATCAATGAGAGAGCAATCTCCTG GTAATGTGATAGATTTCCCAACTTAATGCCAACATACCATAAACCTCCCATTCTGCT AATGCCCAGCCTAAGTTGGGGAGACCACTCCAGATTCCAAGATGTACAGTTTGCTTT GCTGGGCCTTTTTCCCATGCCTGCCTTTACTCTGCCAGAGTTATATTGCTGGGGTTTT GAAGAAGATCCTATTAAATAAAAGAATAAGCAGTATTATTAAGTAGCCCTGCATTTC AGGTTTCCTTGAGTGGCAGGCCAGGCCTGGCCGTGAACGTTCACTGAAATCATGGCC TCTTGGCCAAGATTGATAGCTTGTGCCTGTCCCTGAGTCCCAGTCCATCACGAGCAG CTGGTTTCTAAGATGCTATTTCCCGTATAAAGCATGAGACCGTGACTTGCCAGCCCC ACAGAGCCCCGCCCTTGTCCATCACTGGCATCTGGACTCCAGCCTGGGTTGGGGCAA AGAGGGAAATGAGATCATGTCCTAACCCTGATCCTCTTGTCCCACAGATATCCAGAA CCCTGACCCTGCCGTGTACCAGCTCGAGAAGGATCTG
SEQ ID NO: 89 TRAC Right Homology Arm
ATCATGTCTGGGATCTGAGAGACTCTAAATCCAGTGACAAGTCTGTCTGCCTATTCA CCGATTTTGATTCTCAAACAAATGTGTCACAAAGTAAGGATTCTGATGTGTATATCA CAGACAAAACTGTGCTAGACATGAGGTCTATGGACTTCAAGAGCAACAGTGCTGTG GCCTGGAGCAACAAATCTGACTTTGCATGTGCAAACGCCTTCAACAACAGCATTATT CCAGAAGACACCTTCTTCCCCAGCCCAGGTAAGGGCAGCTTTGGTGCCTTCGCAGGC TGTTTCCTTGCTTCAGGAATGGCCAGGTTCTGCCCAGAGCTCTGGTCAATGATGTCTA AAACTCCTCTGATTGGTGGTCTCGGCCTTATCCATTGCCACCAAAACCCTCTTTTTAC TAAGAAACAGTGAGCCTTGTTCTGGCAGTCCAGAGAATGACACGGGAAAAAAGCAG ATGAAGAGAAGGTGGCAGGAGAGGGCACGTGGCCCAGCCTCAGTCTCTCCAACTGA GTTCCTGCCTGCCTGCCTTTGCTCAGACTGTTTGCCCCTTACTGCTCTTCTAGGCCTC ATTCTAAGCCCCTTCTCCAAGTTGCCTCTCCTTATTTCTCCCTGTCTGCCAAAAAATC TTTCCCAGCTCACTAAGTCAGTCTCACGCAGTCACTCATTAACCCACCAATCACTGA TTGTGCCGGCACATGAATGCACCAGGTGTTGAAGTGGAGGAATTAAAAAGTCAGAT GAGGGGTGTGCCCAGAGGAAGCACCATTCTAGTTGGGGGAGCCCATCTGTCAGCTG GGAAAAGTCCAAATAACTTCAGATTGGAATGTGTTTTAACTCAGGGTTGAGAAAAC AGCTACCTTCAGGACAAAAGTCAGGGAAGGGCTCTCTGAAGAAATGCTACTTGAAG ATACCAGCCCTACCAAGGGCAGGGAGAGGACCCTATAGAGGCCTGGGACAGGAGCT CAATGAGAAAGGAGAAGAGCAGCAGGCATGAGTTGAATGAAGGAGGCAGGGCCGG GTCACAGGGCCTTCTAGGCCATGAGAGGGTAGACAGTATTCTAAGGACGCCAGAAA GCTGTTGATCGGCTTCAAGCAGGGGAGGGACACCTAATTGATCCGATCCGAGCT
SEQ ID NO: 90 HLA-G signal-Peptide-B2M-HLA-G
MVVMAPRTLFLLLSGALTLTETWARIIPRHLQLGGGGSGGGGSIQRTPKIQVYSRHPAEN GKSNFLNCYVSGFHPSDIEVDLLKNGERIEKVEHSDLSFSKDWSFYLLYYTEFTPTEKDE YACRVNHVTLSQPKIVKWDRDMGGGGSGGGGSGGGGSGSHSMRYFSAAVSRPGRGEP RFIAMGYVDDTQFVRFDSDSACPRMEPRAPWVEQEGPEYWEEETRNTKAHAQTDRMN LQTLRGYYNQSEASSHTLQWMIGCDLGSDGRLLRGYEQYAYDGKDYLALNEDLRSWT AADTAAQISKRKCEAANVAEQRRAYLEGTCVEWLHRYLENGKEMLQRADPPKTHVTH HPVFDYEATLRCWALGFYPAEIILTWQRDGEDQTQDVELVETRPAGDGTFQKWAAVVV PSGEEQRYTCHVQHEGLPEPLMLRWKQSSLPTIPIMGIVAGLVVLAAVVTGAAVAAVL WRKKSSD
SEQ ID NO: 91 HLA-G signal-Peptide-B2M-HLA-G
GCCACCATGGTGGTCATGGCGCCCCGAACCCTCTTCCTGCTGCTCTCGGGGGCCCTG ACCCTGACCGAGACCTGGGCGCGGATCATTCCCCGACATCTGCAACTGGGAGGCGG CGGTTCAGGAGGGGGCGGATCGATCCAACGCACCCCCAAGATCCAGGTCTACTCCA GACACCCGGCCGAAAACGGAAAGTCGAACTTCCTGAACTGCTATGTGTCAGGATTC CACCCGTCCGACATCGAGGTGGACCTCCTGAAGAACGGCGAACGCATTGAGAAGGT CGAGCACTCCGATCTGTCGTTCTCCAAGGACTGGTCCTTCTACCTTCTCTACTATACC GAATTCACCCCGACCGAGAAGGACGAATACGCCTGCCGGGTCAACCACGTGACCCT GAGCCAGCCAAAGATCGTGAAATGGGACCGCGATATGGGAGGAGGAGGTTCCGGC GGAGGAGGAAGCGGAGGCGGAGGTTCCGGCTCCCACTCCATGAGGTATTTCAGCGC CGCCGTGTCCCGGCCTGGCCGCGGAGAGCCTCGCTTCATCGCCATGGGATACGTGGA CGACACCCAGTTCGTCAGATTCGACAGCGACAGCGCCTGTCCTCGGATGGAACCTA GAGCACCTTGGGTCGAGCAAGAGGGCCCTGAGTACTGGGAAGAAGAGACACGGAA CACCAAGGCTCACGCCCAGACCGACAGAATGAACCTGCAGACCCTGCGGGGCTACT ACAATCAGTCTGAGGCCAGCAGCCATACTCTGCAGTGGATGATCGGCTGCGATCTGG GCTCTGATGGCAGACTGCTGAGAGGCTACGAGCAGTACGCCTACGACGGCAAGGAT TATCTGGCCCTGAACGAGGACCTGCGGTCTTGGACAGCTGCCGATACAGCCGCTCAG ATCAGCAAGAGAAAGTGCGAGGCCGCCAATGTGGCCGAACAGAGAAGGGCTTACCT GGAAGGCACCTGTGTGGAATGGCTGCACAGATACCTGGAAAACGGCAAAGAGATGC TGCAGCGGGCCGATCCTCCTAAGACACATGTGACCCACCATCCTGTGTTCGACTACG AGGCCACACTGAGATGTTGGGCCCTGGGCTTTTACCCTGCCGAGATCATCCTGACCT GGCAGCGAGATGGCGAGGATCAGACCCAGGATGTGGAACTGGTGGAAACCAGACCT GCCGGCGACGGCACCTTTCAGAAATGGGCTGCTGTGGTGGTGCCCAGCGGAGAGGA ACAGAGATACACCTGTCACGTGCAGCACGAGGGACTGCCTGAACCTCTGATGCTGA GATGGAAGCAGAGCAGCCTGCCTACAATCCCCATCATGGGAATCGTGGCCGGACTG GTGGTTCTGGCCGCTGTTGTTACAGGTGCTGCAGTGGCTGCCGTGCTGTGGCGGAAG AAAAGCAGCGACTGA
SEQ ID NO: 92 CAG promoter
ATTGATTATTGACTAGTTATTAATAGTAATCAATTACGGGGTCATTAGTTCATAGCCC ATATATGGAGTTCCGCGTTACATAACTTACGGTAAATGGCCCGCCTGGCTGACCGCC CAACGACCCCCGCCCATTGACGTCAATAATGACGTATGTTCCCATAGTAACGCCAAT AGGGACTTTCCATTGACGTCAATGGGTGGACTATTTACGGTAAACTGCCCACTTGGC AGTACATCAAGTGTATCATATGCCAAGTACGCCCCCTATTGACGTCAATGACGGTAA ATGGCCCGCCTGGCATTATGCCCAGTACATGACCTTATGGGACTTTCCTACTTGGCA GTACATCTACGTATTAGTCATCGCTATTACCATGGGTCGAGGTGAGCCCCACGTTCT GCTTCACTCTCCCCATCTCCCCCCCCTCCCCACCCCCAATTTTGTATTTATTTATTTTT TAATTATTTTGTGCAGCGATGGGGGCGGGGGGGGGGGGGGCGCGCGCCAGGCGGGG CGGGGCGGGGCGAGGGGCGGGGCGGGGCGAGGCGGAGAGGTGCGGCGGCAGCCAA TCAGAGCGGCGCGCTCCGAAAGTTTCCTTTTATGGCGAGGCGGCGGCGGCGGCGGC CCTATAAAAAGCGAAGCGCGCGGCGGGCGGGAGTCGCTGCGTTGCCTTCGCCCCGT GCCCCGCTCCGCGCCGCCTCGCGCCGCCCGCCCCGGCTCTGACTGACCGCGTTACTC CCACAGGTGAGCGGGCGGGACGGCCCTTCTCCTCCGGGCTGTAATTAGCGCTTGGTT TAATGACGGCTCGTTTCTTTTCTGTGGCTGCGTGAAAGCCTTAAAGGGCTCCGGGAG GGCCCTTTGTGCGGGGGGGAGCGGCTCGGGGGGTGCGTGCGTGTGTGTGTGCGTGG GGAGCGCCGCGTGCGGCCCGCGCTGCCCGGCGGCTGTGAGCGCTGCGGGCGCGGCG CGGGGCTTTGTGCGCTCCGCGTGTGCGCGAGGGGAGCGCGGCCGGGGGCGGTGCCC CGCGGTGCGGGGGGGCTGCGAGGGGAACAAAGGCTGCGTGCGGGGTGTGTGCGTGG GGGGGTGAGCAGGGGGTGTGGGCGCGGCGGTCGGGCTGTAACCCCCCCCTGCACCC CCCTCCCCGAGTTGCTGAGCACGGCCCGGCTTCGGGTGCGGGGCTCCGTGCGGGGC GTGGCGCGGGGCTCGCCGTGCCGGGCGGGGGGTGGCGGCAGGTGGGGGTGCCGGG CGGGGCGGGGCCGCCTCGGGCCGGGGAGGGCTCGGGGGAGGGGCGCGGCGGCCCC GGAGCGCCGGCGGCTGTCGAGGCGCGGCGAGCCGCAGCCATTGCCTTTTATGGTAA TCGTGCGAGAGGGCGCAGGGACTTCCTTTGTCCCAAATCTGGCGGAGCCGAAATCTG GGAGGCGCCGCCGCACCCCCTCTAGCGGGCGCGGGCGAAGCGGTGCGGCGCCGGCA GGAAGGAAATGGGCGGGGAGGGCCTTCGTGCGTCGCCGCGCCGCCGTCCCCTTCTC CATCTCCAGCCTCGGGGCTGCCGCAGGGGGACGGCTGCCTTCGGGGGGGACGGGGC AGGGCGGGGTTCGGCTTCTGGCGTGTGACCGGCGGGATATCTACGAAGCGGCCGCC CTCTGCTAACCATGTTCATGCCTTCTTCTTTTTCCTACAGCTCCTGGGCAACGTGCTG GTTATTGTGCTGTCTCATCATTTTGGCAAA
SEQ ID NO: 93 SV40 terminator
AACTTGTTTATTGCAGCTTATAATGGTTACAAATAAAGCAATAGCATCACAAATTTC ACAAATAAAGCATTTTTTTCACTGCATTCTAGTTGTGGTTTGTCCAAACTCATCAATG TATCTT
SEQ ID NO: 94 CIITA Exon 5 Targeting Domain Sequence
TTTCTGCCCAACTTCTGCTGGCAT
SEQ ID NO: 95 CIITA Exon 5 Left Homology Arm
AAACTTCTACCACCGTCACCTATCTCTCAGGGTTTCCTAAACATACTCTGAACAAGTT TCCTCACTCTGCCACTGTGACCCAGAAAGCTATCTGTTCTCCTTCTCCCAGACTCTGC CTCATCTTTCAAGGCCTGGTTCCAGGATCCCTTCCTCCAGGAAGCCTTCCCTGATTGC CCTATTCCACCATACACCTTTTTTCTCGGACTTCATGTCATGGTGCCTATATTCCAAG GGCTCTGAGCCATGTACCCCTTTATATAGTTACCACTATTTACTGAGTGCCTACTGTA TACCAGCTACTGTGTTGGATGCTCTAGATGTAGAACCTCTAATTATCACCATCGATTC CTGGGTAGTAGGCATTATTTATTCATCTTACACAGATGAGAAAATGGAGGCCCACAG TGGTTAAATAAGTAGCCCAAGATTGCACAGCTAGTAGGGCTAGTGGAAAGTAGAGG TGGAATTTGAACTCAAATCCTGCAGTAACTCTACCATTCTGCCTTGCTCTTCTTTGTA GCAGTAGATAAGTTTTCATGGATACATACCTCATCCTTTTGATTAGATTAAGGGCCC CTGGAGTGTCAGTGTTCATTCATTTGTTTGATCATTCATTCATTCAACAAACATTTCT TGAGTCCCCACTGTGTGCCAGGCCCAGAGGTTCCCCAGCCCAAGGCCTGGCACACA GTGGGCCTTCAGTTAGACCTTGTTGATTGACTGCGCTTTTCCTTGTCTGGGCAGCGGA ACTGGACCAGTATGTCTTCCAGGACTCCCAGCTGGAGGGCCTGAGCAAGGACATTTT CAGTAAGTTTGTGGTGGGTGGGGAGGTCTTGGCTCAGCCTGCATTTCCTGCCTTGTTC CCTGGGGGGTGCCCTAATACCTGACGACCATTCATTGATGGGCAGTCAGACCCCTCT CCCCAAGGTGGGTACAATAGAGACTCACCTTGGGCTTTCATTGATTGTGTGAGTTGG TCTCTGGTTTTTCTCAAAGTAGAGCACATAGGACCAGATGAAGTGATCGGTGAGAGT ATGGAGATGCCAGCAG
SEQ ID NO: 96 CIITA Exon 5 Right Homology Arm
AAGTTGGGCAGAAAAGTCAGAAAAGACGTGAGTGAGCCCCTCCCTGATCCAACCTA GCCTTGCTTGAGACCTGGCCTTTCCTTGACTCCAAAGCCTGCTGTGGGTCCAACTTGC TTCCCTCGCTAAGTCCTGTCTGGTTGGGAGGCCCTTTAAAAGCCAACAGGAGCCTTA AAATGTACATCTGATTATTTCATGGCCCTGATAACCCTCCAATGGCTACAAAATACA TGCCACAAGGCCTGTATGGCCCTTCCTCCCTCTCCAAACCCACTATGAGACACCACA CTTCAGCCACCACCAGCTTCTCCACTCCTATAACCCATGTGCCCTTTCAAGCCTCGGG GCCTTTGCAGTTGCTATAGTCTCTATCTGGAATGCCCTTCCCCCAGTTCTTCCCATGG CTGACTCCTTTGAATCTTTCTGGTGTTGGCTAAACTGTCACCTCTTCCTGGAACCCTT CTCTGACCATCCTTCCATGTAGATTAGCTCAGTTATTCTCACCTTGTGTGTCTTTTTCC TTGCAGTTTAGCACTCATTACCATCTGGACATATTTTACGCCTTGCTCTCCCACTGTG AGGACAGGGACCTTGTCTTTCTTGCTCGTGACTGTTTCCCCAGCATCTAGTGCAGTGC CTGGTATGCAGTAGCACCTCAGTAGATATCTGTTGAATGAAAACATCTGTAAAATGG GTGTAACAGTTAACTGAGTACTTATTATGGGTCTGACCATGTGTAAGTCCTGTATCT ATTTATTCAGTTCTTAAACAGGTGAATCGCACACAGGGTATGAGATTTTAAAAGTGC AAAGAATATTCAGTGAAGGCTGGGCGCAGCGGCTCACACCTGTAATCCCAGCAGTT TGGGAGGCCAAGGGGGACGGATCACTTGAGGTCAGGAGTTTGATACCTGCCTGGCC AACATGGTGAAACCGCGTCTCTACCAAAAAATACAAAAATTAGCCGGGTGTGGTGG TGCACGCCTGTAATCCCAGCTACTCGGGAGGCTGAGGCAGGAGAATCGCTTGAACC CAGGAGGTGGAGGTTGCAGTGAG
SEQ ID NO: 97 CD70 Exon 1 Targeting Domain Sequence
TTTGGTCCCATTGGTCGCGGGCTT
SEQ ID NO: 98 CD70 Exon 1 Left Homology Arm
AAAAATAAAAAATAAAAATATTAACTTAATTTAACTTTAAACAAAAAAGCAGGTGG TCTCCAAGAATGCAGGAGATAACTGACCGGGTGCAGTGTCTCATGCCTTTAATCCCA GCACTTTGGAAGGCCAAGGCGGGTGGATCACCCAAGGTCAGGAGTTCAAGTCCAGC CTGGCCAACATGGTGAAACCCCATCTCTACTAAAAATACAAAAAATTAGCCAGGCA TGGTGGCGCGCGCATGTTACTCCCAGCTACTCGCGAGGCTCAGACAGGAGAATCGC TTGAACCCAGGAGATCGAGGTTGCGGCGAGCTGAGATGGCGCCACTGCACTCCAGC CTGGGTGACAGAGGGAGACCTCCGTCTCAAAAACAAAACAAATCAAAAAAATGCAG GAGAGGGGTACACGAATATTTGGGGAGCACCCCCAATTCTTGGATGTCTGCTGTATC CCCAGTGCACAGCACAATCTAATCCCTAATAAATGTGCAGTGGAGGTTTGTTGAATA AATGAATGGGCCCCAGAAGAATGAGGTGGAGAGGGGAATAGGAAGATTGAATGTC TCCTGCCTGAAGGTCGGGCGGGGAGGGGTTGGGGGCAGGCAACTCTGAGGCTCACC CGGGGCCACTGCCTGCATCCTGGCAACTGCCTCCACCCACTTTAGGATCTTCAGACT GGCAGCGGTTGGAGGGAATTTCCCCTCGCCAATTGCTCAAGTCCCTCCCCTCGACCG GCCGGACATCCCCAGAGAGGGGCAGGCTGGTCCCCTGACAGGTTGAAGCAAGTAGA CGCCCAGGAGCCCCGGGAGGGGGCTGCAGTTTCCTTCCTTCCTTCTCGGCAGCGCTC CGCGCCCCCATCGCCCCTCCTGCGCTAGCGGAGGTGATCGCCGCGGCGATGCCGGA GGAGGGTTCGGGCTGCTCGGTGCGGCGCAGGCCCTATGGGTGCGTCCTGCGGGCTG CTTTGGTCCCATTGGT
SEQ ID NO: 99 CD70 Exon 1 Right Homology Arm
CGCGGGCTTGGTGATCTGCCTCGTGGTGTGCATCCAGCGCTTCGCACAGGCTCAGCA GCAGCTGCCGCTCGAGTCACTTGGGGTGAGTTGAGATGGAAAAGTTGGGAAGAAAA CATAGAGAGGCGCGTGACCGAAAAGACAGAATGAGATGGGTACAAAGAGGCCAGA GAGGAAGATCTGGTAGGGCAGAGACAGAGACCAGAACAGGGAGGCGAGGCGGGGA CCAGGCTGCCCGGTGTAGGGGCTACGAGACAGGCAGCCCTGCCAGGAGGTACAGGG AGATCCCGGGATGGGAAAGGTAGGCACACATGGAAATGGAAGATGACTCGGCTCTG GTGTTCCCCCGGCAGGCTGACTCAGAGGCTGCTGGGGGCTTCACAAGGCTGGGCGT GGGGGCTTCCTGGGGCCTCCTAGGACGGGATGGCCCCAGCCACTCGCTCCGGGTGG GGGAGGGGTCCCTTTGGGGACCGCGCCGGGCGCCTTTGCAGCGTAGAGAGTCCGCT GCGCGCGGTGCTCTCGCGCCCAGTGACATCCAGGAAAACGATTCGGGAAACGAAGA AGTTCTTTTGAAGGTCTCGACTTCACGTTCCCCGCTGGTTCAGACCTGCTTCCTCTTT AAGAAGTCTTAAGAGTAAAAAAAAATAAAATGAAATAAAATCACCAGTGCGCGCCG TGGGATGAGAGGTGGAAAGGAGGATGGACAGAGAAAAGAGAGCTCCTGGCACAGG GGACACATAGAACCTCTCTGCTTACGTCCGTGCCCTGTTTTCTGGTCTTTTCTTCCAG TGGGACGTAGCTGAGCTGCAGCTGAATCACACAGGTAACACGGGGGACGTGGAGGG ACGGGGAGAAGAAGAGGCACAGAGAGAGAAGGAAGGAGAGGTAGAAAGACAAGT GGGGAGAGACAGAGAGAAAGAGACACAGACAGAGACGGAGGGAGAGAGGGAGGG AGAGATAGGGAGGGAAACGGAGAGGGGGAGACAGAGAGAAGACAGAGAGG
SEQ ID NO: 100 CD38 Exon 1 Targeting Domain Sequence
TTTCCCGAGACCGTCCTGGCGCG
SEQ ID NO: 101 CD33 Exon 5 Targeting Domain Sequence
TTTGTCTGCAGGGAAACAAGAGACC
SEQ ID NO: 102 CD33 Exon 3 Targeting Domain Sequence
TTTGGAGTGGCCGGGTTCTAGAGTG
SEQ ID NO: 103 WT MAD7 nucleic acid sequence
ATGAATAATGGAACAAATAACTTTCAGAATTTTATCGGAATTTCTTCTTTGCAGAAG ACTCTTAGGAATGCTCTCATTCCAACCGAAACAACACAGCAATTTATTGTTAAAAAC GGAATAATTAAAGAAGATGAGCTAAGAGGAGAAAATCGTCAGATACTTAAAGATAT CATGGATGATTATTACAGAGGTTTCATTTCAGAAACTTTATCGTCAATTGATGATATT GACTGGACTTCTTTATTTGAGAAAATGGAAATTCAGTTAAAAAATGGAGATAACAA AGACACTCTTATAAAAGAACAGACTGAATACCGTAAGGCAATTCATAAAAAATTTG CAAATGATGATAGATTTAAAAATATGTTCAGTGCAAAATTAATCTCAGATATTCTTC CTGAATTTGTCATTCATAACAATAATTATTCTGCATCAGAAAAGGAAGAAAAAACAC AGGTAATTAAATTATTTTCCAGATTTGCAACGTCATTCAAGGACTATTTTAAAAACA GGGCTAATTGTTTTTCGGCTGATGATATATCTTCATCTTCTTGTCATAGAATAGTTAA TGATAATGCAGAGATATTTTTTAGTAATGCATTGGTGTATAGGAGAATTGTAAAAAG TCTTTCAAATGATGATATAAATAAAATATCCGGAGATATGAAGGATTCATTAAAGGA AATGTCTCTGGAAGAAATTTATTCTTATGAAAAATATGGGGAATTTATTACACAGGA AGGTATATCTTTTTATAATGATATATGTGGTAAAGTAAATTCATTTATGAATTTATAT TGCCAGAAAAATAAAGAAAACAAAAATCTCTATAAGCTGCAAAAGCTTCATAAACA GATACTGTGCATAGCAGATACTTCTTATGAGGTGCCGTATAAATTTGAATCAGATGA AGAGGTTTATCAATCAGTGAATGGATTTTTGGACAATATTAGTTCGAAACATATCGT TGAAAGATTGCGTAAGATTGGAGACAACTATAACGGCTACAATCTTGATAAGATTTA TATTGTTAGTAAATTCTATGAATCAGTTTCACAAAAGACATATAGAGATTGGGAAAC AATAAATACTGCATTAGAAATTCATTACAACAATATATTACCCGGAAATGGTAAATC TAAAGCTGACAAGGTAAAAAAAGCGGTAAAGAATGATCTGCAAAAAAGCATTACTG AAATCAATGAGCTTGTTAGCAATTATAAATTATGTTCGGATGATAATATTAAAGCTG AGACATATATACATGAAATATCACATATTTTGAATAATTTTGAAGCACAGGAGCTTA AGTATAATCCTGAAATTCATCTGGTGGAAAGTGAATTGAAAGCATCTGAATTAAAA AATGTTCTCGATGTAATAATGAATGCTTTTCATTGGTGTTCGGTTTTCATGACAGAGG AGCTGGTAGATAAAGATAATAATTTTTATGCCGAGTTAGAAGAGATATATGACGAA ATATATCCGGTAATTTCATTGTATAATCTTGTGCGTAATTATGTAACGCAGAAGCCA TATAGTACAAAAAAAATTAAATTGAATTTTGGTATTCCTACACTAGCGGATGGATGG AGTAAAAGTAAAGAATATAGTAATAATGCAATTATTCTCATGCGTGATAATTTGTAC TATTTAGGAATATTTAATGCAAAAAATAAGCCTGACAAAAAGATAATTGAAGGTAA TACATCAGAAAATAAAGGGGATTATAAGAAGATGATTTATAATCTTCTGCCAGGAC CAAATAAAATGATCCCCAAGGTATTCCTCTCTTCAAAAACCGGAGTGGAAACATATA AGCCGTCTGCCTATATATTGGAGGGCTATAAACAAAACAAGCATATTAAATCCTCTA AGGATTTTGATATAACATTTTGTCACGATTTGATTGATTATTTTAAGAACTGTATAGC AATACATCCTGAATGGAAGAATTTTGGCTTTGATTTTTCTGACACCTCCACATATGA AGATATCAGCGGATTTTACAGAGAAGTCGAATTACAAGGTTATAAAATCGACTGGA CATATATCAGCGAAAAGGATATTGATTTGTTGCAGGAAAAAGGACAGTTATATTTAT TCCAAATATATAACAAAGATTTTTCCAAGAAAAGTACCGGAAATGATAATCTTCATA CTATGTATTTGAAGAATTTGTTTAGTGAAGAGAATTTAAAGGATATTGTACTGAAAT TAAACGGTGAGGCGGAAATCTTCTTTAGAAAATCAAGCATAAAGAATCCAATAATT CATAAAAAAGGCTCTATTCTTGTTAATAGAACATATGAAGCAGAGGAAAAAGATCA ATTTGGAAATATCCAGATAGTCAGAAAAAACATACCGGAAAATATATATCAGGAGC TTTATAAATATTTCAATGATAAAAGTGATAAAGAACTTTCGGATGAAGCAGCTAAGC TTAAGAATGTAGTAGGTCATCATGAGGCTGCTACAAACATAGTAAAAGATTATAGA TATACATATGATAAATATTTTCTTCATATGCCTATTACAATCAATTTTAAAGCCAATA AGACAGGCTTTATTAATGACAGAATATTACAATATATTGCTAAAGAAAAGGATTTGC ATGTAATAGGCATTGATCGTGGTGAAAGAAACCTGATATATGTTTCAGTAATTGATA CTTGTGGAAATATTGTTGAACAAAAATCGTTTAACATTGTTAATGGATATGATTATC AGATTAAGCTCAAGCAGCAGGAGGGGGCGCGACAAATCGCACGAAAAGAATGGAA AGAAATCGGCAAAATAAAAGAAATTAAAGAAGGCTATTTATCTCTTGTAATTCATG AAATTTCAAAGATGGTTATTAAATATAATGCCATAATTGCAATGGAGGATTTAAGCT ACGGATTTAAAAAAGGTCGTTTCAAGGTTGAGCGACAGGTTTACCAGAAGTTTGAG ACAATGCTTATCAACAAACTCAACTATCTGGTATTTAAAGATATATCCATAACGGAA AACGGTGGTCTTCTAAAGGGATACCAGCTTACATATATTCCAGATAAACTGAAAAAT GTGGGTCATCAATGTGGCTGTATATTTTATGTACCTGCTGCCTATACATCAAAAATA GATCCTACAACCGGATTTGTAAATATATTCAAATTTAAAGATTTAACAGTTGATGCG AAGAGAGAATTTATAAAAAAATTTGACAGTATCAGATATGATTCAGAAAAAAATCT GTTTTGTTTTACATTCGATTATAATAACTTTATTACGCAAAATACTGTTATGTCAAAG TCAAGCTGGAGTGTATATACGTACGGAGTTAGGATAAAAAGAAGATTTGTCAATGG CAGGTTCTCAAATGAATCGGATACAATTGATATAACAAAAGATATGGAAAAAACAC TCGAAATGACAGATATAAATTGGAGAGATGGTCATGATCTGAGGCAGGATATTATT GATTATGAAATCGTACAACACATATTTGAGATTTTTAGATTGACTGTACAAATGAGA AACAGTTTAAGTGAATTAGAAGACAGGGATTATGACCGTTTGATTTCTCCGGTGCTC AATGAAAATAATATATTTTATGATTCAGCTAAAGCAGGAGATGCGTTACCTAAAGAC GCAGATGCTAATGGTGCATATTGTATAGCTCTAAAAGGCTTGTATGAAATCAAACAA ATTACAGAGAATTGGAAAGAAGACGGTAAGTTTTCAAGAGATAAACTTAAAATTTC CAATAAGGACTGGTTTGACTTTATTCAAAATAAAAGGTATTTATAA
SEQ ID NO: 104 MAD7 codon optimized nucleic acid sequence
ATGAACAACGGCACAAATAATTTTCAGAACTTCATCGGGATCTCAAGTTTGCAGAAA ACGCTGCGCAATGCTCTGATCCCCACGGAAACCACGCAACAGTTCATCGTCAAGAA CGGAATAATTAAAGAAGATGAGTTACGTGGCGAGAACCGCCAGATTCTGAAAGATA TCATGGATGACTACTACCGCGGATTCATCTCTGAGACTCTGAGTTCTATTGATGACA TAGATTGGACTAGCCTGTTCGAAAAAATGGAAATTCAGCTGAAAAATGGTGATAAT AAAGATACCTTAATTAAGGAACAGACAGAGTATCGGAAAGCAATCCATAAAAAATT TGCGAACGACGATCGGTTTAAGAACATGTTTAGCGCCAAACTGATTAGTGACATATT ACCTGAATTTGTCATCCACAACAATAATTATTCGGCATCAGAGAAAGAGGAAAAAA CCCAGGTGATAAAATTGTTTTCGCGCTTTGCGACTAGCTTTAAAGATTACTTCAAGA ACCGTGCAAATTGCTTTTCAGCGGACGATATTTCATCAAGCAGCTGCCATCGCATCG TCAACGACAATGCAGAGATATTCTTTTCAAATGCGCTGGTCTACCGCCGGATCGTAA AATCGCTGAGCAATGACGATATCAACAAAATTTCGGGCGATATGAAAGATTCATTA AAAGAAATGAGTCTGGAAGAAATATATTCTTACGAGAAGTATGGGGAATTTATTAC CCAGGAAGGCATTAGCTTCTATAATGATATCTGTGGGAAAGTGAATTCTTTTATGAA CCTGTATTGTCAGAAAAATAAAGAAAACAAAAATTTATACAAACTTCAGAAACTTC ACAAACAGATTCTATGCATTGCGGACACTAGCTATGAGGTCCCGTATAAATTTGAAA GTGACGAGGAAGTGTACCAATCAGTTAACGGCTTCCTTGATAACATTAGCAGCAAA CATATAGTCGAAAGATTACGCAAAATCGGCGATAACTATAACGGCTACAACCTGGA TAAAATTTATATCGTGTCCAAATTTTACGAGAGCGTTAGCCAAAAAACCTACCGCGA CTGGGAAACAATTAATACCGCCCTCGAAATTCATTACAATAATATCTTGCCGGGTAA CGGTAAAAGTAAAGCCGACAAAGTAAAAAAAGCGGTTAAGAATGATTTACAGAAAT CCATCACCGAAATAAATGAACTAGTGTCAAACTATAAGCTGTGCAGTGACGACAAC ATCAAAGCGGAGACTTATATACATGAGATTAGCCATATCTTGAATAACTTTGAAGCA CAGGAATTGAAATACAATCCGGAAATTCACCTAGTTGAATCCGAGCTCAAAGCGAG TGAGCTTAAAAACGTGCTGGACGTGATCATGAATGCGTTTCATTGGTGTTCGGTTTTT ATGACTGAGGAACTTGTTGATAAAGACAACAATTTTTATGCGGAACTGGAGGAGAT TTACGATGAAATTTATCCAGTAATTAGTCTGTACAACCTGGTTCGTAACTACGTTACC CAGAAACCGTACAGCACGAAAAAGATTAAATTGAACTTTGGAATACCGACGTTAGC AGACGGTTGGTCAAAGTCCAAAGAGTATTCTAATAACGCTATCATACTGATGCGCGA CAATCTGTATTATCTGGGCATCTTTAATGCGAAGAATAAACCGGACAAGAAGATTAT CGAGGGTAATACGTCAGAAAATAAGGGTGACTACAAAAAGATGATTTATAATTTGC TCCCGGGTCCCAACAAAATGATCCCGAAAGTTTTCTTGAGCAGCAAGACGGGGGTG GAAACGTATAAACCGAGCGCCTATATCCTAGAGGGGTATAAACAGAATAAACATAT CAAGTCTTCAAAAGACTTTGATATCACTTTCTGTCATGATCTGATCGACTACTTCAAA AACTGTATTGCAATTCATCCCGAGTGGAAAAACTTCGGTTTTGATTTTAGCGACACC AGTACTTATGAAGACATTTCCGGGTTTTATCGTGAGGTAGAGTTACAAGGTTACAAG ATTGATTGGACATACATTAGCGAAAAAGACATTGATCTGCTGCAGGAAAAAGGTCA ACTGTATCTGTTCCAGATATATAACAAAGATTTTTCGAAAAAATCAACCGGGAATGA CAACCTTCACACCATGTACCTGAAAAATCTTTTCTCAGAAGAAAATCTTAAGGATAT CGTCCTGAAACTTAACGGCGAAGCGGAAATCTTCTTCAGGAAGAGCAGCATAAAGA ACCCAATCATTCATAAAAAAGGCTCGATTTTAGTCAACCGTACCTACGAAGCAGAA GAAAAAGACCAGTTTGGCAACATTCAAATTGTGCGTAAAAATATTCCGGAAAACAT TTATCAGGAGCTGTACAAATACTTCAACGATAAAAGCGACAAAGAGCTGTCTGATG AAGCAGCCAAACTGAAGAATGTAGTGGGACACCACGAGGCAGCGACGAATATAGTC AAGGACTATCGCTACACGTATGATAAATACTTCCTTCATATGCCTATTACGATCAAT TTCAAAGCCAATAAAACGGGTTTTATTAATGATAGGATCTTACAGTATATCGCTAAA GAAAAAGACTTACATGTGATCGGCATTGATCGGGGCGAGCGTAACCTGATCTACGT GTCCGTGATTGATACTTGTGGTAATATAGTTGAACAGAAAAGCTTTAACATTGTAAA CGGCTACGACTATCAGATAAAACTGAAACAACAGGAGGGCGCTAGACAGATTGCGC GGAAAGAATGGAAAGAAATTGGTAAAATTAAAGAGATCAAAGAGGGCTACCTGAG CTTAGTAATCCACGAGATCTCTAAAATGGTAATCAAATACAATGCAATTATAGCGAT GGAGGATTTGTCTTATGGTTTTAAAAAAGGGCGCTTTAAGGTCGAACGGCAAGTTTA CCAGAAATTTGAAACCATGCTCATCAATAAACTCAACTATCTGGTATTTAAAGATAT TTCGATTACCGAGAATGGCGGTCTCCTGAAAGGTTATCAGCTGACATACATTCCTGA TAAACTTAAAAACGTGGGTCATCAGTGCGGCTGCATTTTTTATGTGCCTGCTGCATA CACGAGCAAAATTGATCCGACCACCGGCTTTGTGAATATCTTTAAATTTAAAGACCT GACAGTGGACGCAAAACGTGAATTCATTAAAAAATTTGACTCAATTCGTTATGACAG TGAAAAAAATCTGTTCTGCTTTACATTTGACTACAATAACTTTATTACGCAAAACAC GGTCATGAGCAAATCATCGTGGAGTGTGTATACATACGGCGTGCGCATCAAACGTC GCTTTGTGAACGGCCGCTTCTCAAACGAAAGTGATACCATTGACATAACCAAAGATA TGGAGAAAACGTTGGAAATGACGGACATTAACTGGCGCGATGGCCACGATCTTCGT CAAGACATTATAGATTATGAAATTGTTCAGCACATATTCGAAATTTTCCGTTTAACA GTGCAAATGCGTAACTCCTTGTCTGAACTGGAGGACCGTGATTACGATCGTCTCATT TCACCTGTACTGAACGAAAATAACATTTTTTATGACAGCGCGAAAGCGGGGGATGC ACTTCCTAAGGATGCCGATGCAAATGGTGCGTATTGTATTGCATTAAAAGGGTTATA TGAAATTAAACAAATTACCGAAAATTGGAAAGAAGATGGTAAATTTTCGCGCGATA AACTCAAAATCAGCAATAAAGATTGGTTCGACTTTATCCAGAATAAGCGCTATCTCT AA
SEQ ID NO: 105 MAD7 amino acid sequence
MNNGTNNFQNFIGISSLQKTLRNALIPTETTQQFIVKNGIIKEDELRGENRQILKDIMDDY YRGFISETLSSIDDIDWTSLFEKMEIQLKNGDNKDTLIKEQTEYRKAIHKKFANDDRFKN MFSAKLISDILPEFVIHNNNYSASEKEEKTQVIKLFSRFATSFKDYFKNRANCFSADDISSS SCHRIVNDNAEIFFSNALVYRRIVKSLSNDDINKISGDMKDSLKEMSLEEIYSYEKYGEFI TQEGISFYNDICGKVNSFMNLYCQKNKENKNLYKLQKLHKQILCIADTSYEVPYKFESD EEVYQSVNGFLDNISSKHIVERLRKIGDNYNGYNLDKIYIVSKFYESVSQKTYRDWETIN TALEIHYNNILPGNGKSKADKVKKAVKNDLQKSITEINELVSNYKLCSDDNIKAETYIHEI SHILNNFEAQELKYNPEIHLVESELKASELKNVLDVIMNAFHWCSVFMTEELVDKDNNF YAELEEIYDEIYPVISLYNLVRNYVTQKPYSTKKIKLNFGIPTLADGWSKSKEYSNNAIIL MRDNLYYLGIFNAKNKPDKKIIEGNTSENKGDYKKMIYNLLPGPNKMIPKVFLSSKTGV ETYKPSAYILEGYKQNKHIKSSKDFDITFCHDLIDYFKNCIAIHPEWKNFGFDFSDTSTYE DISGFYREVELQGYKIDWTYISEKDIDLLQEKGQLYLFQIYNKDFSKKSTGNDNLHTMYL KNLFSEENLKDIVLKLNGEAEIFFRKSSIKNPIIHKKGSILVNRTYEAEEKDQFGNIQIVRK NIPENIYQELYKYFNDKSDKELSDEAAKLKNVVGHHEAATNIVKDYRYTYDKYFLHMPI TINFKANKTGFINDRILQYIAKEKDLHVIGIDRGERNLIYVSVIDTCGNIVEQKSFNIVNGY DYQIKLKQQEGARQIARKEWKEIGKIKEIKEGYLSLVIHEISKMVIKYNAIIAMEDLSYGF KKGRFKVERQVYQKFETMLINKLNYLVFKDISITENGGLLKGYQLTYIPDKLKNVGHQC GCIFYVPAAYTSKIDPTTGFVNIFKFKDLTVDAKREFIKKFDSIRYDSEKNLFCFTFDYNN FITQNTVMSKSSWSVYTYGVRIKRRFVNGRFSNESDTIDITKDMEKTLEMTDINWRDGH DLRQDIIDYEIVQHIFEIFRLTVQMRNSLSELEDRDYDRLISPVLNENNIFYDSAKAGDALP KDADANGAYCIALKGLYEIKQITENWKEDGKFSRDKLKISNKDWFDFIQNKRYL
SEQ ID NO: 106 Scaffold sequence for guide nucleic acid
GTTAAGTTATATAGAATAATTTCTACTGTTGTAGA
SEQ ID NO: 107 Scaffold sequence for guide nucleic acid
CTCTACAACTGATAAAGAATTTCTACTTTTGTAGAT
SEQ ID NO: 108 Scaffold sequence for guide nucleic acid
GTCTGGCCCCAAATTTTAATTTCTACTGTTGTAGAT
SEQ ID NO: 109 gRNA guide sequence specific for AAVS1 locus
UUUAUCUGUCCCCUCCACCCCACA
SEQ ID NO: 110 gRNA guide sequence specific for B2M locus
UUUACUCACGUCAUCCAGCAGAGA
SEQ ID NO: 111 gRNA guide sequence specific for CIITA locus
UUUACCUUGGGGCUCUGACAGGUA
SEQ ID NO: 112 gRNA guide sequence specific for the CIITA Exon 5 locus
UUUCUGCCCAACUUCUGCUGGCAU
SEQ ID NO: 113 gRNA guide sequence specific for the CLYBL locus
AGAGUGAUCACAGCUCUGACUAAA
SEQ ID NO: 114 gRNA guide sequence specific for the NKG2A locus
CUCAGACCUGAAUCUGCCCCCAAA
SEQ ID NO: 115 gRNA guide sequence specific for the TRAC locus
GUGUACCAGCUGAGAGACUCUAAA
SEQ ID NO: 116 gRNA guide sequence specific for the CD70 locus
UUUGGUCCCAUUGGUCGCGGGCUU
SEQ ID NO: 117 gRNA guide sequence specific for the CD38 locus
UUUCCCGAGACCGUCCUGGCGCG
SEQ ID NO: 118 gRNA guide sequence specific for the CD33 Exon 5 locus
UUUGUCUGCAGGGAAACAAGAGACC
SEQ ID NO: 119 gRNA guide sequence specific for the CD33 Exon 3 locus
UUUGGAGUGGCCGGGUUCUAGAGUG
SEQ ID NO: 120 HLA-E-2A-HLA-G transgene nucleotide sequence with signal and presentation peptides no terminal A
ATGGTGGTCATGGCCCCTAGAACACTGTTCCTGCTGCTGTCTGGCGCCCTGACACTG ACAGAAACATGGGCCGTGATGGCCCCACGGACACTGATTCTTGGAGGCGGAGGATC TGGCGGAGGTGGAAGTGGCGGAGGCGGATCTATTCAGCGGACCCCTAAGATCCAGG TGTACAGCAGACACCCCGCCGAGAACGGCAAGAGCAACTTCCTGAACTGCTACGTG TCCGGCTTTCACCCCAGCGACATTGAGGTGGACCTGCTGAAGAACGGCGAGCGGAT CGAGAAGGTGGAACACAGCGATCTGAGCTTCAGCAAGGACTGGTCCTTCTACCTGCT GTACTACACCGAGTTCACCCCTACCGAGAAGGACGAGTACGCCTGCAGAGTGAACC ACGTGACCCTGAGCCAGCCTAAGATCGTGAAGTGGGACAGAGATATGGGAGGCGGC GGTAGTGGCGGCGGAGGAAGCGGAGGCGGAGGTTCAGGTGGTGGTGGATCTGGAA GCCACAGCCTGAAGTACTTTCACACCTCCGTGTCCAGACCTGGCAGAGGCGAGCCTA GATTCATCAGCGTGGGCTACGTGGACGACACCCAGTTCGTCAGATTCGACAACGAC GCCGCCTCTCCTCGGATGGTTCCTAGAGCACCCTGGATGGAACAAGAGGGCAGCGA GTACTGGGATCGCGAGACAAGAAGCGCCAGAGACACAGCCCAGATCTTCCGCGTGA ACCTGAGAACCCTGCGGGGCTACTACAATCAGTCTGAGGCCGGCTCTCACACCCTGC AGTGGATGCATGGATGTGAACTGGGCCCCGACGGCAGATTCCTGAGAGGCTATGAG CAGTTCGCCTACGACGGCAAGGACTATCTGACCCTGAACGAGGACCTGAGAAGCTG GACCGCCGTGGATACAGCCGCTCAGATCAGCGAGCAGAAGTCTAACGACGCCAGCG AGGCCGAACACCAGAGAGCCTATCTGGAAGATACCTGCGTGGAATGGCTGCACAAG TACCTGGAAAAGGGCAAAGAGACACTGCTGCACCTGGAACCACCTAAGACACACGT GACACATCACCCCATCAGCGACCACGAGGCCACACTGAGATGTTGGGCCCTGGGCT TTTACCCTGCCGAGATCACACTGACATGGCAGCAGGATGGCGAGGGCCACACACAG GATACAGAGCTGGTGGAAACAAGACCTGCCGGCGACGGCACCTTCCAGAAATGGGC TGCTGTGGTGGTTCCCAGCGGCGAGGAACAGAGATACACCTGTCACGTGCAGCACG AGGGACTGCCTGAACCTGTGACTCTGAGATGGAAGCCTGCCAGCCAGCCAACAATC CCCATCGTGGGAATCATTGCCGGCCTGGTGCTGCTGGGATCTGTGGTTTCTGGTGCA GTGGTGGCCGCCGTGATTTGGAGAAAGAAGTCCTCTGGCGGCAAAGGCGGCAGCTA CTCTAAAGCCGAGTGGAGCGATTCTGCCCAGGGCTCTGAATCTCACTCTCTTGGCAG CGGCGCCACCAACTTCAGCCTGCTTAAACAGGCAGGCGACGTGGAAGAGAACCCCG GACCTATGGTTGTGATGGCTCCTAGGACTTTGTTTCTGCTGCTCAGCGGCGCTCTGAC TCTGACCGAGACTTGGGCTAGAATCATCCCCAGACATCTGCAGCTTGGAGGTGGTGG TAGCGGTGGTGGCGGTTCAGGTGGCGGAGGTTCTATCCAGAGAACCCCAAAAATTC AGGTGTACTCTCGGCACCCAGCCGAAAATGGCAAGTCCAATTTTCTCAATTGCTACG TCAGCGGGTTTCACCCTTCCGATATCGAAGTCGATCTGCTCAAGAATGGGGAGCGCA TTGAGAAAGTCGAGCACAGCGACCTGTCATTCTCCAAGGATTGGAGCTTTTACCTCC TCTACTATACGGAATTCACGCCCACAGAGAAGGATGAATATGCCTGCCGGGTCAAC CATGTGACACTGTCTCAGCCCAAGATTGTCAAATGGGATCGAGACATGGGCGGTGG CGGCAGCGGAGGTGGTGGCTCAGGCGGAGGCGGTTCCGGCGGAGGCGGCTCTGGAT CTCATTCCATGAGATACTTCTCTGCCGCCGTGTCTAGACCAGGACGGGGAGAGCCTC GGTTTATCGCCATGGGCTATGTGGATGATACGCAATTTGTTCGCTTCGACAGCGACA GCGCCTGTCCTAGAATGGAACCCAGAGCACCTTGGGTCGAGCAAGAAGGCCCCGAG TATTGGGAAGAAGAGACACGGAACACCAAGGCTCACGCCCAGACCGACAGAATGA ATCTGCAGACACTGCGCGGCTATTACAACCAGAGCGAAGCCAGCAGCCATACACTC CAGTGGATGATCGGCTGCGATCTGGGATCCGATGGCAGACTGCTGAGGGGATACGA ACAGTACGCCTATGACGGGAAAGATTACCTGGCACTGAATGAGGATCTGCGGTCCT GGACAGCCGCCGATACTGCTGCCCAGATCAGCAAGAGAAAGTGCGAGGCCGCCAAT GTGGCCGAGCAGAGAAGGGCTTATCTCGAGGGCACATGCGTCGAGTGGCTGCATCG GTATCTGGAAAACGGAAAAGAAATGCTGCAGCGGGCCGATCCTCCAAAAACACATG TGACCCACCATCCTGTGTTCGACTACGAGGCTACCCTTCGGTGTTGGGCTCTCGGATT CTACCCCGCTGAGATCATCCTCACTTGGCAACGCGACGGCGAAGATCAGACCCAGG ACGTGGAACTGGTCGAGACTAGACCAGCTGGCGACGGGACATTTCAAAAGTGGGCC GCAGTGGTCGTGCCCTCCGGCGAAGAACAACGGTACACATGCCATGTGCAGCATGA AGGCCTGCCAGAGCCTCTGATGCTCAGGTGGAAGCAGAGCAGCCTGCCTACCATTC CTATCATGGGCATCGTGGCCGGACTGGTGGTTCTGGCTGCTGTTGTTACAGGCGCAG CTGTGGCTGCAGTGCTGTGGCGGAAGAAGTCCAGCGATTG
SEQ ID NO: 121 T2A amino acid sequence
EGRGSLLTCGDVEENPGP
SEQ ID NO: 122 Thosea asigna virus 2A amino acid sequence
AEGRGSLLTCGDVEENPGP
SEQ ID NO: 123 Thosea asigna virus 2A amino acid sequence
GSGEGRGSLLTCGDVEENPGP
SEQ ID NO: 124 FMDV2A amino acid sequence
GSGSRVTELLYRMKRAETYCPRPLLAIHPTEARHKQKIVAPVKQLLNFDLLKLAGDVES NPGP
SEQ ID NO: 125 Sponge 2A amino acid sequence
LLCFLLLLLSGDVELNPGP
SEQ ID NO: 126 Sponge 2A amino acid sequence
HHFMFLLLLLAGDIELNPGP
SEQ ID NO: 127 Acorn Worm 2A amino acid sequence
WFLVLLSFILSGDIEVNPGP
SEQ ID NO: 128 Amphioxus 2A amino acid sequence
KNCAMYMLLLSGDVETNPGP
SEQ ID NO: 129 Amphioxus 2A amino acid sequence
MVISQLMLKLAGDVEENPGP
SEQ ID NO: 130 Porcine Teschovirus-1 2A amino acid sequence
GSGATNFSLLKQAGDVEENPGP
SEQ ID NO: 131 Equine Rhinitis A Virus 2A amino acid sequence
GSGQCTNYALLKLAGDVESNPGP
SEQ ID NO: 132 2A consensus sequence amino acid sequence
D-X-E-X-NPGP
SEQ ID NO: 133 GMCSFR Signal Peptide
MLLLVTSLLLCELPHPAFLLIP
SEQ ID NO: 134 FMC63
VHEVKLQESGPGLVAPSQSLSVTCTVSGVSLPDYGVSWIRQPPRKGLEWLGVIWGSETT YYNSALKSRLTIIKDNSKSQVFLKMNSLQTDDTAIYYCAKHYYYGGSYAMDYWGQGTS VTVSS
SEQ ID NO: 135 FMC63
VLDIQMTQTTSSLSASLGDRVTISCRASQDISKYLNWYQQKPDGTVKLLIYHTSRLHSGV PSRFSGSGSGTDYSLTISNLEQEDIATYFCQQGNTLPYTFGGGTKLEIT
SEQ ID NO: 136 CD28 (AA114-220)
IEVMYPPPYLDNEKSNGTIIHVKGKHLCPSPLFPGPSKPFWVLVVVGGVLACYSLLVTVA FIIFWVRSKRSRLLHSDYMNMTPRRPGPTRKHYQPYAPPRDFAAYRS
SEQ ID NO: 137 CD3-zeta isoform 3 (AA52-163)
RVKFSRSADAPAYQQGQNQLYNELNLGRREEYDVLDKRRGRDPEMGGKPRRKNPQEG LYNELQKDKMAEAYSEIGMKGERRRGKGHDGLYQGLSTATKDTYDALHMQALPPR
SEQ ID NO: 138 FMC63scFV
EVKLQESGPGLVAPSQSLSVTCTVSGVSLPDYGVSWIRQPPRKGLEWLGVIWGSETTYY NSALKSRLTIIKDNSKSQVFLKMNSLQTDDTAIYYCAKHYYYGGSYAMDYWGQGTSVT VSSGSTSGSGKPGSGEGSTKGDIQMTQTTSSLSASLGDRVTISCRASQDISKYLNWYQQK PDGTVKLLIYHTSRLHSGVPSRFSGSGSGTDYSLTISNLEQEDIATYFCQQGNTLPYTFGG GTKLEIT
SEQ ID NO: 139 41BB (AA214-255)
KRGRKKLLYIFKQPFMRPVQTTQEEDGCSCRFPEEEEGGCEL
SEQ ID NO: 140 IL2Rb (AA266-551)
NCRNTGPWLKKVLKCNTPDPSKFFSQLSSEHGGDVQKWLSSPFPSSSFSPGGLAPEISPLE VLERDKVTQLLPLNTDAYLSLQELQGQDPTHLV
SEQ ID NO: 141 CD40 (AA216-277)
KKVAKKPTNKAPHPKQEPQEINFPDDLPGSNTAAPVQETLHGCQPVTQEDGKESRISVQ ERQ
SEQ ID NO: 142 OX40 (AA236-277)
ALYLLRRDQRLPPDAHKPPGGGSFRTPIQEEQADAHSTLAKI
SEQ ID NO: 143 CD80 (AA264-288)
TYCFAPRCRERRRNERLRRESVRPV
SEQ ID NO: 144 CD86 (AA269-329)
KWKKKKRPRNSYKCGTNTMEREESEQTKKREKIHIPERSDEAQRVFKSSKTSSCDKSDT CF
SEQ ID NO: 145 CD27 (AA213-260)
QRRKYRSNKGESPVEPAEPCHYSCPREEEGSTIPIQEDYRKPEPACSP
SEQ ID NO: 146 ICOS (AA162-199)
CWLTKKKYSSSVHDPNGEYMFMRAVNTAKKSRLTDVTL
SEQ ID NO: 147 NKG2D (AA1-51)
MGWIRGRRSRHSWEMSEFHNYNLDLKKSDFSTRWQKQRCPVVKSKCRENAS
SEQ ID NO: 148 DAP10 (AA70-93) LCARPRRSPAQEDGKVYINMPGRG
SEQ ID NO: 149 DAP12 (AA62-113)
YFLGRLVPRGRGAAEAATRKQRITETESPYQELQGQRSDVYSDLNTQRPYYK
SEQ ID NO: 150 2B4/CD244 (AA251-370) WRRKRKEKQSETSPKEFLTIYEDVKDLKTRRNHEQEQTFPGGGSTIYSMIQSQSSAPTSQ EPAYTLYSLIQPSRKSGSRKRNHSPSFNSTIYEVIGKSQPKAQNPARLSRKELENFDVYS
SEQ ID NO: 151 CD28 (AA180-220)
RSKRSRLLHSDYMNMTPRRPGPTRKHYQPYAPPRDFAAYRS
SEQ ID NO: 152 CD8 (AA136-182)
TTTPAPRPPTPAPTIASQPLSLRPEACRPAAGGAVHTRGLDFACDIY
SEQ ID NO: 153 CD28 (AA114-151)
IEVMYPPPYLDNEKSNGTIIHVKGKHLCPSPLFPGPSKP
SEQ ID NO: 154 CD8 (AA183-203)
IYIWAPLAGTCGVLLLSLVIT
SEQ ID NO: 155 CD28 (AA153-179)
FWVLVVVGGVLACYSLLVTVAFIIFWV
SEQ ID NO: 156 Cytoplasmic polyhedrosis virus 2A (BmCPV2A)
DVFRSNYDLLKLCGDIESNPGP
SEQ ID NO: 157 Flacherie Virus 2A (BmIFV2A)
TLTRAKIEDELIRAGIESNPGP
SEQ ID NO: 158 CD28 Transmembrane domain amino acid sequence
FWVLVVVGGVLACYSLLVTVAFIIFWVRSKRSRLLH
SEQ ID NO: 159 CD8α Transmembrane domain amino acid sequence
IYIWAPLAGTCGVLLLSLVITLYC
SEQ ID NO: 160
CD3ζ Transmembrane domain amino acid sequence
LCYLLDGILFIYGVILTALFL
SEQ ID NO: 161 HLA-E Transmembrane domain amino acid sequence
VGIIAGLVLLGSVVSGAVVAAVIW
SEQ ID NO: 162 HLA-G Transmembrane domain amino acid sequence
MGIVAGLVVLAAVVTGAAVAAVLW
SEQ ID NO: 163 EGFR Transmembrane domain amino acid sequence
IATGMVGALLLLLVVALGIGLFM
SEQ ID NO: 165 HLA-E-(G4S)₂-HLA-G transgene amino acid sequence with signal and presentation peptides
MVVMAPRTLFLLLSGALTLTETWAVMAPRTLILGGGGSGGGGSIQRTPKIQVYSRHPAE NGKSNFLNCYVSGFHPSDIEVDLLKNGERIEKVEHSDLSFSKDWSFYLLYYTEFTPTEKD EYACRVNHVTLSQPKIVKWDRDMGGGGSGGGGSGGGGSGSHSLKYFHTSVSRPGRGEP RFISVGYVDDTQFVRFDNDAASPRMVPRAPWMEQEGSEYWDRETRSARDTAQIFRVNL RTLRGYYNQSEAGSHTLQWMHGCELGPDGRFLRGYEQFAYDGKDYLTLNEDLRSWTA VDTAAQISEQKSNDASEAEHQRAYLEDTCVEWLHKYLEKGKETLLHLEPPKTHVTHHPI SDHEATLRCWALGFYPAEITLTWQQDGEGHTQDTELVETRPAGDGTFQKWAAVVVPS GEEQRYTCHVQHEGLPEPVTLRWKPASQPTIPIVGIIAGLVLLGSVVSGAVVAAVIWRKK SSGGKGGSYSKAEWSDSAQGSESHSLGSGATNFSLLKQAGDVEENPGPMVVMAPRTLF LLLSGALTLTETWARIIPRHLQLGGGGSGGGGSGGGGSGGGGSIQRTPKIQVYSRHPAEN GKSNFLNCYVSGFHPSDIEVDLLKNGERIEKVEHSDLSFSKDWSFYLLYYTEFTPTEKDE YACRVNHVTLSQPKIVKWDRDMGGGGSGGGGSGGGGSGSHSMRYFSAAVSRPGRGEP RFIAMGYVDDTQFVRFDSDSACPRMEPRAPWVEQEGPEYWEEETRNTKAHAQTDRMN LQTLRGYYNQSEASSHTLQWMIGCDLGSDGRLLRGYEQYAYDGKDYLALNEDLRSWT AADTAAQISKRKCEAANVAEQRRAYLEGTCVEWLHRYLENGKEMLQRADPPKTHVTH HPVFDYEATLRCWALGFYPAEIILTWQRDGEDQTQDVELVETRPAGDGTFQKWAAVVV PSGEEQRYTCHVQHEGLPEPLMLRWKQSSLPTIPIMGIVAGLVVLAAVVTGAAVAAVL WRKKSSD
SEQ ID NO: 166 HLA-E-(G4S)₂-HLA-G transgene nucleotide sequence with signal and presentation peptides
ATGGTGGTCATGGCCCCTAGAACACTGTTCCTGCTGCTGTCTGGCGCCCTGACACTG ACAGAAACATGGGCCGTGATGGCCCCACGGACACTGATTCTTGGAGGCGGAGGATC TGGCGGCGGAGGCTCTATTCAGAGAACCCCTAAGATCCAGGTGTACAGCAGACACC CCGCCGAGAACGGCAAGAGCAACTTCCTGAACTGCTACGTGTCCGGCTTTCACCCCA GCGACATTGAGGTGGACCTGCTGAAGAACGGCGAGCGGATCGAGAAGGTGGAACA CAGCGATCTGAGCTTCAGCAAGGACTGGTCCTTCTACCTGCTGTACTACACCGAGTT CACCCCTACCGAGAAGGACGAGTACGCCTGCAGAGTGAACCACGTGACCCTGAGCC AGCCTAAGATCGTGAAGTGGGACAGAGATATGGGCGGAGGTGGAAGTGGCGGAGG CGGTAGCGGTGGTGGTGGATCTGGATCTCACAGCCTGAAGTACTTTCACACCTCCGT GTCCAGACCTGGCAGAGGCGAGCCTAGATTCATCAGCGTGGGCTACGTGGACGACA CCCAGTTCGTCAGATTCGACAACGACGCCGCCTCTCCTCGGATGGTTCCTAGAGCAC CCTGGATGGAACAAGAGGGCAGCGAGTACTGGGATCGCGAGACAAGAAGCGCCAG AGACACAGCCCAGATCTTCCGCGTGAACCTGAGAACCCTGCGGGGCTACTACAATC AGTCTGAGGCCGGCTCTCACACCCTGCAGTGGATGCATGGATGTGAACTGGGCCCC GACGGCAGATTCCTGAGAGGCTATGAGCAGTTCGCCTACGACGGCAAGGACTATCT GACCCTGAACGAGGACCTGAGAAGCTGGACCGCCGTGGATACAGCCGCTCAGATCA GCGAGCAGAAGTCTAACGACGCCAGCGAGGCCGAACACCAGAGAGCCTATCTGGA AGATACCTGCGTGGAATGGCTGCACAAGTACCTGGAAAAGGGCAAAGAGACACTGC TGCACCTGGAACCTCCAAAGACACACGTGACACATCACCCCATCAGCGACCACGAG GCCACACTGAGATGTTGGGCCCTGGGCTTTTACCCTGCCGAGATCACACTGACATGG CAGCAGGATGGCGAGGGCCACACACAGGATACAGAGCTGGTGGAAACAAGACCTG CCGGCGACGGCACCTTCCAGAAATGGGCTGCTGTGGTGGTTCCCAGCGGCGAGGAA CAGAGATACACCTGTCACGTGCAGCACGAGGGACTGCCTGAACCTGTGACTCTGAG ATGGAAGCCTGCCAGCCAGCCAACAATCCCCATCGTGGGAATCATTGCCGGCCTGG TGCTGCTGGGATCTGTGGTTTCTGGTGCAGTGGTGGCCGCCGTGATTTGGAGAAAGA AGTCCTCTGGCGGCAAAGGCGGCAGCTACTCTAAAGCCGAGTGGAGCGATTCTGCC CAGGGCTCTGAATCTCACTCTCTCGGAAGCGGCGCCACCAACTTCAGCCTGCTTAAA CAGGCAGGCGACGTGGAAGAGAATCCCGGACCTATGGTCGTGATGGCTCCTAGGAC TTTGTTTCTGCTGCTCAGCGGCGCTCTGACTCTGACCGAGACTTGGGCTAGAATCATC CCCAGACATCTGCAGCTCGGAGGTGGCGGAAGCGGAGGCGGTGGTTCTGGCGGAGG CGGATCAGGTGGCGGAGGAAGTATCCAGAGGACACCCAAAATTCAGGTGTACTCTC GGCACCCAGCCGAAAATGGCAAGTCCAATTTTCTCAATTGCTACGTCAGCGGGTTTC ACCCTTCCGATATCGAAGTCGATCTGCTCAAGAATGGGGAGCGCATTGAGAAAGTC GAGCACAGCGACCTGTCATTCTCCAAGGATTGGAGCTTTTACCTCCTCTACTATACG GAATTCACGCCCACAGAGAAGGATGAATATGCCTGCCGGGTCAACCATGTGACACT GTCTCAGCCCAAGATTGTCAAATGGGATCGTGACATGGGAGGCGGAGGTAGCGGAG GCGGCGGTAGTGGTGGCGGAGGCAGCGGATCTCACTCCATGAGATACTTCTCTGCCG CCGTGTCTAGACCAGGACGGGGAGAGCCTCGGTTTATCGCCATGGGCTATGTGGATG ATACGCAATTTGTTCGCTTCGACAGCGACAGCGCCTGTCCTAGAATGGAACCCAGAG CACCTTGGGTCGAGCAAGAAGGCCCCGAGTATTGGGAAGAAGAGACACGGAACAC CAAGGCTCACGCCCAGACCGACAGAATGAATCTGCAGACACTGCGCGGCTATTACA ACCAGAGCGAAGCCAGCAGCCATACACTCCAGTGGATGATCGGCTGCGATCTGGGA TCCGATGGCAGACTGCTGAGGGGATACGAACAGTACGCCTATGACGGGAAAGATTA CCTGGCACTGAATGAGGATCTGCGGTCCTGGACAGCCGCCGATACTGCTGCCCAGAT CAGCAAGAGAAAGTGCGAGGCCGCCAATGTGGCCGAGCAGAGAAGGGCTTATCTCG AGGGCACATGCGTCGAGTGGCTGCATCGGTATCTGGAAAACGGAAAAGAAATGCTG CAGCGGGCCGATCCACCTAAAACACATGTGACCCACCATCCTGTGTTCGACTACGAG GCTACCCTTCGGTGTTGGGCTCTCGGCTTCTATCCCGCTGAGATCATCCTGACTTGGC AACGCGACGGCGAAGATCAGACCCAGGACGTGGAACTGGTCGAGACTAGACCAGCT GGCGACGGGACATTTCAAAAGTGGGCCGCAGTGGTCGTGCCCTCCGGCGAAGAACA ACGGTACACATGCCATGTGCAGCATGAAGGCCTGCCAGAGCCTCTGATGCTCAGGT GGAAGCAGAGCAGCCTGCCTACCATTCCTATCATGGGCATCGTGGCCGGACTGGTTG TGCTGGCTGCTGTTGTTACAGGCGCAGCTGTTGCTGCTGTGCTGTGGCGGAAAAAGA GCAGCGAT
SEQ ID NO: 167 HLA-E-(G4S)₃-HLA-G transgene nucleotide sequence with signal and presentation peptides
ATGGTGGTCATGGCCCCTAGAACACTGTTCCTGCTGCTGTCTGGCGCCCTGACACTG ACAGAAACATGGGCCGTGATGGCCCCACGGACACTGATTCTTGGAGGCGGAGGATC TGGCGGAGGTGGAAGTGGCGGAGGCGGATCTATTCAGCGGACCCCTAAGATCCAGG TGTACAGCAGACACCCCGCCGAGAACGGCAAGAGCAACTTCCTGAACTGCTACGTG TCCGGCTTTCACCCCAGCGACATTGAGGTGGACCTGCTGAAGAACGGCGAGCGGAT CGAGAAGGTGGAACACAGCGATCTGAGCTTCAGCAAGGACTGGTCCTTCTACCTGCT GTACTACACCGAGTTCACCCCTACCGAGAAGGACGAGTACGCCTGCAGAGTGAACC ACGTGACCCTGAGCCAGCCTAAGATCGTGAAGTGGGACAGAGATATGGGAGGCGGC GGTAGTGGCGGCGGAGGAAGCGGAGGCGGAGGTTCAGGTGGTGGTGGATCTGGAA GCCACAGCCTGAAGTACTTTCACACCTCCGTGTCCAGACCTGGCAGAGGCGAGCCTA GATTCATCAGCGTGGGCTACGTGGACGACACCCAGTTCGTCAGATTCGACAACGAC GCCGCCTCTCCTCGGATGGTTCCTAGAGCACCCTGGATGGAACAAGAGGGCAGCGA GTACTGGGATCGCGAGACAAGAAGCGCCAGAGACACAGCCCAGATCTTCCGCGTGA ACCTGAGAACCCTGCGGGGCTACTACAATCAGTCTGAGGCCGGCTCTCACACCCTGC AGTGGATGCATGGATGTGAACTGGGCCCCGACGGCAGATTCCTGAGAGGCTATGAG CAGTTCGCCTACGACGGCAAGGACTATCTGACCCTGAACGAGGACCTGAGAAGCTG GACCGCCGTGGATACAGCCGCTCAGATCAGCGAGCAGAAGTCTAACGACGCCAGCG AGGCCGAACACCAGAGAGCCTATCTGGAAGATACCTGCGTGGAATGGCTGCACAAG TACCTGGAAAAGGGCAAAGAGACACTGCTGCACCTGGAACCACCTAAGACACACGT GACACATCACCCCATCAGCGACCACGAGGCCACACTGAGATGTTGGGCCCTGGGCT TTTACCCTGCCGAGATCACACTGACATGGCAGCAGGATGGCGAGGGCCACACACAG GATACAGAGCTGGTGGAAACAAGACCTGCCGGCGACGGCACCTTCCAGAAATGGGC TGCTGTGGTGGTTCCCAGCGGCGAGGAACAGAGATACACCTGTCACGTGCAGCACG AGGGACTGCCTGAACCTGTGACTCTGAGATGGAAGCCTGCCAGCCAGCCAACAATC CCCATCGTGGGAATCATTGCCGGCCTGGTGCTGCTGGGATCTGTGGTTTCTGGTGCA GTGGTGGCCGCCGTGATTTGGAGAAAGAAGTCCTCTGGCGGCAAAGGCGGCAGCTA CTCTAAAGCCGAGTGGAGCGATTCTGCCCAGGGCTCTGAATCTCACTCTCTTGGCAG CGGCGCCACCAACTTCAGCCTGCTTAAACAGGCAGGCGACGTGGAAGAGAACCCCG GACCTATGGTTGTGATGGCTCCTAGGACTTTGTTTCTGCTGCTCAGCGGCGCTCTGAC TCTGACCGAGACTTGGGCTAGAATCATCCCCAGACATCTGCAGCTTGGAGGTGGTGG TAGCGGTGGTGGCGGTTCAGGTGGCGGAGGTTCTATCCAGAGAACCCCAAAAATTC AGGTGTACTCTCGGCACCCAGCCGAAAATGGCAAGTCCAATTTTCTCAATTGCTACG TCAGCGGGTTTCACCCTTCCGATATCGAAGTCGATCTGCTCAAGAATGGGGAGCGCA TTGAGAAAGTCGAGCACAGCGACCTGTCATTCTCCAAGGATTGGAGCTTTTACCTCC TCTACTATACGGAATTCACGCCCACAGAGAAGGATGAATATGCCTGCCGGGTCAAC CATGTGACACTGTCTCAGCCCAAGATTGTCAAATGGGATCGAGACATGGGCGGTGG CGGCAGCGGAGGTGGTGGCTCAGGCGGAGGCGGTTCCGGCGGAGGCGGCTCTGGAT CTCATTCCATGAGATACTTCTCTGCCGCCGTGTCTAGACCAGGACGGGGAGAGCCTC GGTTTATCGCCATGGGCTATGTGGATGATACGCAATTTGTTCGCTTCGACAGCGACA GCGCCTGTCCTAGAATGGAACCCAGAGCACCTTGGGTCGAGCAAGAAGGCCCCGAG TATTGGGAAGAAGAGACACGGAACACCAAGGCTCACGCCCAGACCGACAGAATGA ATCTGCAGACACTGCGCGGCTATTACAACCAGAGCGAAGCCAGCAGCCATACACTC CAGTGGATGATCGGCTGCGATCTGGGATCCGATGGCAGACTGCTGAGGGGATACGA ACAGTACGCCTATGACGGGAAAGATTACCTGGCACTGAATGAGGATCTGCGGTCCT GGACAGCCGCCGATACTGCTGCCCAGATCAGCAAGAGAAAGTGCGAGGCCGCCAAT GTGGCCGAGCAGAGAAGGGCTTATCTCGAGGGCACATGCGTCGAGTGGCTGCATCG GTATCTGGAAAACGGAAAAGAAATGCTGCAGCGGGCCGATCCTCCAAAAACACATG TGACCCACCATCCTGTGTTCGACTACGAGGCTACCCTTCGGTGTTGGGCTCTCGGATT CTACCCCGCTGAGATCATCCTCACTTGGCAACGCGACGGCGAAGATCAGACCCAGG ACGTGGAACTGGTCGAGACTAGACCAGCTGGCGACGGGACATTTCAAAAGTGGGCC GCAGTGGTCGTGCCCTCCGGCGAAGAACAACGGTACACATGCCATGTGCAGCATGA AGGCCTGCCAGAGCCTCTGATGCTCAGGTGGAAGCAGAGCAGCCTGCCTACCATTC CTATCATGGGCATCGTGGCCGGACTGGTGGTTCTGGCTGCTGTTGTTACAGGCGCAG CTGTGGCTGCAGTGCTGTGGCGGAAGAAGTCCAGCGAT
SEQ ID NO: 168 HLA-E-(G4S)₄-HLA-G transgene amino acid sequence with signal and presentation peptides
MVVMAPRTLFLLLSGALTLTETWAVMAPRTLILGGGGSGGGGSGGGGSGGGGSIQRTP KIQVYSRHPAENGKSNFLNCYVSGFHPSDIEVDLLKNGERIEKVEHSDLSFSKDWSFYLL YYTEFTPTEKDEYACRVNHVTLSQPKIVKWDRDMGGGGSGGGGSGGGGSGSHSLKYF HTSVSRPGRGEPRFISVGYVDDTQFVRFDNDAASPRMVPRAPWMEQEGSEYWDRETRS ARDTAQIFRVNLRTLRGYYNQSEAGSHTLQWMHGCELGPDGRFLRGYEQFAYDGKDY LTLNEDLRSWTAVDTAAQISEQKSNDASEAEHQRAYLEDTCVEWLHKYLEKGKETLLH LEPPKTHVTHHPISDHEATLRCWALGFYPAEITLTWQQDGEGHTQDTELVETRPAGDGT FQKWAAVVVPSGEEQRYTCHVQHEGLPEPVTLRWKPASQPTIPIVGIIAGLVLLGSVVSG AVVAAVIWRKKSSGGKGGSYSKAEWSDSAQGSESHSLGSGATNFSLLKQAGDVEENPG PMVVMAPRTLFLLLSGALTLTETWARIIPRHLQLGGGGSGGGGSGGGGSGGGGSIQRTP KIQVYSRHPAENGKSNFLNCYVSGFHPSDIEVDLLKNGERIEKVEHSDLSFSKDWSFYLL YYTEFTPTEKDEYACRVNHVTLSQPKIVKWDRDMGGGGSGGGGSGGGGSGSHSMRYF SAAVSRPGRGEPRFIAMGYVDDTQFVRFDSDSACPRMEPRAPWVEQEGPEYWEEETRN TKAHAQTDRMNLQTLRGYYNQSEASSHTLQWMIGCDLGSDGRLLRGYEQYAYDGKD YLALNEDLRSWTAADTAAQISKRKCEAANVAEQRRAYLEGTCVEWLHRYLENGKEML QRADPPKTHVTHHPVFDYEATLRCWALGFYPAEIILTWQRDGEDQTQDVELVETRPAG DGTFQKWAAVVVPSGEEQRYTCHVQHEGLPEPLMLRWKQSSLPTIPIMGIVAGLVVLA AVVTGAAVAAVLWRKKSSD
SEQ ID NO: 169 HLA-E-(G4S)₄-HLA-G transgene nucleotide sequence with signal and presentation peptides
ATGGTGGTCATGGCCCCTAGAACACTGTTCCTGCTGCTGTCTGGCGCCCTGACACTG ACAGAAACATGGGCCGTGATGGCCCCACGGACACTGATTCTTGGAGGCGGAGGATC TGGCGGAGGTGGAAGTGGCGGAGGCGGTTCTGGTGGTGGTGGATCCATTCAGCGGA CCCCTAAGATCCAGGTGTACAGCAGACACCCCGCCGAGAACGGCAAGAGCAACTTC CTGAACTGCTACGTGTCCGGCTTTCACCCCAGCGACATTGAGGTGGACCTGCTGAAG AACGGCGAGCGGATCGAGAAGGTGGAACACAGCGATCTGAGCTTCAGCAAGGACTG GTCCTTCTACCTGCTGTACTACACCGAGTTCACCCCTACCGAGAAGGACGAGTACGC CTGCAGAGTGAACCACGTGACCCTGAGCCAGCCTAAGATCGTGAAGTGGGACAGAG ATATGGGAGGCGGCGGTAGTGGTGGCGGCGGTTCAGGCGGCGGAGGAAGTGGATCT CACAGCCTGAAGTACTTTCACACCTCCGTGTCCAGACCTGGCAGAGGCGAGCCTAG ATTCATCAGCGTGGGCTACGTGGACGACACCCAGTTCGTCAGATTCGACAACGACG CCGCCTCTCCTCGGATGGTTCCTAGAGCACCCTGGATGGAACAAGAGGGCAGCGAG TACTGGGATCGCGAGACAAGAAGCGCCAGAGACACAGCCCAGATCTTCCGCGTGAA CCTGAGAACCCTGCGGGGCTACTACAATCAGTCTGAGGCCGGCTCTCACACCCTGCA GTGGATGCATGGATGTGAACTGGGCCCCGACGGCAGATTCCTGAGAGGCTATGAGC AGTTCGCCTACGACGGCAAGGACTATCTGACCCTGAACGAGGACCTGAGAAGCTGG ACCGCCGTGGATACAGCCGCTCAGATCAGCGAGCAGAAGTCTAACGACGCCAGCGA GGCCGAACACCAGAGAGCCTATCTGGAAGATACCTGCGTGGAATGGCTGCACAAGT ACCTGGAAAAGGGCAAAGAGACACTGCTGCACCTGGAACCTCCAAAGACACACGTG ACACATCACCCCATCAGCGACCACGAGGCCACACTGAGATGTTGGGCCCTGGGCTTT TACCCTGCCGAGATCACACTGACATGGCAGCAGGATGGCGAGGGCCACACACAGGA TACAGAGCTGGTGGAAACAAGACCTGCCGGCGACGGCACCTTCCAGAAATGGGCTG CTGTGGTGGTTCCCAGCGGCGAGGAACAGAGATACACCTGTCACGTGCAGCACGAG GGACTGCCTGAACCTGTGACTCTGAGATGGAAGCCTGCCAGCCAGCCAACAATCCC CATCGTGGGAATCATTGCCGGCCTGGTGCTGCTGGGATCTGTGGTTTCTGGTGCAGT GGTGGCCGCCGTGATTTGGAGAAAGAAGTCCTCTGGCGGCAAAGGCGGCAGCTACT CTAAAGCCGAGTGGAGCGATTCTGCCCAGGGCTCTGAATCTCACTCTCTCGGAAGCG GCGCCACCAACTTCAGCCTGCTTAAACAGGCAGGCGACGTGGAAGAGAACCCCGGA CCTATGGTTGTGATGGCTCCTAGGACTTTGTTTCTGCTGCTCAGCGGCGCTCTGACTC TGACCGAGACTTGGGCTAGAATCATCCCCAGACATCTGCAGCTTGGCGGTGGTGGCT CAGGCGGTGGCGGAAGCGGAGGTGGCGGCAGTGGCGGCGGTGGATCTATTCAGAGA ACACCCAAAATTCAGGTGTACTCTCGGCACCCAGCCGAAAATGGCAAGTCCAATTTT CTCAATTGCTACGTCAGCGGGTTTCACCCTTCCGATATCGAAGTCGATCTGCTCAAG AATGGGGAGCGCATTGAGAAAGTCGAGCACAGCGACCTGTCATTCTCCAAGGATTG GAGCTTTTACCTCCTCTACTATACGGAATTCACGCCCACAGAGAAGGATGAATATGC CTGCCGGGTCAACCATGTGACACTGTCTCAGCCCAAGATTGTCAAATGGGATCGAGA CATGGGCGGAGGCGGATCAGGCGGAGGCGGCTCTGGCGGCGGAGGTTCTGGATCTC ATAGCATGCGGTACTTCTCTGCCGCCGTGTCTAGACCCGGAAGGGGAGAGCCTCGGT TTATCGCCATGGGCTATGTGGATGATACGCAATTTGTTCGCTTCGACAGCGACAGCG CCTGTCCTAGAATGGAACCCAGAGCACCTTGGGTCGAGCAAGAAGGCCCCGAGTAT TGGGAAGAAGAGACACGGAACACCAAGGCTCACGCCCAGACCGACAGAATGAATC TGCAGACACTGCGCGGCTATTACAACCAGAGCGAAGCCAGCAGCCATACACTCCAG TGGATGATCGGCTGCGATCTGGGATCCGATGGCAGACTGCTGAGGGGATACGAACA GTACGCCTATGACGGGAAAGATTACCTGGCACTGAATGAGGATCTGCGGTCCTGGA CAGCCGCCGATACTGCTGCCCAGATCAGCAAGAGAAAGTGCGAGGCCGCCAATGTG GCCGAGCAGAGAAGGGCTTATCTCGAGGGCACATGCGTCGAGTGGCTGCATCGGTA TCTGGAAAACGGAAAAGAAATGCTGCAGCGGGCCGATCCACCTAAAACACATGTGA CCCACCATCCTGTGTTCGACTACGAGGCTACCCTTCGGTGTTGGGCTCTCGGCTTCTA TCCCGCTGAGATCATCCTGACTTGGCAACGCGACGGCGAAGATCAGACCCAGGACG TGGAACTGGTCGAGACTAGACCAGCTGGCGACGGGACATTTCAAAAGTGGGCCGCA GTGGTCGTGCCCTCCGGCGAAGAACAACGGTACACATGCCATGTGCAGCATGAAGG CCTGCCAGAGCCTCTGATGCTCAGGTGGAAGCAGAGCAGCCTGCCTACCATTCCTAT CATGGGCATCGTGGCCGGACTGGTGGTTCTGGCTGCTGTTGTTACAGGCGCAGCTGT GGCTGCAGTCCTGTGGCGGAAAAAGAGCAGCGAT

1. A chimeric single-chain HLA-E and HLA-G molecule comprising: (a) a first molecule comprising an HLA-E heavy chain and (b) a second molecule comprising an HLA-G heavy chain, and (c) a linking peptide between (a) and (b), optionally wherein the order of the chimeric single-chain HLA-E and HLA-G molecule is (i) (a)-(c)-(b) or (ii) (b)-(c)-(a).

2. (canceled)

3. The chimeric single-chain HLA-E and HLA-G molecule according to claim 1, wherein the HLA-E heavy chain polypeptide;
(a) comprises the amino acid sequence of SEQ ID NO: 15, or an amino acid sequence having at least 80% sequence identity thereof; or
(b) is encoded by the nucleotide sequence of SEQ ID NO: 16, or a nucleotide sequence having at least 80% sequence identity thereof.

4. (canceled)

5. The chimeric single-chain HLA-E and HLA-G molecule according to claim 3, wherein the HLA-G heavy chain polypeptide;
(a) comprises the amino acid sequence of SEQ ID NO: 25, or an amino acid sequence having at least 80% sequence identity thereof; or
(b) is encoded by the nucleotide sequence of SEQ ID NO: 26, or a nucleotide sequence having at least 80% sequence identity thereof.

6. (canceled)

7. The chimeric single-chain HLA-E and HLA-G molecule according to claim 1, wherein the linking peptide comprises an autoprotease peptide and optionally one or two autoprotease peptide linkers, optionally wherein the autoprotease peptide is a 2A peptide.

8.-11. (canceled)

12. The chimeric single-chain HLA-E and HLA-G molecule according to claim 7, wherein the 2A peptide is a P2A peptide, and wherein the P2A peptide comprises the amino acid sequence of SEQ ID NO: 21, or an amino acid sequence having at least 80% sequence identity thereof.

13.-14. (canceled)

15. The chimeric single-chain HLA-E and HLA-G molecule according to claim 1, wherein (a) the first molecule comprises a first B2M polypeptide fused to the HLA-E heavy chain via a first linker and/or (b) the second molecule comprises a second B2M polypeptide fused to the HLA-G heavy chain via a second linker.

16. The chimeric single-chain HLA-E and HLA-G molecule according to claim 15, wherein the first B2M polypeptide is:
(a) 5′ to the HLA-E heavy chain polypeptide;
(b) 3′ to the HLA-E heavy chain polypeptide;
(c) 5′ to the HLA-G heavy chain polypeptide; or
(d) 3′ to the HLA-G heavy chain polypeptide.

17.-19. (canceled)

20. The chimeric single-chain HLA-E and HLA-G molecule according to claim 15, wherein the first B2M polypeptide and/or the second B2M polypeptide:
(a) comprises the amino acid sequence of SEQ ID NO: 9, or an amino acid sequence having at least 80% sequence identity thereof; or
(b) is encoded by the nucleotide sequence of SEQ ID NO: 10 or 11, or a nucleotide sequence having at least 80% sequence identity thereof.

21. (canceled)

22. The chimeric single-chain HLA-E and HLA-G molecule according to claim 15, wherein (a) the first molecule further comprises a first presentation peptide fused to the first B2M polypeptide via a third linker and/or (b) the second molecule further comprises a second presentation peptide fused to the second B2M polypeptide via a fourth linker.

23. The chimeric single-chain HLA-E and HLA-G molecule according to claim 22, wherein (a) the first presentation peptide is fused to the first B2M polypeptide and (b) the second presentation peptide is fused to the second B2M polypeptide.

24. The chimeric single-chain HLA-E and HLA-G molecule according to claim 22 or claim 23, wherein the first presentation peptide and a second presentation peptide are the same or different.

25. (canceled)

26. The chimeric single-chain HLA-E and HLA-G molecule according to claim 22, wherein the first presentation peptide and/or the second presentation peptide comprises:
(a) the amino acid sequence of SEQ ID NOs: 4 or 23, or an amino acid sequence having at least 80% sequence identity thereof; or
(b) is encoded by the nucleotide sequence of SEQ ID NOs: 5 or 24, or a nucleotide sequence having at least 80% sequence identity thereof.

27. (canceled)

28. The chimeric single-chain HLA-E and HLA-G molecule according to claim 22, wherein the first, second, third, fourth, and/or autoprotease peptide linker each separately comprise an amino acid sequence set forth in Table 4, or an amino acid sequence having at least 80% sequence identity thereof.

29. The chimeric single-chain HLA-E and HLA-G molecule according to claim 22, wherein the first peptide linker and/or the second peptide linker comprises:
(a) the amino acid sequence of SEQ ID NO: 6, 39, or 41, or an amino acid sequence having at least 80% sequence identity to thereof; or
(b) are encoded by the nucleotide sequence of SEQ ID NO: 7 or 8, or a nucleotide sequence having at least 80% sequence identity thereof.

30. (canceled)

31. The chimeric single-chain HLA-E and HLA-G molecule according to claim 22, wherein the third peptide linker and/or the fourth peptide linker comprises:
(a) the amino acid sequence of SEQ ID NO: 12, or an amino acid sequence having at least 80% sequence identity thereof, or
(b) are encoded by the nucleotide sequence of SEQ ID NO: 13 or 14, or a nucleotide sequence having at least 80% sequence identity thereof.

32. (canceled)

33. The chimeric single-chain HLA-E and HLA-G molecule according to claim 1, wherein (a) the first molecule further comprises a first signal peptide operably linked to the HLA-E heavy chain and/or (b) the second molecule further comprises a second signal peptide operably linked to the HLA-G heavy chain.

34. The chimeric single-chain HLA-E and HLA-G molecule according to claim 33, wherein the first signal peptide and the second signal peptides are the same or different.

35. (canceled)

36. The chimeric single-chain HLA-E and HLA-G molecule according to claim 33, wherein the first signal peptide, and/or the second signal peptide, or both, comprise the amino acid sequence of SEQ ID NO: 1, or an amino acid sequence having at least 80% sequence identity thereof.

37.-38. (canceled)

39. The chimeric single-chain HLA-E and HLA-G molecule according to claim 1, wherein the first molecule comprises the amino acid sequence of SEQ ID NO: 17 or 19, or an amino acid sequence having at least 80% sequence identity thereof.

40. The chimeric single-chain HLA-E and HLA-G molecule according to claim 1, wherein the second molecule comprises the amino acid sequence of SEQ ID NO: 27 or 29, or an amino acid sequence having at least 80% sequence identity thereof.

41. The chimeric single-chain HLA-E and HLA-G molecule according to claim 1, wherein the chimeric single-chain HLA-E and HLA-G molecule comprises the amino acid sequence of SEQ ID NO: 31, 165, or 168.

42. A polynucleotide encoding the chimeric single-chain HLA-E and HLA-G molecule according to claim 1.

43.-58. (canceled)

59. An immune-effector cell, or a population thereof, derived from an iPSC comprising the polynucleotide of claim 42, wherein the immune-effector cell is a T cell, a natural killer (NK) cell, a natural killer T cell (NKT cell), a mesenchymal stem cell (MSC), or a macrophage.

60.-107. (canceled)

108. A method for preventing or treating a cancer, the method comprising administering to an individual in need thereof, a therapeutically effective amount of the immune-effector cell according to claim 59.

109.-118. (canceled)


SEQUENCE LISTING (169 sequences)

SEQ ID NO: 1 - protein, 24 aa, synthetic construct
MVVMAPRTLFLLLSGALTLTETWA

SEQ ID NO: 2 - DNA, 72 bp, synthetic construct
  source 1..72: mol_type = other DNA
atggtggtcatggcccctagaacactgttcctgctgctgtctggcgccctgacactgacagaaacatgggcc

SEQ ID NO: 3 - DNA, 72 bp, synthetic construct
  source 1..72: mol_type = other DNA
atggttgtgatggctcctaggactttgtttctgctgctcagcggcgctctgactctgaccgagacttgggct

SEQ ID NO: 4 - protein, 9 aa, synthetic construct
VMAPRTLIL

SEQ ID NO: 5 - DNA, 27 bp, synthetic construct
  source 1..27: mol_type = other DNA
gtgatggccccacggacactgattctt

SEQ ID NO: 6 - protein, 15 aa, synthetic construct
GGGGSGGGGSGGGGS

SEQ ID NO: 7 - DNA, 45 bp, synthetic construct
  source 1..45: mol_type = other DNA
ggaggcggaggatctggcggaggtggaagtggcggaggcggatct

SEQ ID NO: 8 - DNA, 45 bp, synthetic construct
  source 1..45: mol_type = other DNA
ggaggtggtggtagcggtggtggcggttcaggtggcggaggttct

SEQ ID NO: 9 - protein, 99 aa, synthetic construct [sequence omitted]

SEQ ID NO: 10 - DNA, 297 bp, synthetic construct
  source 1..297: mol_type = other DNA
attcagcggacccctaagatccaggtgtacagcagacaccccgccgagaacggcaagagcaacttcctgaactgctacgtgtccggctttcaccccagcgacattgaggtggacctgctgaagaacggcgagcggatcgagaaggtggaacacagcgatctgagcttcagcaaggactggtccttctacctgctgtactacaccgagttcacccctaccgagaaggacgagtacgcctgcagagtgaaccacgtgaccctgagccagcctaagatcgtgaagtgggacagagatatg

SEQ ID NO: 11 - DNA, 297 bp, synthetic construct
  source 1..297: mol_type = other DNA
atccagagaaccccaaaaattcaggtgtactctcggcacccagccgaaaatggcaagtccaattttctcaattgctacgtcagcgggtttcacccttccgatatcgaagtcgatctgctcaagaatggggagcgcattgagaaagtcgagcacagcgacctgtcattctccaaggattggagcttttacctcctctactatacggaattcacgcccacagagaaggatgaatatgcctgccgggtcaaccatgtgacactgtctcagcccaagattgtcaaatgggatcgagacatg

SEQ ID NO: 12 - protein, 22 aa, synthetic construct
GGGGSGGGGSGGGGSGGGGSGS

SEQ ID NO: 13 - DNA, 66 bp, synthetic construct
  source 1..66: mol_type = other DNA
ggaggcggcggtagtggcggcggaggaagcggaggcggaggttcaggtggtggtggatctggaagc

SEQ ID NO: 14 - DNA, 66 bp, synthetic construct
  source 1..66: mol_type = other DNA
ggcggtggcggcagcggaggtggtggctcaggcggaggcggttccggcggaggcggctctggatct

SEQ ID NO: 15 - protein, 335 aa, synthetic construct
HSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 16 - DNA, 1005 bp, synthetic construct
  source 1..1005: mol_type = other DNA
cacagcctgaagtactttcacacctccgtgtccagacctggcagaggcgagcctagattcatcagcgtgggctacgtggacgacacccagttcgtcagattcgacaacgacgccgcctctcctcggatggttcctagagcaccctggatggaacaagagggcagcgagtactgggatcgcgagacaagaagcgccagagacacagcccagatcttccgcgtgaacctgagaaccctgcggggctactacaatcagtctgaggccggctctcacaccctgcagtggatgcatggatgtgaactgggccccgacggcagattcctgagaggctatgagcagttcgcctacgacggcaaggactatctgaccctgaacgaggacctgagaagctggaccgccgtggatacagccgctcagatcagcgagcagaagtctaacgacgccagcgaggccgaacaccagagagcctatctggaagatacctgcgtggaatggctgcacaagtacctggaaaagggcaaagagacactgctgcacctggaaccacctaagacacacgtgacacatcaccccatcagcgaccacgaggccacactgagatgttgggccctgggcttttaccctgccgagatcacactgacatggcagcaggatggcgagggccacacacaggatacagagctggtggaaacaagacctgccggcgacggcaccttccagaaatgggctgctgtggtggttcccagcggcgaggaacagagatacacctgtcacgtgcagcacgagggactgcctgaacctgtgactctgagatggaagcctgccagccagccaacaatccccatcgtgggaatcattgccggcctggtgctgctgggatctgtggtttctggtgcagtggtggccgccgtgatttggagaaagaagtcctctggcggcaaaggcggcagctactctaaagccgagtggagcgattctgcccagggctctgaatctcactctctt

SEQ ID NO: 17 - protein, 456 aa, synthetic construct
IQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 18 - DNA, 1368 bp, synthetic construct
  source 1..1368: mol_type = other DNA
attcagcggacccctaagatccaggtgtacagcagacaccccgccgagaacggcaagagcaacttcctgaactgctacgtgtccggctttcaccccagcgacattgaggtggacctgctgaagaacggcgagcggatcgagaaggtggaacacagcgatctgagcttcagcaaggactggtccttctacctgctgtactacaccgagttcacccctaccgagaaggacgagtacgcctgcagagtgaaccacgtgaccctgagccagcctaagatcgtgaagtgggacagagatatgggaggcggcggtagtggcggcggaggaagcggaggcggaggttcaggtggtggtggatctggaagccacagcctgaagtactttcacacctccgtgtccagacctggcagaggcgagcctagattcatcagcgtgggctacgtggacgacacccagttcgtcagattcgacaacgacgccgcctctcctcggatggttcctagagcaccctggatggaacaagagggcagcgagtactgggatcgcgagacaagaagcgccagagacacagcccagatcttccgcgtgaacctgagaaccctgcggggctactacaatcagtctgaggccggctctcacaccctgcagtggatgcatggatgtgaactgggccccgacggcagattcctgagaggctatgagcagttcgcctacgacggcaaggactatctgaccctgaacgaggacctgagaagctggaccgccgtggatacagccgctcagatcagcgagcagaagtctaacgacgccagcgaggccgaacaccagagagcctatctggaagatacctgcgtggaatggctgcacaagtacctggaaaagggcaaagagacactgctgcacctggaaccacctaagacacacgtgacacatcaccccatcagcgaccacgaggccacactgagatgttgggccctgggcttttaccctgccgagatcacactgacatggcagcaggatggcgagggccacacacaggatacagagctggtggaaacaagacctgccggcgacggcaccttccagaaatgggctgctgtggtggttcccagcggcgaggaacagagatacacctgtcacgtgcagcacgagggactgcctgaacctgtgactctgagatggaagcctgccagccagccaacaatccccatcgtgggaatcattgccggcctggtgctgctgggatctgtggtttctggtgcagtggtggccgccgtgatttggagaaagaagtcctctggcggcaaaggcggcagctactctaaagccgagtggagcgattctgcccagggctctgaatctcactctctt

SEQ ID NO: 19 - protein, 504 aa, synthetic construct
MVVMAPRTLFLLLSGALTLTETWAVMAPRTLILGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 20 - DNA, 1512 bp, synthetic construct
  source 1..1512: mol_type = other DNA
atggtggtcatggcccctagaacactgttcctgctgctgtctggcgccctgacactgacagaaacatgggccgtgatggccccacggacactgattcttggaggcggaggatctggcggaggtggaagtggcggaggcggatctattcagcggacccctaagatccaggtgtacagcagacaccccgccgagaacggcaagagcaacttcctgaactgctacgtgtccggctttcaccccagcgacattgaggtggacctgctgaagaacggcgagcggatcgagaaggtggaacacagcgatctgagcttcagcaaggactggtccttctacctgctgtactacaccgagttcacccctaccgagaaggacgagtacgcctgcagagtgaaccacgtgaccctgagccagcctaagatcgtgaagtgggacagagatatgggaggcggcggtagtggcggcggaggaagcggaggcggaggttcaggtggtggtggatctggaagccacagcctgaagtactttcacacctccgtgtccagacctggcagaggcgagcctagattcatcagcgtgggctacgtggacgacacccagttcgtcagattcgacaacgacgccgcctctcctcggatggttcctagagcaccctggatggaacaagagggcagcgagtactgggatcgcgagacaagaagcgccagagacacagcccagatcttccgcgtgaacctgagaaccctgcggggctactacaatcagtctgaggccggctctcacaccctgcagtggatgcatggatgtgaactgggccccgacggcagattcctgagaggctatgagcagttcgcctacgacggcaaggactatctgaccctgaacgaggacctgagaagctggaccgccgtggatacagccgctcagatcagcgagcagaagtctaacgacgccagcgaggccgaacaccagagagcctatctggaagatacctgcgtggaatggctgcacaagtacctggaaaagggcaaagagacactgctgcacctggaaccacctaagacacacgtgacacatcaccccatcagcgaccacgaggccacactgagatgttgggccctgggcttttaccctgccgagatcacactgacatggcagcaggatggcgagggccacacacaggatacagagctggtggaaacaagacctgccggcgacggcaccttccagaaatgggctgctgtggtggttcccagcggcgaggaacagagatacacctgtcacgtgcagcacgagggactgcctgaacctgtgactctgagatggaagcctgccagccagccaacaatccccatcgtgggaatcattgccggcctggtgctgctgggatctgtggtttctggtgcagtggtggccgccgtgatttggagaaagaagtcctctggcggcaaaggcggcagctactctaaagccgagtggagcgattctgcccagggctctgaatctcactctctt

SEQ ID NO: 21 - protein, 19 aa, synthetic construct
ATNFSLLKQAGDVEENPGP

SEQ ID NO: 22 - DNA, 57 bp, synthetic construct
  source 1..57: mol_type = other DNA
gccaccaacttcagcctgcttaaacaggcaggcgacgtggaagagaaccccggacct

SEQ ID NO: 23 - protein, 9 aa, synthetic construct
RIIPRHLQL

SEQ ID NO: 24 - DNA, 27 bp, synthetic construct
  source 1..27: mol_type = other DNA
agaatcatccccagacatctgcagctt

SEQ ID NO: 25 - protein, 312 aa, synthetic construct
HSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 26 - DNA, 932 bp, synthetic construct
  source 1..932: mol_type = other DNA
atgagatacttctctgccgccgtgtctagaccaggacggggagagcctcggtttatcgccatgggctatgtggatgatacgcaatttgttcgcttcgacagcgacagcgcctgtcctagaatggaacccagagcaccttgggtcgagcaagaaggccccgagtattgggaagaagagacacggaacaccaaggctcacgcccagaccgacagaatgaatctgcagacactgcgcggctattacaaccagagcgaagccagcagccatacactccagtggatgatcggctgcgatctgggatccgatggcagactgctgaggggatacgaacagtacgcctatgacgggaaagattacctggcactgaatgaggatctgcggtcctggacagccgccgatactgctgcccagatcagcaagagaaagtgcgaggccgccaatgtggccgagcagagaagggcttatctcgagggcacatgcgtcgagtggctgcatcggtatctggaaaacggaaaagaaatgctgcagcgggccgatcctccaaaaacacatgtgacccaccatcctgtgttcgactacgaggctacccttcggtgttgggctctcggattctaccccgctgagatcatcctcacttggcaacgcgacggcgaagatcagacccaggacgtggaactggtcgagactagaccagctggcgacgggacatttcaaaagtgggccgcagtggtcgtgccctccggcgaagaacaacggtacacatgccatgtgcagcatgaaggcctgccagagcctctgatgctcaggtggaagcagagcagcctgcctaccattcctatcatgggcatcgtggccggactggtggttctggctgctgttgttacaggcgcagctgtggctgcagtgctgtggcggaagaagtccagcgattg

SEQ ID NO: 27 - protein, 433 aa, synthetic construct
IQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 28 - DNA, 1301 bp, synthetic construct
  source 1..1301: mol_type = other DNA
atccagagaaccccaaaaattcaggtgtactctcggcacccagccgaaaatggcaagtccaattttctcaattgctacgtcagcgggtttcacccttccgatatcgaagtcgatctgctcaagaatggggagcgcattgagaaagtcgagcacagcgacctgtcattctccaaggattggagcttttacctcctctactatacggaattcacgcccacagagaaggatgaatatgcctgccgggtcaaccatgtgacactgtctcagcccaagattgtcaaatgggatcgagacatgggcggtggcggcagcggaggtggtggctcaggcggaggcggttccggcggaggcggctctggatctcattccatgagatacttctctgccgccgtgtctagaccaggacggggagagcctcggtttatcgccatgggctatgtggatgatacgcaatttgttcgcttcgacagcgacagcgcctgtcctagaatggaacccagagcaccttgggtcgagcaagaaggccccgagtattgggaagaagagacacggaacaccaaggctcacgcccagaccgacagaatgaatctgcagacactgcgcggctattacaaccagagcgaagccagcagccatacactccagtggatgatcggctgcgatctgggatccgatggcagactgctgaggggatacgaacagtacgcctatgacgggaaagattacctggcactgaatgaggatctgcggtcctggacagccgccgatactgctgcccagatcagcaagagaaagtgcgaggccgccaatgtggccgagcagagaagggcttatctcgagggcacatgcgtcgagtggctgcatcggtatctggaaaacggaaaagaaatgctgcagcgggccgatcctccaaaaacacatgtgacccaccatcctgtgttcgactacgaggctacccttcggtgttgggctctcggattctaccccgctgagatcatcctcacttggcaacgcgacggcgaagatcagacccaggacgtggaactggtcgagactagaccagctggcgacgggacatttcaaaagtgggccgcagtggtcgtgccctccggcgaagaacaacggtacacatgccatgtgcagcatgaaggcctgccagagcctctgatgctcaggtggaagcagagcagcctgcctaccattcctatcatgggcatcgtggccggactggtggttctggctgctgttgttacaggcgcagctgtggctgcagtgctgtggcggaagaagtccagcgattg

SEQ ID NO: 29 - protein, 481 aa, synthetic construct
MVVMAPRTLFLLLSGALTLTETWARIIPRHLQLGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 30 - DNA, 1445 bp, synthetic construct
  source 1..1445: mol_type = other DNA
atggttgtgatggctcctaggactttgtttctgctgctcagcggcgctctgactctgaccgagacttgggctagaatcatccccagacatctgcagcttggaggtggtggtagcggtggtggcggttcaggtggcggaggttctatccagagaaccccaaaaattcaggtgtactctcggcacccagccgaaaatggcaagtccaattttctcaattgctacgtcagcgggtttcacccttccgatatcgaagtcgatctgctcaagaatggggagcgcattgagaaagtcgagcacagcgacctgtcattctccaaggattggagcttttacctcctctactatacggaattcacgcccacagagaaggatgaatatgcctgccgggtcaaccatgtgacactgtctcagcccaagattgtcaaatgggatcgagacatgggcggtggcggcagcggaggtggtggctcaggcggaggcggttccggcggaggcggctctggatctcattccatgagatacttctctgccgccgtgtctagaccaggacggggagagcctcggtttatcgccatgggctatgtggatgatacgcaatttgttcgcttcgacagcgacagcgcctgtcctagaatggaacccagagcaccttgggtcgagcaagaaggccccgagtattgggaagaagagacacggaacaccaaggctcacgcccagaccgacagaatgaatctgcagacactgcgcggctattacaaccagagcgaagccagcagccatacactccagtggatgatcggctgcgatctgggatccgatggcagactgctgaggggatacgaacagtacgcctatgacgggaaagattacctggcactgaatgaggatctgcggtcctggacagccgccgatactgctgcccagatcagcaagagaaagtgcgaggccgccaatgtggccgagcagagaagggcttatctcgagggcacatgcgtcgagtggctgcatcggtatctggaaaacggaaaagaaatgctgcagcgggccgatcctccaaaaacacatgtgacccaccatcctgtgttcgactacgaggctacccttcggtgttgggctctcggattctaccccgctgagatcatcctcacttggcaacgcgacggcgaagatcagacccaggacgtggaactggtcgagactagaccagctggcgacgggacatttcaaaagtgggccgcagtggtcgtgccctccggcgaagaacaacggtacacatgccatgtgcagcatgaaggcctgccagagcctctgatgctcaggtggaagcagagcagcctgcctaccattcctatcatgggcatcgtggccggactggtggttctggctgctgttgttacaggcgcagctgtggctgcagtgctgtggcggaagaagtccagcgattg

SEQ ID NO: 31 - protein, 1007 aa, synthetic construct
MVVMAPRTLFLLLSGALTLTETWAVMAPRTLILGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSLGSGATNFSLLKQAGDVEENPGPMVVMAPRTLFLLLSGALTLTETWARIIPRHLQLGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 32 - DNA, 3024 bp, synthetic construct
  source 1..3024: mol_type = other DNA
atggtggtcatggcccctagaacactgttcctgctgctgtctggcgccctgacactgacagaaacatgggccgtgatggccccacggacactgattcttggaggcggaggatctggcggaggtggaagtggcggaggcggatctattcagcggacccctaagatccaggtgtacagcagacaccccgccgagaacggcaagagcaacttcctgaactgctacgtgtccggctttcaccccagcgacattgaggtggacctgctgaagaacggcgagcggatcgagaaggtggaacacagcgatctgagcttcagcaaggactggtccttctacctgctgtactacaccgagttcacccctaccgagaaggacgagtacgcctgcagagtgaaccacgtgaccctgagccagcctaagatcgtgaagtgggacagagatatgggaggcggcggtagtggcggcggaggaagcggaggcggaggttcaggtggtggtggatctggaagccacagcctgaagtactttcacacctccgtgtccagacctggcagaggcgagcctagattcatcagcgtgggctacgtggacgacacccagttcgtcagattcgacaacgacgccgcctctcctcggatggttcctagagcaccctggatggaacaagagggcagcgagtactgggatcgcgagacaagaagcgccagagacacagcccagatcttccgcgtgaacctgagaaccctgcggggctactacaatcagtctgaggccggctctcacaccctgcagtggatgcatggatgtgaactgggccccgacggcagattcctgagaggctatgagcagttcgcctacgacggcaaggactatctgaccctgaacgaggacctgagaagctggaccgccgtggatacagccgctcagatcagcgagcagaagtctaacgacgccagcgaggccgaacaccagagagcctatctggaagatacctgcgtggaatggctgcacaagtacctggaaaagggcaaagagacactgctgcacctggaaccacctaagacacacgtgacacatcaccccatcagcgaccacgaggccacactgagatgttgggccctgggcttttaccctgccgagatcacactgacatggcagcaggatggcgagggccacacacaggatacagagctggtggaaacaagacctgccggcgacggcaccttccagaaatgggctgctgtggtggttcccagcggcgaggaacagagatacacctgtcacgtgcagcacgagggactgcctgaacctgtgactctgagatggaagcctgccagccagccaacaatccccatcgtgggaatcattgccggcctggtgctgctgggatctgtggtttctggtgcagtggtggccgccgtgatttggagaaagaagtcctctggcggcaaaggcggcagctactctaaagccgagtggagcgattctgcccagggctctgaatctcactctcttggcagcggcgccaccaacttcagcctgcttaaacaggcaggcgacgtggaagagaaccccggacctatggttgtgatggctcctaggactttgtttctgctgctcagcggcgctctgactctgaccgagacttgggctagaatcatccccagacatctgcagcttggaggtggtggtagcggtggtggcggttcaggtggcggaggttctatccagagaaccccaaaaattcaggtgtactctcggcacccagccgaaaatggcaagtccaattttctcaattgctacgtcagcgggtttcacccttccgatatcgaagtcgatctgctcaagaatggggagcgcattgagaaagtcgagcacagcgacctgtcattctccaaggattggagcttttacctcctctactatacggaattcacgcccacagagaaggatgaatatgcctgccgggtcaaccatgtgacactgtctcagcccaagattgtcaaatgggatcgagacatgggcggtggcggcagcggaggtggtggctcaggcggaggcggttccggcggaggcggctctggatctcattccatgagatacttctctgccgccgtgtctagaccaggacggggagagcctcggtttatcgccatgggctatgtggatgatacgcaatttgttcgcttcgacagcgacagcgcctgtcctagaatggaacccagagcaccttgggtcgagcaagaaggccccgagtattgggaagaagagacacggaacaccaaggctcacgcccagaccgacagaatgaatctgcagacactgcgcggctattacaaccagagcgaagccagcagccatacactccagtggatgatcggctgcgatctgggatccgatggcagactgctgaggggatacgaacagtacgcctatgacgggaaagattacctggcactgaatgaggatctgcggtcctggacagccgccgatactgctgcccagatcagcaagagaaagtgcgaggccgccaatgtggccgagcagagaagggcttatctcgagggcacatgcgtcgagtggctgcatcggtatctggaaaacggaaaagaaatgctgcagcgggccgatcctccaaaaacacatgtgacccaccatcctgtgttcgactacgaggctacccttcggtgttgggctctcggattctaccccgctgagatcatcctcacttggcaacgcgacggcgaagatcagacccaggacgtggaactggtcgagactagaccagctggcgacgggacatttcaaaagtgggccgcagtggtcgtgccctccggcgaagaacaacggtacacatgccatgtgcagcatgaaggcctgccagagcctctgatgctcaggtggaagcagagcagcctgcctaccattcctatcatgggcatcgtggccggactggtggttctggctgctgttgttacaggcgcagctgtggctgcagtgctgtggcggaagaagtccagcgattga

SEQ ID NO: 33 - protein, 18 aa, synthetic construct
GSTSGSGKPGSGEGSTKG

SEQ ID NO: 34 - protein, 20 aa, synthetic construct
GGSEGKSSGSGSESKSTGGS

SEQ ID NO: 35 - protein, 8 aa, synthetic construct
GGGSGGGS

SEQ ID NO: 36 - protein, 12 aa, synthetic construct
GGGSGGGSGGGS

SEQ ID NO: 37 - protein, 16 aa, synthetic construct
GGGSGGGSGGGSGGGS

SEQ ID NO: 38 - protein, 20 aa, synthetic construct
GGGSGGGSGGGSGGGSGGGS

SEQ ID NO: 39 - protein, 20 aa, synthetic construct
GGGGSGGGGSGGGGSGGGGS

SEQ ID NO: 40 - protein, 25 aa, synthetic construct
GGGGSGGGGSGGGGSGGGGSGGGGS

SEQ ID NO: 41 - protein, 10 aa, synthetic construct
GGGGSGGGGS

SEQ ID NO: 42 - protein, 30 aa, synthetic construct
GGGGSGGGGSGGGGSGGGGSGGGGSGGGGS

SEQ ID NO: 43 - protein, 35 aa, synthetic construct
GGGGSGGGGSGGGGSGGGGSGGGGSGGGGSGGGGS

SEQ ID NO: 44 - protein, 40 aa, synthetic construct
GGGGSGGGGSGGGGSGGGGSGGGGSGGGGSGGGGSGGGGS

SEQ ID NO: 45 - protein, 14 aa, synthetic construct
IRPRAIGGSKPRVA

SEQ ID NO: 46 - protein, 15 aa, synthetic construct
GKGGSGKGGSGKGGS

SEQ ID NO: 47 - protein, 15 aa, synthetic construct
GGKGSGGKGSGGKGS

SEQ ID NO: 48 - protein, 15 aa, synthetic construct
GGGKSGGGKSGGGKS

SEQ ID NO: 49 - protein, 15 aa, synthetic construct
GKGKSGKGKSGKGKS

SEQ ID NO: 50 - protein, 15 aa, synthetic construct
GGGKSGGKGSGKGGS

SEQ ID NO: 51 - protein, 15 aa, synthetic construct
GKPGSGKPGSGKPGS

SEQ ID NO: 52 - protein, 20 aa, synthetic construct
GKPGSGKPGSGKPGSGKPGS

SEQ ID NO: 53 - protein, 20 aa, synthetic construct
GKGKSGKGKSGKGKSGKGKS

SEQ ID NO: 54 - protein, 14 aa, synthetic construct
STAGDTHLGGEDFD

SEQ ID NO: 55 - protein, 15 aa, synthetic construct
GEGGSGEGGSGEGGS

SEQ ID NO: 56 - protein, 15 aa, synthetic construct
GGEGSGGEGSGGEGS

SEQ ID NO: 57 - protein, 15 aa, synthetic construct
GEGESGEGESGEGES

SEQ ID NO: 58 - protein, 15 aa, synthetic construct
GGGESGGEGSGEGGS

SEQ ID NO: 59 - protein, 20 aa, synthetic construct
GEGESGEGESGEGESGEGES

SEQ ID NO: 60 - protein, 19 aa, synthetic construct
PRGASKSGSASQTGSAPGS

SEQ ID NO: 61 - DNA, 19 bp, synthetic construct
  source 1..19: mol_type = other DNA
gtaaagagaaggaaagaag

SEQ ID NO: 62 - protein, 19 aa, synthetic construct
GTSGSSGSGSGGSGSGGGG

SEQ ID NO: 63 - protein, 4 aa, synthetic construct
GSGS

SEQ ID NO: 64 - protein, 10 aa, synthetic construct
APAPAPAPAP

SEQ ID NO: 65 - protein, 20 aa, synthetic construct
APAPAPAPAPAPAPAPAPAP

SEQ ID NO: 66 - protein, 32 aa, synthetic construct
AEAAAKEAAAKEAAAAKEAAAAKEAAAAKAAA

SEQ ID NO: 67 - protein, 40 aa, synthetic construct; identical to SEQ ID NO: 44
  VARIANT 1..40: note = This sequence may encompass 1-8 GGGGS repeating units

SEQ ID NO: 68 - protein, 15 aa, synthetic construct
EPKSCDKTHTCPPCP

SEQ ID NO: 69 - protein, 12 aa, synthetic construct
ERKCCVECPPCP

SEQ ID NO: 70 - protein, 62 aa, synthetic construct
ELKTPLGDTTHTCPRCPEPKSCDTPPPCPRCPEPKSCDTPPPCPRCPEPKSCDTPPPCPRCP

SEQ ID NO: 71 - protein, 12 aa, synthetic construct
ESKYGPPCPSCP

SEQ ID NO: 72 - DNA, 24 bp, synthetic construct
  source 1..24: mol_type = other DNA
tttatctgtcccctccaccccaca

SEQ ID NO: 73 - DNA, 1205 bp, synthetic construct
  source 1..1205: mol_type = other DNA
actctgccccaggcctccttaccattccccttcgacctactctcttccgcattggagtcgctttaactggccctggctttggcagcctgtgctgacccatgcagtcctccttaccatccctccctcgacttcccctcttccgatgttgagcccctccagccggtcctggactttgtctccttccctgccctgccctctcctgaacctgagccagctcccatagctcagtctggtctatctgcctggccctggccattgtcactttgcgctgccctcctctcgcccccgagtgcccttgctgtgccgccggaactctgccctctaacgctgccgtctctctcctgagtccggaccactttgagctctactggcttctgcgccgcctctggcccactgtttccccttcccaggcaggtcctgctttctctgacctgcattctctcccctgggcctgtgccgctttctgtctgcagcttgtggcctgggtcacctctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccct

SEQ ID NO: 74 - DNA, 1200 bp, synthetic construct
  source 1..1200: mol_type = other DNA
ccaccccacagtggggccactagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggccgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggggttcctggctctgctcttcagactgagccccgttcccctgcatccccgtacccctgcatcccccttcccctgcatcccccagaggccccaggccacctacttggcctggaccccacgagaggccaccccagccctgtctaccaggctgccttttgggtggattctcctccaactgtggggtgactgcttggcaaactcactcttcggggtatcccaggaggcctggagcattggggtgggctggggttcagagaggagggattcccttctcaggttacgtggccaagaagcaggggagctgggtttgggtcaggtctgggtgtggggtgaccagcttatgctgtttgcccaggacagcctag

SEQ ID NO: 75 - DNA, 24 bp, synthetic construct
  source 1..24: mol_type = other DNA
tttactcacgtcatccagcagaga

SEQ ID NO: 76 - DNA, 1042 bp, synthetic construct
  source 1..1042: mol_type = other DNA
gcatataaaacctcagcagaaataaagaggttttgttgtttggtaagaacataccttgggttggttgggcacggtggctcgtgcctgtaatcccaacactttgggaggccaaggcaggctgatcacttgaagttgggagttcaagaccagcctggccaacatggtgaaatcccgtctctactgaaaatacaaaaattaaccaggcatggtggtgtgtgcctgtagtcccaggaatcacttgaacccaggaggcggaggttgcagtgagctgagatctcaccactgcacactgcactccagcctgggcaatggaatgagattccatcccaaaaaataaaaaaataaaaaaataaagaacataccttgggttgatccacttaggaacctcagataataacatctgccacgtatagagcaattgctatgtcccaggcactctactagacacttcatacagtttagaaaatcagatgggtgtagatcaaggcaggagcaggaaccaaaaagaaaggcataaacataagaaaaaaaatggaaggggtggaaacagagtacaataacatgagtaatttgatgggggctattatgaactgagaaatgaactttgaaaagtatcttggggccaaatcatgtagactcttgagtgatgtgttaaggaatgctatgagtgctgagagggcatcagaagtccttgagagcctccagagaaaggctcttaaaaatgcagcgcaatctccagtgacagaagatactgctagaaatctgctagaaaaaaaacaaaaaaggcatgtatagaggaattatgagggaaagataccaagtcacggtttattcttcaaaatggaggtggcttgttgggaaggtggaagctcatttggccagagtggaaatggaattgggagaaatcgatgaccaaatgtaaacacttggtgcctgatatagcttgacaccaagttagccccaagtgaaataccctggcaatattaatgtgtcttttcccgatattcctcaggtactccaaagattcaggtttactcacgtc

SEQ ID NO: 77 - DNA, 1023 bp, synthetic construct
  source 1..1023: mol_type = other DNA
atccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtggggtaagtcttacattcttttgtaagctgctgaaagttgtgtatgagtagtcatatcataaagctgctttgatataaaaaaggtctatggccatactaccctgaatgagtcccatcccatctgatataaacaatctgcatattgggattgtcagggaatgttcttaaagatcagattagtggcacctgctgagatactgatgcacagcatggtttctgaaccagtagtttccctgcagttgagcagggagcagcagcagcacttgcacaaatacatatacactcttaacacttcttacctactggcttcctctagcttttgtggcagcttcaggtatatttagcactgaacgaacatctcaagaaggtataggcctttgtttgtaagtcctgctgtcctagcatcctataatcctggacttctccagtactttctggctggattggtatctgaggctagtaggaagggcttgttcctgctgggtagctctaaacaatgtattcatgggtaggaacagcagcctattctgccagccttatttctaaccattttagacatttgttagtacatggtattttaaaagtaaaacttaatgtcttccttttttttctccactgtctttttcatagatcgagacatgtaagcagcatcatggaggtaagtttttgaccttgagaaaatgtttttgtttcactgtcctgaggactatttatagacagctctaacatgataaccctcactatgtggagaacattgacagagtaacattttagcag

SEQ ID NO: 78 - DNA, 24 bp, synthetic construct
  source 1..24: mol_type = other DNA
tttaccttggggctctgacaggta

SEQ ID NO: 79 - DNA, 1000 bp, synthetic construct
  source 1..1000: mol_type = other DNA
acaggaggtaaccatttaacaagaaagcagagtgatgttagattatagcaagatactgttgactgtagaaggctctgaggctagagagctgctttctataaaacagagtgatcatatattagaagaggtgttaaagacatgttcacaccaagctgagacttcctccttgataccaccaggaggatgggcagagactggaaaagacactaactttctccctatgggagtcagtattatttagcatcactttggcgggtcaccccaaaccatctgactacaagggtaccatatttgggttaacactcttttggtataatttatgttttagtccaatgtcttgggatgaaaatgacaggtgggccacttatgatctccagagaaattcagggcaatttggtgtgggagtaggcatggtagaggagagcagcatctaagaagtccccagcagaggctctcagcttgtcttgaggcatctgggcggagggctatgatactggccccatcctgcagaaggtggcagatattggcagctggcaccagtgcggttccattgtgatcatcatttctgaacgtcagactgttgaaggttcccccaacagactttctgtgcaactttctgtcttcaccaaattcagtccacagtaaggaagtgaaattaatttcagaggtgtggggagggcttaagggagtgtggtaaaattagagggtgttcagaaacagaaatctgaccgcttggggccaccttgcagggagagtttttttgatgatccctcacttgtttctttgcatgttggcttagcttggcgggctcccaactggtgactggttagtgatgaggctagtgatgaggctgtgtgcttctgagctgggcatccgaaggcatccttggggaagctgagggcacgaggaggggctgccagactccgggagctgctgcctggctgggattcctacacaatgcgttgcctggctccacgccctgctgggtcctacctgtcag

SEQ ID NO: 80 - DNA, 1000 bp, synthetic construct
  source 1..1000: mol_type = other DNA
agccccaaggtaaaaaggccgggaaagcatcttaatttagcgtgcagtctcagctggtcctgccattccagataaacagagaaaccattctgaattggggatgggggtgaggatgggaacaggagtctgtgtcctgctggggcaggccattggaagatgtgaaagagttgtctatttccttccaccggagggagacttcaggtcagccaggtgtctggagtatgaaccatgtatcagcaccgaaaggttctagaagtcagactttcgggcagtgtgtcactaactctcagcatgctggcctggctcggcccacagcaaggtcttctcgcctccctttgggtaaatactgaggggtgcctctgcaggacgggacctctgccagactccactccatacccagagaagcagggaaaccaaaattggagtcagccttgaggtgtagctgttgagccctcagcagctggggagagctggcggatgctgccctccccccagtttcctaatggtgttgtttaaaaagggtcaggggacgggggaacagatggtgggaagagcacagtgcagacacctggcaccggctctgaaggcagcatggcagctacaccgttggctgggaagggtgtgcccctgaagaagtcgtttacattctcgagtcaattttcctggagtgtacaatggacctgtgggaaagcctgtatgaaagggtaatgatgagggacctagcacagtgtccaatattttataggaactggaattgagctcataggagctcaattttattggcattgctgttgttggatggttaaaggggtggtatcccttttctcagactcccctgaaatgtatggtttgctttgaacccagagactgatgacaggtctgccggtgtggttgggtgcagccttaagttgctacgggaaagtgttggagggggagaagtcagaggtaaccttgccccctccctcaattccagatgaggaaattcaggcctgaaaaggga

SEQ ID NO: 81 - DNA, 24 bp, synthetic construct
  source 1..24: mol_type = other DNA
agagtgatcacagctctgactaaa

SEQ ID NO: 82 - DNA, 1046 bp, synthetic construct
  source 1..1046: mol_type = other DNA
gtccagactcaggaggacttagttctcttcataaaaagttgatcacctttgctttctcctttgcatgggcaataaagaagtgaaaaataaattcccagtgagcttgcattctgcttgggaacaacatagcacatccattttctagagagctgtccagtccccatttgagggctgctagccacatgtcaagcaggaacccagagtataaccaggaaaatgtctgtggtagaccccagtgattcatgcctcccatctccacaccctcctgtagtcccctcccacgctgattctggactaggccacatggcttggccagtggaacacatgcaggcttgcacgtctggaactctgccacctaatttaggatcccagactctcctactggagacacaggtccttagtgacagtctgcaccaccattcagacaagtcagtagggccatcttagatcatccagccctagtcaagccaccagataactgtacccacataagtgacccctggcgagaccagcaggagaatcatgccaatgggccaatatacattctgacccacagtttcataataaaataaaatggttgtggttgtaagccactatgtttcagagtggtttgttacacagcaataaataactaatatagtaggcataccatcaagtccaaagtaggtagagaagaatgtaaatagcagagcaaaacagcatgactggtggctgggaggcttaaaactgggacaggatcagagtcatgaaagaagtcaaagaaatggttcagaagtaaggctgagactgacttacaaaagctgaaagtccctttaagttggtgtttggtgcattggcaggggcaggtatggtgacttaaaagagccatgctcaacaagatcaagcacaacacaatcacgggtcaccccagcagaccttagcgagtctagccatttctttggtggtggtcacagtcatgcttcagcccagtttccacttggacaaatggtacatattttcaatgagatgaaaattaagatacaatccatgtgctcagagagtgatca

SEQ ID NO: 83 - DNA, 1053 bp, synthetic construct
  source 1..1053: mol_type = other DNA
atcatgtctgctcgacagctctgactaaacactgtgccccaaagtgttgaggaattgggaaaacctagctgagttagtggtctcttttctgttacaataaagctcataatgaaaattagccttctttgttcttccccaagtctttctttctagacgaaactactttcaactgttttaacttccttactgttaacttccatatttctagatagtatggtcagactgttatttcttgacttttaaatgtaagatattatctactgacttccttctatgtaagatgaggattgagctctcttacccttctcccatttcctcatccttccaacataaatatattttgggattatatcaacattcaatgttacttaaagtgaccttgtaaatattttcacaactgagccatgtttgatttgtatacttatgtttactttactgtttttcctgaagttaataattgccttgaatttatttatttctttaaaaatgtttcattactcaggactgtagtttacattacgattctttgtgttatacagttgatgggtttcttttctttcttaatttctttaaaaaatagagatggggtcttactatattacccaggctggtcttgaagtcctgggctcaagtgatcttcctgtctcagcctaccaagtagctgagactataggtgcaaaaaagccactatacctggctagtttacaggttttaacaaatgcattatgccacgtatccattattacaggatcacacaagatattttcattaccctgaaagcatccctgtgttccaccaattcatcctgcctccatgagccgctggcaaccactgatctctatagttttgccttttctaaaatgtcatataattggaatcatacagtctgtagcattttcagactagcttttaaaatttggcaatatgcatttaaggttcctccttaaatgtgaagggcatagccaatgtggcttgatagctcatttctttttattggtgaatatttcattgtctggatgttccacagtttgtttatcctcgagagcttggcgt

SEQ ID NO: 84 - DNA, 24 bp, synthetic construct
  source 1..24: mol_type = other DNA
ctcagacctgaatctgcccccaaa

SEQ ID NO: 85 - DNA, 1045 bp, synthetic construct
  source 1..1045: mol_type = other DNA
gacctggcctccgttttatgtagtgttcctaatactatttaggttatgcataagttaactgtgtcacatagcattttctgtcatatacaaatatttaattttttataatttttaataaataatttttaataatttcttagaacatttcagccattaagaaaagaccatttaattacctctattgattttcattaatgtgtataaattactaattaaaagtaatgtagtgcaagaaatttttatatatatatatgtcaactttactattactcatgtgcagaccacatagtcttaaccattactggatttcaaaattaacataaaaatgagtgcagaagtttttctaatacaacattttaatttttaacacaggtaaaacatcagactttaagaaatatatttttattcctgaacttctttattccttagtaatttattgcttctcattgccccagcaataatattttgtcaaatgcagaaaatttatcttttttttttgagtcggagtctcactctgtcgcccaggctggagtgcagtggcacaatctccgctcactgcaacctctgccgcccatgttcaagagattctcctgcctcagcctcccgagtagctaggactacaggcgcctgacatcatgcccgactactttttgtatttttagtagagacggagtttcaccgtgttagccaggatggtctcgatctcctgacctcgtgatcggcatgcctcggcctcccaaagtgctgggattacaggcgtgagccaccgcgcccggcctaaaaatctttttttaaaacaaatattcataagaaacgtgtttaggcttgaagaaaatcagagaaagaactttagattatttaatgcaaaatgagctccaatactcgttctccacctcacccttttaattgcactagggaatcctgtatataaaccatttattaacttcttaactactgttattatagagtacagtccctgacatcacacactgcagagatggataaccaaggagtaatctactcagacctgaacggtaccaacgcg

SEQ ID NO: 86 - DNA, 1020 bp, synthetic construct
  source 1..1020: mol_type = other DNA
aatctgcccccaaacccaaagaggcagcaacgaaaacctaaaggcaataaaaactccattttagcaactgaacaggaaataacctatgcggaattaaaccttcaaaaagcttctcaggattttcaagggaatgacaaaacctatcactgcaaaggtaaagcatttaaaagatcctcaatataacagtctaggatgtgcagcttggggtacaggaatgtggggaaagagaagggagtgctcatatatcttctatttgcaaagatcagaattccaagttgagatatgctatttcaatgtaaagtatgaagactgattgaactcattgttgaagtttgtagtctttgtcaaataattcatggagcattatttttcctgaaaattcaatggtatattattctgagaaaaagattacaatgggagatgagggtttggggtccaagtttctctgtatgattcctgtgcattcaggttctcttgtctgtgaatcttctaaacgactgtatccacctctcctttcgcactgttcccatttctctccctgcagatttaccatcagctccagagaagctcattgttgggatcctgggaattatctgtcttatcttaatggcctctgtggtaacgatagttgttattccctgtaagtctattttcgaagattacaaggggaattttcacgttaatgattgaatgtgcctctaaacatttcatattttcagggaatagagttctcattgtaatgtatatatttggactaaatgtggaatgattattctgaatttgtcaaagaataaatgaaagaataattgttgaaagtattcgcttctgatgcaatcgtatgtatatatttggatttcataactcaaaaatatgttctaggagtctgaaaaaccttactgagaaatagaaattaatttttgaaagtagttaaatcaagaattataagaactatatgagatggtgaaatttggttctttagatctatgaaatacttttccaaaaaaccaccattactttatc

SEQ ID NO: 87 - DNA, 24 bp, synthetic construct
  source 1..24: mol_type = other DNA
gtgtaccagctgagagactctaaa

SEQ ID NO: 88 - DNA, 1229 bp, synthetic construct
  source 1..1229: mol_type = other DNA
cgactctagactcgaagctggatgtctgcattgccgaggccaccagggctggctcagcaactgtcggggaatcaccagggtctgagaaatcttgtgcgcatgtgaggggctgtgggagcagagaaccactgggtgggaaattctaatccccaccctgctggaaactctctgggtggccccaacatgctaatcctccggcaaacctctgtttcctcctcaaaaggcaggaggtcggaaagaataaacaatgagagtcacattaaaaacacaaaatcctacggaaatactgaagaatgagtctcagcactaaggaaaagcctccagcagctcctgctttctgagggtgaaggatagacgctgtggctctgcatgactcactagcactctatcacggccatattctggcagggtcagtggctccaactaacatttgtttggtactttacagtttattaaatagatgtttatatggagaagctctcatttctttctcagaagagcctggctaggaaggtggatgaggcaccatattcattttgcaggtgaaattcctgagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctcgagaaggatctg

SEQ ID NO: 89 - DNA, 1130 bp, synthetic construct
  source 1..1130: mol_type = other DNA
atcatgtctgggatctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaaggagaagagcagcaggcatgagttgaatgaaggaggcagggccgggtcacagggccttctaggccatgagagggtagacagtattctaaggacgccagaaagctgttgatcggcttcaagcaggggagggacacctaattgatccgatccgagct

SEQ ID NO: 90 - protein, 471 aa, synthetic construct
MVVMAPRTLFLLLSGALTLTETWARIIPRHLQLGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 91 - DNA, 1422 bp, synthetic construct
  source 1..1422: mol_type = other DNA
gccaccatggtggtcatggcgccccgaaccctcttcctgctgctctcgggggccctgaccctgaccgagacctgggcgcggatcattccccgacatctgcaactgggaggcggcggttcaggagggggcggatcgatccaacgcacccccaagatccaggtctactccagacacccggccgaaaacggaaagtcgaacttcctgaactgctatgtgtcaggattccacccgtccgacatcgaggtggacctcctgaagaacggcgaacgcattgagaaggtcgagcactccgatctgtcgttctccaaggactggtccttctaccttctctactataccgaattcaccccgaccgagaaggacgaatacgcctgccgggtcaaccacgtgaccctgagccagccaaagatcgtgaaatgggaccgcgatatgggaggaggaggttccggcggaggaggaagcggaggcggaggttccggctcccactccatgaggtatttcagcgccgccgtgtcccggcctggccgcggagagcctcgcttcatcgccatgggatacgtggacgacacccagttcgtcagattcgacagcgacagcgcctgtcctcggatggaacctagagcaccttgggtcgagcaagagggccctgagtactgggaagaagagacacggaacaccaaggctcacgcccagaccgacagaatgaacctgcagaccctgcggggctactacaatcagtctgaggccagcagccatactctgcagtggatgatcggctgcgatctgggctctgatggcagactgctgagaggctacgagcagtacgcctacgacggcaaggattatctggccctgaacgaggacctgcggtcttggacagctgccgatacagccgctcagatcagcaagagaaagtgcgaggccgccaatgtggccgaacagagaagggcttacctggaaggcacctgtgtggaatggctgcacagatacctggaaaacggcaaagagatgctgcagcgggccgatcctcctaagacacatgtgacccaccatcctgtgttcgactacgaggccacactgagatgttgggccctgggcttttaccctgccgagatcatcctgacctggcagcgagatggcgaggatcagacccaggatgtggaactggtggaaaccagacctgccggcgacggcacctttcagaaatgggctgctgtggtggtgcccagcggagaggaacagagatacacctgtcacgtgcagcacgagggactgcctgaacctctgatgctgagatggaagcagagcagcctgcctacaatccccatcatgggaatcgtggccggactggtggttctggccgctgttgttacaggtgctgcagtggctgccgtgctgtggcggaagaaaagcagcgactga

SEQ ID NO: 92 - DNA, 1724 bp, synthetic construct
  source 1..1724: mol_type = other DNA
attgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcgggatatctacgaagcggccgccctctgctaaccatgttcatgccttcttctttttcctacagctcctgggcaacgtgctggttattgtgctgtctcatcattttggcaaa

SEQ ID NO: 93 - DNA, 121 bp, synthetic construct
  source 1..121: mol_type = other DNA
aacttgtttattgcagcttataatggttacaaataaagcaatagcatcacaaatttcacaaataaagcatttttttcactgcattctagttgtggtttgtccaaactcatcaatgtatctt

SEQ ID NO: 94 - DNA, 24 bp, synthetic construct
  source 1..24: mol_type = other DNA
tttctgcccaacttctgctggcat

SEQ ID NO: 95 - DNA, 1050 bp, synthetic construct
  source 1..1050: mol_type = other DNA
aaacttctaccaccgtcacctatctctcagggtttcctaaacatactctgaacaagtttcctcactctgccactgtgacccagaaagctatctgttctccttctcccagactctgcctcatctttcaaggcctggttccaggatcccttcctccaggaagccttccctgattgccctattccaccatacaccttttttctcggacttcatgtcatggtgcctatattccaagggctctgagccatgtacccctttatatagttaccactatttactgagtgcctactgtataccagctactgtgttggatgctctagatgtagaacctctaattatcaccatcgattcctgggtagtaggcattatttattcatcttacacagatgagaaaatggaggcccacagtggttaaataagtagcccaagattgcacagctagtagggctagtggaaagtagaggtggaatttgaactcaaatcctgcagtaactctaccattctgccttgctcttctttgtagcagtagataagttttcatggatacatacctcatccttttgattagattaagggcccctggagtgtcagtgttcattcatttgtttgatcattcattcattcaacaaacatttcttgagtccccactgtgtgccaggcccagaggttccccagcccaaggcctggcacacagtgggccttcagttagaccttgttgattgactgcgcttttccttgtctgggcagcggaactggaccagtatgtcttccaggactcccagctggagggcctgagcaaggacattttcagtaagtttgtggtgggtggggaggtcttggctcagcctgcatttcctgccttgttccctggggggtgccctaatacctgacgaccattcattgatgggcagtcagacccctctccccaaggtgggtacaatagagactcaccttgggctttcattgattgtgtgagttggtctctggtttttctcaaagtagagcacataggaccagatgaagtgatcggtgagagtatggagatgccagcag

SEQ ID NO: 96 - DNA, 1052 bp, synthetic construct
  source 1..1052: mol_type = other DNA
aagttgggcagaaaagtcagaaaagacgtgagtgagcccctccctgatccaacctagccttgcttgagacctggcctttccttgactccaaagcctgctgtgggtccaacttgcttccctcgctaagtcctgtctggttgggaggccctttaaaagccaacaggagccttaaaatgtacatctgattatttcatggccctgataaccctccaatggctacaaaatacatgccacaaggcctgtatggcccttcctccctctccaaacccactatgagacaccacacttcagccaccaccagcttctccactcctataacccatgtgccctttcaagcctcggggcctttgcagttgctatagtctctatctggaatgcccttcccccagttcttcccatggctgactcctttgaatctttctggtgttggctaaactgtcacctcttcctggaacccttctctgaccatccttccatgtagattagctcagttattctcaccttgtgtgtctttttccttgcagtttagcactcattaccatctggacatattttacgccttgctctcccactgtgaggacagggaccttgtctttcttgctcgtgactgtttccccagcatctagtgcagtgcctggtatgcagtagcacctcagtagatatctgttgaatgaaaacatctgtaaaatgggtgtaacagttaactgagtacttattatgggtctgaccatgtgtaagtcctgtatctatttattcagttcttaaacaggtgaatcgcacacagggtatgagattttaaaagtgcaaagaatattcagtgaaggctgggcgcagcggctcacacctgtaatcccagcagtttgggaggccaagggggacggatcacttgaggtcaggagtttgatacctgcctggccaacatggtgaaaccgcgtctctaccaaaaaatacaaaaattagccgggtgtggtggtgcacgcctgtaatcccagctactcgggaggctgaggcaggagaatcgcttgaacccaggaggtggaggttgcagtgag

SEQ ID NO: 97 - DNA, 24 bp, synthetic construct
  source 1..24: mol_type = other DNA
tttggtcccattggtcgcgggctt

SEQ ID NO: 98 - DNA, 973 bp, synthetic construct
  source 1..973: mol_type = other DNA
aaaaataaaaaataaaaatattaacttaatttaactttaaacaaaaaagcaggtggtctccaagaatgcaggagataactgaccgggtgcagtgtctcatgcctttaatcccagcactttggaaggccaaggcgggtggatcacccaaggtcaggagttcaagtccagcctggccaacatggtgaaaccccatctctactaaaaatacaaaaaattagccaggcatggtggcgcgcgcatgttactcccagctactcgcgaggctcagacaggagaatcgcttgaacccaggagatcgaggttgcggcgagctgagatggcgccactgcactccagcctgggtgacagagggagacctccgtctcaaaaacaaaacaaatcaaaaaaatgcaggagaggggtacacgaatatttggggagcacccccaattcttggatgtctgctgtatccccagtgcacagcacaatctaatccctaataaatgtgcagtggaggtttgttgaataaatgaatgggccccagaagaatgaggtggagaggggaataggaagattgaatgtctcctgcctgaaggtcgggcggggaggggttgggggcaggcaactctgaggctcacccggggccactgcctgcatcctggcaactgcctccacccactttaggatcttcagactggcagcggttggagggaatttcccctcgccaattgctcaagtccctcccctcgaccggccggacatccccagagaggggcaggctggtcccctgacaggttgaagcaagtagacgcccaggagccccgggagggggctgcagtttccttccttccttctcggcagcgctccgcgcccccatcgcccctcctgcgctagcggaggtgatcgccgcggcgatgccggaggagggttcgggctgctcggtgcggcgcaggccctatgggtgcgtcctgcgggctgctttggtcccattggt

SEQ ID NO: 99 - DNA, 1000 bp, synthetic construct
  source 1..1000: mol_type = other DNA
cgcgggcttggtgatctgcctcgtggtgtgcatccagcgcttcgcacaggctcagcagcagctgccgctcgagtcacttggggtgagttgagatggaaaagttgggaagaaaacatagagaggcgcgtgaccgaaaagacagaatgagatgggtacaaagaggccagagaggaagatctggtagggcagagacagagaccagaacagggaggcgaggcggggaccaggctgcccggtgtaggggctacgagacaggcagccctgccaggaggtacagggagatcccgggatgggaaaggtaggcacacatggaaatggaagatgactcggctctggtgttcccccggcaggctgactcagaggctgctgggggcttcacaaggctgggcgtgggggcttcctggggcctcctaggacgggatggccccagccactcgctccgggtgggggaggggtccctttggggaccgcgccgggcgcctttgcagcgtagagagtccgctgcgcgcggtgctctcgcgcccagtgacatccaggaaaacgattcgggaaacgaagaagttcttttgaaggtctcgacttcacgttccccgctggttcagacctgcttcctctttaagaagtcttaagagtaaaaaaaaataaaatgaaataaaatcaccagtgcgcgccgtgggatgagaggtggaaaggaggatggacagagaaaagagagctcctggcacaggggacacatagaacctctctgcttacgtccgtgccctgttttctggtcttttcttccagtgggacgtagctgagctgcagctgaatcacacaggtaacacgggggacgtggagggacggggagaagaagaggcacagagagagaaggaaggagaggtagaaagacaagtggggagagacagagagaaagagacacagacagagacggagggagagagggagggagagatagggagggaaacggagagggggagacagagagaagacagagagg

SEQ ID NO: 100 - DNA, 23 bp, synthetic construct
  source 1..23: mol_type = other DNA
tttcccgagaccgtcctggcgcg

SEQ ID NO: 101 - DNA, 25 bp, synthetic construct
  source 1..25: mol_type = other DNA
tttgtctgcagggaaacaagagacc

SEQ ID NO: 102 - DNA, 25 bp, synthetic construct
  source 1..25: mol_type = other DNA
tttggagtggccgggttctagagtg

SEQ ID NO: 103 - DNA, 3792 bp, synthetic construct
  source 1..3792: mol_type = other DNA
atgaataatggaacaaataactttcagaattttatcggaatttcttctttgcagaagactcttaggaatgctctcattccaaccgaaacaacacagcaatttattgttaaaaacggaataattaaagaagatgagctaagaggagaaaatcgtcagatacttaaagatatcatggatgattattacagaggtttcatttcagaaactttatcgtcaattgatgatattgactggacttctttatttgagaaaatggaaattcagttaaaaaatggagataacaaagacactcttataaaagaacagactgaataccgtaaggcaattcataaaaaatttgcaaatgatgatagatttaaaaatatgttcagtgcaaaattaatctcagatattcttcctgaatttgtcattcataacaataattattctgcatcagaaaaggaagaaaaaacacaggtaattaaattattttccagatttgcaacgtcattcaaggactattttaaaaacagggctaattgtttttcggctgatgatatatcttcatcttcttgtcatagaatagttaatgataatgcagagatattttttagtaatgcattggtgtataggagaattgtaaaaagtctttcaaatgatgatataaataaaatatccggagatatgaaggattcattaaaggaaatgtctctggaagaaatttattcttatgaaaaatatggggaatttattacacaggaaggtatatctttttataatgatatatgtggtaaagtaaattcatttatgaatttatattgccagaaaaataaagaaaacaaaaatctctataagctgcaaaagcttcataaacagatactgtgcatagcagatacttcttatgaggtgccgtataaatttgaatcagatgaagaggtttatcaatcagtgaatggatttttggacaatattagttcgaaacatatcgttgaaagattgcgtaagattggagacaactataacggctacaatcttgataagatttatattgttagtaaattctatgaatcagtttcacaaaagacatatagagattgggaaacaataaatactgcattagaaattcattacaacaatatattacccggaaatggtaaatctaaagctgacaaggtaaaaaaagcggtaaagaatgatctgcaaaaaagcattactgaaatcaatgagcttgttagcaattataaattatgttcggatgataatattaaagctgagacatatatacatgaaatatcacatattttgaataattttgaagcacaggagcttaagtataatcctgaaattcatctggtggaaagtgaattgaaagcatctgaattaaaaaatgttctcgatgtaataatgaatgcttttcattggtgttcggttttcatgacagaggagctggtagataaagataataatttttatgccgagttagaagagatatatgacgaaatatatccggtaatttcattgtataatcttgtgcgtaattatgtaacgcagaagccatatagtacaaaaaaaattaaattgaattttggtattcctacactagcggatggatggagtaaaagtaaagaatatagtaataatgcaattattctcatgcgtgataatttgtactatttaggaatatttaatgcaaaaaataagcctgacaaaaagataattgaaggtaatacatcagaaaataaaggggattataagaagatgatttataatcttctgccaggaccaaataaaatgatccccaaggtattcctctcttcaaaaaccggagtggaaacatataagccgtctgcctatatattggagggctataaacaaaacaagcatattaaatcctctaaggattttgatataacattttgtcacgatttgattgattattttaagaactgtatagcaatacatcctgaatggaagaattttggctttgatttttctgacacctccacatatgaagatatcagcggattttacagagaagtcgaattacaaggttataaaatcgactggacatatatcagcgaaaaggatattgatttgttgcaggaaaaaggacagttatatttattccaaatatataacaaagatttttccaagaaaagtaccggaaatgataatcttcatactatgtatttgaagaatttgtttagtgaagagaatttaaaggatattgtactgaaattaaacggtgaggcggaaatcttctttagaaaatcaagcataaagaatccaataattcataaaaaaggctctattcttgttaatagaacatatgaagcagaggaaaaagatcaatttggaaatatccagatagtcagaaaaaacataccggaaaatatatatcaggagctttataaatatttcaatgataaaagtgataaagaactttcggatgaagcagctaagcttaagaatgtagtaggtcatcatgaggctgctacaaacatagtaaaagattatagatatacatatgataaatattttcttcatatgcctattacaatcaattttaaagccaataagacaggctttattaatgacagaatattacaatatattgctaaagaaaaggatttgcatgtaataggcattgatcgtggtgaaagaaacctgatatatgtttcagtaattgatacttgtggaaatattgttgaacaaaaatcgtttaacattgttaatggatatgattatcagattaagctcaagcagcaggagggggcgcgacaaatcgcacgaaaagaatggaaagaaatcggcaaaataaaagaaattaaagaaggctatttatctcttgtaattcatgaaatttcaaagatggttattaaatataatgccataattgcaatggaggatttaagctacggatttaaaaaaggtcgtttcaaggttgagcgacaggtttaccagaagtttgagacaatgcttatcaacaaactcaactatctggtatttaaagatatatccataacggaaaacggtggtcttctaaagggataccagcttacatatattccagataaactgaaaaatgtgggtcatcaatgtggctgtatattttatgtacctgctgcctatacatcaaaaatagatcctacaaccggatttgtaaatatattcaaatttaaagatttaacagttgatgcgaagagagaatttataaaaaaatttgacagtatcagatatgattcagaaaaaaatctgttttgttttacattcgattataataactttattacgcaaaatactgttatgtcaaagtcaagctggagtgtatatacgtacggagttaggataaaaagaagatttgtcaatggcaggttctcaaatgaatcggatacaattgatataacaaaagatatggaaaaaacactcgaaatgacagatataaattggagagatggtcatgatctgaggcaggatattattgattatgaaatcgtacaacacatatttgagatttttagattgactgtacaaatgagaaacagtttaagtgaattagaagacagggattatgaccgtttgatttctccggtgctcaatgaaaataatatattttatgattcagctaaagcaggagatgcgttacctaaagacgcagatgctaatggtgcatattgtatagctctaaaaggcttgtatgaaatcaaacaaattacagagaattggaaagaagacggtaagttttcaagagataaacttaaaatttccaataaggactggtttgactttattcaaaataaaaggtatttataa

SEQ ID NO: 104 - DNA, 3792 bp, synthetic construct
  source 1..3792: mol_type = other DNA
atgaacaacggcacaaataattttcagaacttcatcgggatctcaagtttgcagaaaacgctgcgcaatgctctgatccccacggaaaccacgcaacagttcatcgtcaagaacggaataattaaagaagatgagttacgtggcgagaaccgccagattctgaaagatatcatggatgactactaccgcggattcatctctgagactctgagttctattgatgacatagattggactagcctgttcgaaaaaatggaaattcagctgaaaaatggtgataataaagataccttaattaaggaacagacagagtatcggaaagcaatccataaaaaatttgcgaacgacgatcggtttaagaacatgtttagcgccaaactgattagtgacatattacctgaatttgtcatccacaacaataattattcggcatcagagaaagaggaaaaaacccaggtgataaaattgttttcgcgctttgcgactagctttaaagattacttcaagaaccgtgcaaattgcttttcagcggacgatatttcatcaagcagctgccatcgcatcgtcaacgacaatgcagagatattcttttcaaatgcgctggtctaccgccggatcgtaaaatcgctgagcaatgacgatatcaacaaaatttcgggcgatatgaaagattcattaaaagaaatgagtctggaagaaatatattcttacgagaagtatggggaatttattacccaggaaggcattagcttctataatgatatctgtgggaaagtgaattcttttatgaacctgtattgtcagaaaaataaagaaaacaaaaatttatacaaacttcagaaacttcacaaacagattctatgcattgcggacactagctatgaggtcccgtataaatttgaaagtgacgaggaagtgtaccaatcagttaacggcttccttgataacattagcagcaaacatatagtcgaaagattacgcaaaatcggcgataactataacggctacaacctggataaaatttatatcgtgtccaaattttacgagagcgttagccaaaaaacctaccgcgactgggaaacaattaataccgccctcgaaattcattacaataatatcttgccgggtaacggtaaaagtaaagccgacaaagtaaaaaaagcggttaagaatgatttacagaaatccatcaccgaaataaatgaactagtgtcaaactataagctgtgcagtgacgacaacatcaaagcggagacttatatacatgagattagccatatcttgaataactttgaagcacaggaattgaaatacaatccggaaattcacctagttgaatccgagctcaaagcgagtgagcttaaaaacgtgctggacgtgatcatgaatgcgtttcattggtgttcggtttttatgactgaggaacttgttgataaagacaacaatttttatgcggaactggaggagatttacgatgaaatttatccagtaattagtctgtacaacctggttcgtaactacgttacccagaaaccgtacagcacgaaaaagattaaattgaactttggaataccgacgttagcagacggttggtcaaagtccaaagagtattctaataacgctatcatactgatgcgcgacaatctgtattatctgggcatctttaatgcgaagaataaaccggacaagaagattatcgagggtaatacgtcagaaaataagggtgactacaaaaagatgatttataatttgctcccgggtcccaacaaaatgatcccgaaagttttcttgagcagcaagacgggggtggaaacgtataaaccgagcgcctatatcctagaggggtataaacagaataaacatatcaagtcttcaaaagactttgatatcactttctgtcatgatctgatcgactacttcaaaaactgtattgcaattcatcccgagtggaaaaacttcggttttgattttagcgacaccagtacttatgaagacatttccgggttttatcgtgaggtagagttacaaggttacaagattgattggacatacattagcgaaaaagacattgatctgctgcaggaaaaaggtcaactgtatctgttccagatatataacaaagatttttcgaaaaaatcaaccgggaatgacaaccttcacaccatgtacctgaaaaatcttttctcagaagaaaatcttaaggatatcgtcctgaaacttaacggcgaagcggaaatcttcttcaggaagagcagcataaagaacccaatcattcataaaaaaggctcgattttagtcaaccgtacctacgaagcagaagaaaaagaccagtttggcaacattcaaattgtgcgtaaaaatattccggaaaacatttatcaggagctgtacaaatacttcaacgataaaagcgacaaagagctgtctgatgaagcagccaaactgaagaatgtagtgggacaccacgaggcagcgacgaatatagtcaaggactatcgctacacgtatgataaatacttccttcatatgcctattacgatcaatttcaaagccaataaaacgggttttattaatgataggatcttacagtatatcgctaaagaaaaagacttacatgtgatcggcattgatcggggcgagcgtaacctgatctacgtgtccgtgattgatacttgtggtaatatagttgaacagaaaagctttaacattgtaaacggctacgactatcagataaaactgaaacaacaggagggcgctagacagattgcgcggaaagaatggaaagaaattggtaaaattaaagagatcaaagagggctacctgagcttagtaatccacgagatctctaaaatggtaatcaaatacaatgcaattatagcgatggaggatttgtcttatggttttaaaaaagggcgctttaaggtcgaacggcaagtttaccagaaatttgaaaccatgctcatcaataaactcaactatctggtatttaaagatatttcgattaccgagaatggcggtctcctgaaaggttatcagctgacatacattcctgataaacttaaaaacgtgggtcatcagtgcggctgcattttttatgtgcctgctgcatacacgagcaaaattgatccgaccaccggctttgtgaatatctttaaatttaaagacctgacagtggacgcaaaacgtgaattcattaaaaaatttgactcaattcgttatgacagtgaaaaaaatctgttctgctttacatttgactacaataactttattacgcaaaacacggtcatgagcaaatcatcgtggagtgtgtatacatacggcgtgcgcatcaaacgtcgctttgtgaacggccgcttctcaaacgaaagtgataccattgacataaccaaagatatggagaaaacgttggaaatgacggacattaactggcgcgatggccacgatcttcgtcaagacattatagattatgaaattgttcagcacatattcgaaattttccgtttaacagtgcaaatgcgtaactccttgtctgaactggaggaccgtgattacgatcgtctcatttcacctgtactgaacgaaaataacattttttatgacagcgcgaaagcgggggatgcacttcctaaggatgccgatgcaaatggtgcgtattgtattgcattaaaagggttatatgaaattaaacaaattaccgaaaattggaaagaagatggtaaattttcgcgcgataaactcaaaatcagcaataaagattggttcgactttatccagaataagcgctatctctaa

SEQ ID NO: 105 - protein, 1263 aa, synthetic construct
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 106 - DNA, 35 bp, synthetic construct
  source 1..35: mol_type = other DNA
gttaagttatatagaataatttctactgttgtaga

SEQ ID NO: 107 - DNA, 36 bp, synthetic construct
  source 1..36: mol_type = other DNA
ctctacaactgataaagaatttctacttttgtagat

SEQ ID NO: 108 - DNA, 36 bp, synthetic construct
  source 1..36: mol_type = other DNA
gtctggccccaaattttaatttctactgttgtagat

SEQ ID NO: 109 - RNA, 24 nt, synthetic construct
  source 1..24: mol_type = other RNA
tttatctgtcccctccaccccaca

SEQ ID NO: 110 - RNA, 24 nt, synthetic construct
  source 1..24: mol_type = other RNA
tttactcacgtcatccagcagaga

SEQ ID NO: 111 - RNA, 24 nt, synthetic construct
  source 1..24: mol_type = other RNA
tttaccttggggctctgacaggta

SEQ ID NO: 112 - RNA, 24 nt, synthetic construct
  source 1..24: mol_type = other RNA
tttctgcccaacttctgctggcat

SEQ ID NO: 113 - RNA, 24 nt, synthetic construct
  source 1..24: mol_type = other RNA
agagtgatcacagctctgactaaa

SEQ ID NO: 114 - RNA, 24 nt, synthetic construct
  source 1..24: mol_type = other RNA
ctcagacctgaatctgcccccaaa

SEQ ID NO: 115 - RNA, 24 nt, synthetic construct
  source 1..24: mol_type = other RNA
gtgtaccagctgagagactctaaa

SEQ ID NO: 116 - RNA, 24 nt, synthetic construct
  source 1..24: mol_type = other RNA
tttggtcccattggtcgcgggctt

SEQ ID NO: 117 - RNA, 23 nt, synthetic construct
  source 1..23: mol_type = other RNA
tttcccgagaccgtcctggcgcg

SEQ ID NO: 118 - RNA, 25 nt, synthetic construct
  source 1..25: mol_type = other RNA
tttgtctgcagggaaacaagagacc

SEQ ID NO: 119 - RNA, 25 nt, synthetic construct
  source 1..25: mol_type = other RNA
tttggagtggccgggttctagagtg

SEQ ID NO: 120 - DNA, 3023 bp, synthetic construct
  source 1..3023: mol_type = other DNA
atggtggtcatggcccctagaacactgttcctgctgctgtctggcgccctgacactgacagaaacatgggccgtgatggccccacggacactgattcttggaggcggaggatctggcggaggtggaagtggcggaggcggatctattcagcggacccctaagatccaggtgtacagcagacaccccgccgagaacggcaagagcaacttcctgaactgctacgtgtccggctttcaccccagcgacattgaggtggacctgctgaagaacggcgagcggatcgagaaggtggaacacagcgatctgagcttcagcaaggactggtccttctacctgctgtactacaccgagttcacccctaccgagaaggacgagtacgcctgcagagtgaaccacgtgaccctgagccagcctaagatcgtgaagtgggacagagatatgggaggcggcggtagtggcggcggaggaagcggaggcggaggttcaggtggtggtggatctggaagccacagcctgaagtactttcacacctccgtgtccagacctggcagaggcgagcctagattcatcagcgtgggctacgtggacgacacccagttcgtcagattcgacaacgacgccgcctctcctcggatggttcctagagcaccctggatggaacaagagggcagcgagtactgggatcgcgagacaagaagcgccagagacacagcccagatcttccgcgtgaacctgagaaccctgcggggctactacaatcagtctgaggccggctctcacaccctgcagtggatgcatggatgtgaactgggccccgacggcagattcctgagaggctatgagcagttcgcctacgacggcaaggactatctgaccctgaacgaggacctgagaagctggaccgccgtggatacagccgctcagatcagcgagcagaagtctaacgacgccagcgaggccgaacaccagagagcctatctggaagatacctgcgtggaatggctgcacaagtacctggaaaagggcaaagagacactgctgcacctggaaccacctaagacacacgtgacacatcaccccatcagcgaccacgaggccacactgagatgttgggccctgggcttttaccctgccgagatcacactgacatggcagcaggatggcgagggccacacacaggatacagagctggtggaaacaagacctgccggcgacggcaccttccagaaatgggctgctgtggtggttcccagcggcgaggaacagagatacacctgtcacgtgcagcacgagggactgcctgaacctgtgactctgagatggaagcctgccagccagccaacaatccccatcgtgggaatcattgccggcctggtgctgctgggatctgtggtttctggtgcagtggtggccgccgtgatttggagaaagaagtcctctggcggcaaaggcggcagctactctaaagccgagtggagcgattctgcccagggctctgaatctcactctcttggcagcggcgccaccaacttcagcctgcttaaacaggcaggcgacgtggaagagaaccccggacctatggttgtgatggctcctaggactttgtttctgctgctcagcggcgctctgactctgaccgagacttgggctagaatcatccccagacatctgcagcttggaggtggtggtagcggtggtggcggttcaggtggcggaggttctatccagagaaccccaaaaattcaggtgtactctcggcacccagccgaaaatggcaagtccaattttctcaattgctacgtcagcgggtttcacccttccgatatcgaagtcgatctgctcaagaatggggagcgcattgagaaagtcgagcacagcgacctgtcattctccaaggattggagcttttacctcctctactatacggaattcacgcccacagagaaggatgaatatgcctgccgggtcaaccatgtgacactgtctcagcccaagattgtcaaatgggatcgagacatgggcggtggcggcagcggaggtggtggctcaggcggaggcggttccggcggaggcggctctggatctcattccatgagatacttctctgccgccgtgtctagaccaggacggggagagcctcggtttatcgccatgggctatgtggatgatacgcaatttgttcgcttcgacagcgacagcgcctgtcctagaatggaacccagagcaccttgggtcgagcaagaaggccccgagtattgggaagaagagacacggaacaccaaggctcacgcccagaccgacagaatgaatctgcagacactgcgcggctattacaaccagagcgaagccagcagccatacactccagtggatgatcggctgcgatctgggatccgatggcagactgctgaggggatacgaacagtacgcctatgacgggaaagattacctggcactgaatgaggatctgcggtcctggacagccgccgatactgctgcccagatcagcaagagaaagtgcgaggccgccaatgtggccgagcagagaagggcttatctcgagggcacatgcgtcgagtggctgcatcggtatctggaaaacggaaaagaaatgctgcagcgggccgatcctccaaaaacacatgtgacccaccatcctgtgttcgactacgaggctacccttcggtgttgggctctcggattctaccccgctgagatcatcctcacttggcaacgcgacggcgaagatcagacccaggacgtggaactggtcgagactagaccagctggcgacgggacatttcaaaagtgggccgcagtggtcgtgccctccggcgaagaacaacggtacacatgccatgtgcagcatgaaggcctgccagagcctctgatgctcaggtggaagcagagcagcctgcctaccattcctatcatgggcatcgtggccggactggtggttctggctgctgttgttacaggcgcagctgtggctgcagtgctgtggcggaagaagtccagcgattg

SEQ ID NO: 121 - protein, 18 aa, Thosea asigna virus
EGRGSLLTCGDVEENPGP

SEQ ID NO: 122 - protein, 19 aa, Thosea asigna virus
AEGRGSLLTCGDVEENPGP

SEQ ID NO: 123 - protein, 21 aa, Thosea asigna virus
GSGEGRGSLLTCGDVEENPGP

SEQ ID NO: 124 - protein, 63 aa, Foot-and-mouth disease virus
GSGSRVTELLYRMKRAETYCPRPLLAIHPTEARHKQKIVAPVKQLLNFDLLKLAGDVESNPGP

SEQ ID NO: 125 - protein, 19 aa, Amphimedon sp.
LLCFLLLLLSGDVELNPGP

SEQ ID NO: 126 - protein, 20 aa, Amphimedon sp.
HHFMFLLLLLAGDIELNPGP

SEQ ID NO: 127 - protein, 20 aa, Saccoglossus kowalevskii
WFLVLLSFILSGDIEVNPGP

SEQ ID NO: 128 - protein, 20 aa, Branchiostoma floridae
KNCAMYMLLLSGDVETNPGP

SEQ ID NO: 129 - protein, 20 aa, Branchiostoma floridae
MVISQLMLKLAGDVEENPGP

SEQ ID NO: 130 - protein, 22 aa, unidentified
  source 1..22: note = Porcine Teschovirus-1
GSGATNFSLLKQAGDVEENPGP

SEQ ID NO: 131 - protein, 23 aa, Equine Rhinitis A Virus
GSGQCTNYALLKLAGDVESNPGP

SEQ ID NO: 132 - protein, 8 aa, synthetic construct
  VARIANT 2: note = Any amino acid
  VARIANT 4: note = Any amino acid
DXEXNPGP

SEQ ID NO: 133 - protein, 22 aa, synthetic construct
MLLLVTSLLLCELPHPAFLLIP

SEQ ID NO: 134 - protein, 120 aa, synthetic construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 135 - protein, 107 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 136 - protein, 107 aa, synthetic construct
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 137 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 138 - protein, 245 aa, synthetic construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTSGSGKPGSGEGSTKGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 139 - protein, 42 aa, synthetic construct
KRGRKKLLYIFKQPFMRPVQTTQEEDGCSCRFPEEEEGGCEL

SEQ ID NO: 140 - protein, 94 aa, synthetic construct
NCRNTGPWLKKVLKCNTPDPSKFFSQLSSEHGGDVQKWLSSPFPSSSFSPGGLAPEISPLEVLERDKVTQLLPLNTDAYLSLQELQGQDPTHLV

SEQ ID NO: 141 - protein, 62 aa, synthetic construct
KKVAKKPTNKAPHPKQEPQEINFPDDLPGSNTAAPVQETLHGCQPVTQEDGKESRISVQERQ

SEQ ID NO: 142 - protein, 42 aa, synthetic construct
ALYLLRRDQRLPPDAHKPPGGGSFRTPIQEEQADAHSTLAKI

SEQ ID NO: 143 - protein, 25 aa, synthetic construct
TYCFAPRCRERRRNERLRRESVRPV

SEQ ID NO: 144 - protein, 61 aa, synthetic construct
KWKKKKRPRNSYKCGTNTMEREESEQTKKREKIHIPERSDEAQRVFKSSKTSSCDKSDTCF

SEQ ID NO: 145 - protein, 48 aa, synthetic construct
QRRKYRSNKGESPVEPAEPCHYSCPREEEGSTIPIQEDYRKPEPACSP

SEQ ID NO: 146 - protein, 38 aa, synthetic construct
CWLTKKKYSSSVHDPNGEYMFMRAVNTAKKSRLTDVTL

SEQ ID NO: 147 - protein, 51 aa, synthetic construct
MGWIRGRRSRHSWEMSEFHNYNLDLKKSDFSTRWQKQRCPVVKSKCRENAS

SEQ ID NO: 148 - protein, 24 aa, synthetic construct
LCARPRRSPAQEDGKVYINMPGRG

SEQ ID NO: 149 - protein, 52 aa, synthetic construct
YFLGRLVPRGRGAAEAATRKQRITETESPYQELQGQRSDVYSDLNTQRPYYK

SEQ ID NO: 150 - protein, 120 aa, synthetic construct
WRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 151 - protein, 41 aa, synthetic construct
RSKRSRLLHSDYMNMTPRRPGPTRKHYQPYAPPRDFAAYRS

SEQ ID NO: 152 - protein, 47 aa, synthetic construct
TTTPAPRPPTPAPTIASQPLSLRPEACRPAAGGAVHTRGLDFACDIY

SEQ ID NO: 153 - protein, 39 aa, synthetic construct
IEVMYPPPYLDNEKSNGTIIHVKGKHLCPSPLFPGPSKP

SEQ ID NO: 154 - protein, 21 aa, synthetic construct
IYIWAPLAGTCGVLLLSLVIT

SEQ ID NO: 155 - protein, 27 aa, synthetic construct
FWVLVVVGGVLACYSLLVTVAFIIFWV

SEQ ID NO: 156 - protein, 22 aa, Cypovirus sp.
  source 1..22: note = Cytoplasmic polyhedrosis
DVFRSNYDLLKLCGDIESNPGP

SEQ ID NO: 157 - protein, 22 aa, unidentified
  source 1..22: note = Flacherie Virus
TLTRAKIEDELIRAGIESNPGP

SEQ ID NO: 158 - protein, 36 aa, synthetic construct
FWVLVVVGGVLACYSLLVTVAFIIFWVRSKRSRLLH

SEQ ID NO: 159 - protein, 24 aa, synthetic construct
IYIWAPLAGTCGVLLLSLVITLYC

SEQ ID NO: 160 - protein, 21 aa, synthetic construct
LCYLLDGILFIYGVILTALFL

SEQ ID NO: 161 - protein, 24 aa, synthetic construct
VGIIAGLVLLGSVVSGAVVAAVIW

SEQ ID NO: 162 - protein, 24 aa, synthetic construct
MGIVAGLVVLAAVVTGAAVAAVLW

SEQ ID NO: 163 - protein, 23 aa, synthetic construct
IATGMVGALLLLLVVALGIGLFM

SEQ ID NO: 164 - protein, 17 aa, synthetic construct
GGGGSGGGGSGGGGSGS

SEQ ID NO: 165 - protein, 997 aa, synthetic construct
MVVMAPRTLFLLLSGALTLTETWAVMAPRTLILGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSLGSGATNFSLLKQAGDVEENPGPMVVMAPRTLFLLLSGALTLTETWARIIPRHLQLGGGGSGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 166 - DNA, 2991 bp, synthetic construct
  source 1..2991: mol_type = other DNA
atggtggtcatggcccctagaacactgttcctgctgctgtctggcgccctgacactgacagaaacatgggccgtgatggccccacggacactgattcttggaggcggaggatctggcggcggaggctctattcagagaacccctaagatccaggtgtacagcagacaccccgccgagaacggcaagagcaacttcctgaactgctacgtgtccggctttcaccccagcgacattgaggtggacctgctgaagaacggcgagcggatcgagaaggtggaacacagcgatctgagcttcagcaaggactggtccttctacctgctgtactacaccgagttcacccctaccgagaaggacgagtacgcctgcagagtgaaccacgtgaccctgagccagcctaagatcgtgaagtgggacagagatatgggcggaggtggaagtggcggaggcggtagcggtggtggtggatctggatctcacagcctgaagtactttcacacctccgtgtccagacctggcagaggcgagcctagattcatcagcgtgggctacgtggacgacacccagttcgtcagattcgacaacgacgccgcctctcctcggatggttcctagagcaccctggatggaacaagagggcagcgagtactgggatcgcgagacaagaagcgccagagacacagcccagatcttccgcgtgaacctgagaaccctgcggggctactacaatcagtctgaggccggctctcacaccctgcagtggatgcatggatgtgaactgggccccgacggcagattcctgagaggctatgagcagttcgcctacgacggcaaggactatctgaccctgaacgaggacctgagaagctggaccgccgtggatacagccgctcagatcagcgagcagaagtctaacgacgccagcgaggccgaacaccagagagcctatctggaagatacctgcgtggaatggctgcacaagtacctggaaaagggcaaagagacactgctgcacctggaacctccaaagacacacgtgacacatcaccccatcagcgaccacgaggccacactgagatgttgggccctgggcttttaccctgccgagatcacactgacatggcagcaggatggcgagggccacacacaggatacagagctggtggaaacaagacctgccggcgacggcaccttccagaaatgggctgctgtggtggttcccagcggcgaggaacagagatacacctgtcacgtgcagcacgagggactgcctgaacctgtgactctgagatggaagcctgccagccagccaacaatccccatcgtgggaatcattgccggcctggtgctgctgggatctgtggtttctggtgcagtggtggccgccgtgatttggagaaagaagtcctctggcggcaaaggcggcagctactctaaagccgagtggagcgattctgcccagggctctgaatctcactctctcggaagcggcgccaccaacttcagcctgcttaaacaggcaggcgacgtggaagagaatcccggacctatggtcgtgatggctcctaggactttgtttctgctgctcagcggcgctctgactctgaccgagacttgggctagaatcatccccagacatctgcagctcggaggtggcggaagcggaggcggtggttctggcggaggcggatcaggtggcggaggaagtatccagaggacacccaaaattcaggtgtactctcggcacccagccgaaaatggcaagtccaattttctcaattgctacgtcagcgggtttcacccttccgatatcgaagtcgatctgctcaagaatggggagcgcattgagaaagtcgagcacagcgacctgtcattctccaaggattggagcttttacctcctctactatacggaattcacgcccacagagaaggatgaatatgcctgccgggtcaaccatgtgacactgtctcagcccaagattgtcaaatgggatcgtgacatgggaggcggaggtagcggaggcggcggtagtggtggcggaggcagcggatctcactccatgagatacttctctgccgccgtgtctagaccaggacggggagagcctcggtttatcgccatgggctatgtggatgatacgcaatttgttcgcttcgacagcgacagcgcctgtcctagaatggaacccagagcaccttgggtcgagcaagaaggccccgagtattgggaagaagagacacggaacaccaaggctcacgcccagaccgacagaatgaatctgcagacactgcgcggctattacaaccagagcgaagccagcagccatacactccagtggatgatcggctgcgatctgggatccgatggcagactgctgaggggatacgaacagtacgcctatgacgggaaagattacctggcactgaatgaggatctgcggtcctggacagccgccgatactgctgcccagatcagcaagagaaagtgcgaggccgccaatgtggccgagcagagaagggcttatctcgagggcacatgcgtcgagtggctgcatcggtatctggaaaacggaaaagaaatgctgcagcgggccgatccacctaaaacacatgtgacccaccatcctgtgttcgactacgaggctacccttcggtgttgggctctcggcttctatcccgctgagatcatcctgacttggcaacgcgacggcgaagatcagacccaggacgtggaactggtcgagactagaccagctggcgacgggacatttcaaaagtgggccgcagtggtcgtgccctccggcgaagaacaacggtacacatgccatgtgcagcatgaaggcctgccagagcctctgatgctcaggtggaagcagagcagcctgcctaccattcctatcatgggcatcgtggccggactggttgtgctggctgctgttgttacaggcgcagctgttgctgctgtgctgtggcggaaaaagagcagcgat

SEQ ID NO: 167 - DNA, 3021 bp, synthetic construct
  source 1..3021: mol_type = other DNA
atggtggtcatggcccctagaacactgttcctgctgctgtctggcgccctgacactgacagaaacatgggccgtgatggccccacggacactgattcttggaggcggaggatctggcggaggtggaagtggcggaggcggatctattcagcggacccctaagatccaggtgtacagcagacaccccgccgagaacggcaagagcaacttcctgaactgctacgtgtccggctttcaccccagcgacattgaggtggacctgctgaagaacggcgagcggatcgagaaggtggaacacagcgatctgagcttcagcaaggactggtccttctacctgctgtactacaccgagttcacccctaccgagaaggacgagtacgcctgcagagtgaaccacgtgaccctgagccagcctaagatcgtgaagtgggacagagatatgggaggcggcggtagtggcggcggaggaagcggaggcggaggttcaggtggtggtggatctggaagccacagcctgaagtactttcacacctccgtgtccagacctggcagaggcgagcctagattcatcagcgtgggctacgtggacgacacccagttcgtcagattcgacaacgacgccgcctctcctcggatggttcctagagcaccctggatggaacaagagggcagcgagtactgggatcgcgagacaagaagcgccagagacacagcccagatcttccgcgtgaacctgagaaccctgcggggctactacaatcagtctgaggccggctctcacaccctgcagtggatgcatggatgtgaactgggccccgacggcagattcctgagaggctatgagcagttcgcctacgacggcaaggactatctgaccctgaacgaggacctgagaagctggaccgccgtggatacagccgctcagatcagcgagcagaagtctaacgacgccagcgaggccgaacaccagagagcctatctggaagatacctgcgtggaatggctgcacaagtacctggaaaagggcaaagagacactgctgcacctggaaccacctaagacacacgtgacacatcaccccatcagcgaccacgaggccacactgagatgttgggccctgggcttttaccctgccgagatcacactgacatggcagcaggatggcgagggccacacacaggatacagagctggtggaaacaagacctgccggcgacggcaccttccagaaatgggctgctgtggtggttcccagcggcgaggaacagagatacacctgtcacgtgcagcacgagggactgcctgaacctgtgactctgagatggaagcctgccagccagccaacaatccccatcgtgggaatcattgccggcctggtgctgctgggatctgtggtttctggtgcagtggtggccgccgtgatttggagaaagaagtcctctggcggcaaaggcggcagctactctaaagccgagtggagcgattctgcccagggctctgaatctcactctcttggcagcggcgccaccaacttcagcctgcttaaacaggcaggcgacgtggaagagaaccccggacctatggttgtgatggctcctaggactttgtttctgctgctcagcggcgctctgactctgaccgagacttgggctagaatcatccccagacatctgcagcttggaggtggtggtagcggtggtggcggttcaggtggcggaggttctatccagagaaccccaaaaattcaggtgtactctcggcacccagccgaaaatggcaagtccaattttctcaattgctacgtcagcgggtttcacccttccgatatcgaagtcgatctgctcaagaatggggagcgcattgagaaagtcgagcacagcgacctgtcattctccaaggattggagcttttacctcctctactatacggaattcacgcccacagagaaggatgaatatgcctgccgggtcaaccatgtgacactgtctcagcccaagattgtcaaatgggatcgagacatgggcggtggcggcagcggaggtggtggctcaggcggaggcggttccggcggaggcggctctggatctcattccatgagatacttctctgccgccgtgtctagaccaggacggggagagcctcggtttatcgccatgggctatgtggatgatacgcaatttgttcgcttcgacagcgacagcgcctgtcctagaatggaacccagagcaccttgggtcgagcaagaaggccccgagtattgggaagaagagacacggaacaccaaggctcacgcccagaccgacagaatgaatctgcagacactgcgcggctattacaaccagagcgaagccagcagccatacactccagtggatgatcggctgcgatctgggatccgatggcagactgctgaggggatacgaacagtacgcctatgacgggaaagattacctggcactgaatgaggatctgcggtcctggacagccgccgatactgctgcccagatcagcaagagaaagtgcgaggccgccaatgtggccgagcagagaagggcttatctcgagggcacatgcgtcgagtggctgcatcggtatctggaaaacggaaaagaaatgctgcagcgggccgatcctccaaaaacacatgtgacccaccatcctgtgttcgactacgaggctacccttcggtgttgggctctcggattctaccccgctgagatcatcctcacttggcaacgcgacggcgaagatcagacccaggacgtggaactggtcgagactagaccagctggcgacgggacatttcaaaagtgggccgcagtggtcgtgccctccggcgaagaacaacggtacacatgccatgtgcagcatgaaggcctgccagagcctctgatgctcaggtggaagcagagcagcctgcctaccattcctatcatgggcatcgtggccggactggtggttctggctgctgttgttacaggcgcagctgtggctgcagtgctgtggcggaagaagtccagcgat

SEQ ID NO: 168 - protein, 1007 aa, synthetic construct
MVVMAPRTLFLLLSGALTLTETWAVMAPRTLILGGGGSGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSLGSGATNFSLLKQAGDVEENPGPMVVMAPRTLFLLLSGALTLTETWARIIPRHLQLGGGGSGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 169 - DNA, 3021 bp, synthetic construct
  source 1..3021: mol_type = other DNA
atggtggtcatggcccctagaacactgttcctgctgctgtctggcgccctgacactgacagaaacatgggccgtgatggccccacggacactgattcttggaggcggaggatctggcggaggtggaagtggcggaggcggttctggtggtggtggatccattcagcggacccctaagatccaggtgtacagcagacaccccgccgagaacggcaagagcaacttcctgaactgctacgtgtccggctttcaccccagcgacattgaggtggacctgctgaagaacggcgagcggatcgagaaggtggaacacagcgatctgagcttcagcaaggactggtccttctacctgctgtactacaccgagttcacccctaccgagaaggacgagtacgcctgcagagtgaaccacgtgaccctgagccagcctaagatcgtgaagtgggacagagatatgggaggcggcggtagtggtggcggcggttcaggcggcggaggaagtggatctcacagcctgaagtactttcacacctccgtgtccagacctggcagaggcgagcctagattcatcagcgtgggctacgtggacgacacccagttcgtcagattcgacaacgacgccgcctctcctcggatggttcctagagcaccctggatggaacaagagggcagcgagtactgggatcgcgagacaagaagcgccagagacacagcccagatcttccgcgtgaacctgagaaccctgcggggctactacaatcagtctgaggccggctctcacaccctgcagtggatgcatggatgtgaactgggccccgacggcagattcctgagaggctatgagcagttcgcctacgacggcaaggactatctgaccctgaacgaggacctgagaagctggaccgccgtggatacagccgctcagatcagcgagcagaagtctaacgacgccagcgaggccgaacaccagagagcctatctggaagatacctgcgtggaatggctgcacaagtacctggaaaagggcaaagagacactgctgcacctggaacctccaaagacacacgtgacacatcaccccatcagcgaccacgaggccacactgagatgttgggccctgggcttttaccctgccgagatcacactgacatggcagcaggatggcgagggccacacacaggatacagagctggtggaaacaagacctgccggcgacggcaccttccagaaatgggctgctgtggtggttcccagcggcgaggaacagagatacacctgtcacgtgcagcacgagggactgcctgaacctgtgactctgagatggaagcctgccagccagccaacaatccccatcgtgggaatcattgccggcctggtgctgctgggatctgtggtttctggtgcagtggtggccgccgtgatttggagaaagaagtcctctggcggcaaaggcggcagctactctaaagccgagtggagcgattctgcccagggctctgaatctcactctctcggaagcggcgccaccaacttcagcctgcttaaacaggcaggcgacgtggaagagaaccccggacctatggttgtgatggctcctaggactttgtttctgctgctcagcggcgctctgactctgaccgagacttgggctagaatcatccccagacatctgcagcttggcggtggtggctcaggcggtggcggaagcggaggtggcggcagtggcggcggtggatctattcagagaacacccaaaattcaggtgtactctcggcacccagccgaaaatggcaagtccaattttctcaattgctacgtcagcgggtttcacccttccgatatcgaagtcgatctgctcaagaatggggagcgcattgagaaagtcgagcacagcgacctgtcattctccaaggattggagcttttacctcctctactatacggaattcacgcccacagagaaggatgaatatgcctgccgggtcaaccatgtgacactgtctcagcccaagattgtcaaatgggatcgagacatgggcggaggcggatcaggcggaggcggctctggcggcggaggttctggatctcatagcatgcggtacttctctgccgccgtgtctagacccggaaggggagagcctcggtttatcgccatgggctatgtggatgatacgcaatttgttcgcttcgacagcgacagcgcctgtcctagaatggaacccagagcaccttgggtcgagcaagaaggccccgagtattgggaagaagagacacggaacaccaaggctcacgcccagaccgacagaatgaatctgcagacactgcgcggctattacaaccagagcgaagccagcagccatacactccagtggatgatcggctgcgatctgggatccgatggcagactgctgaggggatacgaacagtacgcctatgacgggaaagattacctggcactgaatgaggatctgcggtcctggacagccgccgatactgctgcccagatcagcaagagaaagtgcgaggccgccaatgtggccgagcagagaagggcttatctcgagggcacatgcgtcgagtggctgcatcggtatctggaaaacggaaaagaaatgctgcagcgggccgatccacctaaaacacatgtgacccaccatcctgtgttcgactacgaggctacccttcggtgttgggctctcggcttctatcccgctgagatcatcctgacttggcaacgcgacggcgaagatcagacccaggacgtggaactggtcgagactagaccagctggcgacgggacatttcaaaagtgggccgcagtggtcgtgccctccggcgaagaacaacggtacacatgccatgtgcagcatgaaggcctgccagagcctctgatgctcaggtggaagcagagcagcctgcctaccattcctatcatgggcatcgtggccggactggtggttctggctgctgttgttacaggcgcagctgtggctgcagtcctgtggcggaaaaagagcagcgat